(19) United States
(12) Patent Application Publication
(10) Pub. No.: US 2026/0109997 A1
(43) Pub. Date: Apr. 23, 2026

(54) BDNF GENE THERAPY

(71) Applicant: MeiraGTx UK II Limited, London (GB)

(72) Inventors: Alexandria Forbes, New York, NY (US); Josefa Sullivan, New York, NY (US); Ce Feng Liu, New York, NY (US); Matthew During, Rowayton, CT (US); Natalia Fabela, New York, NY (US)

(21) Appl. No.: 19/117,728

(22) PCT Filed: Oct. 11, 2023
(86) PCT No.: PCT/IB2023/060243

§ 371 Date: Apr. 2, 2025

Related U.S. Application Data
(60) Provisional application No. 63/379,114, filed on Oct. 11, 2022.

(51) Int. Cl. C12N 15/86; A61K 38/18; A61K 48/00; A61P 3/04; C07K 14/475

(52) U.S. Cl. CPC C12N 15/86; A61K 38/185; A61K 48/005; A61P 3/04; C07K 14/475; C12N 2750/14143; C12N 2830/48; C12N 2830/50

(57) ABSTRACT

This disclosure provides BDNF expression constructs and vectors comprising such constructs for treating metabolic disorders, including, but not limited to, obesity. The disclosed BDNF gene therapy is more effective than existing BDNF-based gene therapy constructs.

### CROSS-REFERENCE TO RELATED APPLICATIONS

This application claims the benefit under 35 U.S.C. § 119 (e) of the earlier filing date of U.S. Provisional Patent No. 63/379,114, filed Oct. 11, 2022, which is hereby incorporated by reference in its entirety.

### REFERENCE TO AN ELECTRONIC SEQUENCE LISTING

The contents of the electronic sequence listing (SeqList-162027-53276.xml; Size: 216,128 bytes; and Date of Creation: Oct. 10, 2023) is herein incorporated by reference in its entirety.

### FIELD

The present disclosure relates generally to compositions and methods for gene therapy for treating metabolic disorders and diseases.

### BACKGROUND

Obesity is most commonly caused by excessive food intake coupled with limited energy expenditure and/or lack of physical exercise. Obesity increases the likelihood of developing various diseases, such as diabetes mellitus, hypertension, atherosclerosis, coronary artery disease, sleep apnea, gout, rheumatism, and arthritis. Moreover, mortality risk directly correlates with obesity, such that, for example, a body-mass index in excess of 40 results in an average decreased life expectancy of more than ten years.

Current pharmacological treatment modalities include appetite suppressors targeting receptor classes (e.g., CB 1, 5-HT2c, and NPY); regulators of the appetite circuits in the hypothalamus and the molecular actions of ghrelin; and nutrient-absorption inhibitors targeting lipases. However, none of the current modalities has been shown to effectively treat obesity without causing adverse effects, some of which can be very severe. The incidence of bariatric surgery has increased dramatically, reflecting just how problematic and refractory severe obesity is to alternative, less invasive strategies. However, bariatric surgery is often effective, but comes with significant morbidity in its own right. Pharmacological therapies such as semaglutide (Wilding J P H, et al. N Engl J Med. 2021 Mar. 18; 384 (11): 989-1002) and tirzepatide (Ania M. Jastreboff et al., N Engl J Med 2022; 387:205-216) require patient adherence to regimen and can cause inflammation at injection sites. Further, these therapies can take up to a year for the drugs to reach their full efficacy (e.g., 15-20% weight loss which is less than bariatric surgery).

Accordingly, there is an urgent need for a safer and more effective approaches for treating obesity.

### SUMMARY OF THE DISCLOSURE

This disclosure addresses the need mentioned above in a number of aspects. In one aspect, this disclosure provides an expression construct for the treatment of metabolic disorders and diseases. In some embodiments, the expression construct comprises a promoter sequence operatively linked to a nucleic acid sequence encoding a brain derived neurotrophic factor (BDNF) or variant thereof.

In some embodiments, the promoter sequence comprises a nucleic acid sequence having at least 90% sequence identity to a nucleic acid sequence selected from SEQ ID NOs: 1-10 or comprises a nucleic acid sequence selected from SEQ ID NOs: 1-10. In some embodiments, the promoter sequence comprises a nucleic acid sequence having at least 90% sequence identity to the nucleic acid sequence of SEQ ID NO: 1 or comprises the nucleic acid sequence of SEQ ID NO: 1.

In some embodiments, the promoter sequence comprises a constitutive promoter.

In some embodiments, the nucleic acid sequence encoding the BDNF is a wild type BDNF gene or coding sequence. In some embodiments, the nucleic acid sequence encoding the BDNF is a human BDNF gene or coding sequence. In some embodiments, the nucleic acid sequence encoding the BDNF is a codon-optimized coding sequence.

In some embodiments, the nucleic acid sequence encoding the BDNF comprises a nucleic acid sequence having at least 90% sequence identity to a nucleic acid sequence selected from SEQ ID NOs: 52-57 or 70-79 or comprises a nucleic acid sequence selected from SEQ ID NOs: 52-57 or 70-79.

In some embodiments, the nucleic acid sequence encoding the BDNF comprises a nucleic acid sequence having at least 90% sequence identity to the nucleic acid sequence of SEQ ID NO: 52 or 53 or comprises the nucleic acid sequence of SEQ ID NO: 52 or 53.

In some embodiments, the nucleic acid sequence encoding the BDNF encodes a protein comprising a sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 81 (human wild type BDNF).

In some embodiments, the expression construct further comprises a post-transcriptional regulatory element. In some embodiments, the post-transcriptional regulatory element comprises a woodchuck hepatitis virus post-transcriptional regulatory element (WPRE).

In some embodiments, the post-transcriptional regulatory element comprises a nucleic acid sequence having at least 90% sequence identity to a nucleic acid sequence selected from SEQ ID NOs: 58-61 or comprises a nucleic acid sequence selected from SEQ ID NOs: 58-61.

In some embodiments, the post-transcriptional regulatory element comprises a nucleic acid sequence having at least 90% sequence identity to the nucleic acid sequence of SEQ ID NO: 58 or 59 or comprises the nucleic acid sequence of SEQ ID NO: 58 or 59.

In some embodiments, the expression construct further comprises a polyadenylation signal. In some embodiments, the polyadenylation signal comprises a nucleic acid sequence having at least 90% sequence identity to the nucleic acid sequence of SEQ ID NO: 62 or comprises the nucleic acid sequence of SEQ ID NO: 62 or 66-69.

In some embodiments, the expression construct further comprises a microRNA (miR) binding sequence (miRBS) positioned in the 3′ UTR, for example, between the nucleic acid sequence encoding the BDNF and the post-transcriptional regulatory element. In some embodiments, the microRNA binding sequence comprises a nucleic acid sequence having at least 90% sequence identity to any one of (i) SEQ ID NO: 63, (ii) SEQ ID NO: 64, or (iii) SEQ ID NOs: 63 and 82. In some embodiments, the microRNA binding sequence comprises any one of (i) SEQ ID NO: 63, (ii) SEQ ID NO: 64, or (iii) SEQ ID NOs: 63 and 82.

In some embodiments, the expression construct further comprises a terminator downstream of the post-transcriptional regulatory element. In some embodiments, the expression construct comprises:
(a) a promoter sequence comprising SEQ ID NO: 1;
(b) a nucleic acid sequence encoding BDNF comprising SEQ ID NO: 52;
(c) a post-transcriptional regulatory element comprising SEQ ID NO: 58;
(d) a polyadenylation signal comprising SEQ ID NO: 62; and
(e) a microRNA binding sequence comprising SEQ ID NO: 63.

In some embodiments, the expression construct comprises:
(a) a promoter sequence comprising SEQ ID NO: 1;
(b) a nucleic acid sequence encoding BDNF comprising SEQ ID NO: 53;
(c) a post-transcriptional regulatory element comprising SEQ ID NO: 59;
(d) a polyadenylation signal comprising SEQ ID NO: 62; and
(e) a microRNA binding sequence comprising (i) SEQ ID NO: 64, or (ii) SEQ ID NOs: 63 and 82.

In another aspect, this disclosure also provides a vector comprising the expression construct as described herein. In some embodiments, the vector is a viral vector. In some embodiments, the vector is an adeno-associated virus (AAV) vector. In some embodiments, the viral vector comprises the expression construct as described herein and one or more inverted terminal repeats (ITRs). In some embodiments, the vector comprises a genome derived from AAV serotype AAV2. In some embodiments, the vector comprises a capsid derived from AAV serotype AAV1. In some embodiments, the vector comprises a nucleic acid sequence having at least 90% sequence identity to a nucleic acid sequence selected from SEQ ID NOs: 11-50 or comprises a nucleic acid sequence selected from SEQ ID NOs: 11-50. In some embodiments, the vector comprises a nucleic acid sequence having at least 90% sequence identity to a nucleic acid sequence selected from SEQ ID NOs: 11-50 or comprises least 90% sequence identity to a nucleic acid sequence selected from SEQ ID NOs: 11, 18-22, 27-30, and 42-45. In some embodiments, the vector comprises a nucleic acid sequence selected from SEQ ID NOs: 11, 18-22, 27-30, and 42-45. In some embodiments, the vector comprises a nucleic acid sequence having at least 90% sequence identity to SEQ ID NO: 11 or SEQ ID NO: 22 or the nucleic acid sequence comprises SEQ ID NO: 11 or SEQ ID NO: 22.

Provided herein is a pharmaceutical composition comprising the vector described herein and a pharmaceutically acceptable carrier.

Provided herein is a method for treating or preventing a metabolic disorder in a subject in need thereof. In some embodiments, the method comprises administering to the subject in need thereof a vector or a pharmaceutical composition described herein. In one embodiment, the metabolic disorder is obesity.

Provided herein is a method of increasing a level of BDNF in a subject in need thereof. The method comprises administering to the subject in need thereof a vector or a pharmaceutical composition described herein. In some embodiments, the vector or the pharmaceutical composition is administered by stereotaxic microinjection.

### BRIEF DESCRIPTION OF THE DRAWINGS

FIGS. 1A, 1B, and 1C are a set of diagrams showing higher BDNF expression from an optimized constitutive promoter than tissue-specific or original CAG promoters. FIG. 1A shows FACS-based promoter screening of optimized tissue-specific and constitutive promoters operatively linked to a fluorescent protein reporter (mClover3) identified preferred promoter candidates for driving BDNF expression. FIG. 1B shows extracellular BDNF expression by transiently transfected mouse N2A (left) and human Be2M17 cells (right). FIG. 1C shows extracellular BDNF expression by transiently transfected mouse N2A cells. OSU: reference construct, see Example 2.

FIGS. 2A, 2B, 2C, and 2D show that BDNF expression constructs disclosed herein are more potent than a reference construct in vitro. FIG. 2A shows extracellular BDNF expression by transiently transfected mouse N2A (left) and human SH-SYFY cells (right). FIG. 2B shows BDNF expression in transiently transfected human neural progenitor cells. FIG. 2C shows the results of six independent experiments depicting that expression construct BDNF-1 expresses 4-fold more BDNF than reference construct OSU in N2A cells. FIG. 2D shows that codon-optimization of the BDNF gene does not impair BDNF signaling as indicated by the induction of phospho-ERK1/2 in primary neurons after 30 minutes of treatment. C.O.1=codon-optimized gene sequence #1 (BDNFco CDS); C.O.2=codon-optimized gene sequence #2 (BDNFco2 CDS); C.O.3=codon-optimized gene sequence #3 (BDNFco3 CDS); C.O.2=codon-optimized gene sequence #4 (BDNFco4 CDS).

FIGS. 3A, 3B, and 3C show superior BDNF expression from selected expression constructs in transduced primary neurons. FIG. 3A shows that primary cortical mouse neurons were transduced with AAV2/1 at 10,000 or 50,000 multiplicity of infection (MOI). Media was collected 4 and 7 days post infection (d.p.i) and prepared for western blot. DNA was isolated from neuronal lysates at 7 d.p.i. FIG. 3B shows that neuronal transduction was equivalent across groups as quantified by qPCR for viral genomes using probes for either the transgene or the ITR (Inverted Terminal Repeat) sequences. FIG. 3C shows that transduction with construct BDNFI and construct BDNF12 in vitro leads to significantly higher BDNF expression in primary cortical neurons.

FIGS. 4A, 4B, 4C, and 4D illustrate that over-expression of BDNF from selected expression constructs causes weight loss in a diet-induced obesity mouse model. FIG. 4A shows that AAV2/1 (2E9 GC/hemisphere) was bilaterally injected into the hypothalamus of wild-type C57B16 male mice at 8 weeks of age and weighed weekly. After 7 days, animals were placed on 45% high-fat or matched control diet. After 21 days, the hypothalamus was dissected for protein analysis. FIG. 4B shows a Western blot depicting high expression of BDNF expression constructs in vivo. FIG. 4C shows expression of pro- and mBDNF in the hypothalamus as quantified by ELISA. FIG. 4D shows weight change after AAV2/1 delivery in the diet-induced obesity mouse model. *** p<0.001; **** p<0.0001. One-Way ANOVA repeated measures. Endpoints from top to bottom: HFD, AAV1-eGFP; control diet; AAV1-OSU; AAV-BDNF-12; AAV-BDNF-1.

### DETAILED DESCRIPTION

This disclosure provides BDNF gene therapy expression constructs and vectors for treating metabolic disorders, such as obesity. The disclosed BDNF gene therapy is more effective than the existing BDNF-based gene therapy constructs.

### BDNF Expression Constructs

In one aspect, this disclosure provides an expression construct for treating metabolic disorders and diseases, including obesity.

In some embodiments, the expression construct comprises a promoter sequence operatively linked to a nucleic acid sequence encoding a brain derived neurotrophic factor (BDNF) or variant thereof. As used herein, “operatively linked” refers to a first molecule joined to a second molecule, wherein the molecules are arranged such that the first molecule affects the function of the second molecule. The two molecules may or may not be part of a single contiguous molecule and may or may not be adjacent. For example, a promoter is operatively linked to a transcribable polynucleotide molecule if the promoter modulates transcription of the transcribable polynucleotide molecule of interest in a cell. Additionally, two portions of a transcription regulatory element are operatively linked to one another if they are joined such that the transcription-activating functionality of one portion is not adversely affected by the presence of the other portion. Two transcription regulatory elements may be operatively linked to one another by way of a linker nucleic acid (e.g., an intervening non-coding nucleic acid) or may be operatively linked d to one another with no intervening nucleotides present.

BDNF is a key molecular regulator of metabolic function and body weight. Monogenic mutations in brain-derived neurotrophic factor, BDNF, or its receptor, NTRK2, cause obesity in humans. BDNF signaling is also downstream of the leptin-melanocortin pathway, which controls appetite and body weight. The expression constructs and vectors described herein may encode a BDNF variant. As used herein, a variant is a protein that comprises one or more alterations when compared to the parental protein, including, but not limited to amino acid additions, substitutions, insertions, deletions, or posttranslational modifications, wherein the variant retains at least 10%, at least 20%, at least 30% at least 40% at least 50% at least 60%, at least 70% at least 80%, or at least 90% of the biological activity of the parental protein. In embodiments, a BDNF variant disclosed herein retains at least 10%, at least 20%, at least 30% at least 40% at least 50% at least 60%, at least 70% at least 80%, or at least 90% of BDNF's ability to activate signaling cascades.

In some embodiments, the nucleic acid sequence encoding the BDNF is a coding sequence from a wild type BDNF gene. In some embodiments, the nucleic acid sequence encoding the BDNF is a coding sequence from a human BDNF (e.g., human wild type BDNF) gene. In some embodiments, the nucleic acid sequence encoding the BDNF is a codon-optimized sequence. In some embodiments, the nucleic acid sequence encoding the BDNF encodes a protein comprising a sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 81 (human wild type BDNF).

SEQ ID NO: 81:
MTILFLTMVISYFGCMKAAPMKEANIRGQGGLAYPGVRTHGTLESVNGP
KAGSRGLTSLADTFEHVIEELLDEDQKVRPNEENNKDADLYTSRVMLSS
QVPLEPPLLFLLEEYKNYLDAANMSMRVRRHSDPARRGELSVCDSISEW
VTAADKKTAVDMSGGTVTVLEKVPVSKGQLKQYFYETKCNPMGYTKEGC
RGIDKRHWNSQCRTTQSYVRALTMDSKKRIGWRFIRIDTSCVCTLTIKR
GR

In some embodiments, the nucleic acid sequence encoding the BDNF has at least 80%, at least 85%, at least 90%, at least 91%, at least 92%, at least 93%, at least 94%, at least 95%, at least 96%, at least 97%, at least 98%, or at least 99% sequence identity to a nucleic acid sequence selected from SEQ ID NOs: 52-57 or 70-79. In some embodiments, the nucleic acid sequence encoding the BDNF is encoded by a nucleic acid sequence that comprises a nucleic acid sequence selected from SEQ ID NOs: 52-57 or 70-79.

In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 52. In some embodiments, the nucleic acid sequence encoding the BDNF comprises the nucleic acid sequence of SEQ ID NO: 52.

In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53. In some embodiments, the nucleic acid sequence encoding the BDNF comprises the nucleic acid sequence of SEQ ID NO: 53.

In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 54. In some embodiments, the nucleic acid sequence encoding the BDNF comprises the nucleic acid sequence of SEQ ID NO: 54.

In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 55. In some embodiments, the nucleic acid sequence encoding the BDNF comprises the nucleic acid sequence of SEQ ID NO: 55.

In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56. In some embodiments, the nucleic acid sequence encoding the BDNF comprises the nucleic acid sequence of SEQ ID NO: 56.

In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 57. In some embodiments, the nucleic acid sequence encoding the BDNF comprises the nucleic acid sequence of SEQ ID NO: 57.

A great number of expression control sequences, e.g., native, constitutive, inducible and/or tissue-specific, are known in the art and may be utilized to drive expression of the BDNF transgene, depending upon the type of expression desired. For eukaryotic cells, expression control sequences typically include a promoter, an enhancer, and a polyadenylation sequence which may include splice donor and acceptor sites. The polyadenylation sequence generally is inserted downstream of the sequence encoding BDNF and upstream of the 3′ ITR sequence.

Another regulatory component of the rAAV useful in the methods disclosed herein is an internal ribosome entry site (IRES). An IRES sequence may be used to produce more than one polypeptide from a single gene transcript. An IRES (or other suitable sequence) is used to produce a protein that contains more than one polypeptide chain or to express two different proteins from or within the same cell. In some embodiments, the IRES is located 3′ of the sequence encoding BDNF in the rAAV vector.

As used herein, the term “promoter” or “regulatory sequence” refers to a nucleic acid sequence that is required for expression of a gene product operatively linked to the promoter/regulatory sequence. In some instances, this sequence may be the core promoter sequence, and in other instances, this sequence may also include an enhancer sequence and other regulatory elements that are required for expression of the gene product. The promoter or regulatory sequence may, for example, be one that expresses the gene product in a tissue-specific manner. An “inducible” promoter is a nucleotide sequence that, when operatively linked with a polynucleotide that encodes or specifies a gene product, causes the gene product to be produced in a cell substantially only when an inducer that corresponds to the promoter is present in the cell. The term “enhancer” as used herein refers a cis-acting regulatory sequence (e.g., 50-1,500 base pairs) that bind one or more proteins (e.g., activator proteins or transcription factors) to increase transcriptional activation of a nucleic acid sequence. Enhancers can be positioned up to 1,000,000 base pairs upstream of the gene start site or downstream of the gene start site that they regulate. An enhancer can be positioned within an intronic region or in the exonic region of an unrelated gene.

In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 1-10. In some embodiments, the promoter sequence comprises a sequence selected from SEQ ID NOs: 1-10. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid of SEQ ID NO: 1. In some embodiments, the promoter sequence to a nucleic acid comprises the nucleic acid of SEQ ID NO: 1.

In some embodiments, the promoter sequence comprises a constitutive promoter sequence. “Constitutive promoter” refers to a promoter that is capable of facilitating continuous transcription of a coding sequence or gene under its control and/or to which it is operatively linked.

In some embodiments, the expression construct comprises a post-transcriptional regulatory element. In some embodiments, the expression construct comprises a woodchuck hepatitis virus post-transcriptional regulatory element (WPRE).

In some embodiments, the post-transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 58-61. In some embodiments, the post-transcriptional regulatory element comprises a nucleic acid sequence selected from SEQ ID NOs: 58-61.

In some embodiments, the post-transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 58 or 59. In some embodiments, the post-transcriptional regulatory element comprises the nucleic acid sequence of SEQ ID NO: 58 or 59.

In some embodiments, the expression construct comprises a polyadenylation signal. In some embodiments, the polyadenylation signal comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to the nucleic acid sequence of SEQ ID NO: 62 or 66-69. In some embodiments, the expression construct comprises the nucleic acid sequence of SEQ ID NO: 62 or 66-69.

In some embodiments, the expression construct further comprises a microRNA (miR) binding sequence (miRBS or mirBS) positioned in the 3′ UTR. In embodiments, the miRBS is positioned between the nucleic acid sequence encoding the BDNF and the post-transcriptional regulatory element. In embodiments, the miRBS comprises binding sequence(s) for microRNAs wherein the microRNA is selected from miR-142, miR-185, miR-1, miR-122, or combinations thereof. In embodiments, the miRBS comprises one or more (e.g., 1, 2, 3 or 4) binding sites for miR-142, one or more (e.g., 1, 2, 3 or 4) binding sites for miR-185, one or more (e.g., 1, 2, 3 or 4) binding sites for miR-1, and one or more (e.g., 1, 2, 3 or 4) binding sites for miR-122.

In some embodiments, the microRNA binding sequence comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to (i) SEQ ID NO: 63, (ii) SEQ ID NO: 64, or (iii) SEQ ID NOs: 63 and 82.

In some embodiments, the microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 63. In some embodiments, the microRNA binding sequence comprises the nucleic acid sequence of SEQ ID NO: 63.

In some embodiments, the microRNA binding sequence comprises (i) a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 63 and (ii) a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO:82. In some embodiments, the microRNA binding sequence comprises SEQ ID NOs: 63 and 82.

In some embodiments, the microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 64. In some embodiments, the microRNA binding sequence comprises the nucleic acid sequence of SEQ ID NO: 64. SEQ ID NO:64 comprises a cloning site (nucleotides 202-209 of SEQ ID NO:64, underlined in Table 7). A person skilled in the art may delete of modify this cloning site (e.g., by replacing one or more nucleotides of nucleotides 202-209 of SEQ ID NO:64 with different nucleotides) without abolishing functionality of the microRNA binding sequence.

In some embodiments, the expression construct further comprises a terminator downstream of the post-transcriptional regulatory element. In some embodiments, the expression construct comprises a nucleic acid comprising one or more inverted terminal repeats (ITR). In some embodiments, the ITR sequences are derived from AAV serotype 2 (AVV2).

In some embodiments, the 5′ ITR sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 51. In some embodiments, the 5′ ITR sequence comprises the nucleic acid sequence of SEQ ID NO: 51.

In some embodiments, the 3′ ITR sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 65. In some embodiments, the 3′ ITR sequence comprises the nucleic acid sequence of SEQ ID NO: 65.

In some embodiments, the expression construct comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to a nucleic acid sequence selected from SEQ ID NOs: 11-50. In some embodiments, the expression construct comprises a nucleic acid sequence selected from SEQ ID NOs: 11-50. In some embodiments, the expression construct comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to a nucleic acid sequence selected from SEQ ID NOs: 11, 18-22, 27-30, and 42-45. In some embodiments, the expression construct comprises a nucleic acid sequence selected from SEQ ID NOs: 11, 18-22, 27-30, and 42-45.

In some embodiments, the expression construct comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to the nucleic acid sequence of SEQ ID NO: 11. In some embodiments, the expression construct comprises the nucleic acid sequence of SEQ ID NO: 11.

In some embodiments, the expression construct comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to the nucleic acid sequence of SEQ ID NO: 22. In some embodiments, the expression construct comprises the nucleic acid sequence of SEQ ID NO: 22.

### Vectors

In one aspect, provided are recombinant vectors and their use for the introduction of a transgene or an expression construct into a cell.

In some embodiments, the recombinant vectors comprise recombinant DNA constructs that include additional DNA elements, including DNA segments that provide for the replication of the DNA in a host cell and expression of the target gene in target cells at appropriate levels. The ordinarily skilled artisan appreciates that expression control sequences (promoters, enhancers, and the like) are selected based on their ability to promote expression of the target gene in the target cell.

“Vector,” as used herein, refers to a vehicle that comprises a polynucleotide to be delivered into a host cell, either in vitro or in vivo. Non-limiting examples of vectors include a recombinant plasmid, yeast artificial chromosome (YAC), mini chromosome, DNA mini-circle, or a virus (including virus-derived sequences). A vector may also refer to a virion comprising a nucleic acid to be delivered into a host cell, either in vitro or in vivo. In some embodiments, a vector refers to a virion comprising a recombinant viral genome, wherein the viral genome comprises one or more ITRs and a transgene.

In some embodiments, the recombinant vector is a viral vector or a combination of multiple viral vectors.

In one aspect, provided is a vector comprising any of the expression constructs disclosed herein.

In some embodiments, the vector comprises an expression construct that comprises a promoter sequence operatively linked to a nucleic acid sequence encoding a brain derived neurotrophic factor (BDNF) or variant/fragment thereof.

In some embodiments, the vector comprises a nucleic acid sequence encoding BDNF, wherein the BDNF-encoding sequence is derived from a wild type BDNF gene. In some embodiments, the vector comprises a nucleic acid sequence encoding BDNF, wherein the BDNF-encoding sequence is derived from a human BDNF (e.g., human wild type BDNF) gene. In some embodiments, the vector comprises a nucleic acid sequence encoding BDNF, wherein the BDNF-encoding sequence is a codon-optimized sequence.

In some embodiments, the vector comprises a BDNF-encoding sequence, wherein the BDNF is encoded by a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to a nucleic acid sequence selected from SEQ ID NOs: 52-57 or 70-79. In some embodiments, the BDNF is encoded by a nucleic acid sequence that comprises a nucleic acid sequence selected from SEQ ID NOs: 52-57 or 70-79. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 52. In some embodiments, the nucleic acid sequence encoding the BDNF comprises the nucleic acid sequence of SEQ ID NO: 52. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53. In some embodiments, the nucleic acid sequence encoding the BDNF comprises the nucleic acid sequence of SEQ ID NO: 53. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 54. In some embodiments, the nucleic acid sequence encoding the BDNF comprises the nucleic acid sequence of SEQ ID NO: 54. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 55. In some embodiments, the nucleic acid sequence encoding the BDNF comprises the nucleic acid sequence of SEQ ID NO: 55. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56. In some embodiments, the nucleic acid sequence encoding the BDNF comprises the nucleic acid sequence of SEQ ID NO: 56. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 57. In some embodiments, the nucleic acid sequence encoding the BDNF comprises the nucleic acid sequence of SEQ ID NO: 57.

In some embodiments, the vector comprises a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 1-10. In some embodiments, the promoter sequence comprises a nucleic acid sequence selected from SEQ ID NOs: 1-10. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid of SEQ ID NO: 1. In some embodiments, the promoter sequence to a nucleic acid comprises the nucleic acid of SEQ ID NO: 1. In some embodiments, the promoter sequence comprises a constitutive promoter.

In some embodiments, the vector comprises a nucleic acid comprising a post-transcriptional regulatory element. In some embodiments, the vector comprises a nucleic acid comprising a WPRE. In some embodiments, the post-transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 58-61. In some embodiments, the post-transcriptional regulatory element comprises a nucleic acid sequence selected from SEQ ID NOs: 58-61. In some embodiments, the post-transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 58 or 59. In some embodiments, the post-transcriptional regulatory element comprises the nucleic acid sequence of SEQ ID NO: 58 or 59.

In some embodiments, the vector comprises a nucleic acid comprising a polyadenylation signal. In some embodiments, the polyadenylation signal is derived from a bovine growth hormone (bGH) polyadenylation signal, a human growth hormone (hGH) polyadenylation signal, or a SV40 polyadenylation signal. In some embodiments, the polyadenylation signal comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62 or 66-69. In some embodiments, the polyadenylation signal comprises the nucleic acid sequence of SEQ ID NO: 62 or 66-69.

In some embodiments, the vector comprises a microRNA binding sequence positioned between the nucleic acid sequence encoding the BDNF and the post-transcriptional regulatory element. In some embodiments, the microRNA sequence comprises a nucleic acid sequence that has at least 80%, at least 85%, at least 90%, at least 91%, at least 92%, at least 93%, at least 94%, at least 95%, at least 96%, at least 97%, at least 98%, or at least 99% identity to any one of (i) SEQ ID NO: 63, (ii) SEQ ID NO: 64, or (iii) SEQ ID NOs: 63 and 82. In some embodiments, the microRNA binding sequence comprises any one of (i) SEQ ID NO: 63, (ii) SEQ ID NO: 64, or (iii) SEQ ID NOs: 63 and 82. In some embodiments, the microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 63. In some embodiments, the microRNA binding sequence comprises the nucleic acid sequence of SEQ ID NO: 63. In some embodiments, the microRNA binding sequence comprises (i) a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 63 and (ii) a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO:82. In some embodiments, the microRNA binding sequence comprises SEQ ID NOs: 63 and 82. In some embodiments, the microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 64. In some embodiments, the microRNA binding sequence comprises the nucleic acid sequence of SEQ ID NO: 64.

In some embodiments, the vector further comprises a terminator downstream of the post-transcriptional regulatory element. In some embodiments, the vector comprises a nucleic acid comprising one or more inverted terminal repeats (ITRs). In some embodiments, the ITR sequences are derived from AAV. In some embodiments, the 5′ ITR sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 51. In some embodiments, the 5′ ITR sequence comprises the nucleic acid sequence of SEQ ID NO: 51. In some embodiments, the 3′ ITR sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 65. In some embodiments, the 3′ ITR sequence comprises the nucleic acid sequence of SEQ ID NO: 65.

In some embodiments, the vector comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to a nucleic acid sequence selected from SEQ ID NOs: 11-50.

In some embodiments, the vector comprises a nucleic acid comprising a sequence that is at least 80%, at least 85%, at least 90%, at least 91%, at least 92%, at least 93%, at least 94%, at least 95%, at least 96%, at least 97%, at least 98%, or at least 99% identical to any of the sequences of SEQ ID NOs: 11, 18-22, 27-30, and 42-45.

In some embodiments, the vector comprises a nucleic acid sequence selected from SEQ ID NOs: 11-50. In some embodiments, the expression construct comprises a nucleic acid sequence selected from SEQ ID NOs: 11, 18-22, 27-30, and 42-45.

In some embodiments, the vector comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to the nucleic acid sequence of SEQ ID NO: 11. In some embodiments, the expression construct comprises the nucleic acid sequence of SEQ ID NO: 11.

In some embodiments, the vector comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to the nucleic acid sequence of SEQ ID NO: 22. In some embodiments, the expression construct comprises the nucleic acid sequence of SEQ ID NO: 22.

In some embodiments, the vector comprises a nucleic acid comprising one or more of:
(a) a promoter sequence comprising a C14_L21 promoter, a C26_L21 promoter, a C42b_L21 promoter, a C46_L21 promoter, a B36 promoter, a B46 promoter, a B46 promoter, a B47 promoter, or a B48 promoter;
(b) a nucleic acid sequence encoding BDNF, wherein the nucleic acid sequence encoding BDNF is operatively linked to the promoter sequence;
(c) a post-transcriptional regulatory element;
(d) a polyadenylation signal; and/or
(e) one or more ITRs. In some embodiments, the vector comprises two ITR sequences.

In some embodiments, provided is a vector comprising a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 1-10;
(b) a nucleic acid sequence encoding BDNF, wherein the nucleic acid sequence encoding BDNF is operatively linked to the promoter and wherein the nucleic acid sequence encoding BDNF comprises a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 52-57 or 70-79;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 58-61;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62 or 66-69; and/or
(e) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding BDNF, wherein the nucleic acid sequence encoding BDNF is operatively linked to the promoter and wherein the nucleic acid sequence encoding BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 52;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 58;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 63; and
(f) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53, and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 57 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 60;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62; and
(e) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 54 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 60;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62; and
(e) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 60;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62; and
(e) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 55 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 60;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62; and
(e) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 2;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprising a sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 3;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprising a sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 4;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprising a sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 5;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprising a sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 61; and
(d) a polyadenylation sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 66.

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 9;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 61; and
(d) a polyadenylation sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 66.

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 8;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 61; and
(d) a polyadenylation sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 66.

In some embodiments, the vector comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 10;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 61; and
(d) a polyadenylation sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 66.

In some embodiments, the vector comprises a nucleic acid comprising one or more of:
(a) a promoter sequence comprising the nucleic acid sequence of SEQ ID NO: 1;
(b) a nucleic acid sequence encoding BDNF, wherein the nucleic acid sequence encoding BDNF is operatively linked to the promoter and wherein the nucleic acid sequence encoding BDNF comprises the nucleic acid sequence of SEQ ID NO: 52;
(c) a post-transcriptional regulatory element comprising the nucleic acid sequence of SEQ ID NO: 58;
(d) a polyadenylation signal comprising the nucleic acid sequence of SEQ ID NO: 62;
(e) a microRNA binding sequence comprises the nucleic acid sequence of SEQ ID NO: 63; and
(f) one or more ITRs (e.g., two ITR sequences).

In some embodiments, the vector comprises comprising a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 53, and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62;
(e) a microRNA binding sequence comprises (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 57 and wherein the nucleic acid sequence encoding the BDNF protein is operatively linked to the promoter;
(c) a post-transcriptional regulatory element comprising the nucleic acid sequence of SEQ ID NO: 60;
(d) a polyadenylation signal comprising the nucleic acid sequence of SEQ ID NO: 62; and
(e) one or more ITRs (e.g., two ITR sequences).

In some embodiments, the vector comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 54 and wherein the nucleic acid sequence encoding the BDNF protein is operatively linked to the promoter;
(c) a post-transcriptional regulatory element comprising the nucleic acid sequence of SEQ ID NO: 60;
(d) a polyadenylation signal comprising the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 60;
(d) a polyadenylation signal comprising the nucleic acid sequence of SEQ ID NO: 62; and
(e) one or more ITRs (e.g., two ITR sequences).

In some embodiments, the vector comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 55 and wherein the nucleic acid sequence encoding the BDNF protein is operatively linked to the promoter;
(c) a post-transcriptional regulatory element comprising the nucleic acid sequence of SEQ ID NO: 60;
(d) a polyadenylation signal comprising the nucleic acid sequence of SEQ ID NO: 62; and
(e) one or more ITRs (e.g., two ITR sequences).

In some embodiments, the vector comprises a nucleic acid comprising one or more of:
(a) a promoter sequence comprising the nucleic acid sequence of SEQ ID NO: 2;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62; and
(e) one or more ITRs (e.g., two ITR sequences).

In some embodiments, the vector comprises a nucleic acid comprising one or more of:
(a) a promoter sequence comprising the nucleic acid sequence of SEQ ID NO: 3;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62;
(e) a microRNA binding sequence comprising (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising the nucleic acid sequence of SEQ ID NO: 4;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62;
(e) a microRNA binding sequence comprising (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising the nucleic acid sequence of SEQ ID NO: 5;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62;
(e) a microRNA binding sequence comprising (i) SEQ ID NO: 64 or (ii) SEQ ID NOs: 63 and 82; and
(f) one or more ITRs (e.g., two ITR sequences).

In some embodiments, the vector comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 56 and wherein the nucleic acid sequence encoding the BDNF protein is operatively linked to the promoter;
(c) a post-transcriptional regulatory element comprising the nucleic acid sequence of SEQ ID NO: 61; and
(d) a polyadenylation sequence comprising the nucleic acid sequence of SEQ ID NO: 66.

In some embodiments, the vector comprises a nucleic acid comprising one or more of:
(a) a promoter sequence comprising the nucleic acid sequence of SEQ ID NO: 9;
(b) a nucleic acid sequence encoding a BDNF protein, wherein the nucleic acid sequence encoding the BDNF protein comprises the nucleic acid sequence of SEQ ID NO: 56 and wherein the nucleic acid sequence encoding the BDNF protein is operatively linked to the promoter;
(e) a post-transcriptional regulatory element comprising the nucleic acid sequence of SEQ ID NO: 61; and
(c) a polyadenylation sequence comprising the nucleic acid sequence of SEQ ID NO: 66.

In some embodiments, the vector comprises a nucleic acid comprising one or more of:
(a) a promoter sequence comprising the nucleic acid sequence of SEQ ID NO: 8;
(b) a nucleic acid sequence encoding a BDNF protein, wherein the nucleic acid sequence encoding the BDNF protein comprises the nucleic acid sequence of SEQ ID NO: 56 and wherein the nucleic acid sequence encoding the BDNF protein is operatively linked to the promoter;
(f) a post-transcriptional regulatory element comprising the nucleic acid sequence of SEQ ID NO: 61; and
(c) a polyadenylation sequence comprising the nucleic acid sequence of SEQ ID NO: 66.

In some embodiments, the vector comprises a nucleic acid comprising one or more of:
(a) a promoter sequence comprising the nucleic acid sequence of SEQ ID NO:10;
(b) a nucleic acid sequence encoding a BDNF protein, wherein the nucleic acid sequence encoding the BDNF protein comprises the nucleic acid sequence of SEQ ID NO: 56 and wherein the nucleic acid sequence encoding the BDNF protein is operatively linked to the promoter;
(g) a post-transcriptional regulatory element comprising the nucleic acid sequence of SEQ ID NO: 61; and
(c) a polyadenylation sequence comprising the nucleic acid sequence of SEQ ID NO: 66.

### Viral Vectors

Viral vectors for the expression of a target gene in a target cell, tissue, or organism are known in the art and include, for example, an AAV vector, adenovirus vector, lentivirus vector, retrovirus vector, poxvirus vector, baculovirus vector, herpes simplex virus vector, vaccinia virus vector, or a synthetic virus vector (e.g., a chimeric virus, mosaic virus, or pseudotyped virus, and/or a virus that contains a foreign protein, synthetic polymer, nanoparticle, or small molecule).

### AAV Vectors

As used herein, the term “adeno-associated virus (AAV) vector,” “AAV gene therapy vector,” and “gene therapy vector” refer to a vector having functional or partly functional ITR sequences and transgenes. As used herein, the term “ITR” refers to inverted terminal repeats (ITR).

Adeno-associated viruses (AAV) are small, single-stranded DNA viruses which require helper virus to facilitate efficient replication. The 4.7 kb genome of AAV is characterized by two inverted terminal repeats (ITR) and two open reading frames, which encode the Rep proteins and Cap proteins, respectively. The Rep reading frame encodes four proteins of molecular weight 78 kD, 68 kD, 52 kD, and 40 kD. These proteins function mainly in regulating AAV replication and rescue and integration of the AAV into a host cell's chromosomes. The Cap reading frame encodes three structural proteins of molecular weight 85 kD (VP1), 72 kD (VP2), and 61 kD (VP3), which form the virion capsid. More than 80% of total proteins in AAV virion comprise VP3. Flanking the rep and cap open reading frames at the 5′ and 3′ ends are about 145 bp long inverted terminal repeats (ITRs). The two ITRs are the only cis elements essential for AAV replication, rescue, packaging, and integration of the AAV genome. The entire rep and cap domains can be excised and replaced with a therapeutic or reporter transgene.

Recombinant adeno-associated virus “rAAV” vectors include any vector derived from any adeno-associated virus serotype. rAAV vectors can have one or more of the AAV wild-type genes deleted in whole or in part, In some embodiments the Rep and/or Cap genes, but retain functional flanking ITR sequences.

In some embodiments, the viral vector is an rAAV virion, which comprises an rAAV genome and one or more capsid proteins. In some embodiments, the rAAV genome comprises an expression construct disclosed herein.

In some embodiments, the viral vector disclosed herein comprises a nucleic acid comprising an AAV 5′ ITR and 3′ ITR located 5′ and 3′ to sequence encoding BDNF, respectively. However, in some embodiments, it may be desirable for the nucleic acid to contain the 5′ ITR and 3′ ITR sequences arranged in tandem, e.g., 5′ to 3′ or a head-to-tail, or in another alternative configuration. In still other embodiments, it may be desirable for the nucleic acid to contain multiple copies of the ITRs or to have 5′ ITRs (or conversely, 3′ ITRs) located both 5′ and 3′ to the sequence encoding BDNF. The ITRs sequences may be located immediately upstream and/or downstream of the heterologous molecule, or there may be intervening sequences. The ITRs need not be the wild-type nucleotide sequences, and may be altered (e.g., by the insertion, deletion, or substitution of nucleotides) so long as the sequences provide for functional rescue, replication, and packaging. The ITRs may be selected from AAV2, or from among the other AAV serotypes, as described herein.

In some embodiments, the viral vector is an AAV vector, such as an AAV1 (i.e., an AAV containing AAV1 ITRs and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), or AAVrh.10 (i.e., an AAV containing AAVrh.10 ITRs and AAVrh. 10 capsid proteins).

In some embodiments, the viral vector is a pseudotyped AAV vector, containing ITRs from one AAV serotype and capsid proteins from a different AAV serotype. In some embodiments, the pseudotyped AAV is AAV2/9 (i.e., an AAV containing AAV2 ITRs and AAV9 capsid proteins). In some embodiments, the pseudotyped AAV is AAV2/10 (i.e., an AAV containing AAV2 ITRs and AAV10 capsid proteins). In some embodiments, the pseudotyped AAV is AAV2/1 (i.e., an AAV containing AAV2 ITRs and AAV1 capsid proteins). In some embodiments, the pseudotyped AAVis AAV2/7m8 (i.e., an AAV containing AAV2 ITRs and AAV7m8 capsid proteins).

In some embodiments, the vector is a viral vector. In some embodiments, the vector is an AAV vector. In some embodiments, the AAV vector contains a recombinant capsid protein, such as a capsid protein containing a chimera of one or more of capsid proteins from AAV1, AAV2, AAV3, AAV4, AAV5, AAV6, AAV7, AAV8, AAV9, AAV10, AAVrh74, AAVrh.8, or AAVrh.10. In some embodiments, the vector comprises a genome derived from AAV1. In some embodiments, the vector comprises a genome derived from AAV2.

In one aspect, provided is a viral genome comprising a nucleic acid comprising (a) a promoter sequence operatively linked to a nucleic acid sequence encoding BDNF. In some embodiments, BDNF is encoded by a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to a nucleic acid sequence selected from SEQ ID NOs: 52-57 or 70-79 or comprises a nucleic acid sequence selected from SEQ ID NOs: 52-57 or 70-79. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 52. In some embodiments, the nucleic acid sequence encoding the BDNF comprises the nucleic acid sequence of SEQ ID NO: 52. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53. In some embodiments, the nucleic acid sequence encoding the BDNF comprises the nucleic acid sequence of SEQ ID NO: 53. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 54. In some embodiments, the nucleic acid sequence encoding the BDNF comprises the nucleic acid sequence of SEQ ID NO: 54. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 55. In some embodiments, the nucleic acid sequence encoding the BDNF comprises the nucleic acid sequence of SEQ ID NO: 55. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56. In some embodiments, the nucleic acid sequence encoding the BDNF comprises the nucleic acid sequence of SEQ ID NO: 56. In some embodiments, the nucleic acid sequence encoding the BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 57. In some embodiments, the nucleic acid sequence encoding the BDNF comprises the nucleic acid sequence of SEQ ID NO: 57. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 1-10. In some embodiments, the promoter sequence comprises a sequence selected from SEQ ID NOs: 1-10. In some embodiments, the promoter sequence comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid of SEQ ID NO: 1. In some embodiments, the promoter sequence to a nucleic acid comprises the nucleic acid of SEQ ID NO: 1. In some embodiments, the promoter sequence comprises a constitutive promoter.

In some embodiments, the viral genome comprises a nucleic acid comprising a post-transcriptional regulatory element. In some embodiments, the vector comprises a nucleic acid comprising a WPRE. In some embodiments, the post-transcriptional regulatory element comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to a nucleic acid sequence selected from SEQ ID NOs: 58-61. In some embodiments, the post-transcriptional regulatory element comprises a nucleic acid sequence selected from SEQ ID NOs: 58-61. In some embodiments, the post-transcriptional regulatory element comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 58 or 59. In some embodiments, the post-transcriptional regulatory element comprises the nucleic acid sequence of SEQ ID NO: 58 or 59.

In some embodiments, the viral genome comprises a nucleic acid comprising a polyadenylation signal. In some embodiments, the polyadenylation signal comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to the nucleic acid sequence of SEQ ID NO: 62 or 66-69. In some embodiments, the expression construct comprises the nucleic acid sequence of SEQ ID NO: 62 or 66-69. In some embodiments, the polyadenylation signal comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62. In some embodiments, the polyadenylation signal comprises the nucleic acid sequence of SEQ ID NO: 62.

In some embodiments, the viral genome comprises a microRNA binding sequence positioned in the 3′ UTR, for example, between the nucleic acid sequence encoding the BDNF and the post-transcriptional regulatory element. In some embodiments, the microRNA binding sequence comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to any one of (i) SEQ ID NO: 63, (ii) SEQ ID NO: 64, or (iii) SEQ ID NOs: 63 and 82. In some embodiments, the microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 63. In some embodiments, the microRNA binding sequence comprises the nucleic acid sequence of SEQ ID NO: 63. In some embodiments, the microRNA binding sequence comprises (i) a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 63 and (ii) a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO:82. In some embodiments, the microRNA binding sequence comprises SEQ ID NOs: 63 and 82. In some embodiments, the microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 64. In some embodiments, the microRNA binding sequence comprises the nucleic acid sequence of SEQ ID NO: 64.

In some embodiments, the microRNA binding sequence comprises (i) SEQ ID NO: 63, (ii) SEQ ID NO: 64, or (iii) SEQ ID NOs: 63 and 82.

In some embodiments, the viral genome comprises a terminator downstream of the post-transcriptional regulatory element.

In some embodiments, the viral genome comprises a nucleic acid comprising one or more inverted terminal repeats (ITR). In some embodiments, the ITR sequence is derived from AAV serotype 1 (AVV1). In some embodiments, the 5′ ITR sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 51. In some embodiments, the 5′ ITR sequence comprises the nucleic acid sequence of SEQ ID NO: 51. In some embodiments, the 3′ ITR sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 65. In some embodiments, the 3′ ITR sequence comprises the nucleic acid sequence of SEQ ID NO: 65.

In some embodiments, the vector comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to a nucleic acid sequence selected from SEQ ID NOs: 11-50.

In some embodiments, the viral genome comprises a nucleic acid comprising a sequence that is at least 80%, at least 85%, at least 90%, at least 91%, at least 92%, at least 93%, at least 94%, at least 95%, at least 96%, at least 97%, at least 98%, or at least 99% identical to any of the sequences of SEQ ID NOs: 11, 18-22, 27-30, and 42-45.

In some embodiments, the viral genome comprises a nucleic acid sequence selected from SEQ ID NOs: 11-50. In some embodiments, the expression construct comprises a nucleic acid sequence selected from SEQ ID NOs: 11, 18-22, 27-30, and 42-45.

In some embodiments, the viral genome comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to the nucleic acid sequence of SEQ ID NO: 11. In some embodiments, the expression construct comprises the nucleic acid sequence of SEQ ID NO: 11.

In some embodiments, the viral genome comprises a nucleic acid sequence that has at least 80%, at least 85%, at least 90%, at least 91%, at least 92%, at least 93%, at least 94%, at least 95%, at least 96%, at least 97%, at least 98%, or at least 99% sequence identity to the nucleic acid sequence of SEQ ID NO: 22. In some embodiments, the expression construct comprises the nucleic acid sequence of SEQ ID NO: 22.

In some embodiments, the viral genome comprises a nucleic acid comprising one or more of:
(a) a promoter sequence comprising a C14_L21 promoter, a C26_L21 promoter, a C42b_L21 promoter, a C46_L21 promoter, a B36 promoter, a B46 promoter, a B46 promoter, a B47 promoter, or a B48 promoter;
(b) a nucleic acid sequence encoding BDNF, wherein the nucleic acid sequence encoding BDNF is operatively linked to the promoter sequence;
(c) a WPRE;
(d) a polyadenylation signal; and/or
(e) one or more ITRs. In some embodiments, the viral genome comprises two ITR sequences.

In some embodiments, the viral genome comprising a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 1-10;
(b) a nucleic acid sequence encoding BDNF, wherein the nucleic acid sequence encoding BDNF is operatively linked to the promoter and wherein the nucleic acid sequence encoding BDNF comprises a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 52-57 or 70-79;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to a nucleic acid sequence selected from SEQ ID NOs: 58-61;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62 or 66-69; and
(e) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding BDNF, wherein the nucleic acid sequence encoding BDNF is operatively linked to the promoter and wherein the nucleic acid sequence encoding BDNF comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 52;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 58;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of the nucleic acid sequence of SEQ ID NO: 63; and
(f) one or more ITRs (e.g., two ITR sequences).

In some embodiments, the viral genome comprises comprising a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53, and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprises a nucleic acid sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 57 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 60;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 54 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 60;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62; and one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 55 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 60;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 2;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprising a sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 3;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA sequence comprising a sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 4;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA sequence comprising a sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 5;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 53 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 59;
(d) a polyadenylation signal comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 62;
(e) a microRNA binding sequence comprising a sequence that is at least 80%, at least 85%, at least 90%, at least 91%, at least 92%, at least 93%, at least 94%, at least 95%, at least 96%, at least 97%, at least 98%, or at least 99% identical to (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 1;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56 and wherein the nucleic acid sequence encoding the BDNF protein is operatively linked to the promoter;
(e) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 61; and
(c) a polyadenylation sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 66.

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 9;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56 and wherein the nucleic acid sequence encoding the BDNF protein is operatively linked to the promoter;
(e) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 61; and
(c) a polyadenylation sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 66.

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 8;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56 and wherein the nucleic acid sequence encoding the BDNF protein is operatively linked to the promoter;
(c) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 61; and
(d) a polyadenylation sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 66.

In some embodiments, the viral genome comprises a nucleic acid comprising one or more of:
(a) a promoter sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 10;
(b) a nucleic acid sequence encoding a BDNF protein, wherein the nucleic acid sequence encoding the BDNF protein comprises a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 56 and wherein the nucleic acid sequence encoding the BDNF protein is operatively linked to the promoter;
(e) a post-transcriptional regulatory element comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 61; and
(c) a polyadenylation sequence comprising a sequence that is at least 80%, at least 85%, at least 90%, at least 91%, at least 92%, at least 93%, at least 94%, at least 95%, at least 96%, at least 97%, at least 98%, or at least 99% identical to the nucleic acid sequence of SEQ ID NO: 66.

In some embodiments, the viral genome comprises a nucleic acid comprising one or more of:
(a) a promoter sequence comprising the nucleic acid sequence of SEQ ID NO: 1;
(b) a nucleic acid sequence encoding BDNF, wherein the nucleic acid sequence encoding BDNF is operatively linked to the promoter and wherein the nucleic acid sequence encoding BDNF comprises the nucleic acid sequence of SEQ ID NO: 52;
(c) a post-transcriptional regulatory element comprising the nucleic acid sequence of SEQ ID NO: 58;
(d) a polyadenylation signal comprising the nucleic acid sequence of SEQ ID NO: 62;
(e) a microRNA binding sequence comprises the nucleic acid sequence of SEQ ID NO: 63; and
(f) one or more ITRs (e.g., two ITR sequences).

In some embodiments, the viral genome comprises comprising a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 53, and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62;
(e) a microRNA binding sequence comprises (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 57 and wherein the nucleic acid sequence encoding the BDNF protein is operatively linked to the promoter;
(c) a post-transcriptional regulatory element comprising the nucleic acid sequence of SEQ ID NO: 60;
(d) a polyadenylation signal comprising the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 54 and wherein the nucleic acid sequence encoding the BDNF protein is operatively linked to the promoter;
(c) a post-transcriptional regulatory element comprising the nucleic acid sequence of SEQ ID NO: 60;
(d) a polyadenylation signal comprising the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 60;
(d) a polyadenylation signal comprising the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 55 and wherein the nucleic acid sequence encoding the BDNF protein is operatively linked to the promoter;
(c) a post-transcriptional regulatory element comprising the nucleic acid sequence of SEQ ID NO: 60;
(d) a polyadenylation signal comprising the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising the nucleic acid sequence of SEQ ID NO: 2;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62; and
(e) one or more ITRs (e.g., two ITR sequences).

In some embodiments, the viral genome comprises a nucleic acid comprising one or more of:
(a) a promoter sequence comprising the nucleic acid sequence of SEQ ID NO: 3;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62;
(e) a microRNA binding sequence comprising (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising the nucleic acid sequence of SEQ ID NO: 4;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62;
(e) a microRNA binding sequence comprising (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising the nucleic acid sequence of SEQ ID NO: 5;
(b) a nucleic acid sequence encoding a BDNF protein, wherein the nucleic acid sequence encoding the BDNF protein comprises the nucleic acid sequence of SEQ ID NO: 53 and wherein the nucleic acid sequence encoding the BDNF protein is operatively linked to the promoter;
(c) a post-transcriptional regulatory element comprising the nucleic acid sequence of SEQ ID NO: 59;
(d) a polyadenylation signal comprising the nucleic acid sequence of SEQ ID NO: 62;
(e) a microRNA binding sequence comprising (i) SEQ ID NO: 64 or (ii) SEQ ID NOs: 63 and 82; and
(f) one or more ITRs (e.g., two ITR sequences).

In some embodiments, the viral genome comprises a nucleic acid comprising one or more of:
(a) a promoter sequence comprising the nucleic acid sequence of SEQ ID NO: 1;
(b) a nucleic acid sequence encoding a BDNF protein, wherein the nucleic acid sequence encoding the BDNF protein comprises the nucleic acid sequence of SEQ ID NO: 56 and wherein the nucleic acid sequence encoding the BDNF protein is operatively linked to the promoter;
(h) a post-transcriptional regulatory element comprising the nucleic acid sequence of SEQ ID NO: 61; and
(c) a polyadenylation sequence comprising the nucleic acid sequence of SEQ ID NO: 66.

In some embodiments, the viral genome comprises a nucleic acid comprising one or more of:
(a) a promoter sequence comprising the nucleic acid sequence of SEQ ID NO: 9;
(b) a nucleic acid sequence encoding a BDNF protein, wherein the nucleic acid sequence encoding the BDNF protein comprises the nucleic acid sequence of SEQ ID NO: 56 and wherein the nucleic acid sequence encoding the BDNF protein is operatively linked to the promoter;
(i) a post-transcriptional regulatory element comprising the nucleic acid sequence of SEQ ID NO: 61; and
(c) a polyadenylation sequence comprising the nucleic acid sequence of SEQ ID NO: 66.

In some embodiments, the viral genome comprises a nucleic acid comprising one or more of:
(a) a promoter sequence comprising the nucleic acid sequence of SEQ ID NO: 8;
(b) a nucleic acid sequence encoding a BDNF protein, wherein the nucleic acid sequence encoding the BDNF protein comprises the nucleic acid sequence of SEQ ID NO: 56 and wherein the nucleic acid sequence encoding the BDNF protein is operatively linked to the promoter;
(j) a post-transcriptional regulatory element comprising the nucleic acid sequence of SEQ ID NO: 61; and
(c) a polyadenylation sequence comprising the nucleic acid sequence of SEQ ID NO: 66.

In some embodiments, the viral genome comprises a nucleic acid comprising one or more of:
(a) a promoter sequence comprising the nucleic acid sequence of SEQ ID NO:10;
(b) a nucleic acid sequence encoding a BDNF protein, wherein the nucleic acid sequence encoding the BDNF protein comprises the nucleic acid sequence of SEQ ID NO: 56 and wherein the nucleic acid sequence encoding the BDNF protein is operatively linked to the promoter;
(k) a post-transcriptional regulatory element comprising the nucleic acid sequence of SEQ ID NO: 61; and
(c) a polyadenylation sequence comprising the nucleic acid sequence of SEQ ID NO: 66.

### Other Viral Vectors

Other viral vectors include adenoviral (AV) vectors, for example, those based on human adenovirus type 2 and human adenovirus type 5 that have been made replication defective through deletions in the E1 and E3 regions. The transcriptional cassette can be inserted into the E1 region, yielding a recombinant E1/E3-deleted AV vector. Adenoviral vectors also include helper-dependent high-capacity adenoviral vectors (also known as high-capacity, “gutless” or “gutted” vectors), which do not contain viral coding sequences. These vectors contain the cis-acting elements needed for viral DNA replication and packaging, mainly the inverted terminal repeat sequences (ITR) and the packaging signal (CY). These helper-dependent AV vector genomes have the potential to carry from a few hundred base pairs up to approximately 36 kb of foreign DNA. Alternatively, other systems such as lentiviral vectors can be used. Lentiviral-based systems can transduce nondividing as well as dividing cells making them useful for applications targeting, for examples, the nondividing cells of the CNS. Lentiviral vectors are derived from the human immunodeficiency virus and, like that virus, integrate into the host genome providing the potential for very long-term gene expression. Polynucleotides, including plasmids, YACs, minichromosomes and minicircles, carrying the target gene containing the expression construct can also be introduced into a cell or organism by nonviral vector systems using, for example, cationic lipids, polymers, or both as carriers. Conjugated poly-L-lysine (PLL) polymer and polyethylenimine (PEI) polymer systems can also be used to deliver the vector to cells. Other methods for delivering the vector to cells includes hydrodynamic injection and electroporation and use of ultrasound, both for cell culture and for organisms. For a review of viral and non-viral delivery systems for gene delivery see Nayerossadat, N. et al. (Adv Biomed Res. 2012; 1:27) incorporated herein by reference.

rAAV Virion Production

The rAAV virions disclosed herein may be constructed and produced using the materials and methods described herein, as well as those known to those of skill in the art. Such engineering methods used to construct any embodiment of this disclosure are known to those with skill in nucleic acid manipulation and include genetic engineering, recombinant engineering, and synthetic techniques. See, e.g., Sambrook et al, “Molecular Cloning. A Laboratory Manual”, 2d ed., Cold Spring Harbor Laboratory, New York (1989), and Ausubel et al., Current Protocols in Molecular Biology, John Wiley & Sons, New York, 1989); and International Patent Publication No. WO 95/13598. Further, methods suitable for producing a rAAV construct in an adenoviral capsid have been described in U.S. Pat. Nos. 5,856,152 and 5,871,982. Briefly, in order to package the rAAV genome into a rAAV virion, a host cell is used that contains sequences necessary to express AAV rep and AAV cap or functional fragments thereof as well as helper genes essential for AAV production. The AAV rep and cap sequences are obtained from an AAV source as identified herein. The AAV rep and cap sequences may be introduced into the host cell in any manner known to one in the art, including, without limitation, transfection, electroporation, liposome delivery, membrane fusion techniques, high velocity DNA-coated pellets, viral infection, and protoplast fusion. In some embodiments, the rep and cap sequences may be transfected into the host cell by one or more nucleic acid molecules and exist stably in the cell as an episome. In another embodiment, the rep and cap sequences are stably integrated into the genome of the cell. Another embodiment has the rep and cap sequences transiently expressed in the host cell. For example, a useful nucleic acid molecule for such transfection comprises, from 5′ to 3′, a promoter, an optional spacer interposed between the promoter and the start site of the rep gene sequence, an AAV rep gene sequence, and an AAV cap gene sequence. As used herein, the term “from 5′ to 3′” merely refers to the order of the specific genetic elements in a nucleic sequence. In some embodiments, the specific genetic elements are linked to one another by way of a linker nucleic acid (e.g., an intervening non-coding nucleic acid). In some embodiments, the specific genetic elements are linked to one another with no intervening nucleotides present. In some embodiments, some of the specific genetic elements are linked to one another by way of a linker nucleic acid, while other are linked to one another with no intervening nucleotides present.

The rep and cap sequences, along with their expression control sequences, may be supplied on a single vector, or each sequence may be supplied on its own vector. In some embodiments, the rep and cap sequences are supplied on the same vector. Alternatively, the rep and cap sequences may be supplied on a vector that contains other DNA sequences that are to be introduced into the host cells. In some embodiments, the promoter used in this construct may be any suitable constitutive, inducible or native promoters known to one of skill in the art. The molecule providing the rep and cap proteins may be in any form which transfers these components to the host cell. Desirably, this molecule is in the form of a plasmid, which may contain other non-viral sequences, such as those for marker genes. This molecule does not contain the AAV ITRs and generally does not contain the AAV packaging sequences. To avoid the occurrence of homologous recombination, other virus sequences, particularly those of adenovirus, are avoided in this plasmid. This plasmid is desirably constructed so that it may be stably transfected into a cell.

Although the molecule providing rep and cap may be transiently transfected into the host cell, it is preferred that the host cell be stably transformed with sequences necessary to express functional rep/cap proteins in the host cell, e.g., as an episome or by integration into the chromosome of the host cell. Depending upon the promoter controlling expression of such stably transfected host cell, the rep/cap proteins may be transiently expressed (e.g., through use of an inducible promoter).

The methods employed for constructing embodiments of this disclosure are conventional genetic engineering or recombinant engineering techniques such as those described in the references above. For example, the rAAV may be produced utilizing a triple transfection method using either the calcium phosphate method (Clontech) or Effectene™ reagent (Qiagen, Valencia, Calif.), according to manufacturer's instructions. See, also, Herzog et al, 1999, Nature Medic., 5 (1): 56-63, for the method used in the following examples, employing the plasmid with the transgene, a helper plasmid containing AAV rep and cap, and a plasmid supplying adenovirus helper functions of E2A, E4Orf6 and VA. While this disclosure provides illustrative examples of specific constructs, using the information provided herein, one of skill in the art may select and design other suitable constructs, using a choice of spacers, promoters, and other elements, including at least one translational start and stop signal, and the optional addition of polyadenylation sites.

The rAAV virions are then produced by culturing a host cell containing a rAAV virus as described herein which contains a rAAV genome to be packaged into a rAAV virion, an AAV rep sequence and an AAV cap sequence under the control of regulatory sequences directing expression thereof. Suitable viral helper genes, e.g., adenovirus E2A, E4Orf6 and VA, among other possible helper genes, may be provided to the culture in a variety of ways known to the art. In some embodiments, suitable viral helper genes are provided on a separate plasmid. Thereafter, the recombinant AAV virion which directs expression of the BDNF transgene is isolated from the cell or cell culture in the absence of contaminating helper virus or wild type AAV.

Expression of the BDNF transgene may be measured in ways known in the art. For example, a target cell may be infected in vitro, and the number of copies of the transgene in the cell monitored by Southern blotting or quantitative polymerase chain reaction (PCR). The level of RNA expression may be monitored by Northern blotting or quantitative reverse transcriptase (RT)-PCR; and the level of protein expression may be monitored by Western blotting, immunohistochemistry, enzyme-linked immunosorbent assay (ELISA), radioimmunoassay (RIA) or by the specific methods detailed below in the Examples.

### Pharmaceutical Compositions

Provided herein are pharmaceutical compositions comprising any of the vectors or viral particles disclosed herein and a pharmaceutically acceptable carrier.

In some embodiments, the rAAV comprising the gene encoding BDNF is assessed for contamination by conventional methods and then formulated into a pharmaceutical composition suitable for storage and/or administration to a patient.

Formulations of the vectors disclosed herein involve the use of a pharmaceutically and/or physiologically acceptable vehicle or carrier, particularly one suitable for subretinal injection, such as buffered saline or other buffers, e.g., HEPES, to maintain pH at appropriate physiological levels.

The vector of the disclosure can be formulated into pharmaceutical compositions. These compositions may comprise, in addition to the vector, a pharmaceutically and/or physiologically acceptable excipient, carrier, buffer, stabilizer, antioxidants, preservative, or other additives well known to those skilled in the art. Such materials should be non-toxic and should not interfere with the efficacy of the active ingredient. The precise nature of the carrier or other material may be determined by the skilled person according to the route of administration. The pharmaceutical composition is typically in liquid form. Liquid pharmaceutical compositions generally include a liquid carrier such as water, petroleum, animal or vegetable oils, mineral oil or synthetic oil. Additional carriers are provided in International Patent Publication No. WO 00/15822, incorporated herein by reference. Physiological saline solution, magnesium chloride, dextrose or other saccharide solution or glycols such as ethylene glycol, propylene glycol or polyethylene glycol may be included. In some cases, a surfactant, such as pluronic acid (PF68) 0.001% may be used. In some cases, Ringer's Injection, Lactated Ringer's Injection, or Hartmann's solution is used. Preservatives, stabilizers, buffers, antioxidants and/or other additives may be included, as required.

Pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The agents may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The agents may also be formulated in rectal compositions such as suppositories or retention enemas, e.g., containing conventional suppository bases such as cocoa butter or other glycerides.

“Parenteral” administration of a composition includes, e.g., subcutaneous (s.c.), intravenous (i.v.), intramuscular (i.m.), or intrasternal injection, or infusion techniques.

For delayed release, the vector may be included in a pharmaceutical composition which is formulated for slow release, such as in microcapsules formed from biocompatible polymers or in liposomal carrier systems according to methods known in the art.

If the vector is to be stored long-term, it may be frozen in the presence of glycerol.

### Methods of Use

Provided herein is a method for treating or preventing a metabolic disorder in a subject in need thereof. In some embodiments, the method comprises administering to the subject a therapeutically effective amount of the vector or the pharmaceutical composition, as described herein. In some embodiments, the metabolic disorder is obesity.

Also within the scope of this disclosure is a method of reducing or eliminating metabolic-related disorder symptoms in a subject in need thereof. In some embodiments, the method comprises administering to the subject the vector or the pharmaceutical composition, as described herein. In certain embodiments, the metabolic-related disorder symptoms are one or more of obesity, insulin sensitivity, syndrome X, and diabetes.

“Metabolic disorder” refers to disorders, diseases and conditions caused or characterized by abnormal weight gain, energy use or consumption, altered responses to ingested or endogenous nutrients, energy sources, hormones or other signaling molecules within the body or altered metabolism of carbohydrates, lipids, proteins, nucleic acids or a combination thereof. A metabolic disorder may be associated with either a deficiency or an excess in a metabolic pathway resulting in an imbalance in metabolism of carbohydrates, lipids, proteins and/or nucleic acids. Examples of metabolic disorders include, but are not limited to, metabolic syndrome, insulin-deficiency or insulin-resistance related disorders, Diabetes Mellitus (such as, for example, Type 2 Diabetes), glucose intolerance, abnormal lipid metabolism, atherosclerosis, hypertension, pre-eclampsia, cardiac pathology, stroke, non-alcoholic fatty liver disease (NAFLD), hyperglycemia, hepatic steatosis from different etiology, dyslipidemia, dysfunction of the immune system associated with overweight and obesity, cardiovascular diseases, high cholesterol, elevated triglycerides, asthma, sleep apnea, osteoarthritis, neuro-degeneration, gallbladder disease, syndrome X, inflammatory and immune disorders, atherogenic dyslipidemia, and cancer.

“Obesity” refers to a subject condition wherein said subject has a Body Mass Index (BMI) superior or equal to 30. BMI is typically calculated by dividing a human subject's weight in kilograms (or pounds) by the square of height in meters (or feet).

In some embodiments, the subject is a mammal. The term “mammal” as used herein is intended to include, but is not limited to, humans, laboratory animals, domestic pets, and farm animals. Mammals, include, but are not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of one or more symptoms of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

In yet another aspect, this disclosure additionally provides a method of increasing a level or activity of BDNF in a subject in need thereof. The method comprises administering to the subject the vector or the pharmaceutical composition, as described herein.

The level or activity of BDNF may be measured by determining or estimating the protein level or mRNA level. Methods for determining or estimating the protein level or mRNA level are well-known in the art. For example, the protein level (e.g., protein expression level) of BDNF can be determined by SDS-PAGE, Western blot, or an immunoassay (e.g., immunoblotting assay, immunoprecipitation assay). The mRNA level can be determined by RT-PCR.

Measuring the levels of BDNF can be performed by assaying the proteins themselves (by Western blotting, ELISA, RIA, and other techniques known to one skilled in the art), by assaying the mRNA encoding these proteins (such as quantitative PCR, Northern blotting, RNAse protection assay, RNA dot-blotting, and other techniques known to one skilled in the art), or by assaying the activity of the regulatory elements of the genes for BDNF. For example, the activity of regulatory elements can be assessed by reporter constructs consisting of DNA segments from the promoter, enhancer, and/or intronic elements coupled to cDNAs encoding reporters (such as luciferase, beta-galactosidase, green fluorescent protein, or other reporting genes that can be easily assayed). These reporter constructs can be transfected into cells, either stably, or transiently.

### Route and Methods of Administration

In some embodiments, the vectors or the pharmaceutical compositions disclosed herein are administered to the subject intratumorally, intravenously, subcutaneously, intraosseously, orally, transdermally, intracerebrally, intraparenchymally, in sustained release, in controlled release, in delayed release, as a suppository, or sublingually.

In some embodiments, the vector or the pharmaceutical composition is administered by stereotaxic microinjection.

The dose of a vector of the disclosure may be determined according to various parameters, especially according to the age, weight and condition of the patient to be treated, the particular disorder and the degree to which the disorder, if progressive, has developed, the route of administration; and the required regimen. A physician will be able to determine the required route of administration and dosage for any particular patient.

An effective amount of an rAAV carrying a nucleic acid sequence encoding BDNF under the control of the promoter sequence desirably ranges between about 1×10⁹ to about 2×10¹² (e.g., about 5×10⁹ to about 2×10¹¹) rAAV genome particles or between about 1× 10¹⁰ to about 2×10¹¹ genome particles. A “genome particle” is defined herein as an AAV capsid that contains a single stranded DNA molecule that can be quantified with a sequence specific method (such as real-time PCR). In some embodiments, the about 1×10⁹ to about 2×10¹² (e.g., about 5×10⁹ to about 2×10¹¹) rAAV genome particles may be provided in a volume of between about 150 to about 800 μl (e.g., about 100 to about 500 μl, such as about 100 to about 200 μl). Still other dosages in these ranges may be selected by the attending physician.

In some embodiments, the dose may be provided as one or more doses to be administered bi-laterally into the hypothalamus. In some embodiments, it may be desirable to administer multiple “booster” dosages of a pharmaceutical compositions disclosed herein. For example, depending upon the duration of the transgene within the target cell, one may deliver booster dosages at 6 month intervals, or yearly following the first administration. Other similar tests may be used to determine the status of the treated subject over time. Selection of the appropriate tests may be made by the attending physician.

### Articles of Manufacture and Kits

Also provided are kits or articles of manufacture for use in the methods described herein. In some aspects, the kits comprise the compositions described herein (e.g., compositions for delivery of a BDNF encoding transgene) in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed.

Also provided are kits comprising the compositions described herein. These kits may further comprise instruction(s) on methods of using the composition, such as uses described herein. The kits described herein may further include other materials desirable from a commercial and user standpoint, including buffers, diluents, filters, needles, syringes, and package inserts with instructions for performing the administration of the composition or performing any methods described herein. For example, in some embodiments, the kit comprises an rAAV for the expression of a BDNF encoding transgene in target cells, a pharmaceutically acceptable carrier suitable for injection, and one or more of: a buffer, a diluent, a filter, a needle, a syringe, and a package insert with instructions for performing the injections.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

### Cell Culture

Neuro2A (N2A), BE2-M17, and SH-SY5Y immortalized cell lines were maintained at 37° C. in 5% CO₂. Cells were transiently transfected with plasmids using the TurboFect Transfection Reagent. After two days, the media and cell lysate were collected for protein analysis. After 30 passages, a new aliquot of cells was thawed and passaged twice before use for subsequent experiments.

### Primary Neuronal Culture

Embryonic day 16 (E16) timed-pregnant female mice were anesthetized with CO₂ and sacrificed by cervical dislocation. In a dissection hood, 24-26 embryos per experiment were collected through an incision of the mother's abdomen, taken out of the amniotic sacs, and decapitated in ice-cold Hank's Balanced Salt Solution (HBSS). Using fine scissors and forceps, brains were rapidly dissected, and the cortex was cleared from meninges and isolated under a dissection microscope. Cortices were collected in ice-cold HBSS and kept on ice until all embryos had been dissected. In a tissue culture hood, HBSS was removed, and the cortex tissue was digested by 0.25% Trypsin-EDTA for 12 min at 37° C., followed by DNase1 treatment for 10 min at 37° C. The tissue was dissociated by serial trituration with a 25-ml serological pipette, followed by trituration with 10 and 5 ml serological pipettes. Cell suspension was washed once with DMEM medium, supplemented with 10% FBS and 1% penicillin/streptomycin, and passed through a 40 μM cell strainer before being counted on a hemocytometer. Single cells were seeded on poly-d-lysine (0.1 mg ml-1)-coated wells at a density of 400,000 cells per well on a 24-well plate. Cells were grown in neurobasal medium, supplemented with B-27™ supplement, N-2 supplement, and 0.5 mM L-glutamine and maintained at 37° C. in 5% CO₂. Every 3-4 days, half media changes were performed to feed the cultures.

### Primary Neuron Transduction

On day 1 in vitro, AAV2/1 particles were added to the neuronal media at a multiplicity of infection (MOI) of 10,000 and 50,000. Media was changed 72 hours later according to the standard neuronal protocol.

### BDNF Activity Assay

Media from transfected N2A cells was collected after 2 days and spun at 500 rpg to remove any cellular debris. BDNF levels in this clean media was assessed via ELISA. Media containing a standardized amount of BDNF was added to primary cultures and incubated for 15-30 minutes before the lysate was collected and prepared for western blotting.

### Western Blotting

Cells were gently washed with PBS before the addition of ice-cold lysis buffer (CST) containing DNAse and a protease/phosphatase inhibitor. Cells were lysed on the plate for 15 minutes on ice with agitation and then collected. Samples were vortexed vigorously to ensure complete lysis and then pelleted at top speed on a micro-centrifuge for 10 minutes. The supernatant was collected and protein concentration determined by BCA assay. Protein was then normalized to a standard concentration using 4×LDS, 10×DTT, and lysis buffer. Samples were denatured at 85° C. for 10 min.

For BDNF analysis, 10 μg of protein samples were separated on 4-12% Tris-Glycine Mini-PROTEAN denaturing gels (BioRad) using Tris-Glycine-SDS buffer. For total ERK1/2 and phosphor-ERK1/2 analysis, 10 ug of protein samples were separated on 4-12% Bolt Bis-tris precast denaturing gels (Invitrogen, USA) using MOPS buffer. Gels were then transferred onto nitrocellulose membranes. Membranes were probed with primary antibodies (total ERK1/2, phosphor-ERK1/2, BDNF, Gapdh, beta-Actin) diluted in Licor TBS Blocking Buffer overnight at 4° C. Membranes were then washed and probed with fluorescent conjugated anti-mouse or anti-rabbit IgG secondary antibodies for one hour at room temperature. Membranes were imaged on the LICOR Odyssey system. Protein quantities were normalized using beta-Actin or Gapdh.

### ELISA

Tissue samples were lysed in CST RIPA buffer with no SDS containing DNase and a protease/phosphatase inhibitor cocktail. Samples were sonicated twice and then spun to remove cellular debris. Samples were then used for pro-BDNF and mature BDNF ELISA (Biosensis) according to the manufacturer's protocol.

### Animals

7-wk-old male C57BL/6 mice were purchased from The Charles River Laboratories. Mice were housed at five animals per cage on a 12-h light/dark cycle (lights on from 0700 to 1900 h) at constant temperature (23° C.) with ad libitum access to food and water. All studies were reviewed by the Institutional Animal Care and Use Committee (IA (II (′). Animals were randomized into groups based on weight prior to surgery. At the conclusion of the study, the hypothalamus was dissected rapidly and snap-frozen in liquid nitrogen.

### Stereotaxic Surgery

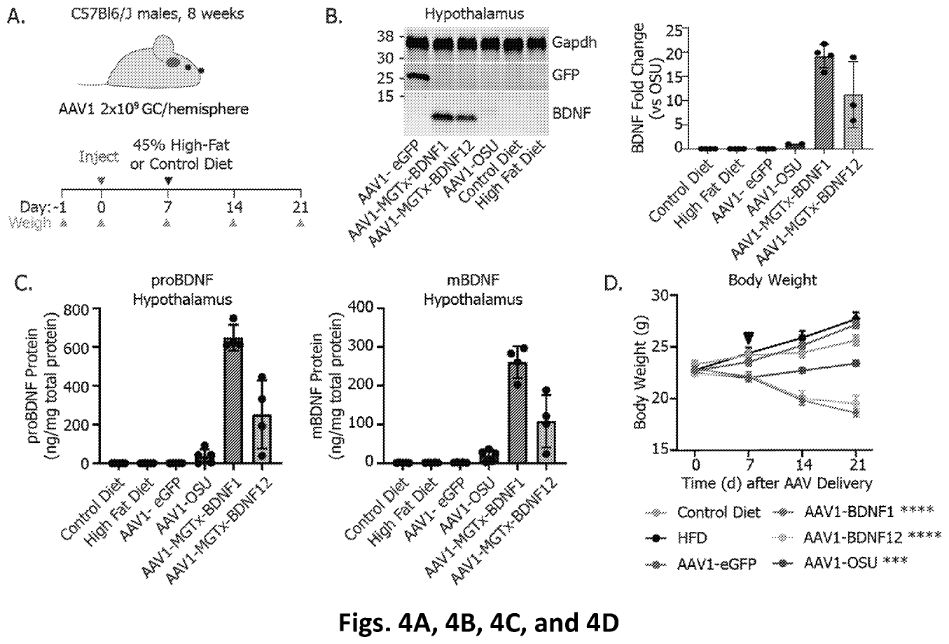

To overexpress BDNF in the adult hypothalamus, adeno-associated virus (0.5 μl, 2e9 GC per hemisphere, serotype 1) was bilaterally injected into 8-week-old male C57Bl/6 mice. Animals were anesthetized with isoflurane and secured via ear bars and an incisor bar onto a Kopf stereotaxic frame. A midline incision through the scalp revealed the skull and two small holes were drilled into the skull with a dental drill above the injection sites (1.2 mm posterior to the bregma, 0.5 mm lateral to the midline, 5.85 mm dorsal to the bregma). rAAV vectors were injected (2e9 genomic particles per site) bilaterally into the hypothalamus at a rate of 0.1 ml per minute with a 5-μl Hamilton Neuro syringe attached to a Micro4 Micro Syringe Pump Controller (World Precision Instruments). At the end of infusion, the syringe was slowly raised from the brain and Vetbond was applied to close the scalp. Mice were placed into a clean cage and carefully monitored until recovery from anesthesia. Mice were fed with normal chow diet (NCD, 11% fat, 28% protein, 61% carbohydrate, caloric density 3.4 kcal per g, Research Diets; also referred to as “Control Diet” in FIG. 4D). On day 7 after rAAV injection, mice of appropriate groups were placed on high-fat diet (HFD, 45% fat, caloric density 4.73 kcal g1, Research Diets) for the duration of the study (21 days after injection).

### Example 2

Several BDNF gene therapy vectors for the treatment of obesity were developed. For an overview of expression constructs, see Tables 1-8. The different expression constructs were designed by altering various cis-regulatory components. As shown herein, BDNF expression constructs achieved significantly higher expression compared to a previously published construct in head-to-head comparisons in primary mouse cortical neurons as well as in various immortalized neuronal cell lines. Furthermore, AAV-mediated delivery of BDNF expression vectors to the hypothalamus of mice was more effective than the published CAG-BDNF construct at preventing weight gain induced by high-fat diet. By designing gene therapy vectors allowing for high levels of BDNF expression, therapeutic efficacy at lower viral vector doses and potentially lower immune responses and decreased safety risks can be achieved. Other advantages of the disclosed gene therapy vectors include: one-time administration leads to long-term treatment without requiring adherence to an injection regimen; it limits peripheral side effects because it would be locally administered to the brain; and it directly targets the mechanistic area. Taken together, these results demonstrate that the disclosed expression constructs provide means for a potent and effective gene therapy for the treatment of rare obesity populations as well as more prevalent forms of obesity that arise from genic or environmental factors.

Higher BDNF Expression from a Constitutive Promoter than Tissue-Specific or Original (AG Promoters

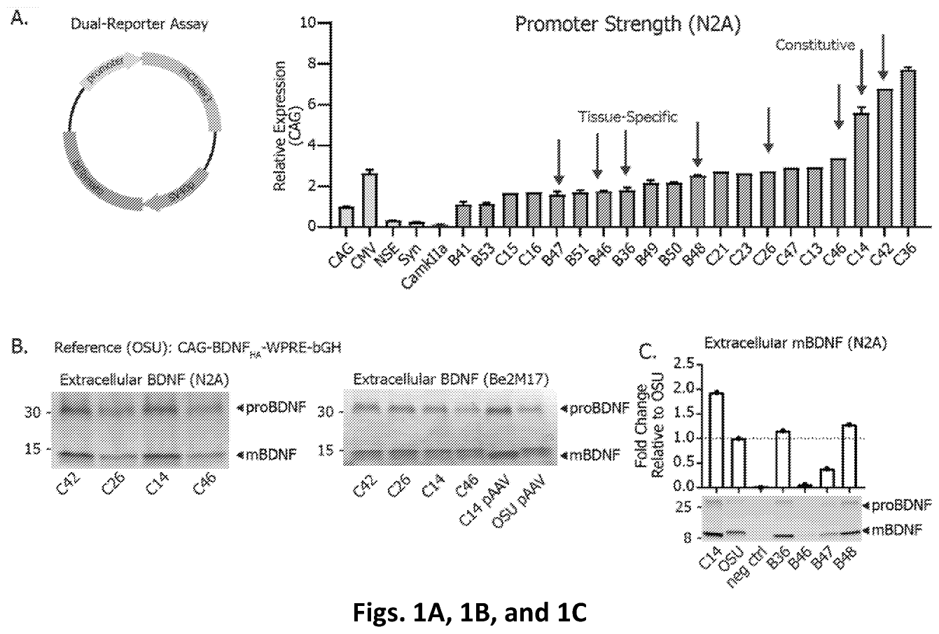

Promoters are integral components of a gene therapy that impact transgene expression level, timing, durability, and cell-type specificity. Therefore, a library of tissue-specific and constitutive promoters that were stronger in mouse N2A cells than commonly used reference promoters (NSE, Syn, CAmkIIa) were developed. These promoters were equal to or greater than CMV, and some were even stronger than CAG. A subset of each class of these promoters were selected and applied to BDNF to increase expression. In both mouse (N2A) and human cells (BE2M17), the C14 promoter drove the some of the highest levels of BDNF expression in vitro of all the promoters tested (FIG. 1). This promoter was then carried forward into subsequent experiments and rounds of expression construct optimization.

BDNF Expression Constructs Disclosed Herein are More Potent than the Reference Construct In Vitro

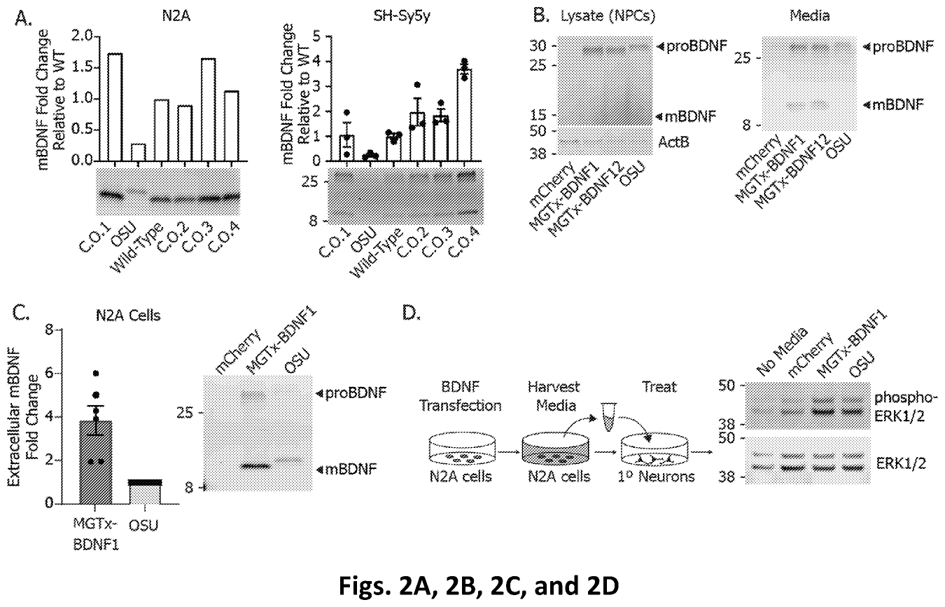

Four different codon optimization algorithms were applied to the BDNF transgenes It was assessed how codon-optimization impacted BDNF expression relative to reference construct CAG-BDNFHA-WPRE-bGH (also referred to herein as OSU construct), which has been described in Cao et al., Molecular therapy of obesity and diabetes by a physiological autoregulatory approach, Nat Med. 2009 April; 15 (4): 447-54 and in U.S. Pat. No. 9,265,843. The codon optimization methods included combinations of (1) removal of rare codons having a usage frequency of less than 25% and replaced with more frequent codons based on the codon usage tables of top genes expressed in the putamen or other subregions of the brain, (2) CpG motif removal, and (3) removal of undesirable motifs (e.g., cryptic splice site, TLR9 activating motifs) and (4) introduction of desirable motifs (e.g., TLR9 inhibitory motifs) when possible. While some species-dependent effect was observed (N2A data vs. SH-SY5Y), increased protein expression was consistently observed with codon optimizations #1 and #3. See Table 5 for sequences. Therefore, codon-optimized genes BDNFco and BDNFco3 were used for further development (FIG. 2A).

Expression constructs BDNF-1 (comprising BDNFco) and BDNF-12 (comprising BDNFco3) showed significantly higher expression than the reference construct in both N2A cells (FIG. 2B) and in human neural progenitor cells (NPCs) (FIG. 2C). It was shown that the codon-optimized sequences have the same signaling activity (ERK1/2 phosphorylation) and, thus, functionality as the wild-type BDNF sequence found in the reference construct (FIG. 2D).

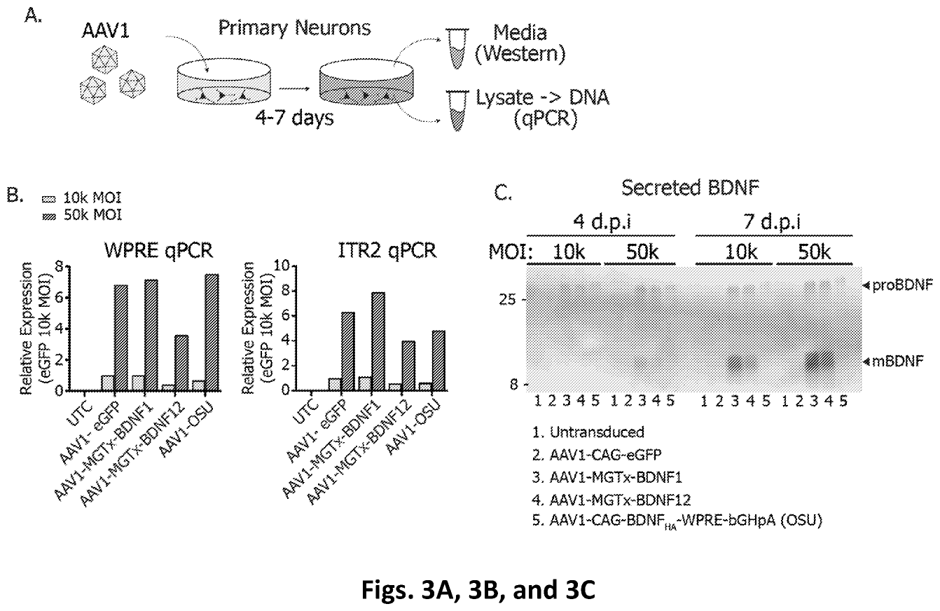

Next, BDNF-1 and BDNF-12 were tested in primary cortical mouse neurons using AAV2/1-mediated transduction (FIG. 3A). Equal transduction was achieved with across constructs (WPRE and ITR2 qPCR) (FIG. 3B). In agreement with the prior data from transiently transfected cells, expression constructs BDNF-1 and BDNF-12 had significantly higher expression of BDNF, as compared to the reference construct, as indicated by Western blot (FIG. 3C).

To test the expression and efficacy of the disclosed constructs in vivo, a high-fat diet (HFD)-induced obesity model in wild-type adult mice was used. Viruses were injected bilaterally into the hypothalamus of each mouse. Animals were placed on high-fat diet 7 days after surgery. Weights were recorded weekly, and tissue was collected 21 days post-surgery (FIG. 4A).

Expression E construct BDNF1 expressed over 1000-fold higher levels of BDNF precursor (proBDNF) (648.1±33.5 vs 0.6±0.1 ng/mg total protein) and 200-fold higher levels of mature BDNF (mBDNF) (260.6±20.7 vs 1.3±0.3 ng/mg total protein) than endogenous levels in the control diet group (FIG. 4C). Expression construct BDNF12 expressed over 400-fold higher levels of proBDNF (648.1±33.5 vs 0.6±0.1 ng/mg total protein) and 84-fold higher levels of mBDNF (260.6±20.7 vs 1.3±0.3 ng/mg total protein) than endogenous levels in the control diet group (FIG. 4C). Compared to the OSU reference construct, expression constructs BDNF-1 and BDNF-12, respectively, expressed 19.2-fold and 11.3-fold more mBDNF protein in the mouse hypothalamus (FIG. 4B).

BDNF over-expression causes a concentration-dependent decrease in body weight in wild-type animals. As shown in FIG. 4D, animals treated with the OSU reference construct (0.9±0.3 g) had less weight gain induced by 45% HFD than control groups (HFD: 4.9±0.4 g; AAV2/1-eGFP+HFD: 4.5±0.3 g) in wild-type mice. In contrast to the control groups and OSU reference group, mice treated with expression constructs- and BDNF-12, not only prevented HFD-induced weight gain, but also showed significant weight loss despite being on HFD (BDNF-1: −4.3±0.4 g; BDNF-12: −2.9±0.9 g; FIG. 4D).

While the foregoing written description of the invention enables one of ordinary skill to make and use what is considered presently to be the best mode thereof, those of ordinary skill will understand and appreciate the existence of variations, combinations, and equivalents of the specific embodiments, methods, and examples herein.

TABLE 1
Sequence elements of representative constructs.

| | | | | | | | Micro | |
|---|---|---|---|---|---|---|---|---|
| Construct | Altern. | | | | | | RNA | Plasmid |
| Name | name | Description | Promoter | Transgene | PTRE | polyA | seq. | Type |
| BDNF-1 | BDNF1 | C14-L21- | C14_L21 | BDNFco | WPRE | sPoly | 4xmir1 | pAAV |
| | | BDNFco- | | | 250 | A2 | 42- | |
| | | 4xmir142- | | | | | 4xmir1 | |
| | | 4xmir185- | | | | | 85 | |
| | | WPRE250- | | | | | | |
| | | sPolyA2 | | | | | | |
| BDNF-2 | BDNF2 | C14-L21- | C14_L21 | BDNFco | WPRE | sPoly | 4xmir1 | pAAV |
| | | BDNFco- | | | mut6 | A2 | 42- | |
| | | 4x4mir- | | | | | 4xmir1 | |
| | | WPREmut6- | | | | | 85- | |
| | | sPolyA2 | | | | | 4xmir1- | |
| | | | | | | | 4xmir1 | |
| | | | | | | | 22 | |
| BDNF-3 | BDNF3 | C14-L21- | C14_L21 | BDNFco | WPRE | sPoly | 4xmir1 | pAAV |
| | | BDNFco- | | | 3 | A2 | 42- | |
| | | 4x4mir- | | | | | 4xmir1 | |
| | | WPRE3- | | | | | 85- | |
| | | sPolyA2 | | | | | 4xmir1- | |
| | | | | | | | 4xmir1 | |
| | | | | | | | 22 | |
| BDNF-4 | BDNF4 | C14-L21- | C14_L21 | BDNFco | WPRE | sPoly | no | pAAV |
| | | BDNFco- | | | mut6 | A2 | | |
| | | WPREmut6- | | | | | | |
| | | sPolyA2 | | | | | | |
| BDNF-5 | BDNF5 | C14-L21- | C14_L21 | BDNFco | WPRE | sPoly | no | pAAV |
| | | BDNFco- | | | 3 | A2 | | |
| | | WPRE3- | | | | | | |
| | | sPolyA2 | | | | | | |
| BDNF-6 | BDNF6 | C14-L21- | C14_L21 | BDNFco | | sPoly | no | pAAV |
| | | BDNFco- | | | | A2 | | |
| | | sPolyA2 | | | | | | |
| BDNF-7 | BDNF7 | C14-L21- | C14_L21 | BDNFco | WPRE | sPoly | 4xmir1 | pAAV |
| | | BDNFco- | | | mut6 | A2 | 42 | |
| | | 4xmir142- | | | | | | |
| | | WPREmut6- | | | | | | |
| | | sPolyA2 | | | | | | |
| BDNF-8 | BDNF8 | C14-L21- | C14_L21 | BDNF | WPRE | sPoly | no | pAAV |
| | | BDNF- | | | mut6 | A2 | | |
| | | WPREmut6- | | | | | | |
| | | sPolyA2 | | | | | | |
| BDNF-9 | BDNF9 | C14-L21- | C14_L21 | BDNFco2 | WPRE | sPoly | no | pAAV |
| | | BDNFco2- | | | mut6 | A2 | | |
| | | WPREmut6- | | | | | | |
| | | sPolyA2 | | | | | | |
| BDNF-10 | BDNF1 | C14-L21- | C14_L21 | BDNFco3 | WPRE | sPoly | no | pAAV |
| | 0 | BDNFco3- | | | mut6 | A2 | | |
| | | WPREmut6- | | | | | | |
| | | sPolyA2 | | | | | | |
| BDNF-11 | BDNF1 | C14-L21- | C14_L21 | BDNFco4 | WPRE | sPoly | no | pAAV |
| | 1 | BDNFco4- | | | mut6 | A2 | | |
| | | WPREmut6- | | | | | | |
| | | sPolyA2 | | | | | | |
| BDNF-12 | BDNF1 | C14-L21- | C14_L21 | BDNFco3 | WPRE | sPoly | 4xmir1 | pAAV |
| | 2 | BDNFco3- | | | 3 | A2 | 42- | |
| | | 4x4mir- | | | | | 4xmir1 | |
| | | WPRE3- | | | | | 85- | |
| | | sPolyA2 | | | | | 4xmir1- | |
| | | | | | | | 4xmir1 | |
| | | | | | | | 22 | |
| BDNF-13 | BDNF1 | C14-L21- | C14_L21 | BDNFco3 | WPRE | bGH | 4xmir1 | pAAV |
| | 3 | BDNFco3- | | | 3 | | 42- | |
| | | 4x4mir- | | | | | 4xmir1 | |
| | | WPRE3- | | | | | 85- | |
| | | bGHpA | | | | | 4xmir1- | |
| | | | | | | | 4xmir1 | |
| | | | | | | | 22 | |
| BDNF-14 | BDNF1 | C14-L21- | C14_L21 | BDNFco3 | WPRE | hGH | 4xmir1 | pAAV |
| | 4 | BDNFco3- | | | 3 | | 42- | |
| | | 4x4mir- | | | | | 4xmir1 | |
| | | WPRE3- | | | | | 85- | |
| | | hGHpA | | | | | 4xmir1- | |
| | | | | | | | 4xmir1 | |
| | | | | | | | 22 | |
| BDNF-15 | BDNF1 | C14-L21- | C14_L21 | BDNFco3 | WPRE | sv40 | 4xmir1 | pAAV |
| | 5 | BDNFco3- | | | 3 | | 42- | |
| | | 4x4mir- | | | | | 4xmir1 | |
| | | WPRE3- | | | | | 85- | |
| | | SV40pA | | | | | 4xmir1- | |
| | | | | | | | 4xmir1 | |
| | | | | | | | 22 | |
| BDNF-16 | BDNF1 | C14-L21- | C14_L21 | BDNFco3 | WPRE | sv40p | 4xmir1 | pAAV |
| | 6 | BDNFco3- | | | 3 | a2 | 42- | |
| | | 4x4mir- | | | | | 4xmir1 | |
| | | WPRE3- | | | | | 85- | |
| | | SV40pA2 | | | | | 4xmir1- | |
| | | | | | | | 4xmir1 | |
| | | | | | | | 22 | |
| BDNF-17 | BDNF1 | B36- | B36 | BDNFco3 | WPRE | sPoly | 4xmir1 | pAAV |
| | 7 | BDNFco3- | | | 3 | A2 | 42- | |
| | | 4x4mir- | | | | | 4xmir1 | |
| | | WPRE3- | | | | | 85- | |
| | | sPolyA2 | | | | | 4xmir1- | |
| | | | | | | | 4xmir1 | |
| | | | | | | | 22 | |
| BDNF-18 | BDNF1 | B46- | B46 | BDNFco3 | WPRE | sPoly | 4xmir1 | pAAV |
| | 8 | BDNFco3- | | | 3 | A2 | 42- | |
| | | 4x4mir- | | | | | 4xmir1 | |
| | | WPRE3- | | | | | 85- | |
| | | sPolyA2 | | | | | 4xmir1- | |
| | | | | | | | 4xmir1 | |
| | | | | | | | 22 | |
| BDNF-19 | BDNF1 | B47- | B47 | BDNFco3 | WPRE | sPoly | 4xmir1 | pAAV |
| | 9 | BDNFco3- | | | 3 | A2 | 42- | |
| | | 4x4mir- | | | | | 4xmir1 | |
| | | WPRE3- | | | | | 85- | |
| | | sPolyA2 | | | | | 4xmir1- | |
| | | | | | | | 4xmir1 | |
| | | | | | | | 22 | |
| BDNF-20 | BDNF2 | B48- | B48 | BDNFco3 | WPRE | sPoly | 4xmir1 | pAAV |
| | 0 | BDNFco3- | | | 3 | A2 | 42- | |
| | | 4x4mir- | | | | | 4xmir1 | |
| | | WPRE3- | | | | | 85- | |
| | | sPolyA2 | | | | | 4xmir1- | |
| | | | | | | | 4xmir1 | |
| | | | | | | | 22 | |
| BDNF-21 | BDNF2 | CAG-BDNF- | CAG | BDNF-HA | WPRE | bGH | | pAAV |
| | 1 | HA- | (OSU) | | mut6 | | | |
| | | WPREmut6- | | | | | | |
| | | bHGpA | | | | | | |
| BDNF-22 | BDNF2 | CAG-BDNF- | CAG | BDNF | WPRE | bGH | | pAAV |
| | 2 | WPREmut6- | (OSU) | | mut6 | | | |
| | | bHGpA | | | | | | |
| BDNF-23 | BDNF2 | CAG- | CAG | BDNFco3 | WPRE | bGH | | pAAV |
| | 3 | BDNFco3- | (OSU) | | mut6 | | | |
| | | WPREmut6- | | | | | | |
| | | bHGpA | | | | | | |
| BDNF-24 | BDNF2 | CAG- | CAG | BDNFco3 | WPRE | bGH | 4xmir1 | pAAV |
| | 4 | BDNFco3- | (OSU) | | mut6 | | 42 | |
| | | 4xmir142- | | | | | | |
| | | WPREmut6- | | | | | | |
| | | bHGpA | | | | | | |
| C42b- | BDNF- | | C42b | BDNF-HA | WPRE | bGH | | pUC57 |
| BDNF-HA | A | | | | | | | mini |
| C42b- | BDNF- | | C42b | mBDNF_H | WPRE | bGH | | pUC57 |
| mBDNF- | B | | | A | | | | mini |
| HA | | | | | | | | |
| C42b-IL2- | BDNF- | | C42b | mIL2_tdB | WPRE | bGH | | pUC57 |
| tdBDNF- | E | | | DNF_HA | | | | mini |
| HA | | | | | | | | |
| Gaussia- | BDNF- | | C42b_L2 | Gaussia- | WPRE | bGH | | pUC57 |
| BDNF-HA | F | | 1 | BDNF-HA | | | | mini |
| mIL2- | BDNF- | | C42b_L2 | mIL2_BD | WPRE | bGH | | pUC57 |
| BDNF-HA | H | | 1 | NF_HA | | | | mini |
| Secrecon2- | BDNF- | | C42b_L2 | Secrecon2- | WPRE | bGH | | pUC57 |
| BDNF-HA | J | | 1 | BDNF-HA | | | | mini |
| C42b-S2b- | BDNF- | | C42b_L2 | S2b- | WPRE | bGH | | pUC57 |
| BDNF-HA | L | | 1 | BDNF-HA | | | | mini |
| C14- | BDNF- | C14-L21- | C14_L21 | BDNF-HA | WPRE | bGH | | pUC57 |
| BDNF-HA | N | BDNF-HA- | | | | | | mini |
| | | WPREmut6- | | | | | | |
| | | bGH | | | | | | |
| C26- | BDNF- | C26-L21- | C26_L21 | BDNF-HA | WPRE | bGH | | pUC57 |
| BDNF-HA | O | BDNF-HA- | | | | | | mini |
| | | WPREmut6- | | | | | | |
| | | bGH | | | | | | |
| C42b-L21- | BDNF- | C42b-L21- | C42b_L2 | BDNF-HA | WPRE | bGH | | pUC57 |
| BDNF-HA | P | BDNF-HA | 1 | | | | | mini |
| C46- | BDNF- | C46-L21- | C46_L21 | BDNF-HA | WPRE | bGH | | pUC57 |
| BDNF-HA | Q | BDNF-HA | | | | | | mini |
| C42b_BD | BDNF- | C42b_BDNF_{—} | C42b | BDNF- | WPRE | bGH | | pUC57 |
| NF_furin_{—} | R | furin_F2A | | furin-F2A- | | | | mini |
| F2A | | | | BDNF | | | | |
| C42b_BD | BDNF- | C42b_BDNF_{—} | C42b | BDNF- | WPRE | bGH | | pUC57 |
| NF_furin_{—} | S | furin_P2A | | furin-P2A- | | | | mini |
| P2A | | | | BDNF | | | | |
| C42b_BD | BDNF- | C42b_BDNF_{—} | C42b | BDNF- | WPRE | bGH | | pUC57 |
| NF_furin_{—} | T | furin_T2A | | furin-T2A- | | | | mini |
| T2A | | | | BDNF | | | | |
| C42b_BD | BDNF- | C42b_BDNF- | C42b | BDNF- | WPRE | bGH | | pUC57 |
| NF- | U | HA_furin_P2 | | HA-furin- | | | | mini |
| HA_furin_{—} | | A | | P2A- | | | | |
| P2A | | | | BDNF | | | | |
| C42b_BD | BDNF- | C42b_BDNF_{—} | C42b | BDNF (2x) | WPRE | bGH | | pUC57 |
| NF_IRES_{—} | V | IRES_BDNF_{—} | | * | 3 | | | mini |
| BDNF_WP | | WPRE3 | | | | | | |
| RE3 | | | | | | | | |

* This construct comprises two copies of an BDNF encoding sequence (SEQ ID NO: 57), separated by an IRES sequence.
Seq: sequence.

TABLE 2
Sequence elements (from 5′ to 3′) of selected,
representative constructs, with SEQ ID NOs.

| | SEQ |
|---|---|
| Vector & Elements | ID NO |
| BDNF1 | 11 |
| ITR (5′) | 51 |
| C14_L21 | 1 |
| BDNFco CDS | 52 |
| WPRE250 | 58 |
| sPolyA2 | 62 |
| mirBS₍₄ₓₘᵢᵣ₁₄₂₋₄ₓₘᵢᵣ₁₈₅₎ | 63 |
| ITR (3′) | 65 |
| BDNF12 | 22 |
| ITR (5′) | 51 |
| C14_L21 | 1 |
| BDNFco3 CDS | 53 |
| WPRE3 | 59 |
| sPolyA2 | 62 |
| mirBS₍₄ₓₘᵢᵣ₁₄₂₋₄ₓₘᵢᵣ₁₈₅₋₄ₓₘᵢᵣ₁₋₄ₓₘᵢᵣ₁₂₂₎ | 64 |
| ITR (3′) | 65 |
| BDNF8 | 18 |
| ITR (5′) | 51 |
| C14_L21 | 1 |
| BDNF CDS | 57 |
| WPREmut6 | 60 |
| sPolyA2 | 62 |
| ITR (3′) | 65 |
| BDNF9 | 19 |
| ITR (5′) | 51 |
| C14_L21 | 1 |
| BDNFco2 CDS | 54 |
| WPREmut6 | 60 |
| sPolyA2 | 62 |
| ITR (3′) | 65 |
| BDNF10 | 20 |
| ITR (5′) | 51 |
| C14_L21 | 1 |
| BDNFco3 CDS | 53 |
| WPREmut6 | 60 |
| sPolyA2 | 62 |
| ITR (3′) | 65 |
| BDNF11 | 21 |
| ITR (5′) | 51 |
| C14_L21 | 1 |
| BDNFco4 CDS | 55 |
| WPREmut6 | 60 |
| sPolyA2 | 62 |
| ITR (3′) | 65 |
| BDNF17 | 27 |
| ITR (5′) | 51 |
| B36 | 2 |
| BDNFco3 CDS | 53 |
| WPRE3 | 59 |
| sPolyA2 | 62 |
| mirBS₍₄ₓₘᵢᵣ₁₄₂₋₄ₓₘᵢᵣ₁₈₅₋₄ₓₘᵢᵣ₁₋₄ₓₘᵢᵣ₁₂₂₎ | 64 |
| ITR (3′) | 65 |
| BDNF18 | 28 |
| ITR (5′) | 51 |
| B46 | 3 |
| BDNFco3 CDS | 53 |
| WPRE3 | 59 |
| sPolyA2 | 62 |
| mirBS₍₄ₓₘᵢᵣ₁₄₂₋₄ₓₘᵢᵣ₁₈₅₋₄ₓₘᵢᵣ₁₋₄ₓₘᵢᵣ₁₂₂₎ | 64 |
| ITR (3′) | 65 |
| BDNF19 | 29 |
| ITR (5′) | 51 |
| B47 | 4 |
| BDNFco3 CDS | 53 |
| WPRE3 | 59 |
| sPolyA2 | 62 |
| mirBS₍₄ₓₘᵢᵣ₁₄₂₋₄ₓₘᵢᵣ₁₈₅₋₄ₓₘᵢᵣ₁₋₄ₓₘᵢᵣ₁₂₂₎ | 64 |
| ITR (3′) | 65 |
| BDNF20 | 30 |
| ITR (5′) | 51 |
| B48 | 5 |
| BDNFco3 CDS | 53 |
| WPRE3 | 59 |
| sPolyA2 | 62 |
| mirBS₍₄ₓₘᵢᵣ₁₄₂₋₄ₓₘᵢᵣ₁₈₅₋₄ₓₘᵢᵣ₁₋₄ₓₘᵢᵣ₁₂₂₎ | 64 |
| ITR (3′) | 65 |
| C14-BDNF-HA | 42 |
| ITR (5′) | Not |
| | applicable |
| C14_L21 | 1 |
| BDNF-HA CDS | 56 |
| WPRE | 61 |
| bGH | 66 |
| ITR (3′) | Not |
| | applicable |
| C26-BDNF-HA | 43 |
| ITR (5′) | Not |
| | applicable |
| C26_L21 | 9 |
| BDNF-HA CDS | 56 |
| WPRE | 61 |
| bGH | 66 |
| ITR (3′) | Not |
| | applicable |
| C42b-L21-BDNF-HA | 44 |
| ITR (5′) | Not |
| | applicable |
| C42b_L21 | 8 |
| BDNF-HA CDS | 56 |
| WPRE | 61 |
| bGH | 66 |
| ITR (3′) | Not |
| | applicable |
| C46-BDNF-HA | 45 |
| ITR (5′) | Not |
| | applicable |
| C46_L21 | 10 |
| BDNF-HA CDS | 56 |
| WPRE | 61 |
| bGH | 66 |
| ITR (3′) | Not |
| | applicable |

TABLE 3
Overview of promoter sequences.

| SEQ | |
|---|---|
| ID NO | Name |
| 1 | C14_L21 |
| 2 | B36 |
| 3 | B46 |
| 4 | B47 |
| 5 | B48 |
| 6 | CAG (OSU) |
| 7 | C42b |
| 8 | C42b_L21 |
| 9 | C26_L21 |
| 10 | C46_L21 |

See Table 6 for sequences.

TABLE 4
Overview of expression construct sequences.

| SEQ | |
|---|---|
| ID NO | Name |
| 11 | BDNF1 |
| 12 | BDNF2 |
| 13 | BDNF3 |
| 14 | BDNF4 |
| 15 | BDNF5 |
| 16 | BDNF6 |
| 17 | BDNF7 |
| 18 | BDNF8 |
| 19 | BDNF9 |
| 20 | BDNF10 |
| 21 | BDNF11 |
| 22 | BDNF12 |
| 23 | BDNF13 |
| 24 | BDNF14 |
| 25 | BDNF15 |
| 26 | BDNF16 |
| 27 | BDNF17 |
| 28 | BDNF18 |
| 29 | BDNF19 |
| 30 | BDNF20 |
| 31 | BDNF21 |
| 32 | BDNF22 |
| 33 | BDNF23 |
| 34 | BDNF24 |
| 35 | BDNF-A |
| 36 | BDNF-B |
| 37 | BDNF-E |
| 38 | BDNF-F |
| 39 | BDNF-H |
| 40 | BDNF-J |
| 41 | BDNF-L |
| 42 | BDNF-N |
| 43 | BDNF-O |
| 44 | BDNF-P |
| 45 | BDNF-Q |
| 46 | BDNF-R |
| 47 | BDNF-S |
| 48 | BDNF-T |
| 49 | BDNF-U |
| 50 | BDNF-V |

See Table 8 for sequences.

TABLE 5
Overview of additional sequences.

| SEQ | | |
|---|---|---|
| ID NO | Type | Vector & Elements |
| 51 | ITR | ITR (5′) |
| 52 | BDNF | BDNFco CDS |
| | encoding sequence | |
| 53 | BDNF | BDNFco3 CDS |
| | encoding sequence | |
| 54 | BDNF | BDNFco2 CDS |
| | encoding sequence | |
| 55 | BDNF | BDNFco4 CDS |
| | encoding sequence | |
| 56 | BDNF | BDNF-HA CDS |
| | encoding sequence | |
| 57 | BDNF | BDNF CDS |
| | encoding sequence | |
| 58 | PTRE | WPRE250 |
| 59 | PTRE | WPRE3 |
| 60 | PTRE | WPREmut6 |
| 61 | PTRE | WPRE |
| 62 | polyA | sPolyA2 |
| 63 | microRNA site | mirBS₍₄ₓₘᵢᵣ₁₄₂₋₄ₓₘᵢᵣ₁₈₅₎ |
| 64 | microRNA site | mirBS₍₄ₓₘᵢᵣ₁₄₂₋₄ₓₘᵢᵣ₁₈₅₋₄ₓₘᵢᵣ₁₋₄ₓₘᵢᵣ₁₂₂₎ |
| 65 | ITR | ITR (3′) |
| 66 | polyA | bGH |
| 67 | polyA | hGH |
| 68 | polyA | sv40 |
| 69 | polyA | sv40pa2 |
| 70 | BDNF | mBDNF_HA |
| | encoding sequence | |
| 71 | BDNF | mIL2_tdBDNF_HA |
| | encoding sequence | |
| 72 | BDNF | Gaussia-BDNF-HA |
| | encoding sequence | |
| 73 | BDNF | mIL2_BDNF_HA |
| | encoding sequence | |
| 74 | BDNF | Secrecon2-BDNF-HA |
| | encoding sequence | |
| 75 | BDNF | S2b-BDNF-HA |
| | encoding sequence | |
| 76 | BDNF | BDNF-furin-F2A-BDNF |
| | encoding sequence | |
| 77 | BDNF | BDNF-furin-P2A-BDNF |
| | encoding sequence | |
| 78 | BDNF | BDNF-furin-T2A-BDNF |
| | encoding sequence | |
| 79 | BDNF | BDNF-HA-furin-P2A-BDNF |
| | encoding sequence | |
| 80 | | IRES |
| 81 | | BDNF |
| | | protein sequence |

ITR = inverted terminal repeat. PTRE = post-transcriptional regulatory element. polyA = polyadenylation sequence. IRES = internal ribosome entry site.
See Table 7 for sequences.

TABLE 6
Promoter sequences.

| SEQ ID NO | Name | Sequence |
|---|---|---|
| 1 | C14_L21 | GAGTAATTCATACAAAAGGACTCGCCCCTGCCTTGGGGAATCCCAGGG |
| | | ACCGTCGTTAAACTCCCACTAACGTAGAACCCAGAGATCGCTGCGTTC |
| | | CCGCCCCCTCACCCGCCCGCTCTCGTCATCACTGAGGTGGAGAAGAGC |
| | | ATGCGTGAGGCTCCGGTGCCCGTCAGTGGGCAGAGCGCACATCGCCCA |
| | | CAGTCCCCGAGAAGTTGGGGGGAGGGGTCGGCAATTGAACCGGTGCC |
| | | TAGAGAAGGTGGCGCGGGGTAAACTGGGAAAGTGATGTCGTGTACTG |
| | | GCTCCGCCTTTTTCCCGAGGGTGGGGGAGAACCGTATATAAGTGCGTA |
| | | GTCGCCGTGAACGTTCTTTTTCGCAACGGGCTCGAGCCGAGTGGTCGT |
| | | TGCCGCCAGAACACAGGTAAGTGCCGTGTGTGGTTCCCGCGGGCCTGG |
| | | CCTCTTTACGGGTTATGGCCCTTGCGTGCCTTGAATTACTTCCACGCCC |
| | | CTGGCTGCAGTACGTGATTCTTGATCCCGAGCTTCGGGTTGGAAGTGG |
| | | GTGGGAGAGTTCGAGGCCTTGCGCTTAAGGAGCCCCTTCGCCTCGTGC |
| | | TTGAGTTGAGGCCTGGCTTGGGCGCTGGGGCCGCCGCGTGCGAATCTG |
| | | GTGGCACCTTCGCGCCTGTCTCGCTGCTTTCGATAAGTCTCTAGCCATT |
| | | TAAAATTTTTGATGACCTGCTGCGACGCTTTTTTTCTGGCAAGATAGTC |
| | | TTGTAAATGCGGGCCAAGATCTGCACACTGGTATTTCGGTTTTTGGGG |
| | | CCGCGGGCGGCGACGGGGCCCGTGCGTCCCAGCGCACATGTTCGGCG |
| | | AGGCGGGGCCTGCGAGCGCGGCCACCGAGAATCGGACGGGGGTAGTC |
| | | TCAAGCTGGCCGGCCTGCTCTGGTGCCTGGCCTCGCGCCGCCGTGTAT |
| | | CGCCCCGCCCTGGGCGGCAAGGCTGGCCCGGTCGGCACCAGTTGCGTG |
| | | AGCGGAAAGATGGCCGCTTCCCGGCCCTGCTGCAGGGAGCTCAAAAT |
| | | GGAGGACGCGGCGCTCGGGAGAGCGGGCGGGTGAGTCACCCACACAA |
| | | AGGAAAAGGGCCTTTCCGTCCTCAGCCGTCGCTTCATGTGACTCCACG |
| | | GAGTACCGGGCGCCGTCCAGGCACCTCGATTAGTTCTCGAGCTTTTGG |
| | | AGTACGTCGTCTTTAGGTTGGGGGGAGGGGTTTTATGCGATGGAGTTT |
| | | CCCCACACTGAGTGGGTGGAGACTGAAGTTAGGCCAGCTTGGCACTTG |
| | | ATGTAATTCTCCTTGGAATTTGCCCTTTTTGAGTTTGGATCTTGGTTCAT |
| | | TCTCAAGCCTCAGACAGTGGTTCAAAGTTTTTTTCTTCCATTTCAGGTG |
| | | TCGTGAGGATCCAACTCCTAAAAAACCGCCACC |
| 2 | B36 | GCCGGGCTGGTAGCCCAGCGGGGGGCGCGGCAGGGCGAGGAGCAGCC |
| | | CGCGCTGCTCCCGCCCCTCGGGTGCCAGCACCGCCCCTGCTGCGGCGG |
| | | GTGAGGGGCGGGGCGGGGCGCGGCGTATATAAGGCTAGGGGGGGGCG |
| | | CCGCTCTTTTGTTTCTTGCTGCAGCAACGCGAGTGGGAGCACCAGGAT |
| | | CTCGGGCTCGGAACGAGACTGCACGGTGAGTGCGGCGCCGGGGGGGG |
| | | GGGCCCACCCAGGGTGTGGTCGGATCCGGTGCACCGGGCGGGCGCGC |
| | | CGCAACCGCGACAGGCGCCCTTCTCGGACCGGACGCAGGGGCCGGCG |
| | | ACCACGCCCTGGGACCGAGAAGAGGGGTGCGGGACGCGCCCAGATCC |
| | | TCGGCCTTGGGGCTGCTCGGCACGCCTTGGCGCGAGTGCCACGTCGAG |
| | | AGGCGTCGGCGGGGAGCGCGGAAGGGGACGCTGGCCCCCAGGCCCAG |
| | | GTCAAGCGCCTTGGTTTGCCCACTAGGATTGTTTTAAGAAAAGGATCC |
| | | GCCACC |
| 3 | B46 | TGTCCCACCTCGTTAGAGAAAACAGCGATGCTCAAAGGCAACCTCCTT |
| | | CCTGACATTGCCTGGTAGGACGCGACGTGGTGTTTGCCCGCGCGGAAT |
| | | GCGGACGCAAGGCTGCTCCTAGGTCTCGGGGACGCGCCATCCCCATTT |
| | | CCGCTCGCGGAGGCGTAGGGTCCGGGCGCGGGACCCCAGTCGACCTTG |
| | | ACTGGCGGCGCGACCTTGAGGCCTGCGTTCGCCTCAGTTGCCCCCTCT |
| | | GTGCAATGGGGAGACGCCTGCCGCCCCGCCCCGAGTCCCATGCCGCAG |
| | | CCACCGCGACGGAGCCCGCAGGCGGGAACCTGCCTCCGCGCGTTAGC |
| | | GCGCACGCGCGCCTCATGTGTCGTCCCCATCAGCGCCGGCTTCCGTCT |
| | | ATAGGCCAGATGCACTGTCACTCTGGCGAAGTCGCAGACCCGATTGGC |
| | | CGGGACGGAGGCGCGAGACCGGGTTGCGGGCGGGGCCGAACGTGGTA |
| | | TAAAAGGGGGGGGAGGCCAGGCTCGTGTCAGTCTGCAGACGCCACCC |
| | | GAGCCGAGTGGTCGTTACTATTAGCCGGATCCGCCACC |
| 4 | B47 | CCCCGGATGCTCACCTCCCCGACTCGCCCCCGCTGTGGCCCCGCCCCC |
| | B48 | GCGCGGCTCTTCGTGCCACGTCACCGCCTGCGTCGCTTCCGGAGGCGC |
| | | AGCGGGCGATGACGTAGAGGGACGTGCCCTCTATATGAGGTTGGGGA |
| | | GCGGCTGAGTCGGTCAGTCCGCCTGGAGACCTCGAGCCGAGTGGTCGT |
| | | CCGCCCGCTCCCCCCTCCCCCCGAGCGCCGCTCCGGCTGCACCGCGCT |
| | | CGCTCCGAGTTTCAGGCTCGTGCTAAGCTAGCGCCGTCGTCGTCTCCCT |
| | | TCAGTCGCCATCGGATCCGCCACC |
| 5 | CAG | TGTCCCACCTCGTTAGAGAAAACAGCGATGCTCAAAGGCAACCTCCTT |
| | | CCTGACATTGCCTGGTAGGACGCGACGTGGTGTTTGCCCGCGCGGAAT |
| | | GCGGACGCAAGGCTGCTCCTAGGTCTCGGGGACGCGCCATCCCCATTT |
| | | CCGCTCGCGGAGGCGTAGGGTCCGGGCGCGGGACCCCAGTCGACCTTG |
| | | ACTGGCGGCGCGACCTTGAGGCCTGCGTTCGCCTCAGTTGCCCCCTCT |
| | | GTGCAATGGGGAGACGCCTGCCGCCCCGCCCCGAGTCCCATGCCGCAG |
| | | CCACCGCGACGGAGCCCGCAGGCGGGAACCTGCCTCCGCGCGTTAGC |
| | | GCGCACGCGCGCCTCATGTGTCGTCCCCATCAGCGCCGGCTTCCGTCT |
| | | ATAGGCCAGATGCACTGTCACTCTGGCGAAGTCGCAGACCCGATTGGC |
| | | CGGGACGGAGGCGCGAGACCGGGTTGCGGGCGGGGCCGAACGTGGTA |
| | | TAAAAGGGGGGGGAGGCCAGGCTCGTGTCAGTCTGCAGACGCCACCC |
| | | GAGCCGAGTGGTCGTTACTATTAGCCGCCGGGCTGGTAGCCCAGCGGG |
| | | GGGCGCGGCAGGGCGAGGAGCAGCCCGCGCTGCTCCCGCCCCTCGGG |
| | | TGCCAGCACCGCCCCTGCTGCGGCGGGTGAGGGGCGGGGCGGGGCGC |
| | | GGCGTATATAAGGCTAGGGGGGGGCGCCGCTCTTCAGTCCGCCTGGAG |
| | | ACCTCGAGCCGAGTGGTCGTCAGGATCTCGGGCTCGGAACGAGACTGC |
| | | ACGGTGAGTGCGGCGCCGGGGCGGGGGGCCCACCCAGGGTGTGGTCG |
| | | GATCCGGTGCACCGGGCGGGCGCGCCGCAACCGCGACAGGCGCCCTT |
| | | CTCGGACCGGACGCAGGGGCCGGCGACCACGCCCTGGGACCGAGAAG |
| | | AGGGGTGCGGGACGCGCCCAGATCCTCGGCCTTGGGGCTGCTCGGCAC |
| | | GCCTTGGCGCGAGTGCCACGTCGAGAGGCGTCGGGGGGAGCGCGGA |
| | | AGGGGACGCTGGCCCCCAGGCCCAGGTCAAGCGCCTTGGTTTGCCCAC |
| | | TAGGATTGTTTTAAGAAAAGGATCCGCCACC |
| 6 | (OSU) | GACATTGATTATTGACTAGTTATTAATAGTAATCAATTACGGGGTCATT |
| | | AGTTCATAGCCCATATATGGAGTTCCGCGTTACATAACTTACGGTAAA |
| | | TGGCCCGCCTGGCTGACCGCCCAACGACCCCCGCCCATTGACGTCAAT |
| | | AATGACGTATGTTCCCATAGTAACGCCAATAGGGACTTTCCATTGACG |
| | | TCAATGGGTGGAGTATTTACGGTAAACTGCCCACTTGGCAGTACATCA |
| | | AGTGTATCATATGCCAAGTACGCCCCCTATTGACGTCAATGACGGTAA |
| | | ATGGCCCGCCTGGCATTATGCCCAGTACATGACCTTATGGGACTTTCCT |
| | | ACTTGGCAGTACATCTACGTATTAGTCATCGCTATTACCATGGGTCGA |
| | | GGTGAGCCCCCGTTCTGCTTCACTCTCCCCATCTCCCCCCCCTCCCCAC |
| | | CCCCAATTTTGTATTTATTTATTTTTTAATTATTTTGTGCAGCGATGGGG |
| | | GCGGGGGGGGGGGGGGGGGGGGGGGGGGGTGGGGCGGAGGGTGGCA |
| | | AGGGGCGGGGCGGGGCAAGGCGGAAAGGTGCGGCGGCAGCCAATCA |
| | | AAGCGGCGCCCTCCAAAAGTTTCCTTTTATGGCAAGGCGGCGGCGGCG |
| | | GCGGCCCTATAAAAAGCGAAGCGCGCGGCGGGCGGGAGTCGCTGCGT |
| | | TGCCTTGGGTCCTCCCCCCCTCTCCAGCCGCCTCGCGCCGCCCGCCCCG |
| | | GCTCTGACTGACCGCGTTACTCCCACAGGTGAGCGGGCGGGACGGCCC |
| | | TTCTCCTCCGGGCTGTAATTAGCGCTTGGTTTAATGACGGCTTGTTTCT |
| | | TTTCTGTGGCTGCGTGAAAGCCTTGAGGGGCTCCGGGAGGGCCCTTTG |
| | | TGCGGGGGGAGCGGCTCGGGGCTGTCCGCGGGGGGACGGCTGCCTTC |
| | | GGGGGGGACGGGGCAGGGCGGGGTTCGGCTTCTGGCGTGTGACCGGC |
| | | GGCTCTAGAGCCTCTGCTAACCATGTTCATGCCTTCTTCTTTTTCCTAC |
| | | AGCTCCTGGGCAACGTGCTGGTTATTGTGCTGTCTCATCATTTTGGCAA |
| | | AGAATTGGATCCACTCGAGTGGAGCTCGCGACTAGTCGATTCGAATTC |
| | | GGCTTGTG |
| 7 | C42b | CTGTGGAATGTGTGTCAGTTAGGGTGTGGAAAGTCCCCAGGCTCCCCA |
| | | GCAGGCAGAAGTATGCAAAGCATGCATCTCAATTAGTCAGCAACCAG |
| | | GTGTGGAAAGTCCCCAGGCTCCCCAGCAGGCAGAAGTATGCAAAGCA |
| | | TGCATCTCAATTAGTCAGCAACCATAGTCCCGCCCCTAACTCCGCCCAT |
| | | CCCGCCCCTAACTCCGCCCAGGACATTGATTATTGACTAGTTATTAATA |
| | | GTAATCAATTACGGGGTCATTAGTTCATAGCCCATATATGGAGTTCCG |
| | | CGTTACATAACTTACGGTAAATGGCCCGCCTGGCTGACCGCCCAACGA |
| | | CCCCCGCCCATTGACGTCAATAATGACGTATGTTCCCATAGTAACGCC |
| | | AATAGGGACTTTCCATTGACGTCAATGGGTGGACTATTTACGGTAAAC |
| | | TGCCCACTTGGCAGTACATCAAGTGTATCATATGCCAAGTACGCCCCC |
| | | TATTGACGTCAATGACGGTAAATGGCCCGCCTGGCATTATGCCCAGTA |
| | | CATGACCTTATGGGACTTTCCTACTTGGCAGTACATCTACGTATTAGTC |
| | | ATCGCTATTACCATGGTGATGCGGTTTTGGCAGTACATCAATGGGCGT |
| | | GGATAGCGGTTTGACTCACGGGGATTTCCAAGTCTCCACCCCATTGAC |
| | | GTCAATGGGAGTTTGTTTTGGCACCAAAATCAACGGGACTTTCCAAAA |
| | | TGTCGTAACAACTCCGCCCCATTGACGCAAATGGGCGGTAGGCGTGTA |
| | | CGGTGGGAGGTCTATATAAGCAGAGCTCGTTTAGTGAACCGTCAGATC |
| | | GCCTGGAGACCTCGAGCCGAGTGGTCGTGCCTCCATAGAAGACACCGG |
| | | GACCGAGGTAAGTTTAAAGCTCAGGTCGAGACCGGGCCTTTGTCCGGC |
| | | GCTCCCTTGGAGCCTACCTAGACTCAGCCGGCTCTCCACGCTTTGCCTG |
| | | ACCCTGCTTGCTCAACTCTAGTTCTCTCGTTAACTTAATGAGACAGATA |
| | | GAAACTGGTCTTGTAGAAACAGAGTAGTCGCCTGCTTTTCTGCCAGGT |
| | | GCTGACTTCTCTCCCCTGGGCTTTTTTCTTTTTCTCAGGTTGAAAAGAA |
| | | GAAGACGAAGAAGACGAAGAAGACAAACCGTCGGATCCGCCACC |
| 8 | C42b_L21 | CTGTGGAATGTGTGTCAGTTAGGGTGTGGAAAGTCCCCAGGCTCCCCA |
| | | GCAGGCAGAAGTATGCAAAGCATGCATCTCAATTAGTCAGCAACCAG |
| | | GTGTGGAAAGTCCCCAGGCTCCCCAGCAGGCAGAAGTATGCAAAGCA |
| | | TGCATCTCAATTAGTCAGCAACCATAGTCCCGCCCCTAACTCCGCCCAT |
| | | CCCGCCCCTAACTCCGCCCAGGACATTGATTATTGACTAGTTATTAATA |
| | | GTAATCAATTACGGGGTCATTAGTTCATAGCCCATATATGGAGTTCCG |
| | | CGTTACATAACTTACGGTAAATGGCCCGCCTGGCTGACCGCCCAACGA |
| | | CCCCCGCCCATTGACGTCAATAATGACGTATGTTCCCATAGTAACGCC |
| | | AATAGGGACTTTCCATTGACGTCAATGGGTGGACTATTTACGGTAAAC |
| | | TGCCCACTTGGCAGTACATCAAGTGTATCATATGCCAAGTACGCCCCC |
| | | TATTGACGTCAATGACGGTAAATGGCCCGCCTGGCATTATGCCCAGTA |
| | | CATGACCTTATGGGACTTTCCTACTTGGCAGTACATCTACGTATTAGTC |
| | | ATCGCTATTACCATGGTGATGCGGTTTTGGCAGTACATCAATGGGCGT |
| | | GGATAGCGGTTTGACTCACGGGGATTTCCAAGTCTCCACCCCATTGAC |
| | | GTCAATGGGAGTTTGTTTTGGCACCAAAATCAACGGGACTTTCCAAAA |
| | | TGTCGTAACAACTCCGCCCCATTGACGCAAATGGGCGGTAGGCGTGTA |
| | | CGGTGGGAGGTCTATATAAGCAGAGCTCGTTTAGTGAACCGTCAGATC |
| | | GCCTGGAGACCTCGAGCCGAGTGGTCGTGCCTCCATAGAAGACACCGG |
| | | GACCGAGGTAAGTTTAAAGCTCAGGTCGAGACCGGGCCTTTGTCCGGC |
| | | GCTCCCTTGGAGCCTACCTAGACTCAGCCGGCTCTCCACGCTTTGCCTG |
| | | ACCCTGCTTGCTCAACTCTAGTTCTCTCGTTAACTTAATGAGACAGATA |
| | | GAAACTGGTCTTGTAGAAACAGAGTAGTCGCCTGCTTTTCTGCCAGGT |
| | | GCTGACTTCTCTCCCCTGGGCTTTTTTCTTTTTCTCAGGTTGAAAAGAA |
| | | GAAGACGAAGAAGACGAAGAAGACAAACCGTCGGATCCAACTCCTAA |
| | | AAAACCGCCACC |
| 9 | C26_L21 | CGAGTCTTCAAACCTTAAGCCTCAGTGCAAAAGGGAGCAGCGAGGAG |
| | | AGGCGGTGGGATTAAAGAAAAAAATTCTTCTCTCCCTGTCACTTCAAA |
| | | AGGTCGCCCAACCAGCGAGCGGGGGGTTTACCCACGACTCTGGCTCGC |
| | | ACCCCTTGCGGGCCTGCGCAGGCGGCCCCGTAGCGCAAGGGAGGGCG |
| | | GGAAAGGAAGGGGCGGGGACGCGAGGGCGAATCTATAAGAAGCGTC |
| | | GTTCAGCGAGTTCGCTGCTCAGAAGCGCCGAGAGCGCGGCCGGGACG |
| | | GTTGGAGAAGAAGGCGGCTCCCGGAAGGGGGAGAGACAAACTGCCGT |
| | | AACCTCTGCCGTTCAGGAACGTCGAGGTGAGCCCCACGTTCTGCTTCA |
| | | CTCTCCCCATCTCCCCCCCCTCCCCACCCCCAATTTTGTATTTATTTATT |
| | | TTTTAATTATTTTGTGCAGCGATGGGGGCGGGGGGGGGGGGGGGGCGC |
| | | GCGCCAGGCGGGGGGGGCGGGGCGAGGGGCGGGGCGGGGCGAGGC |
| | | GGAGAGGTGCGGCGGCAGCCAATCAGAGCGGCGCGCTCCGAAAGTTT |
| | | CCTTTTATGGCGAGGCGGCGGCGGCGGCGGCCCTATAAAAAGCGAAG |
| | | CGCGCGGCGGGCGGGAGTCGCTGCGCGCTGCCTTCGCCCCGTGCCCCG |
| | | CTCCGCCGCCGCCTCGCGCCGCCCGCCCCGGCTCTGACTGACCGCGTT |
| | | ACTCCCACAGGTGAGCGGGCGGGACGGCCCTTCTCCTCCGGGCTGTAA |
| | | TTAGCTGAGCAAGAGGTAAGGGTTTAAGGGATGGTTGGTTGGTGGGGT |
| | | ATTAATGTTTAATTACCTGGAGCACCTGCCTGAAATCACTTTTTTTCAG |
| | | GTTGGACCGGTGCGGATCCAACTCCTAAAAAACCGCCACC |
| 10 | C46_L21 | TTACATCAAGTGCCAAGCTGGCCTAACTTCAGTCTCCACCCACTCAGT |
| | | GTGGGGAAACTCCATCGCATAAAACCCCTCCCCCCAACCTAAAGACGA |
| | | CGTACTCCAAAAGCTCGAGAACTAATCGAGGTGCCTGGACGGCGCCCG |
| | | GTACTCCGTGGAGTCACATGAAGCGACGGCTGAGGACGGAAAGGCCC |
| | | TTTTCCTTTGTGTGGGTGACTCACCCGCCCGCTCTCCCGAGCGCCGCGT |
| | | CCTCCATTTTGAGCTCCCTGCAGCAGGGCCGGGAAGCGGCCATCTTTC |
| | | CGCTCACGCAACTGGTGCCGACCGGGCCAGCCTTGCCGCCCAGGGCGG |
| | | GGCGATACACAATTGGTTTGATCTGATTATAACTAGTGGAGAAGAGCA |
| | | TGCTTGAGGGCTGAGTGCCCGTCAGTGGGCAGAGCGCACATCGCCCAC |
| | | AGTCCCCGAGAAGTTGGGGGGAGGGGTCGGCAATTGAACCGGTGCCT |
| | | AGAGAAGGTGGCGCGGGGTAAACTGGGAAAGTGATGTCGTGTACTGG |
| | | CTCCGCCTTTTTCCCGAGGGTGGGGGAGAACCGTATATAAGTGCAGTA |
| | | GTCGCCGTGAACGTTCTTTTTCGCAACGGGCTCGAGCCGAGTGGTCGT |
| | | TGCCGCCAGAACACAGCTGAAGCTTCGAGGGGCTCGCATCTCTCCTTC |
| | | ACGCGCCCGCCGCCCTACCTGAGGCCGCCATCCACGCCGGTTGAGTCG |
| | | CGTTCTGCCGCCTCCCGCCTGTGGTGCCTCCTGAACTGCGTCCGCCGTC |
| | | TAGGTAAGTTTAAAGCTCAGGTCGAGACCGGGCCTTTGTCCGGCGCTC |
| | | CCTTGGAGCCTACCTAGACTCAGCCGGCTCTCCACGCTTTGCCTGACCC |
| | | TGCTTGCTCAACTCTACGTCTTTGTTTCGTTTTCTGTTCTGCGCCGTTAC |
| | | AGATCCAAGCTGTGACCGGCGCCTACAAACAGTAGTTGACGGATCCAA |
| | | CTCCTAAAAAACCGCCACC |

TABLE 7
Additional sequences.

| SEQ ID NO | Name | Sequence |
|---|---|---|
| 51 | ITR (5′) | TTGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGTG |
| | | AGCGAGCGAGCGCGCAGAGAGGGAGTGGCCAACTCCATCACTAGG |
| | | GGTTCCT |
| 52 | BDNFco | ATGACCATCCTGTTCCTGACCATGGTCATTTCCTACTTTGGCTGCAT |
| | CDS | GAAGGCTGCCCCCATGAAAGAAGCCAACATCCGCGGCCAGGGCGG |
| | | CCTGGCCTACCCTGGCGTGCGCACCCATGGCACCCTGGAGTCCGTG |
| | | AATGGCCCCAAGGCCGGCTCCCGCGGCCTGACCTCCCTGGCTGACA |
| | | CCTTCGAACATGTGATCGAAGAGCTGCTGGATGAGGACCAGAAAGT |
| | | GCGCCCCAATGAAGAAAACAATAAGGACGCCGACCTGTACACCTC |
| | | CCGCGTGATGCTGTCCTCCCAGGTGCCTCTGGAGCCTCCTCTGCTGT |
| | | TTCTGCTGGAGGAATACAAAAATTACCTGGATGCTGCTAACATGTC |
| | | CATGCGCGTCCGCCGCCACTCCGACCCTGCCCGCCGCGGCGAGCTG |
| | | TCCGTGTGCGACTCCATTTCCGAGTGGGTGACCGCTGCCGACAAAA |
| | | AGACCGCCGTGGACATGTCCGGCGGCACCGTCACCGTCCTGGAAAA |
| | | GGTCCCTGTCTCCAAAGGCCAGCTGAAGCAGTACTTCTATGAGACC |
| | | AAGTGCAATCCCATGGGCTACACCAAAGAAGGCTGCCGCGGCATTG |
| | | ACAAACGGCATTGGAACTCCCAGTGCCGCACCACCCAGTCCTACGT |
| | | GCGCGCCCTGACCATGGATTCCAAAAAGCGCATTGGCTGGCGCTTC |
| | | ATCCGCATTGACACCTCCTGCGTCTGTACCCTGACCATCAAACGCG |
| | | GCCGCTGA |
| 53 | BDNFco | ATGACCATCCTGTTCCTGACTATGGTGATTAGCTACTTTGGATGCAT |
| | 3 CDS | GAAGGCTGCCCCTATGAAAGAAGCTAACATCAGAGGACAAGGAGG |
| | | CCTGGCCTACCCAGGAGTGAGAACCCATGGAACTCTGGAGTCTGTG |
| | | AATGGCCCAAAGGCTGGAAGCAGAGGCCTGACAAGCCTGGCTGAC |
| | | ACTTTTGAACATGTGATTGAAGAGCTGCTGGATGAGGACCAGAAAG |
| | | TGAGACCTAATGAAGAAAACAATAAGGATGCTGACCTGTACACCA |
| | | GCAGAGTGATGCTGAGCAGCCAAGTGCCTCTGGAGCCTCCTCTGCT |
| | | GTTTCTGCTGGAGGAATACAAAAATTACCTGGATGCTGCTAACATG |
| | | AGCATGAGAGTCAGAAGACACTCTGACCCTGCCAGAAGAGGAGAG |
| | | CTGTCTGTGTGTGACAGCATTTCTGAGTGGGTGACAGCTGCTGACA |
| | | AAAAGACTGCTGTGGACATGTCTGGAGGAACTGTCACAGTCCTGGA |
| | | AAAGGTCCCTGTGAGCAAAGGCCAACTGAAGCAATACTTCTATGAG |
| | | ACCAAGTGCAATCCAATGGGCTACACAAAAGAAGGCTGCAGAGGC |
| | | ATTGACAAAAGACATTGGAACAGCCAGTGCAGAACTACCCAGAGC |
| | | TATGTGAGAGCCCTGACCATGGATAGCAAAAAGAGAATTGGCTGG |
| | | AGATTCATTAGAATTGACACTAGCTGTGTCTGTACACTGACCATTA |
| | | AAAGAGGAAGATGA |
| 54 | BDNFco | ATGACCATCCTGTTTCTGACAATGGTTATTTCATATTTTGGTTGCAT |
| | 2 CDS | GAAAGCAGCTCCAATGAAGGAAGCAAACATCAGAGGACAAGGGGG |
| | | CCTAGCTTATCCTGGAGTTAGAACCCATGGGACTTTGGAGTCTGTC |
| | | AATGGCCCAAAAGCTGGAAGCAGGGGCCTGACAAGCCTGGCAGAT |
| | | ACTTTTGAACATGTCATTGAGGAACTCCTAGATGAAGACCAAAAAG |
| | | TTAGACCAAATGAAGAGAACAATAAAGATGCTGACCTTTATACTAG |
| | | CAGAGTCATGCTCTCCTCTCAAGTCCCACTAGAACCCCCACTCCTGT |
| | | TCCTACTGGAGGAATACAAAAATTACCTTGATGCAGCCAATATGAG |
| | | TATGAGAGTGAGGAGACACAGTGACCCTGCTAGGAGAGGGGAACT |
| | | CTCAGTATGTGACTCCATTAGTGAATGGGTGACTGCTGCAGACAAA |
| | | AAGACAGCAGTTGACATGAGTGGTGGCACAGTAACTGTACTAGAA |
| | | AAAGTACCAGTGAGTAAAGGTCAGCTTAAGCAATATTTTTATGAGA |
| | | CAAAATGCAATCCAATGGGATATACCAAGGAAGGATGTAGGGGCA |
| | | TTGATAAAAGGCACTGGAATAGCCAATGTAGAACCACACAATCTTA |
| | | TGTTAGGGCCCTTACCATGGACTCCAAGAAAAGAATTGGATGGAGA |
| | | TTTATCAGGATTGATACAAGTTGTGTCTGCACCCTGACCATCAAAA |
| | | GAGGAAGATGA |
| 55 | BDNFco | ATGACCATTCTGTTTCTGACCATGGTTATTAGCTATTTCGGGTGCAT |
| | 4 CDS | GAAAGCCGCCCCGATGAAAGAAGCGAATATCCGGGGCCAGGGCGG |
| | | CCTGGCGTATCCGGGCGTCCGGACCCACGGGACCCTGGAATCCGTG |
| | | AACGGCCCGAAAGCCGGGTCCCGGGGCCTGACCAGCCTGGCGGAT |
| | | ACCTTTGAACATGTGATTGAAGAACTGCTGGATGAAGATCAGAAAG |
| | | TCCGTCCGAATGAAGAAAATAACAAAGATGCGGATCTGTATACCTC |
| | | CCGGGTTATGCTGAGCAGCCAGGTTCCGCTGGAACCGCCGCTGCTG |
| | | TTTCTGCTGGAAGAATATAAGAATTATCTGGACGCCGCCAATATGA |
| | | GCATGCGGGTCCGCCGCCATTCCGATCCGGCGCGCCGCGGCGAACT |
| | | GTCCGTGTGCGATAGCATTTCCGAATGGGTGACCGCCGCCGATAAG |
| | | AAAACCGCGGTGGATATGTCCGGCGGCACCGTGACCGTGCTGGAG |
| | | AAAGTTCCGGTGAGCAAAGGGCAGCTGAAACAGTATTTCTATGAAA |
| | | CCAAATGCAATCCGATGGGCTATACCAAAGAGGGCTGCCGGGGCA |
| | | TTGATAAACGGCATTGGAATAGCCAGTGCCGGACCACCCAGAGCTA |
| | | TGTCCGGGCCCTAACCATGGATAGCAAGAAACGGATCGGGTGGCG |
| | | CTTTATCCGGATTGATACCAGCTGCGTGTGCACCCTAACCATCAAA |
| | | CGGGGCCGGTAA |
| 56 | BDNF- | ATGACCATCCTTTTCCTTACTATGGTTATTTCATACTTTGGTTGCATG |
| | HA CDS | AAGGCTGCCCCCATGAAAGAAGCAAACATCCGAGGACAAGGTGGC |
| | | TTGGCCTACCCAGGTGTGCGGACCCATGGGACTCTGGAGAGCGTGA |
| | | ATGGGCCCAAGGCAGGTTCAAGAGGCTTGACATCATTGGCTGACAC |
| | | TTTCGAACACGTGATAGAAGAGCTGTTGGATGAGGACCAGAAAGTT |
| | | CGGCCCAATGAAGAAAACAATAAGGACGCAGACTTGTACACGTCC |
| | | AGGGTGATGCTCAGTAGTCAAGTGCCTTTGGAGCCTCCTCTTCTCTT |
| | | TCTGCTGGAGGAATACAAAAATTACCTAGATGCTGCAAACATGTCC |
| | | ATGAGGGTCCGGCGCCACTCTGACCCTGCCCGCCGAGGGGAGCTGA |
| | | GCGTGTGTGACAGTATTAGTGAGTGGGTAACGGCGGCAGACAAAA |
| | | AGACTGCAGTGGACATGTCGGGGGGACGGTCACAGTCCTTGAAA |
| | | AGGTCCCTGTATCAAAAGGCCAACTGAAGCAATACTTCTACGAGAC |
| | | CAAGTGCAATCCCATGGGTTACACAAAAGAAGGCTGCAGGGGCAT |
| | | AGACAAAAGGCATTGGAACTCCCAGTGCCGAACTACCCAGTCGTAC |
| | | GTGCGGGCCCTTACCATGGATAGCAAAAAGAGAATTGGCTGGCGAT |
| | | TCATAAGGATAGACACTTCTTGTGTATGTACATTGACCATTAAAAG |
| | | GGGAAGATACCCATACGATGTTCCAGATTACGCTTAG |
| 57 | BDNF | ATGACCATCCTTTTCCTTACTATGGTTATTTCATACTTTGGTTGCATG |
| | CDS | AAGGCTGCCCCCATGAAAGAAGCAAACATCCGAGGACAAGGTGGC |
| | | TTGGCCTACCCAGGTGTGCGGACCCATGGGACTCTGGAGAGCGTGA |
| | | ATGGGCCCAAGGCAGGTTCAAGAGGCTTGACATCATTGGCTGACAC |
| | | TTTCGAACACGTGATAGAAGAGCTGTTGGATGAGGACCAGAAAGTT |
| | | CGGCCCAATGAAGAAAACAATAAGGACGCAGACTTGTACACGTCC |
| | | AGGGTGATGCTCAGTAGTCAAGTGCCTTTGGAGCCTCCTCTTCTCTT |
| | | TCTGCTGGAGGAATACAAAAATTACCTAGATGCTGCAAACATGTCC |
| | | ATGAGGGTCCGGCGCCACTCTGACCCTGCCCGCCGAGGGGAGCTGA |
| | | GCGTGTGTGACAGTATTAGTGAGTGGGTAACGGCGGCAGACAAAA |
| | | AGACTGCAGTGGACATGTCGGGCGGGACGGTCACAGTCCTTGAAA |
| | | AGGTCCCTGTATCAAAAGGCCAACTGAAGCAATACTTCTACGAGAC |
| | | CAAGTGCAATCCCATGGGTTACACAAAAGAAGGCTGCAGGGGCAT |
| | | AGACAAAAGGCATTGGAACTCCCAGTGCCGAACTACCCAGTCGTAC |
| | | GTGCGGGCCCTTACCATGGATAGCAAAAAGAGAATTGGCTGGCGAT |
| | | TCATAAGGATAGACACTTCTTGTGTATGTACATTGACCATTAAAAG |
| | | GGGAAGATAG |
| 58 | WPRE250 | AATCAACCTCTGGATTACAAAATTTGTGAAAGATTGACTGGTATTC |
| | | TTAACTATGTTGCTCCTTTTACGCTATGTGGATACGCTGCTTTAATG |
| | | CCTTTGTATCATGCTATTGCTTCCCGTATGGCTTTCATTTTCTCCTCC |
| | | TTGTATAAATCCTGGTTGCTGTCTCTTTATGAGGAGTTGTGGCCCGT |
| | | TGTCAGGCAACGTGGCGTGGTGTGCACTGTGTTTGCTGACGCAACC |
| | | CCCACTGGTTGGGGCA |
| 59 | WPRE3 | AATCAACCTCTGGATTACAAAATTTGTGAAAGATTGACTGGTATTC |
| | | TTAACTATGTTGCTCCTTTTACGCTATGTGGATACGCTGCTTTAATG |
| | | CCTTTGTATCATGCTATTGCTTCCCGTATGGCTTTCATTTTCTCCTCC |
| | | TTGTATAAATCCTGGTTAGTTCTTGCCACGGCGGAACTCATCGCCGC |
| | | CTGCCTTGCCCGCTGCTGGACAGGGGCTCGGCTGTTGGGCACTGAC |
| | | AATTCCGTGGTGTT |
| 60 | WPREmut6 | AATCAACCTCTGGATTACAAAATTTGTGAAAGATTGACTGGTATTC |
| | | TTAACTATGTTGCTCCTTTTACGCTATGTGGATACGCTGCTTTAATG |
| | | CCTTTGTATCATGCTATTGCTTCCCGTATGGCTTTCATTTTCTCCTCC |
| | | TTGTATAAATCCTGGTTGCTGTCTCTTTATGAGGAGTTGTGGCCCGT |
| | | TGTCAGGCAACGTGGCGTGGTGTGCACTGTGTTTGCTGACGCAACC |
| | | CCCACTGGTTGGGGCATTGCCACCACCTGTCAGCTCCTTTCCGGGAC |
| | | TTTCGCTTTCCCCCTCCCTATTGCCACGGCGGAACTCATCGCCGCCT |
| | | GCCTTGCCCGCTGCTGGACAGGGGCTCGGCTGTTGGGCACTGACAA |
| | | TTCCGTGGTGTTGTCGGGGAAATCATCGTCCTTTCCTTGGCTGCTCG |
| | | CCTGTGTTGCCACCTGGATTCTGCGCGGGACGTCCTTCTGCTACGTC |
| | | CCTTCGGCCCTCAATCCAGCGGACCTTCCTTCCCGCGGCCTGCTGCC |
| | | GGCTCTGCGGCCTCTTCCGCGTCTTCGCCTTCGCCCTCAGACGAGTC |
| | | GGATCTCCCTTTGGGCCGCCTCCCCGC |
| 61 | WPRE | CGATAATCAACCTCTGGATTACAAAATTTGTGAAAGATTGACTGGT |
| | | ATTCTTAACTATGTTGCTCCTTTTACGCTATGTGGATACGCTGCTTT |
| | | AATGCCTTTGTATCATGCTATTGCTTCCCGTATGGCTTTCATTTTCTC |
| | | CTCCTTGTATAAATCCTGGTTGCTGTCTCTTTATGAGGAGTIGTGGC |
| | | CCGTTGTCAGGCAACGTGGCGTGGTGTGCACTGTGTTTGCTGACGC |
| | | AACCCCCACTGGTTGGGGCATTGCCACCACCTGTCAGCTCCTTTCCG |
| | | GGACTTTCGCTTTCCCCCTCCCTATTGCCACGGCGGAACTCATCGCC |
| | | GCCTGCCTTGCCCGCTGCTGGACAGGGGCTCGGCTGTTGGGCACTG |
| | | ACAATTCCGTGGTGTTGTCGGGGAAGCTGACGTCCTTTCCATGGCT |
| | | GCTCGCCTGTGTTGCCACCTGGATTCTGCGCGGGACGTCCTTCTGCT |
| | | ACGTCCCTTCGGCCCTCAATCCAGCGGACCTTCCTTCCCGCGGCCTG |
| | | CTGCCGGCTCTGCGGCCTCTTCCGCGTCTTCGCCTTCGCCCTCAGAC |
| | | GAGTCGGATCTCCCTTTGGGCCGCCTCCCCGCATCGG |
| 62 | sPolyA2 | GCTCGCTTTCTTGCTGTCCAATTTCTATTAAAGGTTCCTTTGTTCCCT |
| | | AAGTCCAACTACTAAACTGGGGGATATTATGAAGGGCCTTGAGCAT |
| | | CTGGATTCTGCCTAATAAAAGATCTTTATTTTCATTAGATCTGTGTG |
| | | TTGGTTTTTTGTGTG |
| 63 | mirBS(4xmir142- | TCCATAAAGTAGGAAACACTACATCATCCATAAAGTAGGAAACACT |
| | 4xmir185) | ACATCATCCATAAAGTAGGAAACACTACATCATCCATAAAGTAGGA |
| | | AACACTACAGAATTCGATATCAGTGAATTCTACCAGTGCCATAAGT |
| | | GAATTCTACCAGTGCCATAAGTGAATTCTACCAGTGCCATAAGTGA |
| | | ATTCTACCAGTGCCATA |
| 64 | mirBS(4xmir142- | TCCATAAAGTAGGAAACACTACATCATCCATAAAGTAGGAAACACTA |
| | 4xmir185- | CATCATCCATAAAGTAGGAAACACTACATCATCCATAAAGTAGGAAA |
| | 4xmir1- | CACTACAGAATTCGATATCAGTGAATTCTACCAGTGCCATAAGTGAAT |
| | rxmir122) | TCTACCAGTGCCATAAGTGAATTCTACCAGTGCCATAAGTGAATTCTA |
| | 5′ portion in | CCAGTGCCATATCAAGCTTATACATACTTCTTTACATTCCAATACATACT |
| | italics and bold | TCTTTACATTCCAATACATACTTCTTTACATTCCACCATGGACTTGTACAA |
| | (corresponding to | ACACCATTGTCACACACTCCAACAAACACCATTGTCACACACTCCAACAA |
| | (SEQ ID NO: 63); | ACACCATTGTCACACACTCCAA |
| | cloning site | |
| | underlined; 3′ | |
| | portion in italics | |
| | (corresponding to | |
| | SEQ ID NO: 82) | |
| 82 | mirBS(4xmir142- | ATACATACTTCTTTACATTCCAATACATACTTCTTTACATTCCAATA |
| | 4xmir185- | CATACTTCTTTACATTCCACCATGGACTTGTACAAACACCATTGTCA |
| | 4xmir122)-3′ | CACACTCCAACAAACACCATTGTCACACACTCCAACAAACACCATT |
| | portion | GTCACACACTCCAA |
| 65 | ITR (3′) | AGGAACCCCTAGTGATGGAGTTGGCCACTCCCTCTCTGCGCGCTCG |
| | | CTCGCTCACTGAGGCCGGGCGACCAAAGGTCGCCCGACGCCCGGGC |
| | | TTTGCCCGGGCGGCCTCAGTGAGCGAGCGAGCGCGCAGagagggagtgg |
| | | ccaa |
| 66 | bGH | CTGTGCCTTCTAGTTGCCAGCCATCTGTTGTTTGCCCCTCCCCCGTG |
| | | CCTTCCTTGACCCTGGAAGGTGCCACTCCCACTGTCCTTTCCTAATA |
| | | AAATGAGGAAATTGCATCGCATTGTCTGAGTAGGTGTCATTCTATT |
| | | CTGGGGGGTGGGGTGGGGCAGGACAGCAAGGGGGAGGATTGGGAA |
| | | GACAATAGCAGGCATGCTGGGGATGGCCCGGGCTCTATGG |
| 67 | hGH | ACGGGTGGCATCCCTGTGACCCCTCCCCAGTGCCTCTCCTGGCCCTG |
| | | GAAGTTGCCACTCCAGTGCCCACCAGCCTTGTCCTAATAAAATTAA |
| | | GTTGCATCATTTTGTCTGACTAGGTGTCCTTCTATAATATTATGGGG |
| | | TGGAGGGGGGTGGTATGGAGCAAGGGGCAAGTTGGGAAGACAACC |
| | | TGTAGGGCCTGCGGGGTCTATTGGGAACCAAGCTGGAGTGCAGTGG |
| | | CACAATCTTGGCTCACTGCAATCTCCGCCTCCTGGGTTCAAGCGATT |
| | | CTCCTGCCTCAGCCTCCCGAGTTGTTGGGATTCCAGGCATGCATGAC |
| | | CAGGCTCAGCTAATTTTTGTTTTTTTGGTAGAGACGGGGTTTCACCA |
| | | TATTGGCCAGGCTGGTCTCCAACTCCTAATCTCAGGTGATCTACCCA |
| | | CCTTGGCCTCCCAAATTGCTGGGATTACAGGCGTGAACCACTGCTC |
| | | CCTTCCCTGTCCTT |
| 68 | sv40 | CAGACATGATAAGATACATTGATGAGTTTGGACAAACCACAACTAG |
| | | AATGCAGTGAAAAAAATGCTTTATTTGTGAAATTTGTGATGCTATT |
| | | GCTTTATTTGTAACCATTATAAGCTGCAATAAACAAGTTAACAACA |
| | | ACAATTGCATTCATTTTATGTTTCAGGTTCAGGGGGAGGTGTGGGA |
| | | GGTTTTTTAAAGCAAGTAAAACCTCTACAAATGTGGTA |
| 69 | sv40pa2 | GTTTATTTGTGAAATTTGTGATGCTATTGCTTTATTTGTAACCATCTA |
| | | GCTTTATTTGTGAAATTTGTGATGCTATTGCTTTATTTGTAACCATTA |
| | | TAAGCTGCAATAAACAAGTTAACAACAACAATTGCATTCATTTTAT |
| | | GTTTCAGGTTCAGGGGGAGATGTGGGAGGTTTTTTAA |
| 70 | mBDNF_HA | ATGACCATCCTTTTCCTTACTATGGTTATTTCATACTTTGGTTGCATG |
| | | AAGGCTCACTCTGACCCTGCCCGCCGAGGGGAGCTGAGCGTGTGTG |
| | | ACAGTATTAGTGAGTGGGTAACGGCGGCAGACAAAAAGACTGCAG |
| | | TGGACATGTCGGGCGGGACGGTCACAGTCCTTGAAAAGGTCCCTGT |
| | | ATCAAAAGGCCAACTGAAGCAATACTTCTACGAGACCAAGTGCAAT |
| | | CCCATGGGTTACACAAAAGAAGGCTGCAGGGGCATAGACAAAAGG |
| | | CATTGGAACTCCCAGTGCCGAACTACCCAGTCGTACGTGCGGGCCC |
| | | TTACCATGGATAGCAAAAAGAGAATTGGCTGGCGATTCATAAGGAT |
| | | AGACACTTCTTGTGTATGTACATTGACCATTAAAAGGGGAAGATAC |
| | | CCATACGATGTTCCAGATTACGCTTAG |
| 71 | mIL2_td | ATGCGGAGAATGCAGCTGCTGCTGCTCATTGCCCTGTCTCTGGCCCT |
| | BDNF_HA | GGTCACCAATAGCCACTCTGACCCTGCCCGCCGAGGGGAGCTGAGC |
| | | GTGTGTGACAGTATTAGTGAGTGGGTAACGGCGGCAGACAAAAAG |
| | | ACTGCAGTGGACATGTCGGGCGGGACGGTCACAGTCCTTGAAAAG |
| | | GTCCCTGTATCAAAAGGCCAACTGAAGCAATACTTCTACGAGACCA |
| | | AGTGCAATCCCATGGGTTACACAAAAGAAGGCTGCAGGGGCATAG |
| | | ACAAAAGGCATTGGAACTCCCAGTGCCGAACTACCCAGTCGTACGT |
| | | GCGGGCCCTTACCATGGATAGCAAAAAGAGAATTGGCTGGCGATTC |
| | | ATAAGGATAGACACTTCTTGTGTATGTACATTGACCATTAAAAGGG |
| | | GAAGAGGAGGCGGAGGATCTGGCGGAGGCGGATCTCACTCTGACC |
| | | CTGCCCGCCGAGGGGAGCTGAGCGTGTGTGACAGTATTAGTGAGTG |
| | | GGTAACGGCGGCAGACAAAAAGACTGCAGTGGACATGTCGGGCGG |
| | | GACGGTCACAGTCCTTGAAAAGGTCCCTGTATCAAAAGGCCAACTG |
| | | AAGCAATACTTCTACGAGACCAAGTGCAATCCCATGGGTTACACAA |
| | | AAGAAGGCTGCAGGGGCATAGACAAAAGGCATTGGAACTCCCAGT |
| | | GCCGAACTACCCAGTCGTACGTGCGGGCCCTTACCATGGATAGCAA |
| | | AAAGAGAATTGGCTGGCGATTCATAAGGATAGACACTTCTTGTGTA |
| | | TGTACATTGACCATTAAAAGGGGAAGATACCCATACGATGTTCCAG |
| | | ATTACGCTTAG |
| 72 | Gaussia- | ATGGGAGTGAAAGTTCTTTTTGCCCTTATTTGTATTGCTGTGGCCGA |
| | BDNF-ha | GGCCGCCCCCATGAAAGAAGCAAACATCCGAGGACAAGGTGGCTT |
| | | GGCCTACCCAGGTGTGCGGACCCATGGGACTCTGGAGAGCGTGAAT |
| | | GGGCCCAAGGCAGGTTCAAGAGGCTTGACATCATTGGCTGACACTT |
| | | TCGAACACGTGATAGAAGAGCTGTTGGATGAGGACCAGAAAGTTC |
| | | GGCCCAATGAAGAAAACAATAAGGACGCAGACTTGTACACGTCCA |
| | | GGGTGATGCTCAGTAGTCAAGTGCCTTTGGAGCCTCCTCTTCTCTTT |
| | | CTGCTGGAGGAATACAAAAATTACCTAGATGCTGCAAACATGTCCA |
| | | TGAGGGTCCGGCGCCACTCTGACCCTGCCCGCCGAGGGGAGCTGAG |
| | | CGTGTGTGACAGTATTAGTGAGTGGGTAACGGCGGCAGACAAAAA |
| | | GACTGCAGTGGACATGTCGGGCGGGACGGTCACAGTCCTTGAAAA |
| | | GGTCCCTGTATCAAAAGGCCAACTGAAGCAATACTTCTACGAGACC |
| | | AAGTGCAATCCCATGGGTTACACAAAAGAAGGCTGCAGGGGCATA |
| | | GACAAAAGGCATTGGAACTCCCAGTGCCGAACTACCCAGTCGTACG |
| | | TGCGGGCCCTTACCATGGATAGCAAAAAGAGAATTGGCTGGCGATT |
| | | CATAAGGATAGACACTTCTTGTGTATGTACATTGACCATTAAAAGG |
| | | GGAAGATACCCATACGATGTTCCAGATTACGCTTAG |
| 73 | mIL2_BDNF_HA | ATGCGGAGGATGCAGCTGCTCTTGCTCATTGCCCTAAGTCTCGCACT |
| | | TGTCACAAACAGTGCCCCCATGAAAGAAGCAAACATCCGAGGACA |
| | | AGGTGGCTTGGCCTACCCAGGTGTGCGGACCCATGGGACTCTGGAG |
| | | AGCGTGAATGGGCCCAAGGCAGGTTCAAGAGGCTTGACATCATTGG |
| | | CTGACACTTTCGAACACGTGATAGAAGAGCTGTTGGATGAGGACCA |
| | | GAAAGTTCGGCCCAATGAAGAAAACAATAAGGACGCAGACTTGTA |
| | | CACGTCCAGGGTGATGCTCAGTAGTCAAGTGCCTTTGGAGCCTCCT |
| | | CTTCTCTTTCTGCTGGAGGAATACAAAAATTACCTAGATGCTGCAA |
| | | ACATGTCCATGAGGGTCCGGCGCCACTCTGACCCTGCCCGCCGAGG |
| | | GGAGCTGAGCGTGTGTGACAGTATTAGTGAGTGGGTAACGGCGGC |
| | | AGACAAAAAGACTGCAGTGGACATGTCGGGCGGGACGGTCACAGT |
| | | CCTTGAAAAGGTCCCTGTATCAAAAGGCCAACTGAAGCAATACTTC |
| | | TACGAGACCAAGTGCAATCCCATGGGTTACACAAAAGAAGGCTGC |
| | | AGGGGCATAGACAAAAGGCATTGGAACTCCCAGTGCCGAACTACC |
| | | CAGTCGTACGTGCGGGCCCTTACCATGGATAGCAAAAAGAGAATTG |
| | | GCTGGCGATTCATAAGGATAGACACTTCTTGTGTATGTACATTGAC |
| | | CATTAAAAGGGGAAGATACCCATACGATGTTCCAGATTACGCTTAG |
| 74 | Secrecon | ATGAGACGACTCTGGTGGCTGTTGCTTCTGCTCTTGCTTCTGTGGCC |
| | 2-BDNF-HA | CATGGTCTGGGCCGCCCCCATGAAAGAAGCAAACATCCGAGGACA |
| | | AGGTGGCTTGGCCTACCCAGGTGTGCGGACCCATGGGACTCTGGAG |
| | | AGCGTGAATGGGCCCAAGGCAGGTTCAAGAGGCTTGACATCATTGG |
| | | CTGACACTTTCGAACACGTGATAGAAGAGCTGTTGGATGAGGACCA |
| | | GAAAGTTCGGCCCAATGAAGAAAACAATAAGGACGCAGACTTGTA |
| | | CACGTCCAGGGTGATGCTCAGTAGTCAAGTGCCTTTGGAGCCTCCT |
| | | CTTCTCTTTCTGCTGGAGGAATACAAAAATTACCTAGATGCTGCAA |
| | | ACATGTCCATGAGGGTCCGGCGCCACTCTGACCCTGCCCGCCGAGG |
| | | GGAGCTGAGCGTGTGTGACAGTATTAGTGAGTGGGTAACGGCGGC |
| | | AGACAAAAAGACTGCAGTGGACATGTCGGGCGGGACGGTCACAGT |
| | | CCTTGAAAAGGTCCCTGTATCAAAAGGCCAACTGAAGCAATACTTC |
| | | TACGAGACCAAGTGCAATCCCATGGGTTACACAAAAGAAGGCTGC |
| | | AGGGGCATAGACAAAAGGCATTGGAACTCCCAGTGCCGAACTACC |
| | | CAGTCGTACGTGCGGGCCCTTACCATGGATAGCAAAAAGAGAATTG |
| | | GCTGGCGATTCATAAGGATAGACACTTCTTGTGTATGTACATTGAC |
| | | CATTAAAAGGGGAAGATACCCATACGATGTTCCAGATTACGCTTAG |
| 75 | S2b-BDNF-HA | ATGAGACGACTCTGGTGGCTGTTGCTTCTGCTCTTGCTTCTGTGGCC |
| | | CTGTATGAAGGCCGCCCCCATGAAAGAAGCAAACATCCGAGGACA |
| | | AGGTGGCTTGGCCTACCCAGGTGTGCGGACCCATGGGACTCTGGAG |
| | | AGCGTGAATGGGCCCAAGGCAGGTTCAAGAGGCTTGACATCATTGG |
| | | CTGACACTTTCGAACACGTGATAGAAGAGCTGTTGGATGAGGACCA |
| | | GAAAGTTCGGCCCAATGAAGAAAACAATAAGGACGCAGACTTGTA |
| | | CACGTCCAGGGTGATGCTCAGTAGTCAAGTGCCTTTGGAGCCTCCT |
| | | CTTCTCTTTCTGCTGGAGGAATACAAAAATTACCTAGATGCTGCAA |
| | | ACATGTCCATGAGGGTCCGGCGCCACTCTGACCCTGCCCGCCGAGG |
| | | GGAGCTGAGCGTGTGTGACAGTATTAGTGAGTGGGTAACGGCGGC |
| | | AGACAAAAAGACTGCAGTGGACATGTCGGGCGGGACGGTCACAGT |
| | | CCTTGAAAAGGTCCCTGTATCAAAAGGCCAACTGAAGCAATACTTC |
| | | TACGAGACCAAGTGCAATCCCATGGGTTACACAAAAGAAGGCTGC |
| | | AGGGGCATAGACAAAAGGCATTGGAACTCCCAGTGCCGAACTACC |
| | | CAGTCGTACGTGCGGGCCCTTACCATGGATAGCAAAAAGAGAATTG |
| | | GCTGGCGATTCATAAGGATAGACACTTCTTGTGTATGTACATTGAC |
| | | CATTAAAAGGGGAAGATACCCATACGATGTTCCAGATTACGCTTAG |
| 76 | BDNF-furin- | ATGACCATCCTTTTCCTTACTATGGTTATTTCATACTTTGGTTGCATG |
| | F2A-BDNF | AAGGCTGCCCCCATGAAAGAAGCAAACATCCGAGGACAAGGTGGC |
| | | TTGGCCTACCCAGGTGTGCGGACCCATGGGACTCTGGAGAGCGTGA |
| | | ATGGGCCCAAGGCAGGTTCAAGAGGCTTGACATCATTGGCTGACAC |
| | | TTTCGAACACGTGATAGAAGAGCTGTTGGATGAGGACCAGAAAGTT |
| | | CGGCCCAATGAAGAAAACAATAAGGACGCAGACTTGTACACGTCC |
| | | AGGGTGATGCTCAGTAGTCAAGTGCCTTTGGAGCCTCCTCTTCTCTT |
| | | TCTGCTGGAGGAATACAAAAATTACCTAGATGCTGCAAACATGTCC |
| | | ATGAGGGTCCGGCGCCACTCTGACCCTGCCCGCCGAGGGGAGCTGA |
| | | GCGTGTGTGACAGTATTAGTGAGTGGGTAACGGCGGCAGACAAAA |
| | | AGACTGCAGTGGACATGTCGGGCGGGACGGTCACAGTCCTTGAAA |
| | | AGGTCCCTGTATCAAAAGGCCAACTGAAGCAATACTTCTACGAGAC |
| | | CAAGTGCAATCCCATGGGTTACACAAAAGAAGGCTGCAGGGGCAT |
| | | AGACAAAAGGCATTGGAACTCCCAGTGCCGAACTACCCAGTCGTAC |
| | | GTGCGGGCCCTTACCATGGATAGCAAAAAGAGAATTGGCTGGCGAT |
| | | TCATAAGGATAGACACTTCTTGTGTATGTACATTGACCATTAAAAG |
| | | GGGAAGACGCAAAAAACGCAGCGTGGGCAGCGGCGCGCCGGTGAA |
| | | ACAGACCCTGAACTTTGATCTGCTGAAACTGGCGGGCGATGTGGAA |
| | | AGCAACCCGGGCCCGATGACCATCCTTTTCCTTACTATGGTTATTTC |
| | | ATACTTTGGTTGCATGAAGGCTGCCCCCATGAAAGAAGCAAACATC |
| | | CGAGGACAAGGTGGCTTGGCCTACCCAGGTGTGCGGACCCATGGG |
| | | ACTCTGGAGAGCGTGAATGGGCCCAAGGCAGGTTCAAGAGGCTTG |
| | | ACATCATTGGCTGACACTTTCGAACACGTGATAGAAGAGCTGTTGG |
| | | ATGAGGACCAGAAAGTTCGGCCCAATGAAGAAAACAATAAGGACG |
| | | CAGACTTGTACACGTCCAGGGTGATGCTCAGTAGTCAAGTGCCTTT |
| | | GGAGCCTCCTCTTCTCTTTCTGCTGGAGGAATACAAAAATTACCTAG |
| | | ATGCTGCAAACATGTCCATGAGGGTCCGGCGCCACTCTGACCCTGC |
| | | CCGCCGAGGGGAGCTGAGCGTGTGTGACAGTATTAGTGAGTGGGTA |
| | | ACGGCGGCAGACAAAAAGACTGCAGTGGACATGTCGGGCGGGACG |
| | | GTCACAGTCCTTGAAAAGGTCCCTGTATCAAAAGGCCAACTGAAGC |
| | | AATACTTCTACGAGACCAAGTGCAATCCCATGGGTTACACAAAAGA |
| | | AGGCTGCAGGGGCATAGACAAAAGGCATTGGAACTCCCAGTGCCG |
| | | AACTACCCAGTCGTACGTGCGGGCCCTTACCATGGATAGCAAAAAG |
| | | AGAATTGGCTGGCGATTCATAAGGATAGACACTTCTTGTGTATGTA |
| | | CATTGACCATTAAAAGGGGAAGATAG |
| 77 | BDNF-furin- | ATGACCATCCTTTTCCTTACTATGGTTATTTCATACTTTGGTTGCATG |
| | P2A-BDNF | AAGGCTGCCCCCATGAAAGAAGCAAACATCCGAGGACAAGGTGGC |
| | | TTGGCCTACCCAGGTGTGCGGACCCATGGGACTCTGGAGAGCGTGA |
| | | ATGGGCCCAAGGCAGGTTCAAGAGGCTTGACATCATTGGCTGACAC |
| | | TTTCGAACACGTGATAGAAGAGCTGTTGGATGAGGACCAGAAAGTT |
| | | CGGCCCAATGAAGAAAACAATAAGGACGCAGACTTGTACACGTCC |
| | | AGGGTGATGCTCAGTAGTCAAGTGCCTTTGGAGCCTCCTCTTCTCTT |
| | | TCTGCTGGAGGAATACAAAAATTACCTAGATGCTGCAAACATGTCC |
| | | ATGAGGGTCCGGCGCCACTCTGACCCTGCCCGCCGAGGGGAGCTGA |
| | | GCGTGTGTGACAGTATTAGTGAGTGGGTAACGGCGGCAGACAAAA |
| | | AGACTGCAGTGGACATGTCGGGCGGGACGGTCACAGTCCTTGAAA |
| | | AGGTCCCTGTATCAAAAGGCCAACTGAAGCAATACTTCTACGAGAC |
| | | CAAGTGCAATCCCATGGGTTACACAAAAGAAGGCTGCAGGGGCAT |
| | | AGACAAAAGGCATTGGAACTCCCAGTGCCGAACTACCCAGTCGTAC |
| | | GTGCGGGCCCTTACCATGGATAGCAAAAAGAGAATTGGCTGGCGAT |
| | | TCATAAGGATAGACACTTCTTGTGTATGTACATTGACCATTAAAAG |
| | | GGGAAGACGCAAAAAACGCAGCGTGGGCAGCGGCGCGACCAACTT |
| | | TAGCCTGCTGAAACAGGCGGGCGATGTGGAAGAAAACCCGGGCCC |
| | | GATGACCATCCTTTTCCTTACTATGGTTATTTCATACTTTGGTTGCAT |
| | | GAAGGCTGCCCCCATGAAAGAAGCAAACATCCGAGGACAAGGTGG |
| | | CTTGGCCTACCCAGGTGTGCGGACCCATGGGACTCTGGAGAGCGTG |
| | | AATGGGCCCAAGGCAGGTTCAAGAGGCTTGACATCATTGGCTGACA |
| | | CTTTCGAACACGTGATAGAAGAGCTGTTGGATGAGGACCAGAAAGT |
| | | TCGGCCCAATGAAGAAAACAATAAGGACGCAGACTTGTACACGTC |
| | | CAGGGTGATGCTCAGTAGTCAAGTGCCTTTGGAGCCTCCTCTTCTCT |
| | | TTCTGCTGGAGGAATACAAAAATTACCTAGATGCTGCAAACATGTC |
| | | CATGAGGGTCCGGCGCCACTCTGACCCTGCCCGCCGAGGGGAGCTG |
| | | AGCGTGTGTGACAGTATTAGTGAGTGGGTAACGGCGGCAGACAAA |
| | | AAGACTGCAGTGGACATGTCGGGCGGGACGGTCACAGTCCTTGAA |
| | | AAGGTCCCTGTATCAAAAGGCCAACTGAAGCAATACTTCTACGAGA |
| | | CCAAGTGCAATCCCATGGGTTACACAAAAGAAGGCTGCAGGGGCA |
| | | TAGACAAAAGGCATTGGAACTCCCAGTGCCGAACTACCCAGTCGTA |
| | | CGTGCGGGCCCTTACCATGGATAGCAAAAAGAGAATTGGCTGGCG |
| | | ATTCATAAGGATAGACACTTCTTGTGTATGTACATTGACCATTAAA |
| | | AGGGGAAGATAG |
| 78 | BDNF-furin- | ATGACCATCCTTTTCCTTACTATGGTTATTTCATACTTTGGTTGCATG |
| | TSA-BDNF | AAGGCTGCCCCCATGAAAGAAGCAAACATCCGAGGACAAGGTGGC |
| | | TTGGCCTACCCAGGTGTGCGGACCCATGGGACTCTGGAGAGCGTGA |
| | | ATGGGCCCAAGGCAGGTTCAAGAGGCTTGACATCATTGGCTGACAC |
| | | TTTCGAACACGTGATAGAAGAGCTGTTGGATGAGGACCAGAAAGTT |
| | | CGGCCCAATGAAGAAAACAATAAGGACGCAGACTTGTACACGTCC |
| | | AGGGTGATGCTCAGTAGTCAAGTGCCTTTGGAGCCTCCTCTTCTCTT |
| | | TCTGCTGGAGGAATACAAAAATTACCTAGATGCTGCAAACATGTCC |
| | | ATGAGGGTCCGGCGCCACTCTGACCCTGCCCGCCGAGGGGAGCTGA |
| | | GCGTGTGTGACAGTATTAGTGAGTGGGTAACGGCGGCAGACAAAA |
| | | AGACTGCAGTGGACATGTCGGGCGGGACGGTCACAGTCCTTGAAA |
| | | AGGTCCCTGTATCAAAAGGCCAACTGAAGCAATACTTCTACGAGAC |
| | | CAAGTGCAATCCCATGGGTTACACAAAAGAAGGCTGCAGGGGCAT |
| | | AGACAAAAGGCATTGGAACTCCCAGTGCCGAACTACCCAGTCGTAC |
| | | GTGCGGGCCCTTACCATGGATAGCAAAAAGAGAATTGGCTGGCGAT |
| | | TCATAAGGATAGACACTTCTTGTGTATGTACATTGACCATTAAAAG |
| | | GGGAAGACGCAAAAAACGCAGCGTGGGCAGCGGCGAAGGCCGCG |
| | | GCAGCCTGCTGACCTGCGGCGATGTGGAAGAAAACCCGGGCCCGA |
| | | TGACCATCCTTTTCCTTACTATGGTTATTTCATACTTTGGTTGCATGA |
| | | AGGCTGCCCCCATGAAAGAAGCAAACATCCGAGGACAAGGTGGCT |
| | | TGGCCTACCCAGGTGTGCGGACCCATGGGACTCTGGAGAGCGTGAA |
| | | TGGGCCCAAGGCAGGTTCAAGAGGCTTGACATCATTGGCTGACACT |
| | | TTCGAACACGTGATAGAAGAGCTGTTGGATGAGGACCAGAAAGTTC |
| | | GGCCCAATGAAGAAAACAATAAGGACGCAGACTTGTACACGTCCA |
| | | GGGTGATGCTCAGTAGTCAAGTGCCTTTGGAGCCTCCTCTTCTCTTT |
| | | CTGCTGGAGGAATACAAAAATTACCTAGATGCTGCAAACATGTCCA |
| | | TGAGGGTCCGGCGCCACTCTGACCCTGCCCGCCGAGGGGAGCTGAG |
| | | CGTGTGTGACAGTATTAGTGAGTGGGTAACGGCGGCAGACAAAAA |
| | | GACTGCAGTGGACATGTCGGGCGGGACGGTCACAGTCCTTGAAAA |
| | | GGTCCCTGTATCAAAAGGCCAACTGAAGCAATACTTCTACGAGACC |
| | | AAGTGCAATCCCATGGGTTACACAAAAGAAGGCTGCAGGGGCATA |
| | | GACAAAAGGCATTGGAACTCCCAGTGCCGAACTACCCAGTCGTACG |
| | | TGCGGGCCCTTACCATGGATAGCAAAAAGAGAATTGGCTGGCGATT |
| | | CATAAGGATAGACACTTCTTGTGTATGTACATTGACCATTAAAAGG |
| | | GGAAGATAG |
| 79 | BDNF-HA-furin- | ATGACCATCCTTTTCCTTACTATGGTTATTTCATACTTTGGTTGCATG |
| | P2A-BDNF | AAGGCTGCCCCCATGAAAGAAGCAAACATCCGAGGACAAGGTGGC |
| | | TTGGCCTACCCAGGTGTGCGGACCCATGGGACTCTGGAGAGCGTGA |
| | | ATGGGCCCAAGGCAGGTTCAAGAGGCTTGACATCATTGGCTGACAC |
| | | TTTCGAACACGTGATAGAAGAGCTGTTGGATGAGGACCAGAAAGTT |
| | | CGGCCCAATGAAGAAAACAATAAGGACGCAGACTTGTACACGTCC |
| | | AGGGTGATGCTCAGTAGTCAAGTGCCTTTGGAGCCTCCTCTTCTCTT |
| | | TCTGCTGGAGGAATACAAAAATTACCTAGATGCTGCAAACATGTCC |
| | | ATGAGGGTCCGGCGCCACTCTGACCCTGCCCGCCGAGGGGAGCTGA |
| | | GCGTGTGTGACAGTATTAGTGAGTGGGTAACGGCGGCAGACAAAA |
| | | AGACTGCAGTGGACATGTCGGGCGGGACGGTCACAGTCCTTGAAA |
| | | AGGTCCCTGTATCAAAAGGCCAACTGAAGCAATACTTCTACGAGAC |
| | | CAAGTGCAATCCCATGGGTTACACAAAAGAAGGCTGCAGGGGCAT |
| | | AGACAAAAGGCATTGGAACTCCCAGTGCCGAACTACCCAGTCGTAC |
| | | GTGCGGGCCCTTACCATGGATAGCAAAAAGAGAATTGGCTGGCGAT |
| | | TCATAAGGATAGACACTTCTTGTGTATGTACATTGACCATTAAAAG |
| | | GGGAAGATACCCATACGATGTTCCAGATTACGCTCGCAAAAAACGC |
| | | AGCGTGGGCAGCGGCGCGACCAACTTTAGCCTGCTGAAACAGGCG |
| | | GGCGATGTGGAAGAAAACCCGGGCCCGATGACCATCCTTTTCCTTA |
| | | CTATGGTTATTTCATACTTTGGTTGCATGAAGGCTGCCCCCATGAAA |
| | | GAAGCAAACATCCGAGGACAAGGTGGCTTGGCCTACCCAGGTGTG |
| | | CGGACCCATGGGACTCTGGAGAGCGTGAATGGGCCCAAGGCAGGT |
| | | TCAAGAGGCTTGACATCATTGGCTGACACTTTCGAACACGTGATAG |
| | | AAGAGCTGTTGGATGAGGACCAGAAAGTTCGGCCCAATGAAGAAA |
| | | ACAATAAGGACGCAGACTTGTACACGTCCAGGGTGATGCTCAGTAG |
| | | TCAAGTGCCTTTGGAGCCTCCTCTTCTCTTTCTGCTGGAGGAATACA |
| | | AAAATTACCTAGATGCTGCAAACATGTCCATGAGGGTCCGGCGCCA |
| | | CTCTGACCCTGCCCGCCGAGGGGAGCTGAGCGTGTGTGACAGTATT |
| | | AGTGAGTGGGTAACGGCGGCAGACAAAAAGACTGCAGTGGACATG |
| | | TCGGGCGGGACGGTCACAGTCCTTGAAAAGGTCCCTGTATCAAAAG |
| | | GCCAACTGAAGCAATACTTCTACGAGACCAAGTGCAATCCCATGGG |
| | | TTACACAAAAGAAGGCTGCAGGGGCATAGACAAAAGGCATTGGAA |
| | | CTCCCAGTGCCGAACTACCCAGTCGTACGTGCGGGCCCTTACCATG |
| | | GATAGCAAAAAGAGAATTGGCTGGCGATTCATAAGGATAGACACT |
| | | TCTTGTGTATGTACATTGACCATTAAAAGGGGAAGATACCCATACG |
| | | ATGTTCCAGATTACGCTTAG |
| 80 | IRES | CCCCTCTCCCTCCCCCCCCCCTAACGTTACTGGCCGAAGCCGCTTGG |
| | | AATAAGGCCGGTGTGCGTTTGTCTATATGTTATTTTCCACCATATTG |
| | | CCGTCTTTTGGCAATGTGAGGGCCCGGAAACCTGGCCCTGTCTTCTT |
| | | GACGAGCATTCCTAGGGGTCTTTCCCCTCTCGCCAAAGGAATGCAA |
| | | GGTCTGTTGAATGTCGTGAAGGAAGCAGTTCCTCTGGAAGCTTCTT |
| | | GAAGACAAACAACGTCTGTAGCGACCCTTTGCAGGCAGCGGAACC |
| | | CCCCACCTGGCGACAGGTGCCTCTGCGGCCAAAAGCCACGTGTATA |
| | | AGATACACCTGCAAAGGCGGCACAACCCCAGTGCCACGTTGTGAGT |
| | | TGGATAGTTGTGGAAAGAGTCAAATGGCTCTCCTCAAGCGTATTCA |
| | | ACAAGGGGCTGAAGGATGCCCAGAAGGTACCCCATTGTATGGGAT |
| | | CTGATCTGGGGCCTCGGTGCACATGCTTTACATGTGTTTAGTCGAGG |
| | | TTAAAAAAACGTCTAGGCCCCCCGAACCACGGGGACGTGGTTTTCC |
| | | TTTGAAAAACACGATGATA |

TABLE 8
Construct sequences.

| SEQ | | |
|---|---|---|
| ID | | |
| NO | Name | Sequence |
| 11 | BDNF1 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACCATGGTCATTTCCTACTTTGGCTGCATGAAGGC |
| | | TGCCCCCATGAAAGAAGCCAACATCCGCGGCCAGGGCGGCCTGGCCTACCCTGGC |
| | | GTGCGCACCCATGGCACCCTGGAGTCCGTGAATGGCCCCAAGGCCGGCTCCCGCG |
| | | GCCTGACCTCCCTGGCTGACACCTTCGAACATGTGATCGAAGAGCTGCTGGATGA |
| | | GGACCAGAAAGTGCGCCCCAATGAAGAAAACAATAAGGACGCCGACCTGTACACC |
| | | TCCCGCGTGATGCTGTCCTCCCAGGTGCCTCTGGAGCCTCCTCTGCTGTTTCTGC |
| | | TGGAGGAATACAAAAATTACCTGGATGCTGCTAACATGTCCATGCGCGTCCGCCG |
| | | CCACTCCGACCCTGCCCGCCGCGGCGAGCTGTCCGTGTGCGACTCCATTTCCGAG |
| | | TGGGTGACCGCTGCCGACAAAAAGACCGCCGTGGACATGTCCGGCGGCACCGTCA |
| | | CCGTCCTGGAAAAGGTCCCTGTCTCCAAAGGCCAGCTGAAGCAGTACTTCTATGA |
| | | GACCAAGTGCAATCCCATGGGCTACACCAAAGAAGGCTGCCGCGGCATTGACAAA |
| | | CGGCATTGGAACTCCCAGTGCCGCACCACCCAGTCCTACGTGCGCGCCCTGACCA |
| | | TGGATTCCAAAAAGCGCATTGGCTGGCGCTTCATCCGCATTGACACCTCCTGCGT |
| | | CTGTACCCTGACCATCAAACGCGGCCGCTGAGGTACCTTCTCCATAAAGTAGGAA |
| | | ACACTACATCATCCATAAAGTAGGAAACACTACATCATCCATAAAGTAGGAAACA |
| | | CTACATCATCCATAAAGTAGGAAACACTACAGAATTCGATATCAGTGAATTCTAC |
| | | CAGTGCCATAAGTGAATTCTACCAGTGCCATAAGTGAATTCTACCAGTGCCATAA |
| | | GTGAATTCTACCAGTGCCATATCAAGCTTATACATACTTCTTTACATTCCAATAC |
| | | ATACTTCTTTACATTCCAATACATACTTCTTTACATTCCACCATGGACTTGTACA |
| | | AACACCATTGTCACACACTCCAACAAACACCATTGTCACACACTCCAACAAACAC |
| | | CATTGTCACACACTCCAAGCTAGCCCTAGGAATCAACCTCTGGATTACAAAATTT |
| | | GTGAAAGATTGACTGGTATTCTTAACTATGTTGCTCCTTTTACGCTATGTGGATA |
| | | CGCTGCTTTAATGCCTTTGTATCATGCTATTGCTTCCCGTATGGCTTTCATTTTC |
| | | TCCTCCTTGTATAAATCCTGGTTGCTGTCTCTTTATGAGGAGTTGTGGCCCGTTG |
| | | TCAGGCAACGTGGCGTGGTGTGCACTGTGTTTGCTGACGCAACCCCCACTGGTTG |
| | | GGGCACCTGCAGGGCTCGCTTTCTTGCTGTCCAATTTCTATTAAAGGTTCCTTTG |
| | | TTCCCTAAGTCCAACTACTAAACTGGGGGATATTATGAAGGGCCTTGAGCATCTG |
| | | GATTCTGCCTAATAAAAGATCTTTATTTTCATTAGATCTGTGTGTTGGTTTTTTG |
| | | TGTG |
| 12 | BDNF2 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACCATGGTCATTTCCTACTTTGGCTGCATGAAGGC |
| | | TGCCCCCATGAAAGAAGCCAACATCCGCGGCCAGGGCGGCCTGGCCTACCCTGGC |
| | | GTGCGCACCCATGGCACCCTGGAGTCCGTGAATGGCCCCAAGGCCGGCTCCCGCG |
| | | GCCTGACCTCCCTGGCTGACACCTTCGAACATGTGATCGAAGAGCTGCTGGATGA |
| | | GGACCAGAAAGTGCGCCCCAATGAAGAAAACAATAAGGACGCCGACCTGTACACC |
| | | TCCCGCGTGATGCTGTCCTCCCAGGTGCCTCTGGAGCCTCCTCTGCTGTTTCTGC |
| | | TGGAGGAATACAAAAATTACCTGGATGCTGCTAACATGTCCATGCGCGTCCGCCG |
| | | CCACTCCGACCCTGCCCGCCGCGGCGAGCTGTCCGTGTGCGACTCCATTTCCGAG |
| | | TGGGTGACCGCTGCCGACAAAAAGACCGCCGTGGACATGTCCGGCGGCACCGTCA |
| | | CCGTCCTGGAAAAGGTCCCTGTCTCCAAAGGCCAGCTGAAGCAGTACTTCTATGA |
| | | GACCAAGTGCAATCCCATGGGCTACACCAAAGAAGGCTGCCGCGGCATTGACAAA |
| | | CGGCATTGGAACTCCCAGTGCCGCACCACCCAGTCCTACGTGCGCGCCCTGACCA |
| | | TGGATTCCAAAAAGCGCATTGGCTGGCGCTTCATCCGCATTGACACCTCCTGCGT |
| | | CTGTACCCTGACCATCAAACGCGGCCGCTGAGGTACCTTCTCCATAAAGTAGGAA |
| | | ACACTACATCATCCATAAAGTAGGAAACACTACATCATCCATAAAGTAGGAAACA |
| | | CTACATCATCCATAAAGTAGGAAACACTACAGAATTCGATATCAGTGAATTCTAC |
| | | CAGTGCCATAAGTGAATTCTACCAGTGCCATAAGTGAATTCTACCAGTGCCATAA |
| | | GTGAATTCTACCAGTGCCATATCAAGCTTATACATACTTCTTTACATTCCAATAC |
| | | ATACTTCTTTACATTCCAATACATACTTCTTTACATTCCACCATGGACTTGTACA |
| | | AACACCATTGTCACACACTCCAACAAACACCATTGTCACACACTCCAACAAACAC |
| | | CATTGTCACACACTCCAAGCTAGCCCTAGGTAATCAACCTCTGGATTACAAAATT |
| | | TGTGAAAGATTGACTGGTATTCTTAACTATGTTGCTCCTTTTACGCTATGTGGAT |
| | | ACGCTGCTTTAATGCCTTTGTATCATGCTATTGCTTCCCGTATGGCTTTCATTTT |
| | | CTCCTCCTTGTATAAATCCTGGTTGCTGTCTCTTTATGAGGAGTTGTGGCCCGTT |
| | | GTCAGGCAACGTGGCGTGGTGTGCACTGTGTTTGCTGACGCAACCCCCACTGGTT |
| | | GGGGCATTGCCACCACCTGTCAGCTCCTTTCCGGGACTTTCGCTTTCCCCCTCCC |
| | | TATTGCCACGGCGGAACTCATCGCCGCCTGCCTTGCCCGCTGCTGGACAGGGGCT |
| | | CGGCTGTTGGGCACTGACAATTCCGTGGTGTTGTCGGGGAAATCATCGTCCTTTC |
| | | CTTGGCTGCTCGCCTGTGTTGCCACCTGGATTCTGCGCGGGACGTCCTTCTGCTA |
| | | CGTCCCTTCGGCCCTCAATCCAGCGGACCTTCCTTCCCGCGGCCTGCTGCCGGCT |
| | | CTGCGGCCTCTTCCGCGTCTTCGCCTTCGCCCTCAGACGAGTCGGATCTCCCTTT |
| | | GGGCCGCCTCCCCGCCCTGCAGGGCTCGCTTTCTTGCTGTCCAATTTCTATTAAA |
| | | GGTTCCTTTGTTCCCTAAGTCCAACTACTAAACTGGGGGATATTATGAAGGGCCT |
| | | TGAGCATCTGGATTCTGCCTAATAAAAGATCTTTATTTTCATTAGATCTGTGTGT |
| | | TGGTTTTTTGTGTG |
| 13 | BDNF3 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGGGGTGAGTCACC |
| | | CACACAAAGGAAAAGGGCCTTTCCGTCCTCAGCCGTCGCTTCATGTGACTCCACG |
| | | GAGTACCGGGCGCCGTCCAGGCACCTCGATTAGTTCTCGAGCTTTTGGAGTACGT |
| | | CGTCTTTAGGTTGGGGGGAGGGGTTTTATGCGATGGAGTTTCCCCACACTGAGTG |
| | | GGTGGAGACTGAAGTTAGGCCAGCTTGGCACTTGATGTAATTCTCCTTGGAATTT |
| | | GCCCTTTTTGAGTTTGGATCTTGGTTCATTCTCAAGCCTCAGACAGTGGTTCAAA |
| | | GTTTTTTTCTTCCATTTCAGGTGTCGTGAGGATCCAACTCCTAAAAAACCGCCAC |
| | | CATGACCATCCTGTTCCTGACCATGGTCATTTCCTACTTTGGCTGCATGAAGGCT |
| | | GCCCCCATGAAAGAAGCCAACATCCGCGGCCAGGGCGGCCTGGCCTACCCTGGCG |
| | | TGCGCACCCATGGCACCCTGGAGTCCGTGAATGGCCCCAAGGCCGGCTCCCGCGG |
| | | CCTGACCTCCCTGGCTGACACCTTCGAACATGTGATCGAAGAGCTGCTGGATGAG |
| | | GACCAGAAAGTGCGCCCCAATGAAGAAAACAATAAGGACGCCGACCTGTACACCT |
| | | CCCGCGTGATGCTGTCCTCCCAGGTGCCTCTGGAGCCTCCTCTGCTGTTTCTGCT |
| | | GGAGGAATACAAAAATTACCTGGATGCTGCTAACATGTCCATGCGCGTCCGCCGC |
| | | CACTCCGACCCTGCCCGCCGCGGCGAGCTGTCCGTGTGCGACTCCATTTCCGAGT |
| | | GGGTGACCGCTGCCGACAAAAAGACCGCCGTGGACATGTCCGGCGGCACCGTCAC |
| | | CGTCCTGGAAAAGGTCCCTGTCTCCAAAGGCCAGCTGAAGCAGTACTTCTATGAG |
| | | ACCAAGTGCAATCCCATGGGCTACACCAAAGAAGGCTGCCGCGGCATTGACAAAC |
| | | GGCATTGGAACTCCCAGTGCCGCACCACCCAGTCCTACGTGCGCGCCCTGACCAT |
| | | GGATTCCAAAAAGCGCATTGGCTGGCGCTTCATCCGCATTGACACCTCCTGCGTC |
| | | TGTACCCTGACCATCAAACGCGGCCGCTGAGGTACCTTCTCCATAAAGTAGGAAA |
| | | CACTACATCATCCATAAAGTAGGAAACACTACATCATCCATAAAGTAGGAAACAC |
| | | TACATCATCCATAAAGTAGGAAACACTACAGAATTCGATATCAGTGAATTCTACC |
| | | AGTGCCATAAGTGAATTCTACCAGTGCCATAAGTGAATTCTACCAGTGCCATAAG |
| | | TGAATTCTACCAGTGCCATATCAAGCTTATACATACTTCTTTACATTCCAATACA |
| | | TACTTCTTTACATTCCAATACATACTTCTTTACATTCCACCATGGACTTGTACAA |
| | | ACACCATTGTCACACACTCCAACAAACACCATTGTCACACACTCCAACAAACACC |
| | | ATTGTCACACACTCCAAGCTAGCCCTAGGAATCAACCTCTGGATTACAAAATTTG |
| | | TGAAAGATTGACTGGTATTCTTAACTATGTTGCTCCTTTTACGCTATGTGGATAC |
| | | GCTGCTTTAATGCCTTTGTATCATGCTATTGCTTCCCGTATGGCTTTCATTTTCT |
| | | CCTCCTTGTATAAATCCTGGTTAGTTCTTGCCACGGCGGAACTCATCGCCGCCTG |
| | | CCTTGCCCGCTGCTGGACAGGGGCTCGGCTGTTGGGCACTGACAATTCCGTGGTG |
| | | TTCCTGCAGGGCTCGCTTTCTTGCTGTCCAATTTCTATTAAAGGTTCCTTTGTTC |
| | | CCTAAGTCCAACTACTAAACTGGGGGATATTATGAAGGGCCTTGAGCATCTGGAT |
| | | TCTGCCTAATAAAAGATCTTTATTTTCATTAGATCTGTGTGTTGGTTTTTTGTGT |
| | | G |
| 14 | BDNF4 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACCATGGTCATTTCCTACTTTGGCTGCATGAAGGC |
| | | TGCCCCCATGAAAGAAGCCAACATCCGCGGCCAGGGCGGCCTGGCCTACCCTGGC |
| | | GTGCGCACCCATGGCACCCTGGAGTCCGTGAATGGCCCCAAGGCCGGCTCCCGCG |
| | | GCCTGACCTCCCTGGCTGACACCTTCGAACATGTGATCGAAGAGCTGCTGGATGA |
| | | GGACCAGAAAGTGCGCCCCAATGAAGAAAACAATAAGGACGCCGACCTGTACACC |
| | | TCCCGCGTGATGCTGTCCTCCCAGGTGCCTCTGGAGCCTCCTCTGCTGTTTCTGC |
| | | TGGAGGAATACAAAAATTACCTGGATGCTGCTAACATGTCCATGCGCGTCCGCCG |
| | | CCACTCCGACCCTGCCCGCCGCGGCGAGCTGTCCGTGTGCGACTCCATTTCCGAG |
| | | TGGGTGACCGCTGCCGACAAAAAGACCGCCGTGGACATGTCCGGCGGCACCGTCA |
| | | CCGTCCTGGAAAAGGTCCCTGTCTCCAAAGGCCAGCTGAAGCAGTACTTCTATGA |
| | | GACCAAGTGCAATCCCATGGGCTACACCAAAGAAGGCTGCCGCGGCATTGACAAA |
| | | CGGCATTGGAACTCCCAGTGCCGCACCACCCAGTCCTACGTGCGCGCCCTGACCA |
| | | TGGATTCCAAAAAGCGCATTGGCTGGCGCTTCATCCGCATTGACACCTCCTGCGT |
| | | CTGTACCCTGACCATCAAACGCGGCCGCTGAGGTACCTTCTAATCAACCTCTGGA |
| | | TTACAAAATTTGTGAAAGATTGACTGGTATTCTTAACTATGTTGCTCCTTTTACG |
| | | CTATGTGGATACGCTGCTTTAATGCCTTTGTATCATGCTATTGCTTCCCGTATGG |
| | | CTTTCATTTTCTCCTCCTTGTATAAATCCTGGTTGCTGTCTCTTTATGAGGAGTT |
| | | GTGGCCCGTTGTCAGGCAACGTGGCGTGGTGTGCACTGTGTTTGCTGACGCAACC |
| | | CCCACTGGTTGGGGCATTGCCACCACCTGTCAGCTCCTTTCCGGGACTTTCGCTT |
| | | TCCCCCTCCCTATTGCCACGGCGGAACTCATCGCCGCCTGCCTTGCCCGCTGCTG |
| | | GACAGGGGCTCGGCTGTTGGGCACTGACAATTCCGTGGTGTTGTCGGGGAAATCA |
| | | TCGTCCTTTCCTTGGCTGCTCGCCTGTGTTGCCACCTGGATTCTGCGCGGGACGT |
| | | CCTTCTGCTACGTCCCTTCGGCCCTCAATCCAGCGGACCTTCCTTCCCGCGGCCT |
| | | GCTGCCGGCTCTGCGGCCTCTTCCGCGTCTTCGCCTTCGCCCTCAGACGAGTCGG |
| | | ATCTCCCTTTGGGCCGCCTCCCCGCCCTGCAGGGCTCGCTTTCTTGCTGTCCAAT |
| | | TTCTATTAAAGGTTCCTTTGTTCCCTAAGTCCAACTACTAAACTGGGGGATATTA |
| | | TGAAGGGCCTTGAGCATCTGGATTCTGCCTAATAAAAGATCTTTATTTTCATTAG |
| | | ATCTGTGTGTTGGTTTTTTGTGTG |
| 15 | BDNF5 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACCATGGTCATTTCCTACTTTGGCTGCATGAAGGC |
| | | TGCCCCCATGAAAGAAGCCAACATCCGCGGCCAGGGCGGCCTGGCCTACCCTGGC |
| | | GTGCGCACCCATGGCACCCTGGAGTCCGTGAATGGCCCCAAGGCCGGCTCCCGCG |
| | | GCCTGACCTCCCTGGCTGACACCTTCGAACATGTGATCGAAGAGCTGCTGGATGA |
| | | GGACCAGAAAGTGCGCCCCAATGAAGAAAACAATAAGGACGCCGACCTGTACACC |
| | | TCCCGCGTGATGCTGTCCTCCCAGGTGCCTCTGGAGCCTCCTCTGCTGTTTCTGC |
| | | TGGAGGAATACAAAAATTACCTGGATGCTGCTAACATGTCCATGCGCGTCCGCCG |
| | | CCACTCCGACCCTGCCCGCCGCGGCGAGCTGTCCGTGTGCGACTCCATTTCCGAG |
| | | TGGGTGACCGCTGCCGACAAAAAGACCGCCGTGGACATGTCCGGCGGCACCGTCA |
| | | CCGTCCTGGAAAAGGTCCCTGTCTCCAAAGGCCAGCTGAAGCAGTACTTCTATGA |
| | | GACCAAGTGCAATCCCATGGGCTACACCAAAGAAGGCTGCCGCGGCATTGACAAA |
| | | CGGCATTGGAACTCCCAGTGCCGCACCACCCAGTCCTACGTGCGCGCCCTGACCA |
| | | TGGATTCCAAAAAGCGCATTGGCTGGCGCTTCATCCGCATTGACACCTCCTGCGT |
| | | CTGTACCCTGACCATCAAACGCGGCCGCTGAGGTACCTTCAATCAACCTCTGGAT |
| | | TACAAAATTTGTGAAAGATTGACTGGTATTCTTAACTATGTTGCTCCTTTTACGC |
| | | TATGTGGATACGCTGCTTTAATGCCTTTGTATCATGCTATTGCTTCCCGTATGGC |
| | | TTTCATTTTCTCCTCCTTGTATAAATCCTGGTTAGTTCTTGCCACGGCGGAACTC |
| | | ATCGCCGCCTGCCTTGCCCGCTGCTGGACAGGGGCTCGGCTGTTGGGCACTGACA |
| | | ATTCCGTGGTGTTCCTGCAGGGCTCGCTTTCTTGCTGTCCAATTTCTATTAAAGG |
| | | TTCCTTTGTTCCCTAAGTCCAACTACTAAACTGGGGGATATTATGAAGGGCCTTG |
| | | AGCATCTGGATTCTGCCTAATAAAAGATCTTTATTTTCATTAGATCTGTGTGTTG |
| | | GTTTTTTGTGTG |
| 16 | BDNF6 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACCATGGTCATTTCCTACTTTGGCTGCATGAAGGC |
| | | TGCCCCCATGAAAGAAGCCAACATCCGCGGCCAGGGCGGCCTGGCCTACCCTGGC |
| | | GTGCGCACCCATGGCACCCTGGAGTCCGTGAATGGCCCCAAGGCCGGCTCCCGCG |
| | | GCCTGACCTCCCTGGCTGACACCTTCGAACATGTGATCGAAGAGCTGCTGGATGA |
| | | GGACCAGAAAGTGCGCCCCAATGAAGAAAACAATAAGGACGCCGACCTGTACACC |
| | | TCCCGCGTGATGCTGTCCTCCCAGGTGCCTCTGGAGCCTCCTCTGCTGTTTCTGC |
| | | TGGAGGAATACAAAAATTACCTGGATGCTGCTAACATGTCCATGCGCGTCCGCCG |
| | | CCACTCCGACCCTGCCCGCCGCGGCGAGCTGTCCGTGTGCGACTCCATTTCCGAG |
| | | TGGGTGACCGCTGCCGACAAAAAGACCGCCGTGGACATGTCCGGCGGCACCGTCA |
| | | CCGTCCTGGAAAAGGTCCCTGTCTCCAAAGGCCAGCTGAAGCAGTACTTCTATGA |
| | | GACCAAGTGCAATCCCATGGGCTACACCAAAGAAGGCTGCCGCGGCATTGACAAA |
| | | CGGCATTGGAACTCCCAGTGCCGCACCACCCAGTCCTACGTGCGCGCCCTGACCA |
| | | TGGATTCCAAAAAGCGCATTGGCTGGCGCTTCATCCGCATTGACACCTCCTGCGT |
| | | CTGTACCCTGACCATCAAACGCGGCCGCTGAGGTACCTTCCCTGCAGGGCTCGCT |
| | | TTCTTGCTGTCCAATTTCTATTAAAGGTTCCTTTGTTCCCTAAGTCCAACTACTA |
| | | AACTGGGGGATATTATGAAGGGCCTTGAGCATCTGGATTCTGCCTAATAAAAGAT |
| | | CTTTATTTTCATTAGATCTGTGTGTTGGTTTTTTGTGTG |
| 17 | BDNF7 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACCATGGTCATTTCCTACTTTGGCTGCATGAAGGC |
| | | TGCCCCCATGAAAGAAGCCAACATCCGCGGCCAGGGCGGCCTGGCCTACCCTGGC |
| | | GTGCGCACCCATGGCACCCTGGAGTCCGTGAATGGCCCCAAGGCCGGCTCCCGCG |
| | | GCCTGACCTCCCTGGCTGACACCTTCGAACATGTGATCGAAGAGCTGCTGGATGA |
| | | GGACCAGAAAGTGCGCCCCAATGAAGAAAACAATAAGGACGCCGACCTGTACACC |
| | | TCCCGCGTGATGCTGTCCTCCCAGGTGCCTCTGGAGCCTCCTCTGCTGTTTCTGC |
| | | TGGAGGAATACAAAAATTACCTGGATGCTGCTAACATGTCCATGCGCGTCCGCCG |
| | | CCACTCCGACCCTGCCCGCCGCGGCGAGCTGTCCGTGTGCGACTCCATTTCCGAG |
| | | TGGGTGACCGCTGCCGACAAAAAGACCGCCGTGGACATGTCCGGCGGCACCGTCA |
| | | CCGTCCTGGAAAAGGTCCCTGTCTCCAAAGGCCAGCTGAAGCAGTACTTCTATGA |
| | | GACCAAGTGCAATCCCATGGGCTACACCAAAGAAGGCTGCCGCGGCATTGACAAA |
| | | CGGCATTGGAACTCCCAGTGCCGCACCACCCAGTCCTACGTGCGCGCCCTGACCA |
| | | TGGATTCCAAAAAGCGCATTGGCTGGCGCTTCATCCGCATTGACACCTCCTGCGT |
| | | CTGTACCCTGACCATCAAACGCGGCCGCTGAGGTACCTTCTCCATAAAGTAGGAA |
| | | ACACTACATCATCCATAAAGTAGGAAACACTACATCATCCATAAAGTAGGAAACA |
| | | CTACATCATCCATAAAGTAGGAAACACTACAGAATTCGATATCTAATCAACCTCT |
| | | GGATTACAAAATTTGTGAAAGATTGACTGGTATTCTTAACTATGTTGCTCCTTTT |
| | | ACGCTATGTGGATACGCTGCTTTAATGCCTTTGTATCATGCTATTGCTTCCCGTA |
| | | TGGCTTTCATTTTCTCCTCCTTGTATAAATCCTGGTTGCTGTCTCTTTATGAGGA |
| | | GTTGTGGCCCGTTGTCAGGCAACGTGGCGTGGTGTGCACTGTGTTTGCTGACGCA |
| | | ACCCCCACTGGTTGGGGCATTGCCACCACCTGTCAGCTCCTTTCCGGGACTTTCG |
| | | CTTTCCCCCTCCCTATTGCCACGGCGGAACTCATCGCCGCCTGCCTTGCCCGCTG |
| | | CTGGACAGGGGCTCGGCTGTTGGGCACTGACAATTCCGTGGTGTTGTCGGGGAAA |
| | | TCATCGTCCTTTCCTTGGCTGCTCGCCTGTGTTGCCACCTGGATTCTGCGCGGGA |
| | | CGTCCTTCTGCTACGTCCCTTCGGCCCTCAATCCAGCGGACCTTCCTTCCCGCGG |
| | | CCTGCTGCCGGCTCTGCGGCCTCTTCCGCGTCTTCGCCTTCGCCCTCAGACGAGT |
| | | CGGATCTCCCTTTGGGCCGCCTCCCCGCCCTGCAGGGCTCGCTTTCTTGCTGTCC |
| | | AATTTCTATTAAAGGTTCCTTTGTTCCCTAAGTCCAACTACTAAACTGGGGGATA |
| | | TTATGAAGGGCCTTGAGCATCTGGATTCTGCCTAATAAAAGATCTTTATTTTCAT |
| | | TAGATCTGTGTGTTGGTTTTTTGTGTG |
| 18 | BDNF8 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTTTTCCTTACTATGGTTATTTCATACTTTGGTTGCATGAAGGC |
| | | TGCCCCCATGAAAGAAGCAAACATCCGAGGACAAGGTGGCTTGGCCTACCCAGGT |
| | | GTGCGGACCCATGGGACTCTGGAGAGCGTGAATGGGCCCAAGGCAGGTTCAAGAG |
| | | GCTTGACATCATTGGCTGACACTTTCGAACACGTGATAGAAGAGCTGTTGGATGA |
| | | GGACCAGAAAGTTCGGCCCAATGAAGAAAACAATAAGGACGCAGACTTGTACACG |
| | | TCCAGGGTGATGCTCAGTAGTCAAGTGCCTTTGGAGCCTCCTCTTCTCTTTCTGC |
| | | TGGAGGAATACAAAAATTACCTAGATGCTGCAAACATGTCCATGAGGGTCCGGCG |
| | | CCACTCTGACCCTGCCCGCCGAGGGGAGCTGAGCGTGTGTGACAGTATTAGTGAG |
| | | TGGGTAACGGCGGCAGACAAAAAGACTGCAGTGGACATGTCGGGGGGACGGTCAC |
| | | AGTCCTTGAAAAGGTCCCTGTATCAAAAGGCCAACTGAAGCAATACTTCTACGAG |
| | | ACCAAGTGCAATCCCATGGGTTACACAAAAGAAGGCTGCAGGGGCATAGACAAAA |
| | | GGCATTGGAACTCCCAGTGCCGAACTACCCAGTCGTACGTGCGGGCCCTTACCAT |
| | | GGATAGCAAAAAGAGAATTGGCTGGCGATTCATAAGGATAGACACTTCTTGTGTA |
| | | TGTACATTGACCATTAAAAGGGGAAGATAGTGAGGTACCTTCTAATCAACCTCTG |
| | | GATTACAAAATTTGTGAAAGATTGACTGGTATTCTTAACTATGTTGCTCCTTTTA |
| | | CGCTATGTGGATACGCTGCTTTAATGCCTTTGTATCATGCTATTGCTTCCCGTAT |
| | | GGCTTTCATTTTCTCCTCCTTGTATAAATCCTGGTTGCTGTCTCTTTATGAGGAG |
| | | TTGTGGCCCGTTGTCAGGCAACGTGGCGTGGTGTGCACTGTGTTTGCTGACGCAA |
| | | CCCCCACTGGTTGGGGCATTGCCACCACCTGTCAGCTCCTTTCCGGGACTTTCGC |
| | | TTTCCCCCTCCCTATTGCCACGGCGGAACTCATCGCCGCCTGCCTTGCCCGCTGC |
| | | TGGACAGGGGCTCGGCTGTTGGGCACTGACAATTCCGTGGTGTTGTCGGGGAAAT |
| | | CATCGTCCTTTCCTTGGCTGCTCGCCTGTGTTGCCACCTGGATTCTGCGCGGGAC |
| | | GTCCTTCTGCTACGTCCCTTCGGCCCTCAATCCAGCGGACCTTCCTTCCCGCGGC |
| | | CTGCTGCCGGCTCTGCGGCCTCTTCCGCGTCTTCGCCTTCGCCCTCAGACGAGTC |
| | | GGATCTCCCTTTGGGCCGCCTCCCCGCCCTGCAGGGCTCGCTTTCTTGCTGTCCA |
| | | ATTTCTATTAAAGGTTCCTTTGTTCCCTAAGTCCAACTACTAAACTGGGGGATAT |
| | | TATGAAGGGCCTTGAGCATCTGGATTCTGCCTAATAAAAGATCTTTATTTTCATT |
| | | AGATCTGTGTGTTGGTTTTTTGTGTG |
| 19 | BDNF9 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTTCTGACAATGGTTATTTCATATTTTGGTTGCATGAAAGC |
| | | AGCTCCAATGAAGGAAGCAAACATCAGAGGACAAGGGGGCCTAGCTTATCCTGGA |
| | | GTTAGAACCCATGGGACTTTGGAGTCTGTCAATGGCCCAAAAGCTGGAAGCAGGG |
| | | GCCTGACAAGCCTGGCAGATACTTTTGAACATGTCATTGAGGAACTCCTAGATGA |
| | | AGACCAAAAAGTTAGACCAAATGAAGAGAACAATAAAGATGCTGACCTTTATACT |
| | | AGCAGAGTCATGCTCTCCTCTCAAGTCCCACTAGAACCCCCACTCCTGTTCCTAC |
| | | TGGAGGAATACAAAAATTACCTTGATGCAGCCAATATGAGTATGAGAGTGAGGAG |
| | | ACACAGTGACCCTGCTAGGAGAGGGGAACTCTCAGTATGTGACTCCATTAGTGAA |
| | | TGGGTGACTGCTGCAGACAAAAAGACAGCAGTTGACATGAGTGGTGGCACAGTAA |
| | | CTGTACTAGAAAAAGTACCAGTGAGTAAAGGTCAGCTTAAGCAATATTTTTATGA |
| | | GACAAAATGCAATCCAATGGGATATACCAAGGAAGGATGTAGGGGCATTGATAAA |
| | | AGGCACTGGAATAGCCAATGTAGAACCACACAATCTTATGTTAGGGCCCTTACCA |
| | | TGGACTCCAAGAAAAGAATTGGATGGAGATTTATCAGGATTGATACAAGTTGTGT |
| | | CTGCACCCTGACCATCAAAAGAGGAAGATGATGAGGTACCTTCTAATCAACCTCT |
| | | GGATTACAAAATTTGTGAAAGATTGACTGGTATTCTTAACTATGTTGCTCCTTTT |
| | | ACGCTATGTGGATACGCTGCTTTAATGCCTTTGTATCATGCTATTGCTTCCCGTA |
| | | TGGCTTTCATTTTCTCCTCCTTGTATAAATCCTGGTTGCTGTCTCTTTATGAGGA |
| | | GTTGTGGCCCGTTGTCAGGCAACGTGGCGTGGTGTGCACTGTGTTTGCTGACGCA |
| | | ACCCCCACTGGTTGGGGCATTGCCACCACCTGTCAGCTCCTTTCCGGGACTTTCG |
| | | CTTTCCCCCTCCCTATTGCCACGGCGGAACTCATCGCCGCCTGCCTTGCCCGCTG |
| | | CTGGACAGGGGCTCGGCTGTTGGGCACTGACAATTCCGTGGTGTTGTCGGGGAAA |
| | | TCATCGTCCTTTCCTTGGCTGCTCGCCTGTGTTGCCACCTGGATTCTGCGCGGGA |
| | | CGTCCTTCTGCTACGTCCCTTCGGCCCTCAATCCAGCGGACCTTCCTTCCCGCGG |
| | | CCTGCTGCCGGCTCTGCGGCCTCTTCCGCGTCTTCGCCTTCGCCCTCAGACGAGT |
| | | CGGATCTCCCTTTGGGCCGCCTCCCCGCCCTGCAGGGCTCGCTTTCTTGCTGTCC |
| | | AATTTCTATTAAAGGTTCCTTTGTTCCCTAAGTCCAACTACTAAACTGGGGGATA |
| | | TTATGAAGGGCCTTGAGCATCTGGATTCTGCCTAATAAAAGATCTTTATTTTCAT |
| | | TAGATCTGTGTGTTGGTTTTTTGTGTG |
| 20 | BDNF10 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACTATGGTGATTAGCTACTTTGGATGCATGAAGGC |
| | | TGCCCCTATGAAAGAAGCTAACATCAGAGGACAAGGAGGCCTGGCCTACCCAGGA |
| | | GTGAGAACCCATGGAACTCTGGAGTCTGTGAATGGCCCAAAGGCTGGAAGCAGAG |
| | | GCCTGACAAGCCTGGCTGACACTTTTGAACATGTGATTGAAGAGCTGCTGGATGA |
| | | GGACCAGAAAGTGAGACCTAATGAAGAAAACAATAAGGATGCTGACCTGTACACC |
| | | AGCAGAGTGATGCTGAGCAGCCAAGTGCCTCTGGAGCCTCCTCTGCTGTTTCTGC |
| | | TGGAGGAATACAAAAATTACCTGGATGCTGCTAACATGAGCATGAGAGTCAGAAG |
| | | ACACTCTGACCCTGCCAGAAGAGGAGAGCTGTCTGTGTGTGACAGCATTTCTGAG |
| | | TGGGTGACAGCTGCTGACAAAAAGACTGCTGTGGACATGTCTGGAGGAACTGTCA |
| | | CAGTCCTGGAAAAGGTCCCTGTGAGCAAAGGCCAACTGAAGCAATACTTCTATGA |
| | | GACCAAGTGCAATCCAATGGGCTACACAAAAGAAGGCTGCAGAGGCATTGACAAA |
| | | AGACATTGGAACAGCCAGTGCAGAACTACCCAGAGCTATGTGAGAGCCCTGACCA |
| | | TGGATAGCAAAAAGAGAATTGGCTGGAGATTCATTAGAATTGACACTAGCTGTGT |
| | | CTGTACACTGACCATTAAAAGAGGAAGATGATGAGGTACCTTCTAATCAACCTCT |
| | | GGATTACAAAATTTGTGAAAGATTGACTGGTATTCTTAACTATGTTGCTCCTTTT |
| | | ACGCTATGTGGATACGCTGCTTTAATGCCTTTGTATCATGCTATTGCTTCCCGTA |
| | | TGGCTTTCATTTTCTCCTCCTTGTATAAATCCTGGTTGCTGTCTCTTTATGAGGA |
| | | GTTGTGGCCCGTTGTCAGGCAACGTGGCGTGGTGTGCACTGTGTTTGCTGACGCA |
| | | ACCCCCACTGGTTGGGGCATTGCCACCACCTGTCAGCTCCTTTCCGGGACTTTCG |
| | | CTTTCCCCCTCCCTATTGCCACGGCGGAACTCATCGCCGCCTGCCTTGCCCGCTG |
| | | CTGGACAGGGGCTCGGCTGTTGGGCACTGACAATTCCGTGGTGTTGTCGGGGAAA |
| | | TCATCGTCCTTTCCTTGGCTGCTCGCCTGTGTTGCCACCTGGATTCTGCGCGGGA |
| | | CGTCCTTCTGCTACGTCCCTTCGGCCCTCAATCCAGCGGACCTTCCTTCCCGCGG |
| | | CCTGCTGCCGGCTCTGCGGCCTCTTCCGCGTCTTCGCCTTCGCCCTCAGACGAGT |
| | | CGGATCTCCCTTTGGGCCGCCTCCCCGCCCTGCAGGGCTCGCTTTCTTGCTGTCC |
| | | AATTTCTATTAAAGGTTCCTTTGTTCCCTAAGTCCAACTACTAAACTGGGGGATA |
| | | TTATGAAGGGCCTTGAGCATCTGGATTCTGCCTAATAAAAGATCTTTATTTTCAT |
| | | TAGATCTGTGTGTTGGTTTTTTGTGTG |
| 21 | BDNF11 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATTCTGTTTCTGACCATGGTTATTAGCTATTTCGGGTGCATGAAAGC |
| | | CGCCCCGATGAAAGAAGCGAATATCCGGGGCCAGGGCGGCCTGGCGTATCCGGGC |
| | | GTCCGGACCCACGGGACCCTGGAATCCGTGAACGGCCCGAAAGCCGGGTCCCGGG |
| | | GCCTGACCAGCCTGGCGGATACCTTTGAACATGTGATTGAAGAACTGCTGGATGA |
| | | AGATCAGAAAGTCCGTCCGAATGAAGAAAATAACAAAGATGCGGATCTGTATACC |
| | | TCCCGGGTTATGCTGAGCAGCCAGGTTCCGCTGGAACCGCCGCTGCTGTTTCTGC |
| | | TGGAAGAATATAAGAATTATCTGGACGCCGCCAATATGAGCATGCGGGTCCGCCG |
| | | CCATTCCGATCCGGCGCGCCGCGGCGAACTGTCCGTGTGCGATAGCATTTCCGAA |
| | | TGGGTGACCGCCGCCGATAAGAAAACCGCGGTGGATATGTCCGGCGGCACCGTGA |
| | | CCGTGCTGGAGAAAGTTCCGGTGAGCAAAGGGCAGCTGAAACAGTATTTCTATGA |
| | | AACCAAATGCAATCCGATGGGCTATACCAAAGAGGGCTGCCGGGGCATTGATAAA |
| | | CGGCATTGGAATAGCCAGTGCCGGACCACCCAGAGCTATGTCCGGGCCCTAACCA |
| | | TGGATAGCAAGAAACGGATCGGGTGGCGCTTTATCCGGATTGATACCAGCTGCGT |
| | | GTGCACCCTAACCATCAAACGGGGCCGGTAATGAGGTACCTTCTAATCAACCTCT |
| | | GGATTACAAAATTTGTGAAAGATTGACTGGTATTCTTAACTATGTTGCTCCTTTT |
| | | ACGCTATGTGGATACGCTGCTTTAATGCCTTTGTATCATGCTATTGCTTCCCGTA |
| | | TGGCTTTCATTTTCTCCTCCTTGTATAAATCCTGGTTGCTGTCTCTTTATGAGGA |
| | | GTTGTGGCCCGTTGTCAGGCAACGTGGCGTGGTGTGCACTGTGTTTGCTGACGCA |
| | | ACCCCCACTGGTTGGGGCATTGCCACCACCTGTCAGCTCCTTTCCGGGACTTTCG |
| | | CTTTCCCCCTCCCTATTGCCACGGCGGAACTCATCGCCGCCTGCCTTGCCCGCTG |
| | | CTGGACAGGGGCTCGGCTGTTGGGCACTGACAATTCCGTGGTGTTGTCGGGGAAA |
| | | TCATCGTCCTTTCCTTGGCTGCTCGCCTGTGTTGCCACCTGGATTCTGCGCGGGA |
| | | CGTCCTTCTGCTACGTCCCTTCGGCCCTCAATCCAGCGGACCTTCCTTCCCGCGG |
| | | CCTGCTGCCGGCTCTGCGGCCTCTTCCGCGTCTTCGCCTTCGCCCTCAGACGAGT |
| | | CGGATCTCCCTTTGGGCCGCCTCCCCGCCCTGCAGGGCTCGCTTTCTTGCTGTCC |
| | | AATTTCTATTAAAGGTTCCTTTGTTCCCTAAGTCCAACTACTAAACTGGGGGATA |
| | | TTATGAAGGGCCTTGAGCATCTGGATTCTGCCTAATAAAAGATCTTTATTTTCAT |
| | | TAGATCTGTGTGTTGGTTTTTTGTGTG |
| 22 | BDNF12 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACTATGGTGATTAGCTACTTTGGATGCATGAAGGC |
| | | TGCCCCTATGAAAGAAGCTAACATCAGAGGACAAGGAGGCCTGGCCTACCCAGGA |
| | | GTGAGAACCCATGGAACTCTGGAGTCTGTGAATGGCCCAAAGGCTGGAAGCAGAG |
| | | GCCTGACAAGCCTGGCTGACACTTTTGAACATGTGATTGAAGAGCTGCTGGATGA |
| | | GGACCAGAAAGTGAGACCTAATGAAGAAAACAATAAGGATGCTGACCTGTACACC |
| | | AGCAGAGTGATGCTGAGCAGCCAAGTGCCTCTGGAGCCTCCTCTGCTGTTTCTGC |
| | | TGGAGGAATACAAAAATTACCTGGATGCTGCTAACATGAGCATGAGAGTCAGAAG |
| | | ACACTCTGACCCTGCCAGAAGAGGAGAGCTGTCTGTGTGTGACAGCATTTCTGAG |
| | | TGGGTGACAGCTGCTGACAAAAAGACTGCTGTGGACATGTCTGGAGGAACTGTCA |
| | | CAGTCCTGGAAAAGGTCCCTGTGAGCAAAGGCCAACTGAAGCAATACTTCTATGA |
| | | GACCAAGTGCAATCCAATGGGCTACACAAAAGAAGGCTGCAGAGGCATTGACAAA |
| | | AGACATTGGAACAGCCAGTGCAGAACTACCCAGAGCTATGTGAGAGCCCTGACCA |
| | | TGGATAGCAAAAAGAGAATTGGCTGGAGATTCATTAGAATTGACACTAGCTGTGT |
| | | CTGTACACTGACCATTAAAAGAGGAAGATGATGAGGTACCTTCTCCATAAAGTAG |
| | | GAAACACTACATCATCCATAAAGTAGGAAACACTACATCATCCATAAAGTAGGAA |
| | | ACACTACATCATCCATAAAGTAGGAAACACTACAGAATTCGATATCAGTGAATTC |
| | | TACCAGTGCCATAAGTGAATTCTACCAGTGCCATAAGTGAATTCTACCAGTGCCA |
| | | TAAGTGAATTCTACCAGTGCCATATCAAGCTTATACATACTTCTTTACATTCCAA |
| | | TACATACTTCTTTACATTCCAATACATACTTCTTTACATTCCACCATGGACTTGT |
| | | ACAAACACCATTGTCACACACTCCAACAAACACCATTGTCACACACTCCAACAAA |
| | | CACCATTGTCACACACTCCAAGCTAGCCCTAGGAATCAACCTCTGGATTACAAAA |
| | | TTTGTGAAAGATTGACTGGTATTCTTAACTATGTTGCTCCTTTTACGCTATGTGG |
| | | ATACGCTGCTTTAATGCCTTTGTATCATGCTATTGCTTCCCGTATGGCTTTCATT |
| | | TTCTCCTCCTTGTATAAATCCTGGTTAGTTCTTGCCACGGCGGAACTCATCGCCG |
| | | CCTGCCTTGCCCGCTGCTGGACAGGGGCTCGGCTGTTGGGCACTGACAATTCCGT |
| | | GGTGTTCCTGCAGGGCTCGCTTTCTTGCTGTCCAATTTCTATTAAAGGTTCCTTT |
| | | GTTCCCTAAGTCCAACTACTAAACTGGGGGATATTATGAAGGGCCTTGAGCATCT |
| | | GGATTCTGCCTAATAAAAGATCTTTATTTTCATTAGATCTGTGTGTTGGTTTTTT |
| | | GTGTG |
| 23 | BDNF13 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACTATGGTGATTAGCTACTTTGGATGCATGAAGGC |
| | | TGCCCCTATGAAAGAAGCTAACATCAGAGGACAAGGAGGCCTGGCCTACCCAGGA |
| | | GTGAGAACCCATGGAACTCTGGAGTCTGTGAATGGCCCAAAGGCTGGAAGCAGAG |
| | | GCCTGACAAGCCTGGCTGACACTTTTGAACATGTGATTGAAGAGCTGCTGGATGA |
| | | GGACCAGAAAGTGAGACCTAATGAAGAAAACAATAAGGATGCTGACCTGTACACC |
| | | AGCAGAGTGATGCTGAGCAGCCAAGTGCCTCTGGAGCCTCCTCTGCTGTTTCTGC |
| | | TGGAGGAATACAAAAATTACCTGGATGCTGCTAACATGAGCATGAGAGTCAGAAG |
| | | ACACTCTGACCCTGCCAGAAGAGGAGAGCTGTCTGTGTGTGACAGCATTTCTGAG |
| | | TGGGTGACAGCTGCTGACAAAAAGACTGCTGTGGACATGTCTGGAGGAACTGTCA |
| | | CAGTCCTGGAAAAGGTCCCTGTGAGCAAAGGCCAACTGAAGCAATACTTCTATGA |
| | | GACCAAGTGCAATCCAATGGGCTACACAAAAGAAGGCTGCAGAGGCATTGACAAA |
| | | AGACATTGGAACAGCCAGTGCAGAACTACCCAGAGCTATGTGAGAGCCCTGACCA |
| | | TGGATAGCAAAAAGAGAATTGGCTGGAGATTCATTAGAATTGACACTAGCTGTGT |
| | | CTGTACACTGACCATTAAAAGAGGAAGATGATGAGGTACCTTCTCCATAAAGTAG |
| | | GAAACACTACATCATCCATAAAGTAGGAAACACTACATCATCCATAAAGTAGGAA |
| | | ACACTACATCATCCATAAAGTAGGAAACACTACAGAATTCGATATCAGTGAATTC |
| | | TACCAGTGCCATAAGTGAATTCTACCAGTGCCATAAGTGAATTCTACCAGTGCCA |
| | | TAAGTGAATTCTACCAGTGCCATATCAAGCTTATACATACTTCTTTACATTCCAA |
| | | TACATACTTCTTTACATTCCAATACATACTTCTTTACATTCCACCATGGACTTGT |
| | | ACAAACACCATTGTCACACACTCCAACAAACACCATTGTCACACACTCCAACAAA |
| | | CACCATTGTCACACACTCCAAGCTAGCCCTAGGAATCAACCTCTGGATTACAAAA |
| | | TTTGTGAAAGATTGACTGGTATTCTTAACTATGTTGCTCCTTTTACGCTATGTGG |
| | | ATACGCTGCTTTAATGCCTTTGTATCATGCTATTGCTTCCCGTATGGCTTTCATT |
| | | TTCTCCTCCTTGTATAAATCCTGGTTAGTTCTTGCCACGGCGGAACTCATCGCCG |
| | | CCTGCCTTGCCCGCTGCTGGACAGGGGCTCGGCTGTTGGGCACTGACAATTCCGT |
| | | GGTGTTCCTGCAGGCTGTGCCTTCTAGTTGCCAGCCATCTGTTGTTTGCCCCTCC |
| | | CCCGTGCCTTCCTTGACCCTGGAAGGTGCCACTCCCACTGTCCTTTCCTAATAAA |
| | | ATGAGGAAATTGCATCGCATTGTCTGAGTAGGTGTCATTCTATTCTGGGGGGTGG |
| | | GGTGGGGCAGGACAGCAAGGGGGAGGATTGGGAAGACAATAGCAGGCATGCTGGG |
| | | GATGGCCCGGGCTCTATGG |
| 24 | BDNF14 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACTATGGTGATTAGCTACTTTGGATGCATGAAGGC |
| | | TGCCCCTATGAAAGAAGCTAACATCAGAGGACAAGGAGGCCTGGCCTACCCAGGA |
| | | GTGAGAACCCATGGAACTCTGGAGTCTGTGAATGGCCCAAAGGCTGGAAGCAGAG |
| | | GCCTGACAAGCCTGGCTGACACTTTTGAACATGTGATTGAAGAGCTGCTGGATGA |
| | | GGACCAGAAAGTGAGACCTAATGAAGAAAACAATAAGGATGCTGACCTGTACACC |
| | | AGCAGAGTGATGCTGAGCAGCCAAGTGCCTCTGGAGCCTCCTCTGCTGTTTCTGC |
| | | TGGAGGAATACAAAAATTACCTGGATGCTGCTAACATGAGCATGAGAGTCAGAAG |
| | | ACACTCTGACCCTGCCAGAAGAGGAGAGCTGTCTGTGTGTGACAGCATTTCTGAG |
| | | TGGGTGACAGCTGCTGACAAAAAGACTGCTGTGGACATGTCTGGAGGAACTGTCA |
| | | CAGTCCTGGAAAAGGTCCCTGTGAGCAAAGGCCAACTGAAGCAATACTTCTATGA |
| | | GACCAAGTGCAATCCAATGGGCTACACAAAAGAAGGCTGCAGAGGCATTGACAAA |
| | | AGACATTGGAACAGCCAGTGCAGAACTACCCAGAGCTATGTGAGAGCCCTGACCA |
| | | TGGATAGCAAAAAGAGAATTGGCTGGAGATTCATTAGAATTGACACTAGCTGTGT |
| | | CTGTACACTGACCATTAAAAGAGGAAGATGATGAGGTACCTTCTCCATAAAGTAG |
| | | GAAACACTACATCATCCATAAAGTAGGAAACACTACATCATCCATAAAGTAGGAA |
| | | ACACTACATCATCCATAAAGTAGGAAACACTACAGAATTCGATATCAGTGAATTC |
| | | TACCAGTGCCATAAGTGAATTCTACCAGTGCCATAAGTGAATTCTACCAGTGCCA |
| | | TAAGTGAATTCTACCAGTGCCATATCAAGCTTATACATACTTCTTTACATTCCAA |
| | | TACATACTTCTTTACATTCCAATACATACTTCTTTACATTCCACCATGGACTTGT |
| | | ACAAACACCATTGTCACACACTCCAACAAACACCATTGTCACACACTCCAACAAA |
| | | CACCATTGTCACACACTCCAAGCTAGCCCTAGGAATCAACCTCTGGATTACAAAA |
| | | TTTGTGAAAGATTGACTGGTATTCTTAACTATGTTGCTCCTTTTACGCTATGTGG |
| | | ATACGCTGCTTTAATGCCTTTGTATCATGCTATTGCTTCCCGTATGGCTTTCATT |
| | | TTCTCCTCCTTGTATAAATCCTGGTTAGTTCTTGCCACGGCGGAACTCATCGCCG |
| | | CCTGCCTTGCCCGCTGCTGGACAGGGGCTCGGCTGTTGGGCACTGACAATTCCGT |
| | | GGTGTTCCTGCAGGACGGGTGGCATCCCTGTGACCCCTCCCCAGTGCCTCTCCTG |
| | | GCCCTGGAAGTTGCCACTCCAGTGCCCACCAGCCTTGTCCTAATAAAATTAAGTT |
| | | GCATCATTTTGTCTGACTAGGTGTCCTTCTATAATATTATGGGGTGGAGGGGGGT |
| | | GGTATGGAGCAAGGGGCAAGTTGGGAAGACAACCTGTAGGGCCTGCGGGGTCTAT |
| | | TGGGAACCAAGCTGGAGTGCAGTGGCACAATCTTGGCTCACTGCAATCTCCGCCT |
| | | CCTGGGTTCAAGCGATTCTCCTGCCTCAGCCTCCCGAGTTGTTGGGATTCCAGGC |
| | | ATGCATGACCAGGCTCAGCTAATTTTTGTTTTTTTGGTAGAGACGGGGTTTCACC |
| | | ATATTGGCCAGGCTGGTCTCCAACTCCTAATCTCAGGTGATCTACCCACCTTGGC |
| | | CTCCCAAATTGCTGGGATTACAGGCGTGAACCACTGCTCCCTTCCCTGTCCTT |
| 25 | BDNF15 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTGTTCCTGACTATGGTGATTAGCTACTTTGGATGCATGAAGGC |
| | | TGCCCCTATGAAAGAAGCTAACATCAGAGGACAAGGAGGCCTGGCCTACCCAGGA |
| | | GTGAGAACCCATGGAACTCTGGAGTCTGTGAATGGCCCAAAGGCTGGAAGCAGAG |
| | | GCCTGACAAGCCTGGCTGACACTTTTGAACATGTGATTGAAGAGCTGCTGGATGA |
| | | GGACCAGAAAGTGAGACCTAATGAAGAAAACAATAAGGATGCTGACCTGTACACC |
| | | AGCAGAGTGATGCTGAGCAGCCAAGTGCCTCTGGAGCCTCCTCTGCTGTTTCTGC |
| | | TGGAGGAATACAAAAATTACCTGGATGCTGCTAACATGAGCATGAGAGTCAGAAG |
| | | ACACTCTGACCCTGCCAGAAGAGGAGAGCTGTCTGTGTGTGACAGCATTTCTGAG |
| | | TGGGTGACAGCTGCTGACAAAAAGACTGCTGTGGACATGTCTGGAGGAACTGTCA |
| | | CAGTCCTGGAAAAGGTCCCTGTGAGCAAAGGCCAACTGAAGCAATACTTCTATGA |
| | | GACCAAGTGCAATCCAATGGGCTACACAAAAGAAGGCTGCAGAGGCATTGACAAA |
| | | AGACATTGGAACAGCCAGTGCAGAACTACCCAGAGCTATGTGAGAGCCCTGACCA |
| | | TGGATAGCAAAAAGAGAATTGGCTGGAGATTCATTAGAATTGACACTAGCTGTGT |
| | | CTGTACACTGACCATTAAAAGAGGAAGATGATGAGGTACCTTCTCCATAAAGTAG |
| | | GAAACACTACATCATCCATAAAGTAGGAAACACTACATCATCCATAAAGTAGGAA |
| | | ACACTACATCATCCATAAAGTAGGAAACACTACAGAATTCGATATCAGTGAATTC |
| | | TACCAGTGCCATAAGTGAATTCTACCAGTGCCATAAGTGAATTCTACCAGTGCCA |
| | | TAAGTGAATTCTACCAGTGCCATATCAAGCTTATACATACTTCTTTACATTCCAA |
| | | TACATACTTCTTTACATTCCAATACATACTTCTTTACATTCCACCATGGACTTGT |
| | | ACAAACACCATTGTCACACACTCCAACAAACACCATTGTCACACACTCCAACAAA |
| | | CACCATTGTCACACACTCCAAGCTAGCCCTAGGAATCAACCTCTGGATTACAAAA |
| | | TTTGTGAAAGATTGACTGGTATTCTTAACTATGTTGCTCCTTTTACGCTATGTGG |
| | | ATACGCTGCTTTAATGCCTTTGTATCATGCTATTGCTTCCCGTATGGCTTTCATT |
| | | TTCTCCTCCTTGTATAAATCCTGGTTAGTTCTTGCCACGGCGGAACTCATCGCCG |
| | | CCTGCCTTGCCCGCTGCTGGACAGGGGCTCGGCTGTTGGGCACTGACAATTCCGT |
| | | GGTGTTCCTGCAGGCAGACATGATAAGATACATTGATGAGTTTGGACAAACCACA |
| | | ACTAGAATGCAGTGAAAAAAATGCTTTATTTGTGAAATTTGTGATGCTATTGCTT |
| | | TATTTGTAACCATTATAAGCTGCAATAAACAAGTTAACAACAACAATTGCATTCA |
| | | TTTTATGTTTCAGGTTCAGGGGGAGGTGTGGGAGGTTTTTTAAAGCAAGTAAAAC |
| | | CTCTACAAATGTGGTA |
| 26 | BDNF16 | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGGGGTGAGTCACC |
| | | CACACAAAGGAAAAGGGCCTTTCCGTCCTCAGCCGTCGCTTCATGTGACTCCACG |
| | | GAGTACCGGGCGCCGTCCAGGCACCTCGATTAGTTCTCGAGCTTTTGGAGTACGT |
| | | CGTCTTTAGGTTGGGGGGAGGGGTTTTATGCGATGGAGTTTCCCCACACTGAGTG |
| | | GGTGGAGACTGAAGTTAGGCCAGCTTGGCACTTGATGTAATTCTCCTTGGAATTT |
| | | GCCCTTTTTGAGTTTGGATCTTGGTTCATTCTCAAGCCTCAGACAGTGGTTCAAA |
| | | GTTTTTTTCTTCCATTTCAGGTGTCGTGAGGATCCAACTCCTAAAAAACCGCCAC |
| | | CATGACCATCCTGTTCCTGACTATGGTGATTAGCTACTTTGGATGCATGAAGGCT |
| | | GCCCCTATGAAAGAAGCTAACATCAGAGGACAAGGAGGCCTGGCCTACCCAGGAG |
| | | TGAGAACCCATGGAACTCTGGAGTCTGTGAATGGCCCAAAGGCTGGAAGCAGAGG |
| | | CCTGACAAGCCTGGCTGACACTTTTGAACATGTGATTGAAGAGCTGCTGGATGAG |
| | | GACCAGAAAGTGAGACCTAATGAAGAAAACAATAAGGATGCTGACCTGTACACCA |
| | | GCAGAGTGATGCTGAGCAGCCAAGTGCCTCTGGAGCCTCCTCTGCTGTTTCTGCT |
| | | GGAGGAATACAAAAATTACCTGGATGCTGCTAACATGAGCATGAGAGTCAGAAGA |
| | | CACTCTGACCCTGCCAGAAGAGGAGAGCTGTCTGTGTGTGACAGCATTTCTGAGT |
| | | GGGTGACAGCTGCTGACAAAAAGACTGCTGTGGACATGTCTGGAGGAACTGTCAC |
| | | AGTCCTGGAAAAGGTCCCTGTGAGCAAAGGCCAACTGAAGCAATACTTCTATGAG |
| | | ACCAAGTGCAATCCAATGGGCTACACAAAAGAAGGCTGCAGAGGCATTGACAAAA |
| | | GACATTGGAACAGCCAGTGCAGAACTACCCAGAGCTATGTGAGAGCCCTGACCAT |
| | | GGATAGCAAAAAGAGAATTGGCTGGAGATTCATTAGAATTGACACTAGCTGTGTC |
| | | TGTACACTGACCATTAAAAGAGGAAGATGATGAGGTACCTTCTCCATAAAGTAGG |
| | | AAACACTACATCATCCATAAAGTAGGAAACACTACATCATCCATAAAGTAGGAAA |
| | | CACTACATCATCCATAAAGTAGGAAACACTACAGAATTCGATATCAGTGAATTCT |
| | | ACCAGTGCCATAAGTGAATTCTACCAGTGCCATAAGTGAATTCTACCAGTGCCAT |
| | | AAGTGAATTCTACCAGTGCCATATCAAGCTTATACATACTTCTTTACATTCCAAT |
| | | ACATACTTCTTTACATTCCAATACATACTTCTTTACATTCCACCATGGACTTGTA |
| | | CAAACACCATTGTCACACACTCCAACAAACACCATTGTCACACACTCCAACAAAC |
| | | ACCATTGTCACACACTCCAAGCTAGCCCTAGGAATCAACCTCTGGATTACAAAAT |
| | | TTGTGAAAGATTGACTGGTATTCTTAACTATGTTGCTCCTTTTACGCTATGTGGA |
| | | TACGCTGCTTTAATGCCTTTGTATCATGCTATTGCTTCCCGTATGGCTTTCATTT |
| | | TCTCCTCCTTGTATAAATCCTGGTTAGTTCTTGCCACGGCGGAACTCATCGCCGC |
| | | CTGCCTTGCCCGCTGCTGGACAGGGGCTCGGCTGTTGGGCACTGACAATTCCGTG |
| | | GTGTTCCTGCAGGGTTTATTTGTGAAATTTGTGATGCTATTGCTTTATTTGTAAC |
| | | CATCTAGCTTTATTTGTGAAATTTGTGATGCTATTGCTTTATTTGTAACCATTAT |
| | | AAGCTGCAATAAACAAGTTAACAACAACAATTGCATTCATTTTATGTTTCAGGTT |
| | | CAGGGGGAGATGTGGGAGGTTTTTTAA |
| 27 | BDNF17 | GCCGGGCTGGTAGCCCAGCGGGGGGCGCGGCAGGGCGAGGAGCAGCCCGCGCTGC |
| | | TCCCGCCCCTCGGGTGCCAGCACCGCCCCTGCTGCGGCGGGTGAGGGGCGGGGCG |
| | | GGGCGCGGCGTATATAAGGCTAGGGGCGGGCGCCGCTCTTTTGTTTCTTGCTGCA |
| | | GCAACGCGAGTGGGAGCACCAGGATCTCGGGCTCGGAACGAGACTGCACGGTGAG |
| | | TGCGGCGCCGGGGCGGGGGGCCCACCCAGGGTGTGGTCGGATCCGGTGCACCGGG |
| | | CGGGCGCGCCGCAACCGCGACAGGCGCCCTTCTCGGACCGGACGCAGGGGCCGGC |
| | | GACCACGCCCTGGGACCGAGAAGAGGGGTGCGGGACGCGCCCAGATCCTCGGCCT |
| | | TGGGGCTGCTCGGCACGCCTTGGCGCGAGTGCCACGTCGAGAGGCGTCGGCGGGG |
| | | AGCGCGGAAGGGGACGCTGGCCCCCAGGCCCAGGTCAAGCGCCTTGGTTTGCCCA |
| | | CTAGGATTGTTTTAAGAAAAGGATCCGCCACCATGACCATCCTGTTCCTGACTAT |
| | | GGTGATTAGCTACTTTGGATGCATGAAGGCTGCCCCTATGAAAGAAGCTAACATC |
| | | AGAGGACAAGGAGGCCTGGCCTACCCAGGAGTGAGAACCCATGGAACTCTGGAGT |
| | | CTGTGAATGGCCCAAAGGCTGGAAGCAGAGGCCTGACAAGCCTGGCTGACACTTT |
| | | TGAACATGTGATTGAAGAGCTGCTGGATGAGGACCAGAAAGTGAGACCTAATGAA |
| | | GAAAACAATAAGGATGCTGACCTGTACACCAGCAGAGTGATGCTGAGCAGCCAAG |
| | | TGCCTCTGGAGCCTCCTCTGCTGTTTCTGCTGGAGGAATACAAAAATTACCTGGA |
| | | TGCTGCTAACATGAGCATGAGAGTCAGAAGACACTCTGACCCTGCCAGAAGAGGA |
| | | GAGCTGTCTGTGTGTGACAGCATTTCTGAGTGGGTGACAGCTGCTGACAAAAAGA |
| | | CTGCTGTGGACATGTCTGGAGGAACTGTCACAGTCCTGGAAAAGGTCCCTGTGAG |
| | | CAAAGGCCAACTGAAGCAATACTTCTATGAGACCAAGTGCAATCCAATGGGCTAC |
| | | ACAAAAGAAGGCTGCAGAGGCATTGACAAAAGACATTGGAACAGCCAGTGCAGAA |
| | | CTACCCAGAGCTATGTGAGAGCCCTGACCATGGATAGCAAAAAGAGAATTGGCTG |
| | | GAGATTCATTAGAATTGACACTAGCTGTGTCTGTACACTGACCATTAAAAGAGGA |
| | | AGATGATGAGGTACCTTCTCCATAAAGTAGGAAACACTACATCATCCATAAAGTA |
| | | GGAAACACTACATCATCCATAAAGTAGGAAACACTACATCATCCATAAAGTAGGA |
| | | AACACTACAGAATTCGATATCAGTGAATTCTACCAGTGCCATAAGTGAATTCTAC |
| | | CAGTGCCATAAGTGAATTCTACCAGTGCCATAAGTGAATTCTACCAGTGCCATAT |
| | | CAAGCTTATACATACTTCTTTACATTCCAATACATACTTCTTTACATTCCAATAC |
| | | ATACTTCTTTACATTCCACCATGGACTTGTACAAACACCATTGTCACACACTCCA |
| | | ACAAACACCATTGTCACACACTCCAACAAACACCATTGTCACACACTCCAAGCTA |
| | | GCCCTAGGAATCAACCTCTGGATTACAAAATTTGTGAAAGATTGACTGGTATTCT |
| | | TAACTATGTTGCTCCTTTTACGCTATGTGGATACGCTGCTTTAATGCCTTTGTAT |
| | | CATGCTATTGCTTCCCGTATGGCTTTCATTTTCTCCTCCTTGTATAAATCCTGGT |
| | | TAGTTCTTGCCACGGCGGAACTCATCGCCGCCTGCCTTGCCCGCTGCTGGACAGG |
| | | GGCTCGGCTGTTGGGCACTGACAATTCCGTGGTGTTCCTGCAGGGCTCGCTTTCT |
| | | TGCTGTCCAATTTCTATTAAAGGTTCCTTTGTTCCCTAAGTCCAACTACTAAACT |
| | | GGGGGATATTATGAAGGGCCTTGAGCATCTGGATTCTGCCTAATAAAAGATCTTT |
| | | ATTTTCATTAGATCTGTGTGTTGGTTTTTTGTGTG |
| 28 | BDNF18 | TGTCCCACCTCGTTAGAGAAAACAGCGATGCTCAAAGGCAACCTCCTTCCTGACA |
| | | TTGCCTGGTAGGACGCGACGTGGTGTTTGCCCGCGCGGAATGCGGACGCAAGGCT |
| | | GCTCCTAGGTCTCGGGGACGCGCCATCCCCATTTCCGCTCGCGGAGGCGTAGGGT |
| | | CCGGGCGCGGGACCCCAGTCGACCTTGACTGGCGGCGCGACCTTGAGGCCTGCGT |
| | | TCGCCTCAGTTGCCCCCTCTGTGCAATGGGGAGACGCCTGCCGCCCCGCCCCGAG |
| | | TCCCATGCCGCAGCCACCGCGACGGAGCCCGCAGGCGGGAACCTGCCTCCGCGCG |
| | | TTAGCGCGCACGCGCGCCTCATGTGTCGTCCCCATCAGCGCCGGCTTCCGTCTAT |
| | | AGGCCAGATGCACTGTCACTCTGGCGAAGTCGCAGACCCGATTGGCCGGGACGGA |
| | | GGCGCGAGACCGGGTTGCGGGCGGGGCCGAACGTGGTATAAAAGGGGGGGGAGGC |
| | | CAGGCTCGTGTCAGTCTGCAGACGCCACCCGAGCCGAGTGGTCGTTACTATTAGC |
| | | CGGATCCGCCACCATGACCATCCTGTTCCTGACTATGGTGATTAGCTACTTTGGA |
| | | TGCATGAAGGCTGCCCCTATGAAAGAAGCTAACATCAGAGGACAAGGAGGCCTGG |
| | | CCTACCCAGGAGTGAGAACCCATGGAACTCTGGAGTCTGTGAATGGCCCAAAGGC |
| | | TGGAAGCAGAGGCCTGACAAGCCTGGCTGACACTTTTGAACATGTGATTGAAGAG |
| | | CTGCTGGATGAGGACCAGAAAGTGAGACCTAATGAAGAAAACAATAAGGATGCTG |
| | | ACCTGTACACCAGCAGAGTGATGCTGAGCAGCCAAGTGCCTCTGGAGCCTCCTCT |
| | | GCTGTTTCTGCTGGAGGAATACAAAAATTACCTGGATGCTGCTAACATGAGCATG |
| | | AGAGTCAGAAGACACTCTGACCCTGCCAGAAGAGGAGAGCTGTCTGTGTGTGACA |
| | | GCATTTCTGAGTGGGTGACAGCTGCTGACAAAAAGACTGCTGTGGACATGTCTGG |
| | | AGGAACTGTCACAGTCCTGGAAAAGGTCCCTGTGAGCAAAGGCCAACTGAAGCAA |
| | | TACTTCTATGAGACCAAGTGCAATCCAATGGGCTACACAAAAGAAGGCTGCAGAG |
| | | GCATTGACAAAAGACATTGGAACAGCCAGTGCAGAACTACCCAGAGCTATGTGAG |
| | | AGCCCTGACCATGGATAGCAAAAAGAGAATTGGCTGGAGATTCATTAGAATTGAC |
| | | ACTAGCTGTGTCTGTACACTGACCATTAAAAGAGGAAGATGATGAGGTACCTTCT |
| | | CCATAAAGTAGGAAACACTACATCATCCATAAAGTAGGAAACACTACATCATCCA |
| | | TAAAGTAGGAAACACTACATCATCCATAAAGTAGGAAACACTACAGAATTCGATA |
| | | TCAGTGAATTCTACCAGTGCCATAAGTGAATTCTACCAGTGCCATAAGTGAATTC |
| | | TACCAGTGCCATAAGTGAATTCTACCAGTGCCATATCAAGCTTATACATACTTCT |
| | | TTACATTCCAATACATACTTCTTTACATTCCAATACATACTTCTTTACATTCCAC |
| | | CATGGACTTGTACAAACACCATTGTCACACACTCCAACAAACACCATTGTCACAC |
| | | ACTCCAACAAACACCATTGTCACACACTCCAAGCTAGCCCTAGGAATCAACCTCT |
| | | GGATTACAAAATTTGTGAAAGATTGACTGGTATTCTTAACTATGTTGCTCCTTTT |
| | | ACGCTATGTGGATACGCTGCTTTAATGCCTTTGTATCATGCTATTGCTTCCCGTA |
| | | TGGCTTTCATTTTCTCCTCCTTGTATAAATCCTGGTTAGTTCTTGCCACGGCGGA |
| | | ACTCATCGCCGCCTGCCTTGCCCGCTGCTGGACAGGGGCTCGGCTGTTGGGCACT |
| | | GACAATTCCGTGGTGTTCCTGCAGGGCTCGCTTTCTTGCTGTCCAATTTCTATTA |
| | | AAGGTTCCTTTGTTCCCTAAGTCCAACTACTAAACTGGGGGATATTATGAAGGGC |
| | | CTTGAGCATCTGGATTCTGCCTAATAAAAGATCTTTATTTTCATTAGATCTGTGT |
| | | GTTGGTTTTTTGTGTG |
| 29 | BDNF19 | CCCCGGATGCTCACCTCCCCGACTCGCCCCCGCTGTGGCCCCGCCCCCGCGCGGC |
| | | TCTTCGTGCCACGTCACCGCCTGCGTCGCTTCCGGAGGCGCAGCGGGCGATGACG |
| | | TAGAGGGACGTGCCCTCTATATGAGGTTGGGGAGCGGCTGAGTCGGTCAGTCCGC |
| | | CTGGAGACCTCGAGCCGAGTGGTCGTCCGCCCGCTCCCCCCTCCCCCCGAGCGCC |
| | | GCTCCGGCTGCACCGCGCTCGCTCCGAGTTTCAGGCTCGTGCTAAGCTAGCGCCG |
| | | TCGTCGTCTCCCTTCAGTCGCCATCGGATCCGCCACCATGACCATCCTGTTCCTG |
| | | ACTATGGTGATTAGCTACTTTGGATGCATGAAGGCTGCCCCTATGAAAGAAGCTA |
| | | ACATCAGAGGACAAGGAGGCCTGGCCTACCCAGGAGTGAGAACCCATGGAACTCT |
| | | GGAGTCTGTGAATGGCCCAAAGGCTGGAAGCAGAGGCCTGACAAGCCTGGCTGAC |
| | | ACTTTTGAACATGTGATTGAAGAGCTGCTGGATGAGGACCAGAAAGTGAGACCTA |
| | | ATGAAGAAAACAATAAGGATGCTGACCTGTACACCAGCAGAGTGATGCTGAGCAG |
| | | CCAAGTGCCTCTGGAGCCTCCTCTGCTGTTTCTGCTGGAGGAATACAAAAATTAC |
| | | CTGGATGCTGCTAACATGAGCATGAGAGTCAGAAGACACTCTGACCCTGCCAGAA |
| | | GAGGAGAGCTGTCTGTGTGTGACAGCATTTCTGAGTGGGTGACAGCTGCTGACAA |
| | | AAAGACTGCTGTGGACATGTCTGGAGGAACTGTCACAGTCCTGGAAAAGGTCCCT |
| | | GTGAGCAAAGGCCAACTGAAGCAATACTTCTATGAGACCAAGTGCAATCCAATGG |
| | | GCTACACAAAAGAAGGCTGCAGAGGCATTGACAAAAGACATTGGAACAGCCAGTG |
| | | CAGAACTACCCAGAGCTATGTGAGAGCCCTGACCATGGATAGCAAAAAGAGAATT |
| | | GGCTGGAGATTCATTAGAATTGACACTAGCTGTGTCTGTACACTGACCATTAAAA |
| | | GAGGAAGATGATGAGGTACCTTCTCCATAAAGTAGGAAACACTACATCATCCATA |
| | | AAGTAGGAAACACTACATCATCCATAAAGTAGGAAACACTACATCATCCATAAAG |
| | | TAGGAAACACTACAGAATTCGATATCAGTGAATTCTACCAGTGCCATAAGTGAAT |
| | | TCTACCAGTGCCATAAGTGAATTCTACCAGTGCCATAAGTGAATTCTACCAGTGC |
| | | CATATCAAGCTTATACATACTTCTTTACATTCCAATACATACTTCTTTACATTCC |
| | | AATACATACTTCTTTACATTCCACCATGGACTTGTACAAACACCATTGTCACACA |
| | | CTCCAACAAACACCATTGTCACACACTCCAACAAACACCATTGTCACACACTCCA |
| | | AGCTAGCCCTAGGAATCAACCTCTGGATTACAAAATTTGTGAAAGATTGACTGGT |
| | | ATTCTTAACTATGTTGCTCCTTTTACGCTATGTGGATACGCTGCTTTAATGCCTT |
| | | TGTATCATGCTATTGCTTCCCGTATGGCTTTCATTTTCTCCTCCTTGTATAAATC |
| | | CTGGTTAGTTCTTGCCACGGCGGAACTCATCGCCGCCTGCCTTGCCCGCTGCTGG |
| | | ACAGGGGCTCGGCTGTTGGGCACTGACAATTCCGTGGTGTTCCTGCAGGGCTCGC |
| | | TTTCTTGCTGTCCAATTTCTATTAAAGGTTCCTTTGTTCCCTAAGTCCAACTACT |
| | | AAACTGGGGGATATTATGAAGGGCCTTGAGCATCTGGATTCTGCCTAATAAAAGA |
| | | TCTTTATTTTCATTAGATCTGTGTGTTGGTTTTTTGTGTG |
| 30 | BDNF20 | TGTCCCACCTCGTTAGAGAAAACAGCGATGCTCAAAGGCAACCTCCTTCCTGACA |
| | | TTGCCTGGTAGGACGCGACGTGGTGTTTGCCCGCGCGGAATGCGGACGCAAGGCT |
| | | GCTCCTAGGTCTCGGGGACGCGCCATCCCCATTTCCGCTCGCGGAGGCGTAGGGT |
| | | CCGGGCGCGGGACCCCAGTCGACCTTGACTGGCGGCGCGACCTTGAGGCCTGCGT |
| | | TCGCCTCAGTTGCCCCCTCTGTGCAATGGGGAGACGCCTGCCGCCCCGCCCCGAG |
| | | TCCCATGCCGCAGCCACCGCGACGGAGCCCGCAGGCGGGAACCTGCCTCCGCGCG |
| | | TTAGCGCGCACGCGCGCCTCATGTGTCGTCCCCATCAGCGCCGGCTTCCGTCTAT |
| | | AGGCCAGATGCACTGTCACTCTGGCGAAGTCGCAGACCCGATTGGCCGGGACGGA |
| | | GGCGCGAGACCGGGTTGCGGGCGGGGCCGAACGTGGTATAAAAGGGGGGGGAGGC |
| | | CAGGCTCGTGTCAGTCTGCAGACGCCACCCGAGCCGAGTGGTCGTTACTATTAGC |
| | | CGCCGGGCTGGTAGCCCAGCGGGGGGCGCGGCAGGGCGAGGAGCAGCCCGCGCTG |
| | | CTCCCGCCCCTCGGGTGCCAGCACCGCCCCTGCTGCGGCGGGTGAGGGGCGGGGC |
| | | GGGGCGCGGCGTATATAAGGCTAGGGGGGGGCGCCGCTCTTCAGTCCGCCTGGAG |
| | | ACCTCGAGCCGAGTGGTCGTCAGGATCTCGGGCTCGGAACGAGACTGCACGGTGA |
| | | GTGCGGCGCCGGGGCGGGGGGCCCACCCAGGGTGTGGTCGGATCCGGTGCACCGG |
| | | GCGGGCGCGCCGCAACCGCGACAGGCGCCCTTCTCGGACCGGACGCAGGGGCCGG |
| | | CGACCACGCCCTGGGACCGAGAAGAGGGGTGCGGGACGCGCCCAGATCCTCGGCC |
| | | TTGGGGCTGCTCGGCACGCCTTGGCGCGAGTGCCACGTCGAGAGGCGTCGGCGGG |
| | | GAGCGCGGAAGGGGACGCTGGCCCCCAGGCCCAGGTCAAGCGCCTTGGTTTGCCC |
| | | ACTAGGATTGTTTTAAGAAAAGGATCCGCCACCATGACCATCCTGTTCCTGACTA |
| | | TGGTGATTAGCTACTTTGGATGCATGAAGGCTGCCCCTATGAAAGAAGCTAACAT |
| | | CAGAGGACAAGGAGGCCTGGCCTACCCAGGAGTGAGAACCCATGGAACTCTGGAG |
| | | TCTGTGAATGGCCCAAAGGCTGGAAGCAGAGGCCTGACAAGCCTGGCTGACACTT |
| | | TTGAACATGTGATTGAAGAGCTGCTGGATGAGGACCAGAAAGTGAGACCTAATGA |
| | | AGAAAACAATAAGGATGCTGACCTGTACACCAGCAGAGTGATGCTGAGCAGCCAA |
| | | GTGCCTCTGGAGCCTCCTCTGCTGTTTCTGCTGGAGGAATACAAAAATTACCTGG |
| | | ATGCTGCTAACATGAGCATGAGAGTCAGAAGACACTCTGACCCTGCCAGAAGAGG |
| | | AGAGCTGTCTGTGTGTGACAGCATTTCTGAGTGGGTGACAGCTGCTGACAAAAAG |
| | | ACTGCTGTGGACATGTCTGGAGGAACTGTCACAGTCCTGGAAAAGGTCCCTGTGA |
| | | GCAAAGGCCAACTGAAGCAATACTTCTATGAGACCAAGTGCAATCCAATGGGCTA |
| | | CACAAAAGAAGGCTGCAGAGGCATTGACAAAAGACATTGGAACAGCCAGTGCAGA |
| | | ACTACCCAGAGCTATGTGAGAGCCCTGACCATGGATAGCAAAAAGAGAATTGGCT |
| | | GGAGATTCATTAGAATTGACACTAGCTGTGTCTGTACACTGACCATTAAAAGAGG |
| | | AAGATGATGAGGTACCTTCTCCATAAAGTAGGAAACACTACATCATCCATAAAGT |
| | | AGGAAACACTACATCATCCATAAAGTAGGAAACACTACATCATCCATAAAGTAGG |
| | | AAACACTACAGAATTCGATATCAGTGAATTCTACCAGTGCCATAAGTGAATTCTA |
| | | CCAGTGCCATAAGTGAATTCTACCAGTGCCATAAGTGAATTCTACCAGTGCCATA |
| | | TCAAGCTTATACATACTTCTTTACATTCCAATACATACTTCTTTACATTCCAATA |
| | | CATACTTCTTTACATTCCACCATGGACTTGTACAAACACCATTGTCACACACTCC |
| | | AACAAACACCATTGTCACACACTCCAACAAACACCATTGTCACACACTCCAAGCT |
| | | AGCCCTAGGAATCAACCTCTGGATTACAAAATTTGTGAAAGATTGACTGGTATTC |
| | | TTAACTATGTTGCTCCTTTTACGCTATGTGGATACGCTGCTTTAATGCCTTTGTA |
| | | TCATGCTATTGCTTCCCGTATGGCTTTCATTTTCTCCTCCTTGTATAAATCCTGG |
| | | TTAGTTCTTGCCACGGCGGAACTCATCGCCGCCTGCCTTGCCCGCTGCTGGACAG |
| | | GGGCTCGGCTGTTGGGCACTGACAATTCCGTGGTGTTCCTGCAGGGCTCGCTTTC |
| | | TTGCTGTCCAATTTCTATTAAAGGTTCCTTTGTTCCCTAAGTCCAACTACTAAAC |
| | | TGGGGGATATTATGAAGGGCCTTGAGCATCTGGATTCTGCCTAATAAAAGATCTT |
| | | TATTTTCATTAGATCTGTGTGTTGGTTTTTTGTGTG |
| 31 | BDNF21 | GACATTGATTATTGACTAGTTATTAATAGTAATCAATTACGGGGTCATTAGTTCA |
| | | TAGCCCATATATGGAGTTCCGCGTTACATAACTTACGGTAAATGGCCCGCCTGGC |
| | | TGACCGCCCAACGACCCCCGCCCATTGACGTCAATAATGACGTATGTTCCCATAG |
| | | TAACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTGAC |
| | | GTCAATGACGGTAAATGGCCCGCCTGGCATTATGCCCAGTACATGACCTTATGGG |
| | | ACTTTCCTACTTGGCAGTACATCTACGTATTAGTCATCGCTATTACCATGGGTCG |
| | | AGGTGAGCCCCCGTTCTGCTTCACTCTCCCCATCTCCCCCCCCTCCCCACCCCCA |
| | | ATTTTGTATTTATTTATTTTTTAATTATTTTGTGCAGCGATGGGGGCGGGGGGGG |
| | | GGGGGGGGGGGGGGGGGGGTGGGGCGGAGGGTGGCAAGGGGCGGGGCGGGGCAAG |
| | | GCGGAAAGGTGCGGCGGCAGCCAATCAAAGCGGCGCCCTCCAAAAGTTTCCTTTT |
| | | ATGGCAAGGCGGCGGCGGCGGCGGCCCTATAAAAAGCGAAGCGCGCGGCGGGCGG |
| | | GAGTCGCTGCGTTGCCTTGGGTCCTCCCCCCCTCTCCAGCCGCCTCGCGCCGCCC |
| | | GCCCCGGCTCTGACTGACCGCGTTACTCCCACAGGTGAGCGGGCGGGACGGCCCT |
| | | TCTCCTCCGGGCTGTAATTAGCGCTTGGTTTAATGACGGCTTGTTTCTTTTCTGT |
| | | GGCTGCGTGAAAGCCTTGAGGGGCTCCGGGAGGGCCCTTTGTGCGGGGGGAGCGG |
| | | CTCGGGGCTGTCCGCGGGGGGACGGCTGCCTTCGGGGGGGACGGGGCAGGGCGGG |
| | | GTTCGGCTTCTGGCGTGTGACCGGCGGCTCTAGAGCCTCTGCTAACCATGTTCAT |
| | | GCCTTCTTCTTTTTCCTACAGCTCCTGGGCAACGTGCTGGTTATTGTGCTGTCTC |
| | | ATCATTTTGGCAAAGAATTGGATCCACTCGAGTGGAGCTCGCGACTAGTCGATTC |
| | | GAATTCGGCTTGTGATGACCATCCTTTTCCTTACTATGGTTATTTCATACTTTGG |
| | | TTGCATGAAGGCTGCCCCCATGAAAGAAGCAAACATCCGAGGACAAGGTGGCTTG |
| | | GCCTACCCAGGTGTGCGGACCCATGGGACTCTGGAGAGCGTGAATGGGCCCAAGG |
| | | CAGGTTCAAGAGGCTTGACATCATTGGCTGACACTTTCGAACACGTGATAGAAGA |
| | | GCTGTTGGATGAGGACCAGAAAGTTCGGCCCAATGAAGAAAACAATAAGGACGCA |
| | | GACTTGTACACGTCCAGGGTGATGCTCAGTAGTCAAGTGCCTTTGGAGCCTCCTC |
| | | TTCTCTTTCTGCTGGAGGAATACAAAAATTACCTAGATGCTGCAAACATGTCCAT |
| | | GAGGGTCCGGCGCCACTCTGACCCTGCCCGCCGAGGGGAGCTGAGCGTGTGTGAC |
| | | AGTATTAGTGAGTGGGTAACGGCGGCAGACAAAAAGACTGCAGTGGACATGTCGG |
| | | GCGGGACGGTCACAGTCCTTGAAAAGGTCCCTGTATCAAAAGGCCAACTGAAGCA |
| | | ATACTTCTACGAGACCAAGTGCAATCCCATGGGTTACACAAAAGAAGGCTGCAGG |
| | | GGCATAGACAAAAGGCATTGGAACTCCCAGTGCCGAACTACCCAGTCGTACGTGC |
| | | GGGCCCTTACCATGGATAGCAAAAAGAGAATTGGCTGGCGATTCATAAGGATAGA |
| | | CACTTCTTGTGTATGTACATTGACCATTAAAAGGGGAAGATATCCGTATGATGTT |
| | | CCTGATTATTGAGAATTCGATATCAAGCTTATCGATAATCAACCTCTGGATTACA |
| | | AAATTTGTGAAAGATTGACTGGTATTCTTAACTATGTTGCTCCTTTTACGCTATG |
| | | TGGATACGCTGCTTTAATGCCTTTGTATCATGCTATTGCTTCCCGTATGGCTTTC |
| | | ATTTTCTCCTCCTTGTATAAATCCTGGTTGCTGTCTCTTTATGAGGAGTTGTGGC |
| | | CCGTTGTCAGGCAACGTGGCGTGGTGTGCACTGTGTTTGCTGACGCAACCCCCAC |
| | | TGGTTGGGGCATTGCCACCACCTGTCAGCTCCTTTCCGGGACTTTCGCTTTCCCC |
| | | CTCCCTATTGCCACGGCGGAACTCATCGCCGCCTGCCTTGCCCGCTGCTGGACAG |
| | | GGGCTCGGCTGTTGGGCACTGACAATTCCGTGGTGTTGTCGGGGAAATCATCGTC |
| | | CTTTCCTTGGCTGCTCGCCTGTGTTGCCACCTGGATTCTGCGCGGGACGTCCTTC |
| | | TGCTACGTCCCTTCGGCCCTCAATCCAGCGGACCTTCCTTCCCGCGGCCTGCTGC |
| | | CGGCTCTGCGGCCTCTTCCGCGTCTTCGCCTTCGCCCTCAGACGAGTCGGATCTC |
| | | CCTTTGGGCCGCCTCCCCGCATCGATACCGTCGACTCGCTGATCAGCCTCGACTG |
| | | TGCCTTCTAGTTGCCAGCCATCTGTTGTTTGCCCCTCCCCCGTGCCTTCCTTGAC |
| | | CCTGGAAGGTGCCACTCCCACTGTCCTTTCCTAATAAAATGAGGAAATTGCATCG |
| | | CATTGTCTGAGTAGGTGTCATTCTATTCTGGGGGGTGGGGTGGGGCAGGACAGCA |
| | | AGGGGGAGGATTGGGAAGACAATAGCAGGCATGCTGGGGATGCGGTGGGCTCTAT |
| | | GG |
| 32 | BDNF22 | GACATTGATTATTGACTAGTTATTAATAGTAATCAATTACGGGGTCATTAGTTCA |
| | | TAGCCCATATATGGAGTTCCGCGTTACATAACTTACGGTAAATGGCCCGCCTGGC |
| | | TGACCGCCCAACGACCCCCGCCCATTGACGTCAATAATGACGTATGTTCCCATAG |
| | | TAACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTGAC |
| | | GTCAATGACGGTAAATGGCCCGCCTGGCATTATGCCCAGTACATGACCTTATGGG |
| | | ACTTTCCTACTTGGCAGTACATCTACGTATTAGTCATCGCTATTACCATGGGTCG |
| | | AGGTGAGCCCCCGTTCTGCTTCACTCTCCCCATCTCCCCCCCCTCCCCACCCCCA |
| | | ATTTTGTATTTATTTATTTTTTAATTATTTTGTGCAGCGATGGGGGCGGGGGGGG |
| | | GGGGGGGGGGGGGGGGGGGTGGGGCGGAGGGTGGCAAGGGGCGGGGCGGGGCAAG |
| | | GCGGAAAGGTGCGGCGGCAGCCAATCAAAGCGGCGCCCTCCAAAAGTTTCCTTTT |
| | | ATGGCAAGGCGGCGGCGGCGGCGGCCCTATAAAAAGCGAAGCGCGCGGCGGGCGG |
| | | GAGTCGCTGCGTTGCCTTGGGTCCTCCCCCCCTCTCCAGCCGCCTCGCGCCGCCC |
| | | GCCCCGGCTCTGACTGACCGCGTTACTCCCACAGGTGAGCGGGCGGGACGGCCCT |
| | | TCTCCTCCGGGCTGTAATTAGCGCTTGGTTTAATGACGGCTTGTTTCTTTTCTGT |
| | | GGCTGCGTGAAAGCCTTGAGGGGCTCCGGGAGGGCCCTTTGTGCGGGGGGAGCGG |
| | | CTCGGGGCTGTCCGCGGGGGGACGGCTGCCTTCGGGGGGGACGGGGCAGGGCGGG |
| | | GTTCGGCTTCTGGCGTGTGACCGGCGGCTCTAGAGCCTCTGCTAACCATGTTCAT |
| | | GCCTTCTTCTTTTTCCTACAGCTCCTGGGCAACGTGCTGGTTATTGTGCTGTCTC |
| | | ATCATTTTGGCAAAGAATTGGATCCACTCGAGTGGAGCTCGCGACTAGTCGATTC |
| | | GAATTCGGCTTGTGATGACCATCCTTTTCCTTACTATGGTTATTTCATACTTTGG |
| | | TTGCATGAAGGCTGCCCCCATGAAAGAAGCAAACATCCGAGGACAAGGTGGCTTG |
| | | GCCTACCCAGGTGTGCGGACCCATGGGACTCTGGAGAGCGTGAATGGGCCCAAGG |
| | | CAGGTTCAAGAGGCTTGACATCATTGGCTGACACTTTCGAACACGTGATAGAAGA |
| | | GCTGTTGGATGAGGACCAGAAAGTTCGGCCCAATGAAGAAAACAATAAGGACGCA |
| | | GACTTGTACACGTCCAGGGTGATGCTCAGTAGTCAAGTGCCTTTGGAGCCTCCTC |
| | | TTCTCTTTCTGCTGGAGGAATACAAAAATTACCTAGATGCTGCAAACATGTCCAT |
| | | GAGGGTCCGGCGCCACTCTGACCCTGCCCGCCGAGGGGAGCTGAGCGTGTGTGAC |
| | | AGTATTAGTGAGTGGGTAACGGCGGCAGACAAAAAGACTGCAGTGGACATGTCGG |
| | | GCGGGACGGTCACAGTCCTTGAAAAGGTCCCTGTATCAAAAGGCCAACTGAAGCA |
| | | ATACTTCTACGAGACCAAGTGCAATCCCATGGGTTACACAAAAGAAGGCTGCAGG |
| | | GGCATAGACAAAAGGCATTGGAACTCCCAGTGCCGAACTACCCAGTCGTACGTGC |
| | | GGGCCCTTACCATGGATAGCAAAAAGAGAATTGGCTGGCGATTCATAAGGATAGA |
| | | CACTTCTTGTGTATGTACATTGACCATTAAAAGGGGAAGATGAGAATTCGATATC |
| | | AAGCTTATCGATAATCAACCTCTGGATTACAAAATTTGTGAAAGATTGACTGGTA |
| | | TTCTTAACTATGTTGCTCCTTTTACGCTATGTGGATACGCTGCTTTAATGCCTTT |
| | | GTATCATGCTATTGCTTCCCGTATGGCTTTCATTTTCTCCTCCTTGTATAAATCC |
| | | TGGTTGCTGTCTCTTTATGAGGAGTTGTGGCCCGTTGTCAGGCAACGTGGCGTGG |
| | | TGTGCACTGTGTTTGCTGACGCAACCCCCACTGGTTGGGGCATTGCCACCACCTG |
| | | TCAGCTCCTTTCCGGGACTTTCGCTTTCCCCCTCCCTATTGCCACGGCGGAACTC |
| | | ATCGCCGCCTGCCTTGCCCGCTGCTGGACAGGGGCTCGGCTGTTGGGCACTGACA |
| | | ATTCCGTGGTGTTGTCGGGGAAATCATCGTCCTTTCCTTGGCTGCTCGCCTGTGT |
| | | TGCCACCTGGATTCTGCGCGGGACGTCCTTCTGCTACGTCCCTTCGGCCCTCAAT |
| | | CCAGCGGACCTTCCTTCCCGCGGCCTGCTGCCGGCTCTGCGGCCTCTTCCGCGTC |
| | | TTCGCCTTCGCCCTCAGACGAGTCGGATCTCCCTTTGGGCCGCCTCCCCGCATCG |
| | | ATACCGTCGACTCGCTGATCAGCCTCGACTGTGCCTTCTAGTTGCCAGCCATCTG |
| | | TTGTTTGCCCCTCCCCCGTGCCTTCCTTGACCCTGGAAGGTGCCACTCCCACTGT |
| | | CCTTTCCTAATAAAATGAGGAAATTGCATCGCATTGTCTGAGTAGGTGTCATTCT |
| | | ATTCTGGGGGGTGGGGTGGGGCAGGACAGCAAGGGGGAGGATTGGGAAGACAATA |
| | | GCAGGCATGCTGGGGATGCGGTGGGCTCTATGG |
| 33 | BDNF23 | GACATTGATTATTGACTAGTTATTAATAGTAATCAATTACGGGGTCATTAGTTCA |
| | | TAGCCCATATATGGAGTTCCGCGTTACATAACTTACGGTAAATGGCCCGCCTGGC |
| | | TGACCGCCCAACGACCCCCGCCCATTGACGTCAATAATGACGTATGTTCCCATAG |
| | | TAACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTGAC |
| | | GTCAATGACGGTAAATGGCCCGCCTGGCATTATGCCCAGTACATGACCTTATGGG |
| | | ACTTTCCTACTTGGCAGTACATCTACGTATTAGTCATCGCTATTACCATGGGTCG |
| | | AGGTGAGCCCCCGTTCTGCTTCACTCTCCCCATCTCCCCCCCCTCCCCACCCCCA |
| | | ATTTTGTATTTATTTATTTTTTAATTATTTTGTGCAGCGATGGGGGCGGGGGGGG |
| | | GGGGGGGGGGGGGGGGGGGTGGGGCGGAGGGTGGCAAGGGGCGGGGCGGGGCAAG |
| | | GCGGAAAGGTGCGGCGGCAGCCAATCAAAGCGGCGCCCTCCAAAAGTTTCCTTTT |
| | | ATGGCAAGGCGGCGGCGGCGGCGGCCCTATAAAAAGCGAAGCGCGCGGCGGGCGG |
| | | GAGTCGCTGCGTTGCCTTGGGTCCTCCCCCCCTCTCCAGCCGCCTCGCGCCGCCC |
| | | GCCCCGGCTCTGACTGACCGCGTTACTCCCACAGGTGAGCGGGCGGGACGGCCCT |
| | | TCTCCTCCGGGCTGTAATTAGCGCTTGGTTTAATGACGGCTTGTTTCTTTTCTGT |
| | | GGCTGCGTGAAAGCCTTGAGGGGCTCCGGGAGGGCCCTTTGTGCGGGGGGAGCGG |
| | | CTCGGGGCTGTCCGCGGGGGGACGGCTGCCTTCGGGGGGGACGGGGCAGGGCGGG |
| | | GTTCGGCTTCTGGCGTGTGACCGGCGGCTCTAGAGCCTCTGCTAACCATGTTCAT |
| | | GCCTTCTTCTTTTTCCTACAGCTCCTGGGCAACGTGCTGGTTATTGTGCTGTCTC |
| | | ATCATTTTGGCAAAGAATTGGATCCACTCGAGTGGAGCTCGCGACTAGTCGATTC |
| | | GAATTCGGCTTGTGATGACCATCCTGTTCCTGACTATGGTGATTAGCTACTTTGG |
| | | ATGCATGAAGGCTGCCCCTATGAAAGAAGCTAACATCAGAGGACAAGGAGGCCTG |
| | | GCCTACCCAGGAGTGAGAACCCATGGAACTCTGGAGTCTGTGAATGGCCCAAAGG |
| | | CTGGAAGCAGAGGCCTGACAAGCCTGGCTGACACTTTTGAACATGTGATTGAAGA |
| | | GCTGCTGGATGAGGACCAGAAAGTGAGACCTAATGAAGAAAACAATAAGGATGCT |
| | | GACCTGTACACCAGCAGAGTGATGCTGAGCAGCCAAGTGCCTCTGGAGCCTCCTC |
| | | TGCTGTTTCTGCTGGAGGAATACAAAAATTACCTGGATGCTGCTAACATGAGCAT |
| | | GAGAGTCAGAAGACACTCTGACCCTGCCAGAAGAGGAGAGCTGTCTGTGTGTGAC |
| | | AGCATTTCTGAGTGGGTGACAGCTGCTGACAAAAAGACTGCTGTGGACATGTCTG |
| | | GAGGAACTGTCACAGTCCTGGAAAAGGTCCCTGTGAGCAAAGGCCAACTGAAGCA |
| | | ATACTTCTATGAGACCAAGTGCAATCCAATGGGCTACACAAAAGAAGGCTGCAGA |
| | | GGCATTGACAAAAGACATTGGAACAGCCAGTGCAGAACTACCCAGAGCTATGTGA |
| | | GAGCCCTGACCATGGATAGCAAAAAGAGAATTGGCTGGAGATTCATTAGAATTGA |
| | | CACTAGCTGTGTCTGTACACTGACCATTAAAAGAGGAAGATGAGAATTCGATATC |
| | | AAGCTTATCGATAATCAACCTCTGGATTACAAAATTTGTGAAAGATTGACTGGTA |
| | | TTCTTAACTATGTTGCTCCTTTTACGCTATGTGGATACGCTGCTTTAATGCCTTT |
| | | GTATCATGCTATTGCTTCCCGTATGGCTTTCATTTTCTCCTCCTTGTATAAATCC |
| | | TGGTTGCTGTCTCTTTATGAGGAGTTGTGGCCCGTTGTCAGGCAACGTGGCGTGG |
| | | TGTGCACTGTGTTTGCTGACGCAACCCCCACTGGTTGGGGCATTGCCACCACCTG |
| | | TCAGCTCCTTTCCGGGACTTTCGCTTTCCCCCTCCCTATTGCCACGGCGGAACTC |
| | | ATCGCCGCCTGCCTTGCCCGCTGCTGGACAGGGGCTCGGCTGTTGGGCACTGACA |
| | | ATTCCGTGGTGTTGTCGGGGAAATCATCGTCCTTTCCTTGGCTGCTCGCCTGTGT |
| | | TGCCACCTGGATTCTGCGCGGGACGTCCTTCTGCTACGTCCCTTCGGCCCTCAAT |
| | | CCAGCGGACCTTCCTTCCCGCGGCCTGCTGCCGGCTCTGCGGCCTCTTCCGCGTC |
| | | TTCGCCTTCGCCCTCAGACGAGTCGGATCTCCCTTTGGGCCGCCTCCCCGCATCG |
| | | ATACCGTCGACTCGCTGATCAGCCTCGACTGTGCCTTCTAGTTGCCAGCCATCTG |
| | | TTGTTTGCCCCTCCCCCGTGCCTTCCTTGACCCTGGAAGGTGCCACTCCCACTGT |
| | | CCTTTCCTAATAAAATGAGGAAATTGCATCGCATTGTCTGAGTAGGTGTCATTCT |
| | | ATTCTGGGGGGTGGGGTGGGGCAGGACAGCAAGGGGGAGGATTGGGAAGACAATA |
| | | GCAGGCATGCTGGGGATGCGGTGGGCTCTATGG |
| 34 | BDNF24 | GACATTGATTATTGACTAGTTATTAATAGTAATCAATTACGGGGTCATTAGTTCA |
| | | TAGCCCATATATGGAGTTCCGCGTTACATAACTTACGGTAAATGGCCCGCCTGGC |
| | | TGACCGCCCAACGACCCCCGCCCATTGACGTCAATAATGACGTATGTTCCCATAG |
| | | TAACGCCAATAGGGACTTTCCATTGACGTCAATGGGTGGAGTATTTACGGTAAAC |
| | | TGCCCACTTGGCAGTACATCAAGTGTATCATATGCCAAGTACGCCCCCTATTGAC |
| | | GTCAATGACGGTAAATGGCCCGCCTGGCATTATGCCCAGTACATGACCTTATGGG |
| | | ACTTTCCTACTTGGCAGTACATCTACGTATTAGTCATCGCTATTACCATGGGTCG |
| | | AGGTGAGCCCCCGTTCTGCTTCACTCTCCCCATCTCCCCCCCCTCCCCACCCCCA |
| | | ATTTTGTATTTATTTATTTTTTAATTATTTTGTGCAGCGATGGGGGCGGGGGGGG |
| | | GGGGGGGGGGGGGGGGGGGTGGGGCGGAGGGTGGCAAGGGGCGGGGCGGGGCAAG |
| | | GCGGAAAGGTGCGGCGGCAGCCAATCAAAGCGGCGCCCTCCAAAAGTTTCCTTTT |
| | | ATGGCAAGGCGGCGGCGGCGGCGGCCCTATAAAAAGCGAAGCGCGCGGCGGGCGG |
| | | GAGTCGCTGCGTTGCCTTGGGTCCTCCCCCCCTCTCCAGCCGCCTCGCGCCGCCC |
| | | GCCCCGGCTCTGACTGACCGCGTTACTCCCACAGGTGAGCGGGCGGGACGGCCCT |
| | | TCTCCTCCGGGCTGTAATTAGCGCTTGGTTTAATGACGGCTTGTTTCTTTTCTGT |
| | | GGCTGCGTGAAAGCCTTGAGGGGCTCCGGGAGGGCCCTTTGTGCGGGGGGAGCGG |
| | | CTCGGGGCTGTCCGCGGGGGGACGGCTGCCTTCGGGGGGGACGGGGCAGGGCGGG |
| | | GTTCGGCTTCTGGCGTGTGACCGGCGGCTCTAGAGCCTCTGCTAACCATGTTCAT |
| | | GCCTTCTTCTTTTTCCTACAGCTCCTGGGCAACGTGCTGGTTATTGTGCTGTCTC |
| | | ATCATTTTGGCAAAGAATTGGATCCACTCGAGTGGAGCTCGCGACTAGTCGATTC |
| | | GAATTCGGCTTGTGATGACCATCCTGTTCCTGACTATGGTGATTAGCTACTTTGG |
| | | ATGCATGAAGGCTGCCCCTATGAAAGAAGCTAACATCAGAGGACAAGGAGGCCTG |
| | | GCCTACCCAGGAGTGAGAACCCATGGAACTCTGGAGTCTGTGAATGGCCCAAAGG |
| | | CTGGAAGCAGAGGCCTGACAAGCCTGGCTGACACTTTTGAACATGTGATTGAAGA |
| | | GCTGCTGGATGAGGACCAGAAAGTGAGACCTAATGAAGAAAACAATAAGGATGCT |
| | | GACCTGTACACCAGCAGAGTGATGCTGAGCAGCCAAGTGCCTCTGGAGCCTCCTC |
| | | TGCTGTTTCTGCTGGAGGAATACAAAAATTACCTGGATGCTGCTAACATGAGCAT |
| | | GAGAGTCAGAAGACACTCTGACCCTGCCAGAAGAGGAGAGCTGTCTGTGTGTGAC |
| | | AGCATTTCTGAGTGGGTGACAGCTGCTGACAAAAAGACTGCTGTGGACATGTCTG |
| | | GAGGAACTGTCACAGTCCTGGAAAAGGTCCCTGTGAGCAAAGGCCAACTGAAGCA |
| | | ATACTTCTATGAGACCAAGTGCAATCCAATGGGCTACACAAAAGAAGGCTGCAGA |
| | | GGCATTGACAAAAGACATTGGAACAGCCAGTGCAGAACTACCCAGAGCTATGTGA |
| | | GAGCCCTGACCATGGATAGCAAAAAGAGAATTGGCTGGAGATTCATTAGAATTGA |
| | | CACTAGCTGTGTCTGTACACTGACCATTAAAAGAGGAAGATGAGAATTCGATATC |
| | | AAGCTTATTCCATAAAGTAGGAAACACTACATCATCCATAAAGTAGGAAACACTA |
| | | CATCATCCATAAAGTAGGAAACACTACATCATCCATAAAGTAGGAAACACTACAC |
| | | GATAATCAACCTCTGGATTACAAAATTTGTGAAAGATTGACTGGTATTCTTAACT |
| | | ATGTTGCTCCTTTTACGCTATGTGGATACGCTGCTTTAATGCCTTTGTATCATGC |
| | | TATTGCTTCCCGTATGGCTTTCATTTTCTCCTCCTTGTATAAATCCTGGTTGCTG |
| | | TCTCTTTATGAGGAGTTGTGGCCCGTTGTCAGGCAACGTGGCGTGGTGTGCACTG |
| | | TGTTTGCTGACGCAACCCCCACTGGTTGGGGCATTGCCACCACCTGTCAGCTCCT |
| | | TTCCGGGACTTTCGCTTTCCCCCTCCCTATTGCCACGGCGGAACTCATCGCCGCC |
| | | TGCCTTGCCCGCTGCTGGACAGGGGCTCGGCTGTTGGGCACTGACAATTCCGTGG |
| | | TGTTGTCGGGGAAATCATCGTCCTTTCCTTGGCTGCTCGCCTGTGTTGCCACCTG |
| | | GATTCTGCGCGGGACGTCCTTCTGCTACGTCCCTTCGGCCCTCAATCCAGCGGAC |
| | | CTTCCTTCCCGCGGCCTGCTGCCGGCTCTGCGGCCTCTTCCGCGTCTTCGCCTTC |
| | | GCCCTCAGACGAGTCGGATCTCCCTTTGGGCCGCCTCCCCGCATCGATACCGTCG |
| | | ACTCGCTGATCAGCCTCGACTGTGCCTTCTAGTTGCCAGCCATCTGTTGTTTGCC |
| | | CCTCCCCCGTGCCTTCCTTGACCCTGGAAGGTGCCACTCCCACTGTCCTTTCCTA |
| | | ATAAAATGAGGAAATTGCATCGCATTGTCTGAGTAGGTGTCATTCTATTCTGGGG |
| | | GGTGGGGTGGGGCAGGACAGCAAGGGGGAGGATTGGGAAGACAATAGCAGGCATG |
| | | CTGGGGATGCGGTGGGCTCTATGG |
| 35 | BDNF-A | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCGCCACCATG |
| | | ACCATCCTTTTCCTTACTATGGTTATTTCATACTTTGGTTGCATGAAGGCTGCCC |
| | | CCATGAAAGAAGCAAACATCCGAGGACAAGGTGGCTTGGCCTACCCAGGTGTGCG |
| | | GACCCATGGGACTCTGGAGAGCGTGAATGGGCCCAAGGCAGGTTCAAGAGGCTTG |
| | | ACATCATTGGCTGACACTTTCGAACACGTGATAGAAGAGCTGTTGGATGAGGACC |
| | | AGAAAGTTCGGCCCAATGAAGAAAACAATAAGGACGCAGACTTGTACACGTCCAG |
| | | GGTGATGCTCAGTAGTCAAGTGCCTTTGGAGCCTCCTCTTCTCTTTCTGCTGGAG |
| | | GAATACAAAAATTACCTAGATGCTGCAAACATGTCCATGAGGGTCCGGCGCCACT |
| | | CTGACCCTGCCCGCCGAGGGGAGCTGAGCGTGTGTGACAGTATTAGTGAGTGGGT |
| | | AACGGCGGCAGACAAAAAGACTGCAGTGGACATGTCGGGCGGGACGGTCACAGTC |
| | | CTTGAAAAGGTCCCTGTATCAAAAGGCCAACTGAAGCAATACTTCTACGAGACCA |
| | | AGTGCAATCCCATGGGTTACACAAAAGAAGGCTGCAGGGGCATAGACAAAAGGCA |
| | | TTGGAACTCCCAGTGCCGAACTACCCAGTCGTACGTGCGGGCCCTTACCATGGAT |
| | | AGCAAAAAGAGAATTGGCTGGCGATTCATAAGGATAGACACTTCTTGTGTATGTA |
| | | CATTGACCATTAAAAGGGGAAGATACCCATACGATGTTCCAGATTACGCTTAGAC |
| | | CCAGCTTTCTTGTACAAAGTGGGAATTCCGATAATCAACCTCTGGATTACAAAAT |
| | | TTGTGAAAGATTGACTGGTATTCTTAACTATGTTGCTCCTTTTACGCTATGTGGA |
| | | TACGCTGCTTTAATGCCTTTGTATCATGCTATTGCTTCCCGTATGGCTTTCATTT |
| | | TCTCCTCCTTGTATAAATCCTGGTTGCTGTCTCTTTATGAGGAGTTGTGGCCCGT |
| | | TGTCAGGCAACGTGGCGTGGTGTGCACTGTGTTTGCTGACGCAACCCCCACTGGT |
| | | TGGGGCATTGCCACCACCTGTCAGCTCCTTTCCGGGACTTTCGCTTTCCCCCTCC |
| | | CTATTGCCACGGCGGAACTCATCGCCGCCTGCCTTGCCCGCTGCTGGACAGGGGC |
| | | TCGGCTGTTGGGCACTGACAATTCCGTGGTGTTGTCGGGGAAGCTGACGTCCTTT |
| | | CCATGGCTGCTCGCCTGTGTTGCCACCTGGATTCTGCGCGGGACGTCCTTCTGCT |
| | | ACGTCCCTTCGGCCCTCAATCCAGCGGACCTTCCTTCCCGCGGCCTGCTGCCGGC |
| | | TCTGCGGCCTCTTCCGCGTCTTCGCCTTCGCCCTCAGACGAGTCGGATCTCCCTT |
| | | TGGGCCGCCTCCCCGCATCGGGAATTCCTAGAGCTCGCTGATCAGCCTCGACTGT |
| | | GCCTTCTAGTTGCCAGCCATCTGTTGTTTGCCCCTCCCCCGTGCCTTCCTTGACC |
| | | CTGGAAGGTGCCACTCCCACTGTCCTTTCCTAATAAAATGAGGAAATTGCATCGC |
| | | ATTGTCTGAGTAGGTGTCATTCTATTCTGGGGGGTGGGGTGGGGCAGGACAGCAA |
| | | GGGGGAGGATTGGGAAGACAATAGCAGGCATGCTGGGGATGGCCCGGGCTCTATG |
| | | G |
| 36 | BDNF-B | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCGCCACCATG |
| | | ACCATCCTTTTCCTTACTATGGTTATTTCATACTTTGGTTGCATGAAGGCTCACT |
| | | CTGACCCTGCCCGCCGAGGGGAGCTGAGCGTGTGTGACAGTATTAGTGAGTGGGT |
| | | AACGGCGGCAGACAAAAAGACTGCAGTGGACATGTCGGGCGGGACGGTCACAGTC |
| | | CTTGAAAAGGTCCCTGTATCAAAAGGCCAACTGAAGCAATACTTCTACGAGACCA |
| | | AGTGCAATCCCATGGGTTACACAAAAGAAGGCTGCAGGGGCATAGACAAAAGGCA |
| | | TTGGAACTCCCAGTGCCGAACTACCCAGTCGTACGTGCGGGCCCTTACCATGGAT |
| | | AGCAAAAAGAGAATTGGCTGGCGATTCATAAGGATAGACACTTCTTGTGTATGTA |
| | | CATTGACCATTAAAAGGGGAAGATACCCATACGATGTTCCAGATTACGCTTAGAC |
| | | CCAGCTTTCTTGTACAAAGTGGGAATTCCGATAATCAACCTCTGGATTACAAAAT |
| | | TTGTGAAAGATTGACTGGTATTCTTAACTATGTTGCTCCTTTTACGCTATGTGGA |
| | | TACGCTGCTTTAATGCCTTTGTATCATGCTATTGCTTCCCGTATGGCTTTCATTT |
| | | TCTCCTCCTTGTATAAATCCTGGTTGCTGTCTCTTTATGAGGAGTTGTGGCCCGT |
| | | TGTCAGGCAACGTGGCGTGGTGTGCACTGTGTTTGCTGACGCAACCCCCACTGGT |
| | | TGGGGCATTGCCACCACCTGTCAGCTCCTTTCCGGGACTTTCGCTTTCCCCCTCC |
| | | CTATTGCCACGGCGGAACTCATCGCCGCCTGCCTTGCCCGCTGCTGGACAGGGGC |
| | | TCGGCTGTTGGGCACTGACAATTCCGTGGTGTTGTCGGGGAAGCTGACGTCCTTT |
| | | CCATGGCTGCTCGCCTGTGTTGCCACCTGGATTCTGCGCGGGACGTCCTTCTGCT |
| | | ACGTCCCTTCGGCCCTCAATCCAGCGGACCTTCCTTCCCGCGGCCTGCTGCCGGC |
| | | TCTGCGGCCTCTTCCGCGTCTTCGCCTTCGCCCTCAGACGAGTCGGATCTCCCTT |
| | | TGGGCCGCCTCCCCGCATCGGGAATTCCTAGAGCTCGCTGATCAGCCTCGACTGT |
| | | GCCTTCTAGTTGCCAGCCATCTGTTGTTTGCCCCTCCCCCGTGCCTTCCTTGACC |
| | | CTGGAAGGTGCCACTCCCACTGTCCTTTCCTAATAAAATGAGGAAATTGCATCGC |
| | | ATTGTCTGAGTAGGTGTCATTCTATTCTGGGGGGTGGGGTGGGGCAGGACAGCAA |
| | | GGGGGAGGATTGGGAAGACAATAGCAGGCATGCTGGGGATGGCCCGGGCTCTATG |
| | | G |
| 37 | BDNF-E | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCGCCACCATG |
| | | CGGAGAATGCAGCTGCTGCTGCTCATTGCCCTGTCTCTGGCCCTGGTCACCAATA |
| | | GCCACTCTGACCCTGCCCGCCGAGGGGAGCTGAGCGTGTGTGACAGTATTAGTGA |
| | | GTGGGTAACGGCGGCAGACAAAAAGACTGCAGTGGACATGTCGGGCGGGACGGTC |
| | | ACAGTCCTTGAAAAGGTCCCTGTATCAAAAGGCCAACTGAAGCAATACTTCTACG |
| | | AGACCAAGTGCAATCCCATGGGTTACACAAAAGAAGGCTGCAGGGGCATAGACAA |
| | | AAGGCATTGGAACTCCCAGTGCCGAACTACCCAGTCGTACGTGCGGGCCCTTACC |
| | | ATGGATAGCAAAAAGAGAATTGGCTGGCGATTCATAAGGATAGACACTTCTTGTG |
| | | TATGTACATTGACCATTAAAAGGGGAAGAGGAGGCGGAGGATCTGGCGGAGGCGG |
| | | ATCTCACTCTGACCCTGCCCGCCGAGGGGAGCTGAGCGTGTGTGACAGTATTAGT |
| | | GAGTGGGTAACGGCGGCAGACAAAAAGACTGCAGTGGACATGTCGGGCGGGACGG |
| | | TCACAGTCCTTGAAAAGGTCCCTGTATCAAAAGGCCAACTGAAGCAATACTTCTA |
| | | CGAGACCAAGTGCAATCCCATGGGTTACACAAAAGAAGGCTGCAGGGGCATAGAC |
| | | AAAAGGCATTGGAACTCCCAGTGCCGAACTACCCAGTCGTACGTGCGGGCCCTTA |
| | | CCATGGATAGCAAAAAGAGAATTGGCTGGCGATTCATAAGGATAGACACTTCTTG |
| | | TGTATGTACATTGACCATTAAAAGGGGAAGATACCCATACGATGTTCCAGATTAC |
| | | GCTTAGACCCAGCTTTCTTGTACAAAGTGGGAATTCCGATAATCAACCTCTGGAT |
| | | TACAAAATTTGTGAAAGATTGACTGGTATTCTTAACTATGTTGCTCCTTTTACGC |
| | | TATGTGGATACGCTGCTTTAATGCCTTTGTATCATGCTATTGCTTCCCGTATGGC |
| | | TTTCATTTTCTCCTCCTTGTATAAATCCTGGTTGCTGTCTCTTTATGAGGAGTTG |
| | | TGGCCCGTTGTCAGGCAACGTGGCGTGGTGTGCACTGTGTTTGCTGACGCAACCC |
| | | CCACTGGTTGGGGCATTGCCACCACCTGTCAGCTCCTTTCCGGGACTTTCGCTTT |
| | | CCCCCTCCCTATTGCCACGGCGGAACTCATCGCCGCCTGCCTTGCCCGCTGCTGG |
| | | ACAGGGGCTCGGCTGTTGGGCACTGACAATTCCGTGGTGTTGTCGGGGAAGCTGA |
| | | CGTCCTTTCCATGGCTGCTCGCCTGTGTTGCCACCTGGATTCTGCGCGGGACGTC |
| | | CTTCTGCTACGTCCCTTCGGCCCTCAATCCAGCGGACCTTCCTTCCCGCGGCCTG |
| | | CTGCCGGCTCTGCGGCCTCTTCCGCGTCTTCGCCTTCGCCCTCAGACGAGTCGGA |
| | | TCTCCCTTTGGGCCGCCTCCCCGCATCGGGAATTCCTAGAGCTCGCTGATCAGCC |
| | | TCGACTGTGCCTTCTAGTTGCCAGCCATCTGTTGTTTGCCCCTCCCCCGTGCCTT |
| | | CCTTGACCCTGGAAGGTGCCACTCCCACTGTCCTTTCCTAATAAAATGAGGAAAT |
| | | TGCATCGCATTGTCTGAGTAGGTGTCATTCTATTCTGGGGGGTGGGGTGGGGCAG |
| | | GACAGCAAGGGGGAGGATTGGGAAGACAATAGCAGGCATGCTGGGGATGGCCCGG |
| | | GCTCTATGG |
| 38 | BDNF-F | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCAACTCCTAA |
| | | AAAACCGCCACCATGGGAGTGAAAGTTCTTTTTGCCCTTATTTGTATTGCTGTGG |
| | | CCGAGGCCGCCCCCATGAAAGAAGCAAACATCCGAGGACAAGGTGGCTTGGCCTA |
| | | CCCAGGTGTGCGGACCCATGGGACTCTGGAGAGCGTGAATGGGCCCAAGGCAGGT |
| | | TCAAGAGGCTTGACATCATTGGCTGACACTTTCGAACACGTGATAGAAGAGCTGT |
| | | TGGATGAGGACCAGAAAGTTCGGCCCAATGAAGAAAACAATAAGGACGCAGACTT |
| | | GTACACGTCCAGGGTGATGCTCAGTAGTCAAGTGCCTTTGGAGCCTCCTCTTCTC |
| | | TTTCTGCTGGAGGAATACAAAAATTACCTAGATGCTGCAAACATGTCCATGAGGG |
| | | TCCGGCGCCACTCTGACCCTGCCCGCCGAGGGGAGCTGAGCGTGTGTGACAGTAT |
| | | TAGTGAGTGGGTAACGGCGGCAGACAAAAAGACTGCAGTGGACATGTCGGGCGGG |
| | | ACGGTCACAGTCCTTGAAAAGGTCCCTGTATCAAAAGGCCAACTGAAGCAATACT |
| | | TCTACGAGACCAAGTGCAATCCCATGGGTTACACAAAAGAAGGCTGCAGGGGCAT |
| | | AGACAAAAGGCATTGGAACTCCCAGTGCCGAACTACCCAGTCGTACGTGCGGGCC |
| | | CTTACCATGGATAGCAAAAAGAGAATTGGCTGGCGATTCATAAGGATAGACACTT |
| | | CTTGTGTATGTACATTGACCATTAAAAGGGGAAGATACCCATACGATGTTCCAGA |
| | | TTACGCTTAGACCCAGCTTTCTTGTACAAAGTGGGAATTCCGATAATCAACCTCT |
| | | GGATTACAAAATTTGTGAAAGATTGACTGGTATTCTTAACTATGTTGCTCCTTTT |
| | | ACGCTATGTGGATACGCTGCTTTAATGCCTTTGTATCATGCTATTGCTTCCCGTA |
| | | TGGCTTTCATTTTCTCCTCCTTGTATAAATCCTGGTTGCTGTCTCTTTATGAGGA |
| | | GTTGTGGCCCGTTGTCAGGCAACGTGGCGTGGTGTGCACTGTGTTTGCTGACGCA |
| | | ACCCCCACTGGTTGGGGCATTGCCACCACCTGTCAGCTCCTTTCCGGGACTTTCG |
| | | CTTTCCCCCTCCCTATTGCCACGGCGGAACTCATCGCCGCCTGCCTTGCCCGCTG |
| | | CTGGACAGGGGCTCGGCTGTTGGGCACTGACAATTCCGTGGTGTTGTCGGGGAAG |
| | | CTGACGTCCTTTCCATGGCTGCTCGCCTGTGTTGCCACCTGGATTCTGCGCGGGA |
| | | CGTCCTTCTGCTACGTCCCTTCGGCCCTCAATCCAGCGGACCTTCCTTCCCGCGG |
| | | CCTGCTGCCGGCTCTGCGGCCTCTTCCGCGTCTTCGCCTTCGCCCTCAGACGAGT |
| | | CGGATCTCCCTTTGGGCCGCCTCCCCGCATCGGGAATTCCTAGAGCTCGCTGATC |
| | | AGCCTCGACTGTGCCTTCTAGTTGCCAGCCATCTGTTGTTTGCCCCTCCCCCGTG |
| | | CCTTCCTTGACCCTGGAAGGTGCCACTCCCACTGTCCTTTCCTAATAAAATGAGG |
| | | AAATTGCATCGCATTGTCTGAGTAGGTGTCATTCTATTCTGGGGGGTGGGGTGGG |
| | | GCAGGACAGCAAGGGGGAGGATTGGGAAGACAATAGCAGGCATGCTGGGGATGGC |
| | | CCGGGCTCTATGG |
| 39 | BDNF-H | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCAACTCCTAA |
| | | AAAACCGCCACCATGCGGAGGATGCAGCTGCTCTTGCTCATTGCCCTAAGTCTCG |
| | | CACTTGTCACAAACAGTGCCCCCATGAAAGAAGCAAACATCCGAGGACAAGGTGG |
| | | CTTGGCCTACCCAGGTGTGCGGACCCATGGGACTCTGGAGAGCGTGAATGGGCCC |
| | | AAGGCAGGTTCAAGAGGCTTGACATCATTGGCTGACACTTTCGAACACGTGATAG |
| | | AAGAGCTGTTGGATGAGGACCAGAAAGTTCGGCCCAATGAAGAAAACAATAAGGA |
| | | CGCAGACTTGTACACGTCCAGGGTGATGCTCAGTAGTCAAGTGCCTTTGGAGCCT |
| | | CCTCTTCTCTTTCTGCTGGAGGAATACAAAAATTACCTAGATGCTGCAAACATGT |
| | | CCATGAGGGTCCGGCGCCACTCTGACCCTGCCCGCCGAGGGGAGCTGAGCGTGTG |
| | | TGACAGTATTAGTGAGTGGGTAACGGCGGCAGACAAAAAGACTGCAGTGGACATG |
| | | TCGGGGGGACGGTCACAGTCCTTGAAAAGGTCCCTGTATCAAAAGGCCAACTGAA |
| | | GCAATACTTCTACGAGACCAAGTGCAATCCCATGGGTTACACAAAAGAAGGCTGC |
| | | AGGGGCATAGACAAAAGGCATTGGAACTCCCAGTGCCGAACTACCCAGTCGTACG |
| | | TGCGGGCCCTTACCATGGATAGCAAAAAGAGAATTGGCTGGCGATTCATAAGGAT |
| | | AGACACTTCTTGTGTATGTACATTGACCATTAAAAGGGGAAGATACCCATACGAT |
| | | GTTCCAGATTACGCTTAGACCCAGCTTTCTTGTACAAAGTGGGAATTCCGATAAT |
| | | CAACCTCTGGATTACAAAATTTGTGAAAGATTGACTGGTATTCTTAACTATGTTG |
| | | CTCCTTTTACGCTATGTGGATACGCTGCTTTAATGCCTTTGTATCATGCTATTGC |
| | | TTCCCGTATGGCTTTCATTTTCTCCTCCTTGTATAAATCCTGGTTGCTGTCTCTT |
| | | TATGAGGAGTTGTGGCCCGTTGTCAGGCAACGTGGCGTGGTGTGCACTGTGTTTG |
| | | CTGACGCAACCCCCACTGGTTGGGGCATTGCCACCACCTGTCAGCTCCTTTCCGG |
| | | GACTTTCGCTTTCCCCCTCCCTATTGCCACGGCGGAACTCATCGCCGCCTGCCTT |
| | | GCCCGCTGCTGGACAGGGGCTCGGCTGTTGGGCACTGACAATTCCGTGGTGTTGT |
| | | CGGGGAAGCTGACGTCCTTTCCATGGCTGCTCGCCTGTGTTGCCACCTGGATTCT |
| | | GCGCGGGACGTCCTTCTGCTACGTCCCTTCGGCCCTCAATCCAGCGGACCTTCCT |
| | | TCCCGCGGCCTGCTGCCGGCTCTGCGGCCTCTTCCGCGTCTTCGCCTTCGCCCTC |
| | | AGACGAGTCGGATCTCCCTTTGGGCCGCCTCCCCGCATCGGGAATTCCTAGAGCT |
| | | CGCTGATCAGCCTCGACTGTGCCTTCTAGTTGCCAGCCATCTGTTGTTTGCCCCT |
| | | CCCCCGTGCCTTCCTTGACCCTGGAAGGTGCCACTCCCACTGTCCTTTCCTAATA |
| | | AAATGAGGAAATTGCATCGCATTGTCTGAGTAGGTGTCATTCTATTCTGGGGGGT |
| | | GGGGTGGGGCAGGACAGCAAGGGGGAGGATTGGGAAGACAATAGCAGGCATGCTG |
| | | GGGATGGCCCGGGCTCTATGG |
| 40 | BDNF-J | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCAACTCCTAA |
| | | AAAACCGCCACCATGAGACGACTCTGGTGGCTGTTGCTTCTGCTCTTGCTTCTGT |
| | | GGCCCATGGTCTGGGCCGCCCCCATGAAAGAAGCAAACATCCGAGGACAAGGTGG |
| | | CTTGGCCTACCCAGGTGTGCGGACCCATGGGACTCTGGAGAGCGTGAATGGGCCC |
| | | AAGGCAGGTTCAAGAGGCTTGACATCATTGGCTGACACTTTCGAACACGTGATAG |
| | | AAGAGCTGTTGGATGAGGACCAGAAAGTTCGGCCCAATGAAGAAAACAATAAGGA |
| | | CGCAGACTTGTACACGTCCAGGGTGATGCTCAGTAGTCAAGTGCCTTTGGAGCCT |
| | | CCTCTTCTCTTTCTGCTGGAGGAATACAAAAATTACCTAGATGCTGCAAACATGT |
| | | CCATGAGGGTCCGGCGCCACTCTGACCCTGCCCGCCGAGGGGAGCTGAGCGTGTG |
| | | TGACAGTATTAGTGAGTGGGTAACGGCGGCAGACAAAAAGACTGCAGTGGACATG |
| | | TCGGGCGGGACGGTCACAGTCCTTGAAAAGGTCCCTGTATCAAAAGGCCAACTGA |
| | | AGCAATACTTCTACGAGACCAAGTGCAATCCCATGGGTTACACAAAAGAAGGCTG |
| | | CAGGGGCATAGACAAAAGGCATTGGAACTCCCAGTGCCGAACTACCCAGTCGTAC |
| | | GTGCGGGCCCTTACCATGGATAGCAAAAAGAGAATTGGCTGGCGATTCATAAGGA |
| | | TAGACACTTCTTGTGTATGTACATTGACCATTAAAAGGGGAAGATACCCATACGA |
| | | TGTTCCAGATTACGCTTAGACCCAGCTTTCTTGTACAAAGTGGGAATTCCGATAA |
| | | TCAACCTCTGGATTACAAAATTTGTGAAAGATTGACTGGTATTCTTAACTATGTT |
| | | GCTCCTTTTACGCTATGTGGATACGCTGCTTTAATGCCTTTGTATCATGCTATTG |
| | | CTTCCCGTATGGCTTTCATTTTCTCCTCCTTGTATAAATCCTGGTTGCTGTCTCT |
| | | TTATGAGGAGTTGTGGCCCGTTGTCAGGCAACGTGGCGTGGTGTGCACTGTGTTT |
| | | GCTGACGCAACCCCCACTGGTTGGGGCATTGCCACCACCTGTCAGCTCCTTTCCG |
| | | GGACTTTCGCTTTCCCCCTCCCTATTGCCACGGCGGAACTCATCGCCGCCTGCCT |
| | | TGCCCGCTGCTGGACAGGGGCTCGGCTGTTGGGCACTGACAATTCCGTGGTGTTG |
| | | TCGGGGAAGCTGACGTCCTTTCCATGGCTGCTCGCCTGTGTTGCCACCTGGATTC |
| | | TGCGCGGGACGTCCTTCTGCTACGTCCCTTCGGCCCTCAATCCAGCGGACCTTCC |
| | | TTCCCGCGGCCTGCTGCCGGCTCTGCGGCCTCTTCCGCGTCTTCGCCTTCGCCCT |
| | | CAGACGAGTCGGATCTCCCTTTGGGCCGCCTCCCCGCATCGGGAATTCCTAGAGC |
| | | TCGCTGATCAGCCTCGACTGTGCCTTCTAGTTGCCAGCCATCTGTTGTTTGCCCC |
| | | TCCCCCGTGCCTTCCTTGACCCTGGAAGGTGCCACTCCCACTGTCCTTTCCTAAT |
| | | AAAATGAGGAAATTGCATCGCATTGTCTGAGTAGGTGTCATTCTATTCTGGGGGG |
| | | TGGGGTGGGGCAGGACAGCAAGGGGGAGGATTGGGAAGACAATAGCAGGCATGCT |
| | | GGGGATGGCCCGGGCTCTATGG |
| 41 | BDNF-L | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCAACTCCTAA |
| | | AAAACCGCCACCATGAGACGACTCTGGTGGCTGTTGCTTCTGCTCTTGCTTCTGT |
| | | GGCCCTGTATGAAGGCCGCCCCCATGAAAGAAGCAAACATCCGAGGACAAGGTGG |
| | | CTTGGCCTACCCAGGTGTGCGGACCCATGGGACTCTGGAGAGCGTGAATGGGCCC |
| | | AAGGCAGGTTCAAGAGGCTTGACATCATTGGCTGACACTTTCGAACACGTGATAG |
| | | AAGAGCTGTTGGATGAGGACCAGAAAGTTCGGCCCAATGAAGAAAACAATAAGGA |
| | | CGCAGACTTGTACACGTCCAGGGTGATGCTCAGTAGTCAAGTGCCTTTGGAGCCT |
| | | CCTCTTCTCTTTCTGCTGGAGGAATACAAAAATTACCTAGATGCTGCAAACATGT |
| | | CCATGAGGGTCCGGCGCCACTCTGACCCTGCCCGCCGAGGGGAGCTGAGCGTGTG |
| | | TGACAGTATTAGTGAGTGGGTAACGGCGGCAGACAAAAAGACTGCAGTGGACATG |
| | | TCGGGCGGGACGGTCACAGTCCTTGAAAAGGTCCCTGTATCAAAAGGCCAACTGA |
| | | AGCAATACTTCTACGAGACCAAGTGCAATCCCATGGGTTACACAAAAGAAGGCTG |
| | | CAGGGGCATAGACAAAAGGCATTGGAACTCCCAGTGCCGAACTACCCAGTCGTAC |
| | | GTGCGGGCCCTTACCATGGATAGCAAAAAGAGAATTGGCTGGCGATTCATAAGGA |
| | | TAGACACTTCTTGTGTATGTACATTGACCATTAAAAGGGGAAGATACCCATACGA |
| | | TGTTCCAGATTACGCTTAGACCCAGCTTTCTTGTACAAAGTGGGAATTCCGATAA |
| | | TCAACCTCTGGATTACAAAATTTGTGAAAGATTGACTGGTATTCTTAACTATGTT |
| | | GCTCCTTTTACGCTATGTGGATACGCTGCTTTAATGCCTTTGTATCATGCTATTG |
| | | CTTCCCGTATGGCTTTCATTTTCTCCTCCTTGTATAAATCCTGGTTGCTGTCTCT |
| | | TTATGAGGAGTTGTGGCCCGTTGTCAGGCAACGTGGCGTGGTGTGCACTGTGTTT |
| | | GCTGACGCAACCCCCACTGGTTGGGGCATTGCCACCACCTGTCAGCTCCTTTCCG |
| | | GGACTTTCGCTTTCCCCCTCCCTATTGCCACGGCGGAACTCATCGCCGCCTGCCT |
| | | TGCCCGCTGCTGGACAGGGGCTCGGCTGTTGGGCACTGACAATTCCGTGGTGTTG |
| | | TCGGGGAAGCTGACGTCCTTTCCATGGCTGCTCGCCTGTGTTGCCACCTGGATTC |
| | | TGCGCGGGACGTCCTTCTGCTACGTCCCTTCGGCCCTCAATCCAGCGGACCTTCC |
| | | TTCCCGCGGCCTGCTGCCGGCTCTGCGGCCTCTTCCGCGTCTTCGCCTTCGCCCT |
| | | CAGACGAGTCGGATCTCCCTTTGGGCCGCCTCCCCGCATCGGGAATTCCTAGAGC |
| | | TCGCTGATCAGCCTCGACTGTGCCTTCTAGTTGCCAGCCATCTGTTGTTTGCCCC |
| | | TCCCCCGTGCCTTCCTTGACCCTGGAAGGTGCCACTCCCACTGTCCTTTCCTAAT |
| | | AAAATGAGGAAATTGCATCGCATTGTCTGAGTAGGTGTCATTCTATTCTGGGGGG |
| | | TGGGGTGGGGCAGGACAGCAAGGGGGAGGATTGGGAAGACAATAGCAGGCATGCT |
| | | GGGGATGGCCCGGGCTCTATGG |
| 42 | BDNF-N | GAGTAATTCATACAAAAGGACTCGCCCCTGCCTTGGGGAATCCCAGGGACCGTCG |
| | | TTAAACTCCCACTAACGTAGAACCCAGAGATCGCTGCGTTCCCGCCCCCTCACCC |
| | | GCCCGCTCTCGTCATCACTGAGGTGGAGAAGAGCATGCGTGAGGCTCCGGTGCCC |
| | | GTCAGTGGGCAGAGCGCACATCGCCCACAGTCCCCGAGAAGTTGGGGGGAGGGGT |
| | | CGGCAATTGAACCGGTGCCTAGAGAAGGTGGCGCGGGGTAAACTGGGAAAGTGAT |
| | | GTCGTGTACTGGCTCCGCCTTTTTCCCGAGGGTGGGGGAGAACCGTATATAAGTG |
| | | CGTAGTCGCCGTGAACGTTCTTTTTCGCAACGGGCTCGAGCCGAGTGGTCGTTGC |
| | | CGCCAGAACACAGGTAAGTGCCGTGTGTGGTTCCCGCGGGCCTGGCCTCTTTACG |
| | | GGTTATGGCCCTTGCGTGCCTTGAATTACTTCCACGCCCCTGGCTGCAGTACGTG |
| | | ATTCTTGATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCTTGGGCGCTGGGG |
| | | CCGCCGCGTGCGAATCTGGTGGCACCTTCGCGCCTGTCTCGCTGCTTTCGATAAG |
| | | TCTCTAGCCATTTAAAATTTTTGATGACCTGCTGCGACGCTTTTTTTCTGGCAAG |
| | | ATAGTCTTGTAAATGCGGGCCAAGATCTGCACACTGGTATTTCGGTTTTTGGGGC |
| | | CGCGGGCGGCGACGGGGCCCGTGCGTCCCAGCGCACATGTTCGGCGAGGCGGGGC |
| | | CTGCGAGCGCGGCCACCGAGAATCGGACGGGGGTAGTCTCAAGCTGGCCGGCCTG |
| | | CTCTGGTGCCTGGCCTCGCGCCGCCGTGTATCGCCCCGCCCTGGGCGGCAAGGCT |
| | | GGCCCGGTCGGCACCAGTTGCGTGAGCGGAAAGATGGCCGCTTCCCGGCCCTGCT |
| | | GCAGGGAGCTCAAAATGGAGGACGCGGCGCTCGGGAGAGCGGGCGGGTGAGTCAC |
| | | CCACACAAAGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTTGGAGTACG |
| | | TCGTCTTTAGGTTGGGGGGAGGGGTTTTATGCGATGGAGTTTCCCCACACTGAGT |
| | | GGGTGGAGACTGAAGTTAGGCCAGCTTGGCACTTGATGTAATTCTCCTTGGAATT |
| | | TGCCCTTTTTGAGTTTGGATCTTGGTTCATTCTCAAGCCTCAGACAGTGGTTCAA |
| | | AGTTTTTTTCTTCCATTTCAGGTGTCGTGAGGATCCAACTCCTAAAAAACCGCCA |
| | | CCATGACCATCCTTTTCCTTACTATGGTTATTTCATACTTTGGTTGCATGAAGGC |
| | | TGCCCCCATGAAAGAAGCAAACATCCGAGGACAAGGTGGCTTGGCCTACCCAGGT |
| | | GTGCGGACCCATGGGACTCTGGAGAGCGTGAATGGGCCCAAGGCAGGTTCAAGAG |
| | | GCTTGACATCATTGGCTGACACTTTCGAACACGTGATAGAAGAGCTGTTGGATGA |
| | | GGACCAGAAAGTTCGGCCCAATGAAGAAAACAATAAGGACGCAGACTTGTACACG |
| | | TCCAGGGTGATGCTCAGTAGTCAAGTGCCTTTGGAGCCTCCTCTTCTCTTTCTGC |
| | | TGGAGGAATACAAAAATTACCTAGATGCTGCAAACATGTCCATGAGGGTCCGGCG |
| | | CCACTCTGACCCTGCCCGCCGAGGGGAGCTGAGCGTGTGTGACAGTATTAGTGAG |
| | | TGGGTAACGGCGGCAGACAAAAAGACTGCAGTGGACATGTCGGGGGGACGGTCAC |
| | | AGTCCTTGAAAAGGTCCCTGTATCAAAAGGCCAACTGAAGCAATACTTCTACGAG |
| | | ACCAAGTGCAATCCCATGGGTTACACAAAAGAAGGCTGCAGGGGCATAGACAAAA |
| | | GGCATTGGAACTCCCAGTGCCGAACTACCCAGTCGTACGTGCGGGCCCTTACCAT |
| | | GGATAGCAAAAAGAGAATTGGCTGGCGATTCATAAGGATAGACACTTCTTGTGTA |
| | | TGTACATTGACCATTAAAAGGGGAAGATACCCATACGATGTTCCAGATTACGCTT |
| | | AGACCCAGCTTTCTTGTACAAAGTGGGAATTCCGATAATCAACCTCTGGATTACA |
| | | AAATTTGTGAAAGATTGACTGGTATTCTTAACTATGTTGCTCCTTTTACGCTATG |
| | | TGGATACGCTGCTTTAATGCCTTTGTATCATGCTATTGCTTCCCGTATGGCTTTC |
| | | ATTTTCTCCTCCTTGTATAAATCCTGGTTGCTGTCTCTTTATGAGGAGTTGTGGC |
| | | CCGTTGTCAGGCAACGTGGCGTGGTGTGCACTGTGTTTGCTGACGCAACCCCCAC |
| | | TGGTTGGGGCATTGCCACCACCTGTCAGCTCCTTTCCGGGACTTTCGCTTTCCCC |
| | | CTCCCTATTGCCACGGCGGAACTCATCGCCGCCTGCCTTGCCCGCTGCTGGACAG |
| | | GGGCTCGGCTGTTGGGCACTGACAATTCCGTGGTGTTGTCGGGGAAGCTGACGTC |
| | | CTTTCCATGGCTGCTCGCCTGTGTTGCCACCTGGATTCTGCGCGGGACGTCCTTC |
| | | TGCTACGTCCCTTCGGCCCTCAATCCAGCGGACCTTCCTTCCCGCGGCCTGCTGC |
| | | CGGCTCTGCGGCCTCTTCCGCGTCTTCGCCTTCGCCCTCAGACGAGTCGGATCTC |
| | | CCTTTGGGCCGCCTCCCCGCATCGGGAATTCCTAGAGCTCGCTGATCAGCCTCGA |
| | | CTGTGCCTTCTAGTTGCCAGCCATCTGTTGTTTGCCCCTCCCCCGTGCCTTCCTT |
| | | GACCCTGGAAGGTGCCACTCCCACTGTCCTTTCCTAATAAAATGAGGAAATTGCA |
| | | TCGCATTGTCTGAGTAGGTGTCATTCTATTCTGGGGGGTGGGGTGGGGCAGGACA |
| | | GCAAGGGGGAGGATTGGGAAGACAATAGCAGGCATGCTGGGGATGGCCCGGGCTC |
| | | TATGG |
| 43 | BDNF-O | CGAGTCTTCAAACCTTAAGCCTCAGTGCAAAAGGGAGCAGCGAGGAGAGGCGGTG |
| | | GGATTAAAGAAAAAAATTCTTCTCTCCCTGTCACTTCAAAAGGTCGCCCAACCAG |
| | | CGAGCGGGGGGTTTACCCACGACTCTGGCTCGCACCCCTTGCGGGCCTGCGCAGG |
| | | CGGCCCCGTAGCGCAAGGGAGGGCGGGAAAGGAAGGGGCGGGGACGCGAGGGCGA |
| | | ATCTATAAGAAGCGTCGTTCAGCGAGTTCGCTGCTCAGAAGCGCCGAGAGCGCGG |
| | | CCGGGACGGTTGGAGAAGAAGGCGGCTCCCGGAAGGGGGAGAGACAAACTGCCGT |
| | | AACCTCTGCCGTTCAGGAACGTCGAGGTGAGCCCCACGTTCTGCTTCACTCTCCC |
| | | CATCTCCCCCCCCTCCCCACCCCCAATTTTGTATTTATTTATTTTTTAATTATTT |
| | | TGTGCAGCGATGGGGGCGGGGGGGGGGGGGGGGCGCGCGCCAGGCGGGGCGGGGC |
| | | GGGGCGAGGGGCGGGGCGGGGCGAGGCGGAGAGGTGCGGCGGCAGCCAATCAGAG |
| | | CGGCGCGCTCCGAAAGTTTCCTTTTATGGCGAGGCGGCGGCGGCGGCGGCCCTAT |
| | | AAAAAGCGAAGCGCGCGGCGGGCGGGAGTCGCTGCGCGCTGCCTTCGCCCCGTGC |
| | | CCCGCTCCGCCGCCGCCTCGCGCCGCCCGCCCCGGCTCTGACTGACCGCGTTACT |
| | | CCCACAGGTGAGCGGGCGGGACGGCCCTTCTCCTCCGGGCTGTAATTAGCTGAGC |
| | | AAGAGGTAAGGGTTTAAGGGATGGTTGGTTGGTGGGGTATTAATGTTTAATTACC |
| | | TGGAGCACCTGCCTGAAATCACTTTTTTTCAGGTTGGACCGGTGCGGATCCAACT |
| | | CCTAAAAAACCGCCACCATGACCATCCTTTTCCTTACTATGGTTATTTCATACTT |
| | | TGGTTGCATGAAGGCTGCCCCCATGAAAGAAGCAAACATCCGAGGACAAGGTGGC |
| | | TTGGCCTACCCAGGTGTGCGGACCCATGGGACTCTGGAGAGCGTGAATGGGCCCA |
| | | AGGCAGGTTCAAGAGGCTTGACATCATTGGCTGACACTTTCGAACACGTGATAGA |
| | | AGAGCTGTTGGATGAGGACCAGAAAGTTCGGCCCAATGAAGAAAACAATAAGGAC |
| | | GCAGACTTGTACACGTCCAGGGTGATGCTCAGTAGTCAAGTGCCTTTGGAGCCTC |
| | | CTCTTCTCTTTCTGCTGGAGGAATACAAAAATTACCTAGATGCTGCAAACATGTC |
| | | CATGAGGGTCCGGCGCCACTCTGACCCTGCCCGCCGAGGGGAGCTGAGCGTGTGT |
| | | GACAGTATTAGTGAGTGGGTAACGGCGGCAGACAAAAAGACTGCAGTGGACATGT |
| | | CGGGCGGGACGGTCACAGTCCTTGAAAAGGTCCCTGTATCAAAAGGCCAACTGAA |
| | | GCAATACTTCTACGAGACCAAGTGCAATCCCATGGGTTACACAAAAGAAGGCTGC |
| | | AGGGGCATAGACAAAAGGCATTGGAACTCCCAGTGCCGAACTACCCAGTCGTACG |
| | | TGCGGGCCCTTACCATGGATAGCAAAAAGAGAATTGGCTGGCGATTCATAAGGAT |
| | | AGACACTTCTTGTGTATGTACATTGACCATTAAAAGGGGAAGATACCCATACGAT |
| | | GTTCCAGATTACGCTTAGACCCAGCTTTCTTGTACAAAGTGGGAATTCCGATAAT |
| | | CAACCTCTGGATTACAAAATTTGTGAAAGATTGACTGGTATTCTTAACTATGTTG |
| | | CTCCTTTTACGCTATGTGGATACGCTGCTTTAATGCCTTTGTATCATGCTATTGC |
| | | TTCCCGTATGGCTTTCATTTTCTCCTCCTTGTATAAATCCTGGTTGCTGTCTCTT |
| | | TATGAGGAGTTGTGGCCCGTTGTCAGGCAACGTGGCGTGGTGTGCACTGTGTTTG |
| | | CTGACGCAACCCCCACTGGTTGGGGCATTGCCACCACCTGTCAGCTCCTTTCCGG |
| | | GACTTTCGCTTTCCCCCTCCCTATTGCCACGGCGGAACTCATCGCCGCCTGCCTT |
| | | GCCCGCTGCTGGACAGGGGCTCGGCTGTTGGGCACTGACAATTCCGTGGTGTTGT |
| | | CGGGGAAGCTGACGTCCTTTCCATGGCTGCTCGCCTGTGTTGCCACCTGGATTCT |
| | | GCGCGGGACGTCCTTCTGCTACGTCCCTTCGGCCCTCAATCCAGCGGACCTTCCT |
| | | TCCCGCGGCCTGCTGCCGGCTCTGCGGCCTCTTCCGCGTCTTCGCCTTCGCCCTC |
| | | AGACGAGTCGGATCTCCCTTTGGGCCGCCTCCCCGCATCGGGAATTCCTAGAGCT |
| | | CGCTGATCAGCCTCGACTGTGCCTTCTAGTTGCCAGCCATCTGTTGTTTGCCCCT |
| | | CCCCCGTGCCTTCCTTGACCCTGGAAGGTGCCACTCCCACTGTCCTTTCCTAATA |
| | | AAATGAGGAAATTGCATCGCATTGTCTGAGTAGGTGTCATTCTATTCTGGGGGGT |
| | | GGGGTGGGGCAGGACAGCAAGGGGGAGGATTGGGAAGACAATAGCAGGCATGCTG |
| | | GGGATGGCCCGGGCTCTATGG |
| 44 | BDNF-P | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCAACTCCTAA |
| | | AAAACCGCCACCATGACCATCCTTTTCCTTACTATGGTTATTTCATACTTTGGTT |
| | | GCATGAAGGCTGCCCCCATGAAAGAAGCAAACATCCGAGGACAAGGTGGCTTGGC |
| | | CTACCCAGGTGTGCGGACCCATGGGACTCTGGAGAGCGTGAATGGGCCCAAGGCA |
| | | GGTTCAAGAGGCTTGACATCATTGGCTGACACTTTCGAACACGTGATAGAAGAGC |
| | | TGTTGGATGAGGACCAGAAAGTTCGGCCCAATGAAGAAAACAATAAGGACGCAGA |
| | | CTTGTACACGTCCAGGGTGATGCTCAGTAGTCAAGTGCCTTTGGAGCCTCCTCTT |
| | | CTCTTTCTGCTGGAGGAATACAAAAATTACCTAGATGCTGCAAACATGTCCATGA |
| | | GGGTCCGGCGCCACTCTGACCCTGCCCGCCGAGGGGAGCTGAGCGTGTGTGACAG |
| | | TATTAGTGAGTGGGTAACGGCGGCAGACAAAAAGACTGCAGTGGACATGTCGGGC |
| | | GGGACGGTCACAGTCCTTGAAAAGGTCCCTGTATCAAAAGGCCAACTGAAGCAAT |
| | | ACTTCTACGAGACCAAGTGCAATCCCATGGGTTACACAAAAGAAGGCTGCAGGGG |
| | | CATAGACAAAAGGCATTGGAACTCCCAGTGCCGAACTACCCAGTCGTACGTGCGG |
| | | GCCCTTACCATGGATAGCAAAAAGAGAATTGGCTGGCGATTCATAAGGATAGACA |
| | | CTTCTTGTGTATGTACATTGACCATTAAAAGGGGAAGATACCCATACGATGTTCC |
| | | AGATTACGCTTAGACCCAGCTTTCTTGTACAAAGTGGGAATTCCGATAATCAACC |
| | | TCTGGATTACAAAATTTGTGAAAGATTGACTGGTATTCTTAACTATGTTGCTCCT |
| | | TTTACGCTATGTGGATACGCTGCTTTAATGCCTTTGTATCATGCTATTGCTTCCC |
| | | GTATGGCTTTCATTTTCTCCTCCTTGTATAAATCCTGGTTGCTGTCTCTTTATGA |
| | | GGAGTTGTGGCCCGTTGTCAGGCAACGTGGCGTGGTGTGCACTGTGTTTGCTGAC |
| | | GCAACCCCCACTGGTTGGGGCATTGCCACCACCTGTCAGCTCCTTTCCGGGACTT |
| | | TCGCTTTCCCCCTCCCTATTGCCACGGCGGAACTCATCGCCGCCTGCCTTGCCCG |
| | | CTGCTGGACAGGGGCTCGGCTGTTGGGCACTGACAATTCCGTGGTGTTGTCGGGG |
| | | AAGCTGACGTCCTTTCCATGGCTGCTCGCCTGTGTTGCCACCTGGATTCTGCGCG |
| | | GGACGTCCTTCTGCTACGTCCCTTCGGCCCTCAATCCAGCGGACCTTCCTTCCCG |
| | | CGGCCTGCTGCCGGCTCTGCGGCCTCTTCCGCGTCTTCGCCTTCGCCCTCAGACG |
| | | AGTCGGATCTCCCTTTGGGCCGCCTCCCCGCATCGGGAATTCCTAGAGCTCGCTG |
| | | ATCAGCCTCGACTGTGCCTTCTAGTTGCCAGCCATCTGTTGTTTGCCCCTCCCCC |
| | | GTGCCTTCCTTGACCCTGGAAGGTGCCACTCCCACTGTCCTTTCCTAATAAAATG |
| | | AGGAAATTGCATCGCATTGTCTGAGTAGGTGTCATTCTATTCTGGGGGGTGGGGT |
| | | GGGGCAGGACAGCAAGGGGGAGGATTGGGAAGACAATAGCAGGCATGCTGGGGAT |
| | | GGCCCGGGCTCTATGG |
| 45 | BDNF-Q | TTACATCAAGTGCCAAGCTGGCCTAACTTCAGTCTCCACCCACTCAGTGTGGGGA |
| | | AACTCCATCGCATAAAACCCCTCCCCCCAACCTAAAGACGACGTACTCCAAAAGC |
| | | TCGAGAACTAATCGAGGTGCCTGGACGGCGCCCGGTACTCCGTGGAGTCACATGA |
| | | AGCGACGGCTGAGGACGGAAAGGCCCTTTTCCTTTGTGTGGGTGACTCACCCGCC |
| | | CGCTCTCCCGAGCGCCGCGTCCTCCATTTTGAGCTCCCTGCAGCAGGGCCGGGAA |
| | | GCGGCCATCTTTCCGCTCACGCAACTGGTGCCGACCGGGCCAGCCTTGCCGCCCA |
| | | GGGCGGGGCGATACACAATTGGTTTGATCTGATTATAACTAGTGGAGAAGAGCAT |
| | | GCTTGAGGGCTGAGTGCCCGTCAGTGGGCAGAGCGCACATCGCCCACAGTCCCCG |
| | | AGAAGTTGGGGGGAGGGGTCGGCAATTGAACCGGTGCCTAGAGAAGGTGGCGCGG |
| | | GGTAAACTGGGAAAGTGATGTCGTGTACTGGCTCCGCCTTTTTCCCGAGGGTGGG |
| | | GGAGAACCGTATATAAGTGCAGTAGTCGCCGTGAACGTTCTTTTTCGCAACGGGC |
| | | TCGAGCCGAGTGGTCGTTGCCGCCAGAACACAGCTGAAGCTTCGAGGGGCTCGCA |
| | | TCTCTCCTTCACGCGCCCGCCGCCCTACCTGAGGCCGCCATCCACGCCGGTTGAG |
| | | TCGCGTTCTGCCGCCTCCCGCCTGTGGTGCCTCCTGAACTGCGTCCGCCGTCTAG |
| | | GTAAGTTTAAAGCTCAGGTCGAGACCGGGCCTTTGTCCGGCGCTCCCTTGGAGCC |
| | | TACCTAGACTCAGCCGGCTCTCCACGCTTTGCCTGACCCTGCTTGCTCAACTCTA |
| | | CGTCTTTGTTTCGTTTTCTGTTCTGCGCCGTTACAGATCCAAGCTGTGACCGGCG |
| | | CCTACAAACAGTAGTTGACGGATCCAACTCCTAAAAAACCGCCACCATGACCATC |
| | | CTTTTCCTTACTATGGTTATTTCATACTTTGGTTGCATGAAGGCTGCCCCCATGA |
| | | AAGAAGCAAACATCCGAGGACAAGGTGGCTTGGCCTACCCAGGTGTGCGGACCCA |
| | | TGGGACTCTGGAGAGCGTGAATGGGCCCAAGGCAGGTTCAAGAGGCTTGACATCA |
| | | TTGGCTGACACTTTCGAACACGTGATAGAAGAGCTGTTGGATGAGGACCAGAAAG |
| | | TTCGGCCCAATGAAGAAAACAATAAGGACGCAGACTTGTACACGTCCAGGGTGAT |
| | | GCTCAGTAGTCAAGTGCCTTTGGAGCCTCCTCTTCTCTTTCTGCTGGAGGAATAC |
| | | AAAAATTACCTAGATGCTGCAAACATGTCCATGAGGGTCCGGCGCCACTCTGACC |
| | | CTGCCCGCCGAGGGGAGCTGAGCGTGTGTGACAGTATTAGTGAGTGGGTAACGGC |
| | | GGCAGACAAAAAGACTGCAGTGGACATGTCGGGCGGGACGGTCACAGTCCTTGAA |
| | | AAGGTCCCTGTATCAAAAGGCCAACTGAAGCAATACTTCTACGAGACCAAGTGCA |
| | | ATCCCATGGGTTACACAAAAGAAGGCTGCAGGGGCATAGACAAAAGGCATTGGAA |
| | | CTCCCAGTGCCGAACTACCCAGTCGTACGTGCGGGCCCTTACCATGGATAGCAAA |
| | | AAGAGAATTGGCTGGCGATTCATAAGGATAGACACTTCTTGTGTATGTACATTGA |
| | | CCATTAAAAGGGGAAGATACCCATACGATGTTCCAGATTACGCTTAGACCCAGCT |
| | | TTCTTGTACAAAGTGGGAATTCCGATAATCAACCTCTGGATTACAAAATTTGTGA |
| | | AAGATTGACTGGTATTCTTAACTATGTTGCTCCTTTTACGCTATGTGGATACGCT |
| | | GCTTTAATGCCTTTGTATCATGCTATTGCTTCCCGTATGGCTTTCATTTTCTCCT |
| | | CCTTGTATAAATCCTGGTTGCTGTCTCTTTATGAGGAGTTGTGGCCCGTTGTCAG |
| | | GCAACGTGGCGTGGTGTGCACTGTGTTTGCTGACGCAACCCCCACTGGTTGGGGC |
| | | ATTGCCACCACCTGTCAGCTCCTTTCCGGGACTTTCGCTTTCCCCCTCCCTATTG |
| | | CCACGGCGGAACTCATCGCCGCCTGCCTTGCCCGCTGCTGGACAGGGGCTCGGCT |
| | | GTTGGGCACTGACAATTCCGTGGTGTTGTCGGGGAAGCTGACGTCCTTTCCATGG |
| | | CTGCTCGCCTGTGTTGCCACCTGGATTCTGCGCGGGACGTCCTTCTGCTACGTCC |
| | | CTTCGGCCCTCAATCCAGCGGACCTTCCTTCCCGCGGCCTGCTGCCGGCTCTGCG |
| | | GCCTCTTCCGCGTCTTCGCCTTCGCCCTCAGACGAGTCGGATCTCCCTTTGGGCC |
| | | GCCTCCCCGCATCGGGAATTCCTAGAGCTCGCTGATCAGCCTCGACTGTGCCTTC |
| | | TAGTTGCCAGCCATCTGTTGTTTGCCCCTCCCCCGTGCCTTCCTTGACCCTGGAA |
| | | GGTGCCACTCCCACTGTCCTTTCCTAATAAAATGAGGAAATTGCATCGCATTGTC |
| | | TGAGTAGGTGTCATTCTATTCTGGGGGGTGGGGTGGGGCAGGACAGCAAGGGGGA |
| | | GGATTGGGAAGACAATAGCAGGCATGCTGGGGATGGCCCGGGCTCTATGG |
| 46 | BDNF-R | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCGCCACCATG |
| | | ACCATCCTTTTCCTTACTATGGTTATTTCATACTTTGGTTGCATGAAGGCTGCCC |
| | | CCATGAAAGAAGCAAACATCCGAGGACAAGGTGGCTTGGCCTACCCAGGTGTGCG |
| | | GACCCATGGGACTCTGGAGAGCGTGAATGGGCCCAAGGCAGGTTCAAGAGGCTTG |
| | | ACATCATTGGCTGACACTTTCGAACACGTGATAGAAGAGCTGTTGGATGAGGACC |
| | | AGAAAGTTCGGCCCAATGAAGAAAACAATAAGGACGCAGACTTGTACACGTCCAG |
| | | GGTGATGCTCAGTAGTCAAGTGCCTTTGGAGCCTCCTCTTCTCTTTCTGCTGGAG |
| | | GAATACAAAAATTACCTAGATGCTGCAAACATGTCCATGAGGGTCCGGCGCCACT |
| | | CTGACCCTGCCCGCCGAGGGGAGCTGAGCGTGTGTGACAGTATTAGTGAGTGGGT |
| | | AACGGCGGCAGACAAAAAGACTGCAGTGGACATGTCGGGCGGGACGGTCACAGTC |
| | | CTTGAAAAGGTCCCTGTATCAAAAGGCCAACTGAAGCAATACTTCTACGAGACCA |
| | | AGTGCAATCCCATGGGTTACACAAAAGAAGGCTGCAGGGGCATAGACAAAAGGCA |
| | | TTGGAACTCCCAGTGCCGAACTACCCAGTCGTACGTGCGGGCCCTTACCATGGAT |
| | | AGCAAAAAGAGAATTGGCTGGCGATTCATAAGGATAGACACTTCTTGTGTATGTA |
| | | CATTGACCATTAAAAGGGGAAGACGCAAAAAACGCAGCGTGGGCAGCGGCGCGCC |
| | | GGTGAAACAGACCCTGAACTTTGATCTGCTGAAACTGGCGGGCGATGTGGAAAGC |
| | | AACCCGGGCCCGATGACCATCCTTTTCCTTACTATGGTTATTTCATACTTTGGTT |
| | | GCATGAAGGCTGCCCCCATGAAAGAAGCAAACATCCGAGGACAAGGTGGCTTGGC |
| | | CTACCCAGGTGTGCGGACCCATGGGACTCTGGAGAGCGTGAATGGGCCCAAGGCA |
| | | GGTTCAAGAGGCTTGACATCATTGGCTGACACTTTCGAACACGTGATAGAAGAGC |
| | | TGTTGGATGAGGACCAGAAAGTTCGGCCCAATGAAGAAAACAATAAGGACGCAGA |
| | | CTTGTACACGTCCAGGGTGATGCTCAGTAGTCAAGTGCCTTTGGAGCCTCCTCTT |
| | | CTCTTTCTGCTGGAGGAATACAAAAATTACCTAGATGCTGCAAACATGTCCATGA |
| | | GGGTCCGGCGCCACTCTGACCCTGCCCGCCGAGGGGAGCTGAGCGTGTGTGACAG |
| | | TATTAGTGAGTGGGTAACGGCGGCAGACAAAAAGACTGCAGTGGACATGTCGGGC |
| | | GGGACGGTCACAGTCCTTGAAAAGGTCCCTGTATCAAAAGGCCAACTGAAGCAAT |
| | | ACTTCTACGAGACCAAGTGCAATCCCATGGGTTACACAAAAGAAGGCTGCAGGGG |
| | | CATAGACAAAAGGCATTGGAACTCCCAGTGCCGAACTACCCAGTCGTACGTGCGG |
| | | GCCCTTACCATGGATAGCAAAAAGAGAATTGGCTGGCGATTCATAAGGATAGACA |
| | | CTTCTTGTGTATGTACATTGACCATTAAAAGGGGAAGATAGACCCAGCTTTCTTG |
| | | TACAAAGTGGGAATTCCGATAATCAACCTCTGGATTACAAAATTTGTGAAAGATT |
| | | GACTGGTATTCTTAACTATGTTGCTCCTTTTACGCTATGTGGATACGCTGCTTTA |
| | | ATGCCTTTGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGTCAGGCAACG |
| | | TGGCGTGGTGTGCACTGTGTTTGCTGACGCAACCCCCACTGGTTGGGGCATTGCC |
| | | ACCACCTGTCAGCTCCTTTCCGGGACTTTCGCTTTCCCCCTCCCTATTGCCACGG |
| | | CGGAACTCATCGCCGCCTGCCTTGCCCGCTGCTGGACAGGGGCTCGGCTGTTGGG |
| | | CACTGACAATTCCGTGGTGTTGTCGGGGAAGCTGACGTCCTTTCCATGGCTGCTC |
| | | GCCTGTGTTGCCACCTGGATTCTGCGCGGGACGTCCTTCTGCTACGTCCCTTCGG |
| | | CCCTCAATCCAGCGGACCTTCCTTCCCGCGGCCTGCTGCCGGCTCTGCGGCCTCT |
| | | TCCGCGTCTTCGCCTTCGCCCTCAGACGAGTCGGATCTCCCTTTGGGCCGCCTCC |
| | | CCGCATCGGGAATTCCTAGAGCTCGCTGATCAGCCTCGACTGTGCCTTCTAGTTG |
| | | CCAGCCATCTGTTGTTTGCCCCTCCCCCGTGCCTTCCTTGACCCTGGAAGGTGCC |
| | | ACTCCCACTGTCCTTTCCTAATAAAATGAGGAAATTGCATCGCATTGTCTGAGTA |
| | | GGTGTCATTCTATTCTGGGGGGTGGGGTGGGGCAGGACAGCAAGGGGGAGGATTG |
| | | GGAAGACAATAGCAGGCATGCTGGGGATGGCCCGGGCTCTATGG |
| 47 | BDNF-S | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCGCCACCATG |
| | | ACCATCCTTTTCCTTACTATGGTTATTTCATACTTTGGTTGCATGAAGGCTGCCC |
| | | CCATGAAAGAAGCAAACATCCGAGGACAAGGTGGCTTGGCCTACCCAGGTGTGCG |
| | | GACCCATGGGACTCTGGAGAGCGTGAATGGGCCCAAGGCAGGTTCAAGAGGCTTG |
| | | ACATCATTGGCTGACACTTTCGAACACGTGATAGAAGAGCTGTTGGATGAGGACC |
| | | AGAAAGTTCGGCCCAATGAAGAAAACAATAAGGACGCAGACTTGTACACGTCCAG |
| | | GGTGATGCTCAGTAGTCAAGTGCCTTTGGAGCCTCCTCTTCTCTTTCTGCTGGAG |
| | | GAATACAAAAATTACCTAGATGCTGCAAACATGTCCATGAGGGTCCGGCGCCACT |
| | | CTGACCCTGCCCGCCGAGGGGAGCTGAGCGTGTGTGACAGTATTAGTGAGTGGGT |
| | | AACGGCGGCAGACAAAAAGACTGCAGTGGACATGTCGGGCGGGACGGTCACAGTC |
| | | CTTGAAAAGGTCCCTGTATCAAAAGGCCAACTGAAGCAATACTTCTACGAGACCA |
| | | AGTGCAATCCCATGGGTTACACAAAAGAAGGCTGCAGGGGCATAGACAAAAGGCA |
| | | TTGGAACTCCCAGTGCCGAACTACCCAGTCGTACGTGCGGGCCCTTACCATGGAT |
| | | AGCAAAAAGAGAATTGGCTGGCGATTCATAAGGATAGACACTTCTTGTGTATGTA |
| | | CATTGACCATTAAAAGGGGAAGACGCAAAAAACGCAGCGTGGGCAGCGGCGCGAC |
| | | CAACTTTAGCCTGCTGAAACAGGCGGGCGATGTGGAAGAAAACCCGGGCCCGATG |
| | | ACCATCCTTTTCCTTACTATGGTTATTTCATACTTTGGTTGCATGAAGGCTGCCC |
| | | CCATGAAAGAAGCAAACATCCGAGGACAAGGTGGCTTGGCCTACCCAGGTGTGCG |
| | | GACCCATGGGACTCTGGAGAGCGTGAATGGGCCCAAGGCAGGTTCAAGAGGCTTG |
| | | ACATCATTGGCTGACACTTTCGAACACGTGATAGAAGAGCTGTTGGATGAGGACC |
| | | AGAAAGTTCGGCCCAATGAAGAAAACAATAAGGACGCAGACTTGTACACGTCCAG |
| | | GGTGATGCTCAGTAGTCAAGTGCCTTTGGAGCCTCCTCTTCTCTTTCTGCTGGAG |
| | | GAATACAAAAATTACCTAGATGCTGCAAACATGTCCATGAGGGTCCGGCGCCACT |
| | | CTGACCCTGCCCGCCGAGGGGAGCTGAGCGTGTGTGACAGTATTAGTGAGTGGGT |
| | | AACGGCGGCAGACAAAAAGACTGCAGTGGACATGTCGGGCGGGACGGTCACAGTC |
| | | CTTGAAAAGGTCCCTGTATCAAAAGGCCAACTGAAGCAATACTTCTACGAGACCA |
| | | AGTGCAATCCCATGGGTTACACAAAAGAAGGCTGCAGGGGCATAGACAAAAGGCA |
| | | TTGGAACTCCCAGTGCCGAACTACCCAGTCGTACGTGCGGGCCCTTACCATGGAT |
| | | AGCAAAAAGAGAATTGGCTGGCGATTCATAAGGATAGACACTTCTTGTGTATGTA |
| | | CATTGACCATTAAAAGGGGAAGATAGACCCAGCTTTCTTGTACAAAGTGGGAATT |
| | | CCGATAATCAACCTCTGGATTACAAAATTTGTGAAAGATTGACTGGTATTCTTAA |
| | | CTATGTTGCTCCTTTTACGCTATGTGGATACGCTGCTTTAATGCCTTTGTATCAT |
| | | GCTATTGCTTCCCGTATGGCTTTCATTTTCTCCTCCTTGTATAAATCCTGGTTGC |
| | | TGTCTCTTTATGAGGAGTTGTGGCCCGTTGTCAGGCAACGTGGCGTGGTGTGCAC |
| | | TGTGTTTGCTGACGCAACCCCCACTGGTTGGGGCATTGCCACCACCTGTCAGCTC |
| | | CTTTCCGGGACTTTCGCTTTCCCCCTCCCTATTGCCACGGCGGAACTCATCGCCG |
| | | CCTGCCTTGCCCGCTGCTGGACAGGGGCTCGGCTGTTGGGCACTGACAATTCCGT |
| | | GGTGTTGTCGGGGAAGCTGACGTCCTTTCCATGGCTGCTCGCCTGTGTTGCCACC |
| | | TGGATTCTGCGCGGGACGTCCTTCTGCTACGTCCCTTCGGCCCTCAATCCAGCGG |
| | | ACCTTCCTTCCCGCGGCCTGCTGCCGGCTCTGCGGCCTCTTCCGCGTCTTCGCCT |
| | | TCGCCCTCAGACGAGTCGGATCTCCCTTTGGGCCGCCTCCCCGCATCGGGAATTC |
| | | CTAGAGCTCGCTGATCAGCCTCGACTGTGCCTTCTAGTTGCCAGCCATCTGTTGT |
| | | TTGCCCCTCCCCCGTGCCTTCCTTGACCCTGGAAGGTGCCACTCCCACTGTCCTT |
| | | TCCTAATAAAATGAGGAAATTGCATCGCATTGTCTGAGTAGGTGTCATTCTATTC |
| | | TGGGGGGTGGGGTGGGGCAGGACAGCAAGGGGGAGGATTGGGAAGACAATAGCAG |
| | | GCATGCTGGGGATGGCCCGGGCTCTATGG |
| 48 | BDNF-T | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCGCCACCATG |
| | | ACCATCCTTTTCCTTACTATGGTTATTTCATACTTTGGTTGCATGAAGGCTGCCC |
| | | CCATGAAAGAAGCAAACATCCGAGGACAAGGTGGCTTGGCCTACCCAGGTGTGCG |
| | | GACCCATGGGACTCTGGAGAGCGTGAATGGGCCCAAGGCAGGTTCAAGAGGCTTG |
| | | ACATCATTGGCTGACACTTTCGAACACGTGATAGAAGAGCTGTTGGATGAGGACC |
| | | AGAAAGTTCGGCCCAATGAAGAAAACAATAAGGACGCAGACTTGTACACGTCCAG |
| | | GGTGATGCTCAGTAGTCAAGTGCCTTTGGAGCCTCCTCTTCTCTTTCTGCTGGAG |
| | | GAATACAAAAATTACCTAGATGCTGCAAACATGTCCATGAGGGTCCGGCGCCACT |
| | | CTGACCCTGCCCGCCGAGGGGAGCTGAGCGTGTGTGACAGTATTAGTGAGTGGGT |
| | | AACGGCGGCAGACAAAAAGACTGCAGTGGACATGTCGGGCGGGACGGTCACAGTC |
| | | CTTGAAAAGGTCCCTGTATCAAAAGGCCAACTGAAGCAATACTICTACGAGACCA |
| | | AGTGCAATCCCATGGGTTACACAAAAGAAGGCTGCAGGGGCATAGACAAAAGGCA |
| | | TTGGAACTCCCAGTGCCGAACTACCCAGTCGTACGTGCGGGCCCTTACCATGGAT |
| | | AGCAAAAAGAGAATTGGCTGGCGATTCATAAGGATAGACACTTCTTGTGTATGTA |
| | | CATTGACCATTAAAAGGGGAAGACGCAAAAAACGCAGCGTGGGCAGCGGCGAAGG |
| | | CCGCGGCAGCCTGCTGACCTGCGGCGATGTGGAAGAAAACCCGGGCCCGATGACC |
| | | ATCCTTTTCCTTACTATGGTTATTTCATACTTTGGTTGCATGAAGGCTGCCCCCA |
| | | TGAAAGAAGCAAACATCCGAGGACAAGGTGGCTTGGCCTACCCAGGTGTGCGGAC |
| | | CCATGGGACTCTGGAGAGCGTGAATGGGCCCAAGGCAGGTTCAAGAGGCTTGACA |
| | | TCATTGGCTGACACTTTCGAACACGTGATAGAAGAGCTGTTGGATGAGGACCAGA |
| | | AAGTTCGGCCCAATGAAGAAAACAATAAGGACGCAGACTTGTACACGTCCAGGGT |
| | | GATGCTCAGTAGTCAAGTGCCTTTGGAGCCTCCTCTTCTCTTTCTGCTGGAGGAA |
| | | TACAAAAATTACCTAGATGCTGCAAACATGTCCATGAGGGTCCGGCGCCACTCTG |
| | | ACCCTGCCCGCCGAGGGGAGCTGAGCGTGTGTGACAGTATTAGTGAGTGGGTAAC |
| | | GGCGGCAGACAAAAAGACTGCAGTGGACATGTCGGGCGGGACGGTCACAGTCCTT |
| | | GAAAAGGTCCCTGTATCAAAAGGCCAACTGAAGCAATACTTCTACGAGACCAAGT |
| | | GCAATCCCATGGGTTACACAAAAGAAGGCTGCAGGGGCATAGACAAAAGGCATTG |
| | | GAACTCCCAGTGCCGAACTACCCAGTCGTACGTGCGGGCCCTTACCATGGATAGC |
| | | AAAAAGAGAATTGGCTGGCGATTCATAAGGATAGACACTTCTTGTGTATGTACAT |
| | | TGACCATTAAAAGGGGAAGATAGACCCAGCTTTCTTGTACAAAGTGGGAATTCCG |
| | | ATAATCAACCTCTGGATTACAAAATTTGTGAAAGATTGACTGGTATTCTTAACTA |
| | | TGTTGCTCCTTTTACGCTATGTGGATACGCTGCTTTAATGCCTTTGTATCATGCT |
| | | ATTGCTTCCCGTATGGCTTTCATTTTCTCCTCCTTGTATAAATCCTGGTTGCTGT |
| | | CTCTTTATGAGGAGTTGTGGCCCGTTGTCAGGCAACGTGGCGTGGTGTGCACTGT |
| | | GTTTGCTGACGCAACCCCCACTGGTTGGGGCATTGCCACCACCTGTCAGCTCCTT |
| | | TCCGGGACTTTCGCTTTCCCCCTCCCTATTGCCACGGCGGAACTCATCGCCGCCT |
| | | GCCTTGCCCGCTGCTGGACAGGGGCTCGGCTGTTGGGCACTGACAATTCCGTGGT |
| | | GTTGTCGGGGAAGCTGACGTCCTTTCCATGGCTGCTCGCCTGTGTTGCCACCTGG |
| | | ATTCTGCGCGGGACGTCCTTCTGCTACGTCCCTTCGGCCCTCAATCCAGCGGACC |
| | | TTCCTTCCCGCGGCCTGCTGCCGGCTCTGCGGCCTCTTCCGCGTCTTCGCCTTCG |
| | | CCCTCAGACGAGTCGGATCTCCCTTTGGGCCGCCTCCCCGCATCGGGAATTCCTA |
| | | GAGCTCGCTGATCAGCCTCGACTGTGCCTTCTAGTTGCCAGCCATCTGTTGTTTG |
| | | CCCCTCCCCCGTGCCTTCCTTGACCCTGGAAGGTGCCACTCCCACTGTCCTTTCC |
| | | TAATAAAATGAGGAAATTGCATCGCATTGTCTGAGTAGGTGTCATTCTATTCTGG |
| | | GGGGTGGGGTGGGGCAGGACAGCAAGGGGGAGGATTGGGAAGACAATAGCAGGCA |
| | | TGCTGGGGATGGCCCGGGCTCTATGG |
| 49 | BDNF-U | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCGCCACCATG |
| | | ACCATCCTTTTCCTTACTATGGTTATTTCATACTTTGGTTGCATGAAGGCTGCCC |
| | | CCATGAAAGAAGCAAACATCCGAGGACAAGGTGGCTTGGCCTACCCAGGTGTGCG |
| | | GACCCATGGGACTCTGGAGAGCGTGAATGGGCCCAAGGCAGGTTCAAGAGGCTTG |
| | | ACATCATTGGCTGACACTTTCGAACACGTGATAGAAGAGCTGTTGGATGAGGACC |
| | | AGAAAGTTCGGCCCAATGAAGAAAACAATAAGGACGCAGACTTGTACACGTCCAG |
| | | GGTGATGCTCAGTAGTCAAGTGCCTTTGGAGCCTCCTCTTCTCTTTCTGCTGGAG |
| | | GAATACAAAAATTACCTAGATGCTGCAAACATGTCCATGAGGGTCCGGCGCCACT |
| | | CTGACCCTGCCCGCCGAGGGGAGCTGAGCGTGTGTGACAGTATTAGTGAGTGGGT |
| | | AACGGCGGCAGACAAAAAGACTGCAGTGGACATGTCGGGCGGGACGGTCACAGTC |
| | | CTTGAAAAGGTCCCTGTATCAAAAGGCCAACTGAAGCAATACTTCTACGAGACCA |
| | | AGTGCAATCCCATGGGTTACACAAAAGAAGGCTGCAGGGGCATAGACAAAAGGCA |
| | | TTGGAACTCCCAGTGCCGAACTACCCAGTCGTACGTGCGGGCCCTTACCATGGAT |
| | | AGCAAAAAGAGAATTGGCTGGCGATTCATAAGGATAGACACTTCTTGTGTATGTA |
| | | CATTGACCATTAAAAGGGGAAGATACCCATACGATGTTCCAGATTACGCTCGCAA |
| | | AAAACGCAGCGTGGGCAGCGGCGCGACCAACTTTAGCCTGCTGAAACAGGCGGGC |
| | | GATGTGGAAGAAAACCCGGGCCCGATGACCATCCTTTTCCTTACTATGGTTATTT |
| | | CATACTTTGGTTGCATGAAGGCTGCCCCCATGAAAGAAGCAAACATCCGAGGACA |
| | | AGGTGGCTTGGCCTACCCAGGTGTGCGGACCCATGGGACTCTGGAGAGCGTGAAT |
| | | GGGCCCAAGGCAGGTTCAAGAGGCTTGACATCATTGGCTGACACTTTCGAACACG |
| | | TGATAGAAGAGCTGTTGGATGAGGACCAGAAAGTTCGGCCCAATGAAGAAAACAA |
| | | TAAGGACGCAGACTTGTACACGTCCAGGGTGATGCTCAGTAGTCAAGTGCCTTTG |
| | | GAGCCTCCTCTTCTCTTTCTGCTGGAGGAATACAAAAATTACCTAGATGCTGCAA |
| | | ACATGTCCATGAGGGTCCGGCGCCACTCTGACCCTGCCCGCCGAGGGGAGCTGAG |
| | | CGTGTGTGACAGTATTAGTGAGTGGGTAACGGCGGCAGACAAAAAGACTGCAGTG |
| | | GACATGTCGGGCGGGACGGTCACAGTCCTTGAAAAGGTCCCTGTATCAAAAGGCC |
| | | AACTGAAGCAATACTTCTACGAGACCAAGTGCAATCCCATGGGTTACACAAAAGA |
| | | AGGCTGCAGGGGCATAGACAAAAGGCATTGGAACTCCCAGTGCCGAACTACCCAG |
| | | TCGTACGTGCGGGCCCTTACCATGGATAGCAAAAAGAGAATTGGCTGGCGATTCA |
| | | TAAGGATAGACACTTCTTGTGTATGTACATTGACCATTAAAAGGGGAAGATACCC |
| | | ATACGATGTTCCAGATTACGCTTAGACCCAGCTTTCTTGTACAAAGTGGGAATTC |
| | | CGATAATCAACCTCTGGATTACAAAATTTGTGAAAGATTGACTGGTATTCTTAAC |
| | | TATGTTGCTCCTTTTACGCTATGTGGATACGCTGCTTTAATGCCTTTGTATCATG |
| | | CTATTGCTTCCCGTATGGCTTTCATTTTCTCCTCCTTGTATAAATCCTGGTTGCT |
| | | GTCTCTTTATGAGGAGTTGTGGCCCGTTGTCAGGCAACGTGGCGTGGTGTGCACT |
| | | GTGTTTGCTGACGCAACCCCCACTGGTTGGGGCATTGCCACCACCTGTCAGCTCC |
| | | TTTCCGGGACTTTCGCTTTCCCCCTCCCTATTGCCACGGCGGAACTCATCGCCGC |
| | | CTGCCTTGCCCGCTGCTGGACAGGGGCTCGGCTGTTGGGCACTGACAATTCCGTG |
| | | GTGTTGTCGGGGAAGCTGACGTCCTTTCCATGGCTGCTCGCCTGTGTTGCCACCT |
| | | GGATTCTGCGCGGGACGTCCTTCTGCTACGTCCCTTCGGCCCTCAATCCAGCGGA |
| | | CCTTCCTTCCCGCGGCCTGCTGCCGGCTCTGCGGCCTCTTCCGCGTCTTCGCCTT |
| | | CGCCCTCAGACGAGTCGGATCTCCCTTTGGGCCGCCTCCCCGCATCGGGAATTCC |
| | | TAGAGCTCGCTGATCAGCCTCGACTGTGCCTTCTAGTTGCCAGCCATCTGTTGTT |
| | | TGCCCCTCCCCCGTGCCTTCCTTGACCCTGGAAGGTGCCACTCCCACTGTCCTTT |
| | | CCTAATAAAATGAGGAAATTGCATCGCATTGTCTGAGTAGGTGTCATTCTATTCT |
| | | GGGGGGTGGGGTGGGGCAGGACAGCAAGGGGGAGGATTGGGAAGACAATAGCAGG |
| | | CATGCTGGGGATGGCCCGGGCTCTATGG |
| 50 | BDNF-V | CTGTGGAATGTGTGTCAGTTAGGGTGTGGAAAGTCCCCAGGCTCCCCAGCAGGCA |
| | | GAAGTATGCAAAGCATGCATCTCAATTAGTCAGCAACCAGGTGTGGAAAGTCCCC |
| | | AGGCTCCCCAGCAGGCAGAAGTATGCAAAGCATGCATCTCAATTAGTCAGCAACC |
| | | ATAGTCCCGCCCCTAACTCCGCCCATCCCGCCCCTAACTCCGCCCAGGACATTGA |
| | | TTATTGACTAGTTATTAATAGTAATCAATTACGGGGTCATTAGTTCATAGCCCAT |
| | | ATATGGAGTTCCGCGTTACATAACTTACGGTAAATGGCCCGCCTGGCTGACCGCC |
| | | CAACGACCCCCGCCCATTGACGTCAATAATGACGTATGTTCCCATAGTAACGCCA |
| | | ATAGGGACTTTCCATTGACGTCAATGGGTGGACTATTTACGGTAAACTGCCCACT |
| | | TGGCAGTACATCAAGTGTATCATATGCCAAGTACGCCCCCTATTGACGTCAATGA |
| | | CGGTAAATGGCCCGCCTGGCATTATGCCCAGTACATGACCTTATGGGACTTTCCT |
| | | ACTTGGCAGTACATCTACGTATTAGTCATCGCTATTACCATGGTGATGCGGTTTT |
| | | GGCAGTACATCAATGGGCGTGGATAGCGGTTTGACTCACGGGGATTTCCAAGTCT |
| | | CCACCCCATTGACGTCAATGGGAGTTTGTTTTGGCACCAAAATCAACGGGACTTT |
| | | CCAAAATGTCGTAACAACTCCGCCCCATTGACGCAAATGGGCGGTAGGCGTGTAC |
| | | GGTGGGAGGTCTATATAAGCAGAGCTCGTTTAGTGAACCGTCAGATCGCCTGGAG |
| | | ACCTCGAGCCGAGTGGTCGTGCCTCCATAGAAGACACCGGGACCGAGGTAAGTTT |
| | | AAAGCTCAGGTCGAGACCGGGCCTTTGTCCGGCGCTCCCTTGGAGCCTACCTAGA |
| | | CTCAGCCGGCTCTCCACGCTTTGCCTGACCCTGCTTGCTCAACTCTAGTTCTCTC |
| | | GTTAACTTAATGAGACAGATAGAAACTGGTCTTGTAGAAACAGAGTAGTCGCCTG |
| | | CTTTTCTGCCAGGTGCTGACTTCTCTCCCCTGGGCTTTTTTCTTTTTCTCAGGTT |
| | | GAAAAGAAGAAGACGAAGAAGACGAAGAAGACAAACCGTCGGATCCGCCACCATG |
| | | ACCATCCTTTTCCTTACTATGGTTATTTCATACTTTGGTTGCATGAAGGCTGCCC |
| | | CCATGAAAGAAGCAAACATCCGAGGACAAGGTGGCTTGGCCTACCCAGGTGTGCG |
| | | GACCCATGGGACTCTGGAGAGCGTGAATGGGCCCAAGGCAGGTTCAAGAGGCTTG |
| | | ACATCATTGGCTGACACTTTCGAACACGTGATAGAAGAGCTGTTGGATGAGGACC |
| | | AGAAAGTTCGGCCCAATGAAGAAAACAATAAGGACGCAGACTTGTACACGTCCAG |
| | | GGTGATGCTCAGTAGTCAAGTGCCTTTGGAGCCTCCTCTTCTCTTTCTGCTGGAG |
| | | GAATACAAAAATTACCTAGATGCTGCAAACATGTCCATGAGGGTCCGGCGCCACT |
| | | CTGACCCTGCCCGCCGAGGGGAGCTGAGCGTGTGTGACAGTATTAGTGAGTGGGT |
| | | AACGGCGGCAGACAAAAAGACTGCAGTGGACATGTCGGGCGGGACGGTCACAGTC |
| | | CTTGAAAAGGTCCCTGTATCAAAAGGCCAACTGAAGCAATACTTCTACGAGACCA |
| | | AGTGCAATCCCATGGGTTACACAAAAGAAGGCTGCAGGGGCATAGACAAAAGGCA |
| | | TTGGAACTCCCAGTGCCGAACTACCCAGTCGTACGTGCGGGCCCTTACCATGGAT |
| | | AGCAAAAAGAGAATTGGCTGGCGATTCATAAGGATAGACACTTCTTGTGTATGTA |
| | | CATTGACCATTAAAAGGGGAAGATAGCCCCTCTCCCTCCCCCCCCCCTAACGTTA |
| | | CTGGCCGAAGCCGCTTGGAATAAGGCCGGTGTGCGTTTGTCTATATGTTATTTTC |
| | | CACCATATTGCCGTCTTTTGGCAATGTGAGGGCCCGGAAACCTGGCCCTGTCTTC |
| | | TTGACGAGCATTCCTAGGGGTCTTTCCCCTCTCGCCAAAGGAATGCAAGGTCTGT |
| | | TGAATGTCGTGAAGGAAGCAGTTCCTCTGGAAGCTTCTTGAAGACAAACAACGTC |
| | | TGTAGCGACCCTTTGCAGGCAGCGGAACCCCCCACCTGGCGACAGGTGCCTCTGC |
| | | GGCCAAAAGCCACGTGTATAAGATACACCTGCAAAGGCGGCACAACCCCAGTGCC |
| | | ACGTTGTGAGTTGGATAGTTGTGGAAAGAGTCAAATGGCTCTCCTCAAGCGTATT |
| | | CAACAAGGGGCTGAAGGATGCCCAGAAGGTACCCCATTGTATGGGATCTGATCTG |
| | | GGGCCTCGGTGCACATGCTTTACATGTGTTTAGTCGAGGTTAAAAAAACGTCTAG |
| | | GCCCCCCGAACCACGGGGACGTGGTTTTCCTTTGAAAAACACGATGATACGCAAA |
| | | AAACGCAGCGTGATGACCATCCTTTTCCTTACTATGGTTATTTCATACTTTGGTT |
| | | GCATGAAGGCTGCCCCCATGAAAGAAGCAAACATCCGAGGACAAGGTGGCTTGGC |
| | | CTACCCAGGTGTGCGGACCCATGGGACTCTGGAGAGCGTGAATGGGCCCAAGGCA |
| | | GGTTCAAGAGGCTTGACATCATTGGCTGACACTTTCGAACACGTGATAGAAGAGC |
| | | TGTTGGATGAGGACCAGAAAGTTCGGCCCAATGAAGAAAACAATAAGGACGCAGA |
| | | CTTGTACACGTCCAGGGTGATGCTCAGTAGTCAAGTGCCTTTGGAGCCTCCTCTT |
| | | CTCTTTCTGCTGGAGGAATACAAAAATTACCTAGATGCTGCAAACATGTCCATGA |
| | | GGGTCCGGCGCCACTCTGACCCTGCCCGCCGAGGGGAGCTGAGCGTGTGTGACAG |
| | | TATTAGTGAGTGGGTAACGGCGGCAGACAAAAAGACTGCAGTGGACATGTCGGGC |
| | | GGGACGGTCACAGTCCTTGAAAAGGTCCCTGTATCAAAAGGCCAACTGAAGCAAT |
| | | ACTTCTACGAGACCAAGTGCAATCCCATGGGTTACACAAAAGAAGGCTGCAGGGG |
| | | CATAGACAAAAGGCATTGGAACTCCCAGTGCCGAACTACCCAGTCGTACGTGCGG |
| | | GCCCTTACCATGGATAGCAAAAAGAGAATTGGCTGGCGATTCATAAGGATAGACA |
| | | CTTCTTGTGTATGTACATTGACCATTAAAAGGGGAAGATAGACCCAGCTTTCTTG |
| | | TACAAAGTGGGAATTCCGATAATCAACCTCTGGATTACAAAATTTGTGAAAGATT |
| | | GACTGGTATTCTTAACTATGTTGCTCCTTTTACGCTATGTGGATACGCTGCTTTA |
| | | ATGCCTTTGTATCATGCTATTGCTTCCCGTATGGCTTTCATTTTCTCCTCCTTGT |
| | | ATAAATCCTGGTTAGTTCTTGCCACGGCGGAACTCATCGCCGCCTGCCTTGCCCG |
| | | CTGCTGGACAGGGGCTCGGCTGTTGGGCACTGACAATTCCGTGGTGTTCAATCGG |
| | | GAATTCCTAGAGCTCGCTGATCAGCCTCGACTGTGCCTTCTAGTTGCCAGCCATC |
| | | TGTTGTTTGCCCCTCCCCCGTGCCTTCCTTGACCCTGGAAGGTGCCACTCCCACT |
| | | GTCCTTTCCTAATAAAATGAGGAAATTGCATCGCATTGTCTGAGTAGGTGTCATT |
| | | CTATTCTGGGGGGTGGGGTGGGGCAGGACAGCAAGGGGGAGGATTGGGAAGACAA |
| | | TAGCAGGCATGCTGGGGATGGCCCGGGCTCTATGG |

We claim:

1. An expression construct comprising a promoter sequence operatively linked to a nucleic acid sequence encoding a brain derived neurotrophic factor (BDNF) or variant thereof.

2. The expression construct of claim 1, wherein the promoter sequence (i) comprises a nucleic acid sequence having at least 90% sequence identity to a nucleic acid sequence selected from SEQ ID NOs: 1-10 or (ii) comprises a nucleic acid sequence selected from SEQ ID NOs: 1-10.

3. The expression construct of claim 1, wherein the promoter sequence (i) comprises a nucleic acid sequence having at least 90% sequence identity to the nucleic acid sequence of SEQ ID NO: 1 or (ii) comprises the nucleic acid sequence of SEQ ID NO: 1.

4. The expression construct of claim 1, wherein the promoter sequence comprises a constitutive promoter.

5. The expression construct of any one of the preceding claims, wherein the nucleic acid sequence encoding the BDNF is a wild type BDNF gene.

6. The expression construct of any one of the preceding claims, wherein the nucleic acid sequence encoding the BDNF is a human BDNF gene.

7. The expression construct of any one of the preceding claims, wherein the nucleic acid sequence encoding the BDNF is a codon-optimized sequence.

8. The expression construct of any one of the preceding claims, wherein the nucleic acid sequence encoding the BDNF (i) comprises a nucleic acid sequence having at least 90% sequence identity to a nucleic acid sequence selected from SEQ ID NOs: 52-57 or 70-79 or (ii) comprises a nucleic acid sequence selected from SEQ ID NOs: 52-57 or 70-79.

9. The expression construct of any one of the preceding claims, wherein the nucleic acid sequence encoding the BDNF (i) comprises a nucleic acid sequence having at least 90% sequence identity to the nucleic acid sequence of SEQ ID NO: 52 or 53 or (ii) comprises the nucleic acid sequence of SEQ ID NO: 52 or 53.

10. The expression construct of any one of the preceding claims, wherein the expression construct further comprises a post-transcriptional regulatory element.

11. The expression construct of claim 10, wherein the post-transcriptional regulatory element comprises a woodchuck hepatitis virus post-transcriptional regulatory element (WPRE).

12. The expression construct of claim 10, wherein the post-transcriptional regulatory element (i) comprises a nucleic acid sequence having at least 90% sequence identity to a nucleic acid sequence selected from SEQ ID NOs: 58-61 or (ii) comprises a nucleic acid sequence selected from SEQ ID NOs: 58-61.

13. The expression construct of claim 10, wherein the post-transcriptional regulatory element (i) comprises a nucleic acid sequence having at least 90% sequence identity to the nucleic acid sequence of SEQ ID NO: 58 or 59 or (ii) comprises the nucleic acid sequence of SEQ ID NO: 58 or 59.

14. The expression construct of any one of the preceding claims, wherein the expression construct further comprises a polyadenylation signal.

15. The expression construct of claim 14, wherein the polyadenylation signal (i) comprises a nucleic acid sequence having at least 90% sequence identity to any one of SEQ ID NOs: 62 or 66-69 or (ii) comprises any one of SEQ ID NOs: 62 or 66-69.

16. The expression construct of claim 15, wherein the polyadenylation signal (i) comprises a nucleic acid sequence having at least 90% sequence identity to SEQ ID NO: 62 or (ii) comprises SEQ ID NOs: 62.

17. The expression construct of any one of the preceding claims, wherein the expression construct further comprises a microRNA binding sequence positioned between the nucleic acid sequence encoding the BDNF and the post-transcriptional regulatory element.

18. The expression construct of claim 17, wherein the microRNA binding sequence comprises:
(a) a nucleic acid sequence having at least 90% sequence identity to (i) SEQ ID NO: 63, (ii) SEQ ID NO: 64, or (iii) SEQ ID NOs: 63 and 82; or
(b) (i) SEQ ID NO: 63, (ii) SEQ ID NO: 64, or (iii) SEQ ID NOs: 63 and 82.

19. The expression construct of claim 1, wherein the expression construct comprises:
(a) a promoter sequence comprising SEQ ID NO: 1;
(b) a nucleic acid sequence encoding BDNF comprising SEQ ID NO: 52;
(c) a post-transcriptional regulatory element comprising SEQ ID NO: 58;
(d) a polyadenylation signal comprising SEQ ID NO: 62; and
(e) a microRNA binding sequence comprising SEQ ID NO: 63.

20. The expression construct of claim 1, wherein the expression construct comprises:
(a) a promoter sequence comprising SEQ ID NO: 1;
(b) a nucleic acid sequence encoding BDNF comprising SEQ ID NO: 53;
(c) a post-transcriptional regulatory element comprising SEQ ID NO: 59;
(d) a polyadenylation signal comprising SEQ ID NO: 62; and
(e) a microRNA binding sequence comprising SEQ ID NO: 64.

21. A vector comprising the expression construct of any one of claims 1-20.

22. The vector of claim 21, wherein the vector is a viral vector.

23. The vector of claim 22, wherein the viral vector is an adeno-associated virus (AAV) vector.

24. A viral vector comprising (i) the expression construct of any one of claims 1-20 and (ii) one or more inverted terminal repeats (ITRs).

25. The viral vector of claim 23 or 25, wherein the viral vector (i) comprises a nucleic acid sequence having at least 90% sequence identity to a nucleic acid sequence selected from SEQ ID NOs: 11-50 or (ii) comprises a nucleic acid sequence selected from SEQ ID NOs: 11-50.

26. The viral vector of claim 25, wherein the viral vector (i) comprises a nucleic acid sequence having at least 90% sequence identity to a nucleic acid sequence selected from SEQ ID NOs: 11, 18-22, 27-30, and 42-45 or (ii) comprises a nucleic acid sequence selected from SEQ ID NOs: 11, 18-22, 27-30, and 42-45.

27. The viral vector of claim 26, wherein the expression construct (i) comprises a nucleic acid sequence having at least 90% sequence identity to SEQ ID NO: 11 or 22 or (ii) SEQ ID NO: 11 or 22.

28. The viral vector of claim 23 or 24, wherein the vector comprises a genome derived from AAV serotype AAV2.

29. The viral vector of any one of claims 23-28, wherein the vector comprises a capsid derived from AAV serotype AAV1.

30. A pharmaceutical composition comprising the vector of any one of claims 21-30 and a pharmaceutically acceptable carrier.

31. A method for treating a metabolic disorder in a subject in need thereof, comprising administering to the subject the vector of any one of claims 21-30 or the pharmaceutical composition of claim 30.

32. The method of claim 31, wherein the metabolic disorder is obesity.

33. A method of increasing a level of BDNF in a subject in need thereof, comprising administering to the subject the vector of any one of claims 21-30 or the pharmaceutical composition of claim 30.

34. The method of any one of claims 31-33, wherein the vector or the pharmaceutical composition is administered by stereotaxic microinjection.


SEQUENCE LISTING (82 sequences)

SEQ ID NO: 1 - DNA, 1377 bp, synthetic construct
  source 1..1377: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccacc

SEQ ID NO: 2 - DNA, 527 bp, synthetic construct
  source 1..527: mol_type = other DNA
gccgggctggtagcccagcggggggcgcggcagggcgaggagcagcccgcgctgctcccgcccctcgggtgccagcaccgcccctgctgcggcgggtgaggggcggggcggggcgcggcgtatataaggctaggggcgggcgccgctcttttgtttcttgctgcagcaacgcgagtgggagcaccaggatctcgggctcggaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaaggatccgccacc

SEQ ID NO: 3 - DNA, 563 bp, synthetic construct
  source 1..563: mol_type = other DNA
tgtcccacctcgttagagaaaacagcgatgctcaaaggcaacctccttcctgacattgcctggtaggacgcgacgtggtgtttgcccgcgcggaatgcggacgcaaggctgctcctaggtctcggggacgcgccatccccatttccgctcgcggaggcgtagggtccgggcgcgggaccccagtcgaccttgactggcggcgcgaccttgaggcctgcgttcgcctcagttgccccctctgtgcaatggggagacgcctgccgccccgccccgagtcccatgccgcagccaccgcgacggagcccgcaggcgggaacctgcctccgcgcgttagcgcgcacgcgcgcctcatgtgtcgtccccatcagcgccggcttccgtctataggccagatgcactgtcactctggcgaagtcgcagacccgattggccgggacggaggcgcgagaccgggttgcgggcggggccgaacgtggtataaaaggggcgggaggccaggctcgtgtcagtctgcagacgccacccgagccgagtggtcgttactattagccggatccgccacc

SEQ ID NO: 4 - DNA, 312 bp, synthetic construct
  source 1..312: mol_type = other DNA
ccccggatgctcacctccccgactcgcccccgctgtggccccgcccccgcgcggctcttcgtgccacgtcaccgcctgcgtcgcttccggaggcgcagcgggcgatgacgtagagggacgtgccctctatatgaggttggggagcggctgagtcggtcagtccgcctggagacctcgagccgagtggtcgtccgcccgctcccccctccccccgagcgccgctccggctgcaccgcgctcgctccgagtttcaggctcgtgctaagctagcgccgtcgtcgtctcccttcagtcgccatcggatccgccacc

SEQ ID NO: 5 - DNA, 1078 bp, synthetic construct
  source 1..1078: mol_type = other DNA
tgtcccacctcgttagagaaaacagcgatgctcaaaggcaacctccttcctgacattgcctggtaggacgcgacgtggtgtttgcccgcgcggaatgcggacgcaaggctgctcctaggtctcggggacgcgccatccccatttccgctcgcggaggcgtagggtccgggcgcgggaccccagtcgaccttgactggcggcgcgaccttgaggcctgcgttcgcctcagttgccccctctgtgcaatggggagacgcctgccgccccgccccgagtcccatgccgcagccaccgcgacggagcccgcaggcgggaacctgcctccgcgcgttagcgcgcacgcgcgcctcatgtgtcgtccccatcagcgccggcttccgtctataggccagatgcactgtcactctggcgaagtcgcagacccgattggccgggacggaggcgcgagaccgggttgcgggcggggccgaacgtggtataaaaggggcgggaggccaggctcgtgtcagtctgcagacgccacccgagccgagtggtcgttactattagccgccgggctggtagcccagcggggggcgcggcagggcgaggagcagcccgcgctgctcccgcccctcgggtgccagcaccgcccctgctgcggcgggtgaggggcggggcggggcgcggcgtatataaggctaggggcgggcgccgctcttcagtccgcctggagacctcgagccgagtggtcgtcaggatctcgggctcggaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaaggatccgccacc

SEQ ID NO: 6 - DNA, 1114 bp, synthetic construct
  source 1..1114: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagcccccgttctgcttcactctccccatctcccccccctccccacccccaattttgtatttatttattttttaattattttgtgcagcgatgggggcgggggggggggggggggggggggggggtggggcggagggtggcaaggggcggggcggggcaaggcggaaaggtgcggcggcagccaatcaaagcggcgccctccaaaagtttccttttatggcaaggcggcggcggcggcggccctataaaaagcgaagcgcgcggcgggcgggagtcgctgcgttgccttgggtcctccccccctctccagccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagtggagctcgcgactagtcgattcgaattcggcttgtg

SEQ ID NO: 7 - DNA, 1152 bp, synthetic construct
  source 1..1152: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccacc

SEQ ID NO: 8 - DNA, 1167 bp, synthetic construct
  source 1..1167: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccaactcctaaaaaaccgccacc

SEQ ID NO: 9 - DNA, 897 bp, synthetic construct
  source 1..897: mol_type = other DNA
cgagtcttcaaaccttaagcctcagtgcaaaagggagcagcgaggagaggcggtgggattaaagaaaaaaattcttctctccctgtcacttcaaaaggtcgcccaaccagcgagcggggggtttacccacgactctggctcgcaccccttgcgggcctgcgcaggcggccccgtagcgcaagggagggcgggaaaggaaggggcggggacgcgagggcgaatctataagaagcgtcgttcagcgagttcgctgctcagaagcgccgagagcgcggccgggacggttggagaagaaggcggctcccggaagggggagagacaaactgccgtaacctctgccgttcaggaacgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccaactcctaaaaaaccgccacc

SEQ ID NO: 10 - DNA, 981 bp, synthetic construct
  source 1..981: mol_type = other DNA
ttacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacaattggtttgatctgattataactagtggagaagagcatgcttgagggctgagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaaacagtagttgacggatccaactcctaaaaaaccgccacc

SEQ ID NO: 11 - DNA, 3316 bp, synthetic construct
  source 1..3316: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtctagacatatggagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgaccatggtcatttcctactttggctgcatgaaggctgcccccatgaaagaagccaacatccgcggccagggcggcctggcctaccctggcgtgcgcacccatggcaccctggagtccgtgaatggccccaaggccggctcccgcggcctgacctccctggctgacaccttcgaacatgtgatcgaagagctgctggatgaggaccagaaagtgcgccccaatgaagaaaacaataaggacgccgacctgtacacctcccgcgtgatgctgtcctcccaggtgcctctggagcctcctctgctgtttctgctggaggaatacaaaaattacctggatgctgctaacatgtccatgcgcgtccgccgccactccgaccctgcccgccgcggcgagctgtccgtgtgcgactccatttccgagtgggtgaccgctgccgacaaaaagaccgccgtggacatgtccggcggcaccgtcaccgtcctggaaaaggtccctgtctccaaaggccagctgaagcagtacttctatgagaccaagtgcaatcccatgggctacaccaaagaaggctgccgcggcattgacaaacggcattggaactcccagtgccgcaccacccagtcctacgtgcgcgccctgaccatggattccaaaaagcgcattggctggcgcttcatccgcattgacacctcctgcgtctgtaccctgaccatcaaacgcggccgctgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcacctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtgactagttctagagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 12 - DNA, 3259 bp, synthetic construct
  source 1..3259: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgaccatggtcatttcctactttggctgcatgaaggctgcccccatgaaagaagccaacatccgcggccagggcggcctggcctaccctggcgtgcgcacccatggcaccctggagtccgtgaatggccccaaggccggctcccgcggcctgacctccctggctgacaccttcgaacatgtgatcgaagagctgctggatgaggaccagaaagtgcgccccaatgaagaaaacaataaggacgccgacctgtacacctcccgcgtgatgctgtcctcccaggtgcctctggagcctcctctgctgtttctgctggaggaatacaaaaattacctggatgctgctaacatgtccatgcgcgtccgccgccactccgaccctgcccgccgcggcgagctgtccgtgtgcgactccatttccgagtgggtgaccgctgccgacaaaaagaccgccgtggacatgtccggcggcaccgtcaccgtcctggaaaaggtccctgtctccaaaggccagctgaagcagtacttctatgagaccaagtgcaatcccatgggctacaccaaagaaggctgccgcggcattgacaaacggcattggaactcccagtgccgcaccacccagtcctacgtgcgcgccctgaccatggattccaaaaagcgcattggctggcgcttcatccgcattgacacctcctgcgtctgtaccctgaccatcaaacgcggccgctgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 13 - DNA, 2917 bp, synthetic construct
  source 1..2917: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgaccatggtcatttcctactttggctgcatgaaggctgcccccatgaaagaagccaacatccgcggccagggcggcctggcctaccctggcgtgcgcacccatggcaccctggagtccgtgaatggccccaaggccggctcccgcggcctgacctccctggctgacaccttcgaacatgtgatcgaagagctgctggatgaggaccagaaagtgcgccccaatgaagaaaacaataaggacgccgacctgtacacctcccgcgtgatgctgtcctcccaggtgcctctggagcctcctctgctgtttctgctggaggaatacaaaaattacctggatgctgctaacatgtccatgcgcgtccgccgccactccgaccctgcccgccgcggcgagctgtccgtgtgcgactccatttccgagtgggtgaccgctgccgacaaaaagaccgccgtggacatgtccggcggcaccgtcaccgtcctggaaaaggtccctgtctccaaaggccagctgaagcagtacttctatgagaccaagtgcaatcccatgggctacaccaaagaaggctgccgcggcattgacaaacggcattggaactcccagtgccgcaccacccagtcctacgtgcgcgccctgaccatggattccaaaaagcgcattggctggcgcttcatccgcattgacacctcctgcgtctgtaccctgaccatcaaacgcggccgctgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 14 - DNA, 2884 bp, synthetic construct
  source 1..2884: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgaccatggtcatttcctactttggctgcatgaaggctgcccccatgaaagaagccaacatccgcggccagggcggcctggcctaccctggcgtgcgcacccatggcaccctggagtccgtgaatggccccaaggccggctcccgcggcctgacctccctggctgacaccttcgaacatgtgatcgaagagctgctggatgaggaccagaaagtgcgccccaatgaagaaaacaataaggacgccgacctgtacacctcccgcgtgatgctgtcctcccaggtgcctctggagcctcctctgctgtttctgctggaggaatacaaaaattacctggatgctgctaacatgtccatgcgcgtccgccgccactccgaccctgcccgccgcggcgagctgtccgtgtgcgactccatttccgagtgggtgaccgctgccgacaaaaagaccgccgtggacatgtccggcggcaccgtcaccgtcctggaaaaggtccctgtctccaaaggccagctgaagcagtacttctatgagaccaagtgcaatcccatgggctacaccaaagaaggctgccgcggcattgacaaacggcattggaactcccagtgccgcaccacccagtcctacgtgcgcgccctgaccatggattccaaaaagcgcattggctggcgcttcatccgcattgacacctcctgcgtctgtaccctgaccatcaaacgcggccgctgaggtaccttctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 15 - DNA, 2542 bp, synthetic construct
  source 1..2542: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgaccatggtcatttcctactttggctgcatgaaggctgcccccatgaaagaagccaacatccgcggccagggcggcctggcctaccctggcgtgcgcacccatggcaccctggagtccgtgaatggccccaaggccggctcccgcggcctgacctccctggctgacaccttcgaacatgtgatcgaagagctgctggatgaggaccagaaagtgcgccccaatgaagaaaacaataaggacgccgacctgtacacctcccgcgtgatgctgtcctcccaggtgcctctggagcctcctctgctgtttctgctggaggaatacaaaaattacctggatgctgctaacatgtccatgcgcgtccgccgccactccgaccctgcccgccgcggcgagctgtccgtgtgcgactccatttccgagtgggtgaccgctgccgacaaaaagaccgccgtggacatgtccggcggcaccgtcaccgtcctggaaaaggtccctgtctccaaaggccagctgaagcagtacttctatgagaccaagtgcaatcccatgggctacaccaaagaaggctgccgcggcattgacaaacggcattggaactcccagtgccgcaccacccagtcctacgtgcgcgccctgaccatggattccaaaaagcgcattggctggcgcttcatccgcattgacacctcctgcgtctgtaccctgaccatcaaacgcggccgctgaggtaccttcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 16 - DNA, 2294 bp, synthetic construct
  source 1..2294: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgaccatggtcatttcctactttggctgcatgaaggctgcccccatgaaagaagccaacatccgcggccagggcggcctggcctaccctggcgtgcgcacccatggcaccctggagtccgtgaatggccccaaggccggctcccgcggcctgacctccctggctgacaccttcgaacatgtgatcgaagagctgctggatgaggaccagaaagtgcgccccaatgaagaaaacaataaggacgccgacctgtacacctcccgcgtgatgctgtcctcccaggtgcctctggagcctcctctgctgtttctgctggaggaatacaaaaattacctggatgctgctaacatgtccatgcgcgtccgccgccactccgaccctgcccgccgcggcgagctgtccgtgtgcgactccatttccgagtgggtgaccgctgccgacaaaaagaccgccgtggacatgtccggcggcaccgtcaccgtcctggaaaaggtccctgtctccaaaggccagctgaagcagtacttctatgagaccaagtgcaatcccatgggctacaccaaagaaggctgccgcggcattgacaaacggcattggaactcccagtgccgcaccacccagtcctacgtgcgcgccctgaccatggattccaaaaagcgcattggctggcgcttcatccgcattgacacctcctgcgtctgtaccctgaccatcaaacgcggccgctgaggtaccttccctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 17 - DNA, 2997 bp, synthetic construct
  source 1..2997: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgaccatggtcatttcctactttggctgcatgaaggctgcccccatgaaagaagccaacatccgcggccagggcggcctggcctaccctggcgtgcgcacccatggcaccctggagtccgtgaatggccccaaggccggctcccgcggcctgacctccctggctgacaccttcgaacatgtgatcgaagagctgctggatgaggaccagaaagtgcgccccaatgaagaaaacaataaggacgccgacctgtacacctcccgcgtgatgctgtcctcccaggtgcctctggagcctcctctgctgtttctgctggaggaatacaaaaattacctggatgctgctaacatgtccatgcgcgtccgccgccactccgaccctgcccgccgcggcgagctgtccgtgtgcgactccatttccgagtgggtgaccgctgccgacaaaaagaccgccgtggacatgtccggcggcaccgtcaccgtcctggaaaaggtccctgtctccaaaggccagctgaagcagtacttctatgagaccaagtgcaatcccatgggctacaccaaagaaggctgccgcggcattgacaaacggcattggaactcccagtgccgcaccacccagtcctacgtgcgcgccctgaccatggattccaaaaagcgcattggctggcgcttcatccgcattgacacctcctgcgtctgtaccctgaccatcaaacgcggccgctgaggtaccttctccataaagtaggaaacactacatcatccataaagtaggaaacactacatcatccataaagtaggaaacactacatcatccataaagtaggaaacactacagaattcgatatctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 18 - DNA, 2887 bp, synthetic construct
  source 1..2887: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagtgaggtaccttctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 19 - DNA, 2887 bp, synthetic construct
  source 1..2887: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgtttctgacaatggttatttcatattttggttgcatgaaagcagctccaatgaaggaagcaaacatcagaggacaagggggcctagcttatcctggagttagaacccatgggactttggagtctgtcaatggcccaaaagctggaagcaggggcctgacaagcctggcagatacttttgaacatgtcattgaggaactcctagatgaagaccaaaaagttagaccaaatgaagagaacaataaagatgctgacctttatactagcagagtcatgctctcctctcaagtcccactagaacccccactcctgttcctactggaggaatacaaaaattaccttgatgcagccaatatgagtatgagagtgaggagacacagtgaccctgctaggagaggggaactctcagtatgtgactccattagtgaatgggtgactgctgcagacaaaaagacagcagttgacatgagtggtggcacagtaactgtactagaaaaagtaccagtgagtaaaggtcagcttaagcaatatttttatgagacaaaatgcaatccaatgggatataccaaggaaggatgtaggggcattgataaaaggcactggaatagccaatgtagaaccacacaatcttatgttagggcccttaccatggactccaagaaaagaattggatggagatttatcaggattgatacaagttgtgtctgcaccctgaccatcaaaagaggaagatgatgaggtaccttctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 20 - DNA, 2887 bp, synthetic construct
  source 1..2887: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 21 - DNA, 2887 bp, synthetic construct
  source 1..2887: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccattctgtttctgaccatggttattagctatttcgggtgcatgaaagccgccccgatgaaagaagcgaatatccggggccagggcggcctggcgtatccgggcgtccggacccacgggaccctggaatccgtgaacggcccgaaagccgggtcccggggcctgaccagcctggcggatacctttgaacatgtgattgaagaactgctggatgaagatcagaaagtccgtccgaatgaagaaaataacaaagatgcggatctgtatacctcccgggttatgctgagcagccaggttccgctggaaccgccgctgctgtttctgctggaagaatataagaattatctggacgccgccaatatgagcatgcgggtccgccgccattccgatccggcgcgccgcggcgaactgtccgtgtgcgatagcatttccgaatgggtgaccgccgccgataagaaaaccgcggtggatatgtccggcggcaccgtgaccgtgctggagaaagttccggtgagcaaagggcagctgaaacagtatttctatgaaaccaaatgcaatccgatgggctataccaaagagggctgccggggcattgataaacggcattggaatagccagtgccggaccacccagagctatgtccgggccctaaccatggatagcaagaaacggatcgggtggcgctttatccggattgataccagctgcgtgtgcaccctaaccatcaaacggggccggtaatgaggtaccttctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 22 - DNA, 2920 bp, synthetic construct
  source 1..2920: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 23 - DNA, 2989 bp, synthetic construct
  source 1..2989: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 24 - DNA, 3243 bp, synthetic construct
  source 1..3243: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcaggacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 25 - DNA, 2986 bp, synthetic construct
  source 1..2986: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcaggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 26 - DNA, 2943 bp, synthetic construct
  source 1..2943: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcagggtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaa

SEQ ID NO: 27 - DNA, 2070 bp, synthetic construct
  source 1..2070: mol_type = other DNA
gccgggctggtagcccagcggggggcgcggcagggcgaggagcagcccgcgctgctcccgcccctcgggtgccagcaccgcccctgctgcggcgggtgaggggcggggcggggcgcggcgtatataaggctaggggcgggcgccgctcttttgtttcttgctgcagcaacgcgagtgggagcaccaggatctcgggctcggaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaaggatccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 28 - DNA, 2106 bp, synthetic construct
  source 1..2106: mol_type = other DNA
tgtcccacctcgttagagaaaacagcgatgctcaaaggcaacctccttcctgacattgcctggtaggacgcgacgtggtgtttgcccgcgcggaatgcggacgcaaggctgctcctaggtctcggggacgcgccatccccatttccgctcgcggaggcgtagggtccgggcgcgggaccccagtcgaccttgactggcggcgcgaccttgaggcctgcgttcgcctcagttgccccctctgtgcaatggggagacgcctgccgccccgccccgagtcccatgccgcagccaccgcgacggagcccgcaggcgggaacctgcctccgcgcgttagcgcgcacgcgcgcctcatgtgtcgtccccatcagcgccggcttccgtctataggccagatgcactgtcactctggcgaagtcgcagacccgattggccgggacggaggcgcgagaccgggttgcgggcggggccgaacgtggtataaaaggggcgggaggccaggctcgtgtcagtctgcagacgccacccgagccgagtggtcgttactattagccggatccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 29 - DNA, 1855 bp, synthetic construct
  source 1..1855: mol_type = other DNA
ccccggatgctcacctccccgactcgcccccgctgtggccccgcccccgcgcggctcttcgtgccacgtcaccgcctgcgtcgcttccggaggcgcagcgggcgatgacgtagagggacgtgccctctatatgaggttggggagcggctgagtcggtcagtccgcctggagacctcgagccgagtggtcgtccgcccgctcccccctccccccgagcgccgctccggctgcaccgcgctcgctccgagtttcaggctcgtgctaagctagcgccgtcgtcgtctcccttcagtcgccatcggatccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 30 - DNA, 2621 bp, synthetic construct
  source 1..2621: mol_type = other DNA
tgtcccacctcgttagagaaaacagcgatgctcaaaggcaacctccttcctgacattgcctggtaggacgcgacgtggtgtttgcccgcgcggaatgcggacgcaaggctgctcctaggtctcggggacgcgccatccccatttccgctcgcggaggcgtagggtccgggcgcgggaccccagtcgaccttgactggcggcgcgaccttgaggcctgcgttcgcctcagttgccccctctgtgcaatggggagacgcctgccgccccgccccgagtcccatgccgcagccaccgcgacggagcccgcaggcgggaacctgcctccgcgcgttagcgcgcacgcgcgcctcatgtgtcgtccccatcagcgccggcttccgtctataggccagatgcactgtcactctggcgaagtcgcagacccgattggccgggacggaggcgcgagaccgggttgcgggcggggccgaacgtggtataaaaggggcgggaggccaggctcgtgtcagtctgcagacgccacccgagccgagtggtcgttactattagccgccgggctggtagcccagcggggggcgcggcagggcgaggagcagcccgcgctgctcccgcccctcgggtgccagcaccgcccctgctgcggcgggtgaggggcggggcggggcgcggcgtatataaggctaggggcgggcgccgctcttcagtccgcctggagacctcgagccgagtggtcgtcaggatctcgggctcggaacgagactgcacggtgagtgcggcgccggggcggggggcccacccagggtgtggtcggatccggtgcaccgggcgggcgcgccgcaaccgcgacaggcgcccttctcggaccggacgcaggggccggcgaccacgccctgggaccgagaagaggggtgcgggacgcgcccagatcctcggccttggggctgctcggcacgccttggcgcgagtgccacgtcgagaggcgtcggcggggagcgcggaaggggacgctggcccccaggcccaggtcaagcgccttggtttgcccactaggattgttttaagaaaaggatccgccaccatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgatgaggtaccttctccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaagctagccctaggaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcctgcagggctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 31 - DNA, 2752 bp, synthetic construct
  source 1..2752: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagcccccgttctgcttcactctccccatctcccccccctccccacccccaattttgtatttatttattttttaattattttgtgcagcgatgggggcgggggggggggggggggggggggggggtggggcggagggtggcaaggggcggggcggggcaaggcggaaaggtgcggcggcagccaatcaaagcggcgccctccaaaagtttccttttatggcaaggcggcggcggcggcggccctataaaaagcgaagcgcgcggcgggcgggagtcgctgcgttgccttgggtcctccccccctctccagccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagtggagctcgcgactagtcgattcgaattcggcttgtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatatccgtatgatgttcctgattattgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 32 - DNA, 2728 bp, synthetic construct
  source 1..2728: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagcccccgttctgcttcactctccccatctcccccccctccccacccccaattttgtatttatttattttttaattattttgtgcagcgatgggggcgggggggggggggggggggggggggggtggggcggagggtggcaaggggcggggcggggcaaggcggaaaggtgcggcggcagccaatcaaagcggcgccctccaaaagtttccttttatggcaaggcggcggcggcggcggccctataaaaagcgaagcgcgcggcgggcgggagtcgctgcgttgccttgggtcctccccccctctccagccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagtggagctcgcgactagtcgattcgaattcggcttgtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 33 - DNA, 2728 bp, synthetic construct
  source 1..2728: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagcccccgttctgcttcactctccccatctcccccccctccccacccccaattttgtatttatttattttttaattattttgtgcagcgatgggggcgggggggggggggggggggggggggggtggggcggagggtggcaaggggcggggcggggcaaggcggaaaggtgcggcggcagccaatcaaagcggcgccctccaaaagtttccttttatggcaaggcggcggcggcggcggccctataaaaagcgaagcgcgcggcgggcgggagtcgctgcgttgccttgggtcctccccccctctccagccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagtggagctcgcgactagtcgattcgaattcggcttgtgatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 34 - DNA, 2829 bp, synthetic construct
  source 1..2829: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagcccccgttctgcttcactctccccatctcccccccctccccacccccaattttgtatttatttattttttaattattttgtgcagcgatgggggcgggggggggggggggggggggggggggtggggcggagggtggcaaggggcggggcggggcaaggcggaaaggtgcggcggcagccaatcaaagcggcgccctccaaaagtttccttttatggcaaggcggcggcggcggcggccctataaaaagcgaagcgcgcggcgggcgggagtcgctgcgttgccttgggtcctccccccctctccagccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccactcgagtggagctcgcgactagtcgattcgaattcggcttgtgatgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatgagaattcgatatcaagcttattccataaagtaggaaacactacatcatccataaagtaggaaacactacatcatccataaagtaggaaacactacatcatccataaagtaggaaacactacacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 35 - DNA, 2806 bp, synthetic construct
  source 1..2806: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 36 - DNA, 2476 bp, synthetic construct
  source 1..2476: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccaccatgaccatccttttccttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 37 - DNA, 2869 bp, synthetic construct
  source 1..2869: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccaccatgcggagaatgcagctgctgctgctcattgccctgtctctggccctggtcaccaatagccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagaggaggcggaggatctggcggaggcggatctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 38 - DNA, 2818 bp, synthetic construct
  source 1..2818: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccaactcctaaaaaaccgccaccatgggagtgaaagttctttttgcccttatttgtattgctgtggccgaggccgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 39 - DNA, 2827 bp, synthetic construct
  source 1..2827: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccaactcctaaaaaaccgccaccatgcggaggatgcagctgctcttgctcattgccctaagtctcgcacttgtcacaaacagtgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 40 - DNA, 2827 bp, synthetic construct
  source 1..2827: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccaactcctaaaaaaccgccaccatgagacgactctggtggctgttgcttctgctcttgcttctgtggcccatggtctgggccgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 41 - DNA, 2827 bp, synthetic construct
  source 1..2827: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccaactcctaaaaaaccgccaccatgagacgactctggtggctgttgcttctgctcttgcttctgtggccctgtatgaaggccgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 42 - DNA, 3031 bp, synthetic construct
  source 1..3031: mol_type = other DNA
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcgtagtcgccgtgaacgttctttttcgcaacgggctcgagccgagtggtcgttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatccaactcctaaaaaaccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 43 - DNA, 2551 bp, synthetic construct
  source 1..2551: mol_type = other DNA
cgagtcttcaaaccttaagcctcagtgcaaaagggagcagcgaggagaggcggtgggattaaagaaaaaaattcttctctccctgtcacttcaaaaggtcgcccaaccagcgagcggggggtttacccacgactctggctcgcaccccttgcgggcctgcgcaggcggccccgtagcgcaagggagggcgggaaaggaaggggcggggacgcgagggcgaatctataagaagcgtcgttcagcgagttcgctgctcagaagcgccgagagcgcggccgggacggttggagaagaaggcggctcccggaagggggagagacaaactgccgtaacctctgccgttcaggaacgtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgcggatccaactcctaaaaaaccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 44 - DNA, 2821 bp, synthetic construct
  source 1..2821: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccaactcctaaaaaaccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 45 - DNA, 2635 bp, synthetic construct
  source 1..2635: mol_type = other DNA
ttacatcaagtgccaagctggcctaacttcagtctccacccactcagtgtggggaaactccatcgcataaaacccctccccccaacctaaagacgacgtactccaaaagctcgagaactaatcgaggtgcctggacggcgcccggtactccgtggagtcacatgaagcgacggctgaggacggaaaggcccttttcctttgtgtgggtgactcacccgcccgctctcccgagcgccgcgtcctccattttgagctccctgcagcagggccgggaagcggccatctttccgctcacgcaactggtgccgaccgggccagccttgccgcccagggcggggcgatacacaattggtttgatctgattataactagtggagaagagcatgcttgagggctgagtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggctcgagccgagtggtcgttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacaaacagtagttgacggatccaactcctaaaaaaccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 46 - DNA, 3619 bp, synthetic construct
  source 1..3619: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagacgcaaaaaacgcagcgtgggcagcggcgcgccggtgaaacagaccctgaactttgatctgctgaaactggcgggcgatgtggaaagcaacccgggcccgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 47 - DNA, 3604 bp, synthetic construct
  source 1..3604: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagacgcaaaaaacgcagcgtgggcagcggcgcgaccaactttagcctgctgaaacaggcgggcgatgtggaagaaaacccgggcccgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 48 - DNA, 3601 bp, synthetic construct
  source 1..3601: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagacgcaaaaaacgcagcgtgggcagcggcgaaggccgcggcagcctgctgacctgcggcgatgtggaagaaaacccgggcccgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 49 - DNA, 3658 bp, synthetic construct
  source 1..3658: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgctcgcaaaaaacgcagcgtgggcagcggcgcgaccaactttagcctgctgaaacaggcgggcgatgtggaagaaaacccgggcccgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 50 - DNA, 3775 bp, synthetic construct
  source 1..3775: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacctcgagccgagtggtcgtgcctccatagaagacaccgggaccgaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagacaaaccgtcggatccgccaccatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgatacgcaaaaaacgcagcgtgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatagacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttcaatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 51 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 52 - DNA, 744 bp, synthetic construct
  source 1..744: mol_type = other DNA
atgaccatcctgttcctgaccatggtcatttcctactttggctgcatgaaggctgcccccatgaaagaagccaacatccgcggccagggcggcctggcctaccctggcgtgcgcacccatggcaccctggagtccgtgaatggccccaaggccggctcccgcggcctgacctccctggctgacaccttcgaacatgtgatcgaagagctgctggatgaggaccagaaagtgcgccccaatgaagaaaacaataaggacgccgacctgtacacctcccgcgtgatgctgtcctcccaggtgcctctggagcctcctctgctgtttctgctggaggaatacaaaaattacctggatgctgctaacatgtccatgcgcgtccgccgccactccgaccctgcccgccgcggcgagctgtccgtgtgcgactccatttccgagtgggtgaccgctgccgacaaaaagaccgccgtggacatgtccggcggcaccgtcaccgtcctggaaaaggtccctgtctccaaaggccagctgaagcagtacttctatgagaccaagtgcaatcccatgggctacaccaaagaaggctgccgcggcattgacaaacggcattggaactcccagtgccgcaccacccagtcctacgtgcgcgccctgaccatggattccaaaaagcgcattggctggcgcttcatccgcattgacacctcctgcgtctgtaccctgaccatcaaacgcggccgctga

SEQ ID NO: 53 - DNA, 744 bp, synthetic construct
  source 1..744: mol_type = other DNA
atgaccatcctgttcctgactatggtgattagctactttggatgcatgaaggctgcccctatgaaagaagctaacatcagaggacaaggaggcctggcctacccaggagtgagaacccatggaactctggagtctgtgaatggcccaaaggctggaagcagaggcctgacaagcctggctgacacttttgaacatgtgattgaagagctgctggatgaggaccagaaagtgagacctaatgaagaaaacaataaggatgctgacctgtacaccagcagagtgatgctgagcagccaagtgcctctggagcctcctctgctgtttctgctggaggaatacaaaaattacctggatgctgctaacatgagcatgagagtcagaagacactctgaccctgccagaagaggagagctgtctgtgtgtgacagcatttctgagtgggtgacagctgctgacaaaaagactgctgtggacatgtctggaggaactgtcacagtcctggaaaaggtccctgtgagcaaaggccaactgaagcaatacttctatgagaccaagtgcaatccaatgggctacacaaaagaaggctgcagaggcattgacaaaagacattggaacagccagtgcagaactacccagagctatgtgagagccctgaccatggatagcaaaaagagaattggctggagattcattagaattgacactagctgtgtctgtacactgaccattaaaagaggaagatga

SEQ ID NO: 54 - DNA, 744 bp, synthetic construct
  source 1..744: mol_type = other DNA
atgaccatcctgtttctgacaatggttatttcatattttggttgcatgaaagcagctccaatgaaggaagcaaacatcagaggacaagggggcctagcttatcctggagttagaacccatgggactttggagtctgtcaatggcccaaaagctggaagcaggggcctgacaagcctggcagatacttttgaacatgtcattgaggaactcctagatgaagaccaaaaagttagaccaaatgaagagaacaataaagatgctgacctttatactagcagagtcatgctctcctctcaagtcccactagaacccccactcctgttcctactggaggaatacaaaaattaccttgatgcagccaatatgagtatgagagtgaggagacacagtgaccctgctaggagaggggaactctcagtatgtgactccattagtgaatgggtgactgctgcagacaaaaagacagcagttgacatgagtggtggcacagtaactgtactagaaaaagtaccagtgagtaaaggtcagcttaagcaatatttttatgagacaaaatgcaatccaatgggatataccaaggaaggatgtaggggcattgataaaaggcactggaatagccaatgtagaaccacacaatcttatgttagggcccttaccatggactccaagaaaagaattggatggagatttatcaggattgatacaagttgtgtctgcaccctgaccatcaaaagaggaagatga

SEQ ID NO: 55 - DNA, 744 bp, synthetic construct
  source 1..744: mol_type = other DNA
atgaccattctgtttctgaccatggttattagctatttcgggtgcatgaaagccgccccgatgaaagaagcgaatatccggggccagggcggcctggcgtatccgggcgtccggacccacgggaccctggaatccgtgaacggcccgaaagccgggtcccggggcctgaccagcctggcggatacctttgaacatgtgattgaagaactgctggatgaagatcagaaagtccgtccgaatgaagaaaataacaaagatgcggatctgtatacctcccgggttatgctgagcagccaggttccgctggaaccgccgctgctgtttctgctggaagaatataagaattatctggacgccgccaatatgagcatgcgggtccgccgccattccgatccggcgcgccgcggcgaactgtccgtgtgcgatagcatttccgaatgggtgaccgccgccgataagaaaaccgcggtggatatgtccggcggcaccgtgaccgtgctggagaaagttccggtgagcaaagggcagctgaaacagtatttctatgaaaccaaatgcaatccgatgggctataccaaagagggctgccggggcattgataaacggcattggaatagccagtgccggaccacccagagctatgtccgggccctaaccatggatagcaagaaacggatcgggtggcgctttatccggattgataccagctgcgtgtgcaccctaaccatcaaacggggccggtaa

SEQ ID NO: 56 - DNA, 771 bp, synthetic construct
  source 1..771: mol_type = other DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttag

SEQ ID NO: 57 - DNA, 744 bp, Homo sapiens
  source 1..744: mol_type = unassigned DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 58 - DNA, 250 bp, synthetic construct
  source 1..250: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggca

SEQ ID NO: 59 - DNA, 248 bp, synthetic construct
  source 1..248: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 60 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 61 - DNA, 598 bp, synthetic construct
  source 1..598: mol_type = other DNA
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgg

SEQ ID NO: 62 - DNA, 156 bp, synthetic construct
  source 1..156: mol_type = other DNA
gctcgctttcttgctgtccaatttctattaaaggttcctttgttccctaagtccaactactaaactgggggatattatgaagggccttgagcatctggattctgcctaataaaagatctttattttcattagatctgtgtgttggttttttgtgtg

SEQ ID NO: 63 - DNA, 201 bp, synthetic construct
  source 1..201: mol_type = other DNA
tccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccata

SEQ ID NO: 64 - DNA, 363 bp, synthetic construct
  source 1..363: mol_type = other DNA
tccataaagtaggaaacactacatcatccataaagtaggaaacactacatcatccataaagtaggaaacactacatcatccataaagtaggaaacactacagaattcgatatcagtgaattctaccagtgccataagtgaattctaccagtgccataagtgaattctaccagtgccataagtgaattctaccagtgccatatcaagcttatacatacttctttacattccaatacatacttctttacattccaatacatacttctttacattccaccatggacttgtacaaacaccattgtcacacactccaacaaacaccattgtcacacactccaacaaacaccattgtcacacactccaa

SEQ ID NO: 65 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 66 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatggcccgggctctatgg

SEQ ID NO: 67 - DNA, 479 bp, synthetic construct
  source 1..479: mol_type = other DNA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 68 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 69 - DNA, 179 bp, synthetic construct
  source 1..179: mol_type = other DNA
gtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaa

SEQ ID NO: 70 - DNA, 441 bp, synthetic construct
  source 1..441: mol_type = other DNA
atgaccatccttttccttactatggttatttcatactttggttgcatgaaggctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttag

SEQ ID NO: 71 - DNA, 834 bp, synthetic construct
  source 1..834: mol_type = other DNA
atgcggagaatgcagctgctgctgctcattgccctgtctctggccctggtcaccaatagccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagaggaggcggaggatctggcggaggcggatctcactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttag

SEQ ID NO: 72 - DNA, 768 bp, synthetic construct
  source 1..768: mol_type = other DNA
atgggagtgaaagttctttttgcccttatttgtattgctgtggccgaggccgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttag

SEQ ID NO: 73 - DNA, 777 bp, synthetic construct
  source 1..777: mol_type = other DNA
atgcggaggatgcagctgctcttgctcattgccctaagtctcgcacttgtcacaaacagtgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttag

SEQ ID NO: 74 - DNA, 777 bp, synthetic construct
  source 1..777: mol_type = other DNA
atgagacgactctggtggctgttgcttctgctcttgcttctgtggcccatggtctgggccgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttag

SEQ ID NO: 75 - DNA, 777 bp, synthetic construct
  source 1..777: mol_type = other DNA
atgagacgactctggtggctgttgcttctgctcttgcttctgtggccctgtatgaaggccgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttag

SEQ ID NO: 76 - DNA, 1584 bp, synthetic construct
  source 1..1584: mol_type = other DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagacgcaaaaaacgcagcgtgggcagcggcgcgccggtgaaacagaccctgaactttgatctgctgaaactggcgggcgatgtggaaagcaacccgggcccgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 77 - DNA, 1569 bp, synthetic construct
  source 1..1569: mol_type = other DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagacgcaaaaaacgcagcgtgggcagcggcgcgaccaactttagcctgctgaaacaggcgggcgatgtggaagaaaacccgggcccgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 78 - DNA, 1566 bp, synthetic construct
  source 1..1566: mol_type = other DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagacgcaaaaaacgcagcgtgggcagcggcgaaggccgcggcagcctgctgacctgcggcgatgtggaagaaaacccgggcccgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatag

SEQ ID NO: 79 - DNA, 1623 bp, synthetic construct
  source 1..1623: mol_type = other DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgctcgcaaaaaacgcagcgtgggcagcggcgcgaccaactttagcctgctgaaacaggcgggcgatgtggaagaaaacccgggcccgatgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaagatacccatacgatgttccagattacgcttag

SEQ ID NO: 80 - DNA, 573 bp, synthetic construct
  source 1..573: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgata

SEQ ID NO: 81 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 82 - DNA, 154 bp, synthetic construct
  source 1..154: mol_type = other DNA
atacatacttctttacattccaatacatacttctttacattccaatacatacttctttacattccaccatggacttgtacaaacaccattgtcacacactccaacaaacaccattgtcacacactccaacaaacaccattgtcacacactccaa